SEQUENCE LISTING (2 sequences)

SEQ ID NO: 1 - protein, 30 aa, Homo sapiens
HAEGTFTSDVSSYLEGQAAKEFIAWLVKGR

SEQ ID NO: 2 - protein, 33 aa, Homo sapiens
HADGSFSDEMNTILDNLAARDFINWLIQTKITD

(19) United States
(12) Patent Application Publication
(10) Pub. No.: US 2018/0000768 A1
(43) Pub. Date: Jan. 4, 2018

(54) INTESTINAL FXR AGONISM ENHANCES GLP-1 SIGNALING TO RESTORE PANCREATIC BETA CELL FUNCTIONS

(71) Applicant: Salk Institute for Biological Studies, La Jolla, CA (US)

(72) Inventors: Sungsoon Fang, La Jolla, CA (US); Eiji Yoshihara, San Diego, CA (US); Ruth T. Yu, La Jolla, CA (US); Annette Atkins, San Diego, CA (US); Michael Downes, San Diego, CA (US); Ronald M. Evans, La Jolla, CA (US)

(73) Assignee: Salk Institute for Biological Studies, La Jolla, CA (US)

(21) Appl. No.: 15/697,271

(22) Filed: Sep. 6, 2017

Related U.S. Application Data
(63) Continuation of application No. PCT/US2016/022082, filed on Mar. 11, 2016.
(60) Provisional application No. 62/133,042, filed on Mar. 13, 2015.

(51) Int. Cl. A61K 31/216; A61K 45/06

(52) U.S. Cl. CPC A61K 31/216; A61K 45/06

(57) ABSTRACT

Disclosed are embodiments of a method of treating or preventing latent autoimmune diabetes of adults (LADA) in a subject. Such embodiments include administering to a subject (e.g., via the gastrointestinal tract) a therapeutically effective amount of one or farnesoid X receptor (FXR) agonist compounds, thereby activating FXR receptors in the intestines, and treating or preventing latent autoimmune diabetes of adults (LADA) in the subject.

### CROSS REFERENCE TO RELATED APPLICATIONS

This application is a continuation of International Application No. PCT/US2016/022082, filed on Mar. 11, 2016, which was published in English under PCT Article 21(2), which in turn which claims priority to U.S. Provisional Application No. 62/133,042, filed on Mar. 13, 2015, both herein incorporated by reference.

### ACKNOWLEDGMENT OF GOVERNMENT SUPPORT

This invention was made with government support under Grant No. R24-DK090962 awarded by the National Institutes of Health (NIH). The government has certain rights in the invention.

### FIELD

This disclosure concerns methods for using farnesoid X receptor (FXR) agonists to treat or prevent latent autoimmune diabetes of adults (LADA).

### BACKGROUND

Type II diabetes (T2D) is characterized by progressive failure of pancreatic β cell function and the glucagon-like peptide-1 (GLP1) signaling targeted drugs such as GLP1 analogues and dipeptidyl peptidase-4 (DPP-4) inhibitors are widely used for treatment of T2D. Evidence has shown that lipids, especially saturated fatty acids, accumulate in 3 cells to induce 3 cell dysfunction and lipotoxic apoptosis. GLPs and glucagon are hormones encoded by the same gene, proglucagon, each having different physiological activities. Due to alternative processing by prohormone convertases, glucagon is predominantly produced in endocrine pancreatic α cells whereas GLPs are predominantly produced in the intestine. GLP-1 is primarily produced in L cells during the postprandial state to promote insulin secretion in pancreatic β cells to reduce blood glucose levels. Administration of a GLP-1 antagonist leads to increased blood glucose levels in humans, whereas disruption of the GLP-1 receptor gene results in glucose intolerance in a rodent model. Treatment of diabetic patients with GLP-1 or GLP-1 analogues increases meal-stimulated insulin secretion and suppresses postprandial hyperglycemia without causing hypoglycemia. Thus, approaches to increase endogenous GLP-1 levels through modulation of GLP-1 secretion from enteroendocrine L cells would be a potential therapeutic intervention to treat or prevent LADA.

Fexaramine (Fex) is a non-systemic FXR agonist that mimics food activation of intestinal FXR, resulting in intestinally-restricted FXR activation. Fex or FXR agonist treatment produces a novel means to treat or prevent LADA.

### SUMMARY

It is shown herein that intestinally-restricted FXR activation enhances GLP-1 secretion of enteroendocrine L cells, which contributes to the improvement of glucose tolerance in diabetic rodent models. The data demonstrate that Fex increases mitochondrial activities to enhance GLP-1 secretion in L cells, leading to improved pancreatic β cell physiology. Concomitantly, Fex also increases expression of GLP-1 receptor (GLP-1R) in pancreatic β cells to enhance glucose-stimulated insulin secretion. Furthermore, a structurally modified Fex analog, Fex-D remarkably improved metabolic parameters with relatively low dosage. The beneficial efficacy achieved with Fex and its analog, Fex-D, indicates that intestinal FXR activation is a safer approach in the treatment or prevention of latent autoimmune diabetes of adults (LADA). In some embodiments, intestinal FXR activation is used in the treatment or prevention of latent autoimmune diabetes of adults (LADA). In some embodiments, intestinal FXR activation is accomplished by administering at least one FXR agonist to the adult with LADA. In some embodiments, the FXR agonist is a FXR agonist as described herein. In some embodiments, the FXR agonist is administered to an adult with LADA at a dose that results in a systemic level of the FXR agonist below the EC₅₀ for the FXR agonist (e.g., such that there is minimal activation outside the intestines). In one example, the FXR agonist is administered to an adult with LADA at a dose that results in less than 30 nM of the FXR agonist in the serum 1 hour following the administration.

In some cases, patients with LADA, sometimes referred to as type 1.5 diabetes, exhibit typical noninsulin-dependent diabetes mellitus symptoms (NIDDM, e.g., hyperglycemia in the context of insulin resistance and relative lack of insulin), but patients also have some of the immunological and clinical features of insulin-dependent diabetes mellitus (IDDM, e.g., no insulin due to breakdown of islet cells in the pancreas). Thus, changes in lifestyle (e.g., maintaining a healthy weight, exercising, eating sensibly) are not effective for such patients. The diagnosis of LADA can be based on a high blood sugar in combination with the clinical impression that islet failure rather than insulin resistance is the main cause. In some examples, such patients may have a low C-peptide levels and antibodies against the islets of Langerhans, islet cell antibodies (ICA), glutamic acid decarboxylase autoantibodies (GADA), insulinoma-associated (IA-2) autoantibodies, and/or zinc transporter autoantibodies (ZnT8).

Provided herein are methods for treating or preventing LADA in a subject, such as a mammal. In some examples, such subjects have a fasting blood glucose level of 126 mg/dl or more (e.g., are hyperglycemic), are not overweight (e.g., normal weight or have a body mass index of 18.5 to 25 or 16 to 18.5) or are overweight or are obese (e.g., have a body mass index of at least 25, at least 30, at least 35 or at least 40, such as 29-29, 30-34, or 35-40, or greater than 40), are insulin-resistant or produce no insulin, have persistent islet cell antibodies, have high frequency of thyroid and gastric autoimmunity, have DR3 and DR4 human leukocyte antigen haplotypes, show progressive loss of beta cells, adult disease onset, defective glycaemic control, without tendency to ketoacidosis, have low levels of C-peptide, or combinations thereof. In some examples, such methods include administering to the subject a therapeutically effective amount of one or more farnesoid X receptor (FXR) agonists (such as, but not limited to, deuterated Fex or a Fex analog) (such as 1, 2, 3, 4, or 5 of such compounds). Fex and its analogs are substantially absorbed in the gastrointestinal tract, thereby activating FXR receptors in the intestines to treat or prevent LADA in the subject. In some examples, absorption of the compounds is substantially limited to the intestines. In other examples, the FXR agonist substantially enhances FXR target gene expression in the intestines while not substantially enhancing FXR target gene expression in the liver or kidney.

In some embodiments, administration of one or more farnesoid X receptor (FXR) agonists results in no substantial change in food intake and/or fat consumption in the subject, and/or no substantial change in appetite in the subject. In some examples, administering one or more FXR agonists can also restore pancreatic beta cell function, increase glucose stimulated insulin secretion (GSIS) without significantly changing body weight, increase GLP-1 secretion in enteroendocrine L cells, increase expression of GLP-1R in pancreatic beta cells, or combinations thereof, for example as compared to no administration of one or more FXR agonists. Thus in some embodiments, the methods improve glucose homeostasis in the subject.

In some embodiments, the method further includes administering to the subject an insulin sensitizing drug, an insulin secretagogue, an alpha-glucosidase inhibitor, an amylin agonist, a dipeptidyl-peptidase 4 (DPP-4) inhibitor, a glucagon-like peptide (GLP) agonist, meglitinide, sulfonylurea, a peroxisome proliferator-activated receptor (PPAR)-gamma agonist, or combinations thereof.

Exemplary Farnesoid X receptor (FXR) agonist compounds, and compositions comprising such compounds, that target intestinal FXR that can be used in the disclosed method embodiments are provided. Certain exemplary compounds have the following general formula

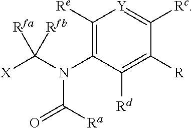

With reference to this general formula, R is selected from

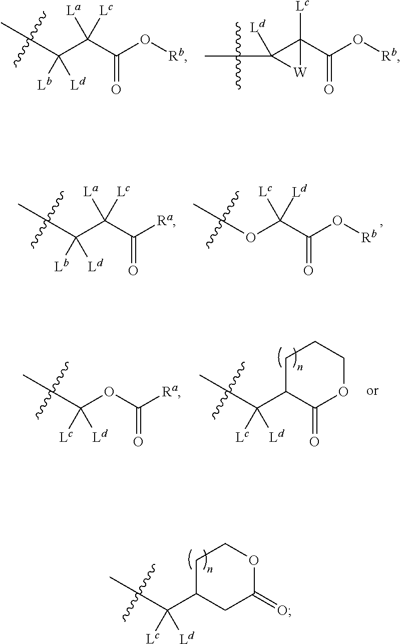

R^{a} is selected from aryl, heteroaryl, alkyl, alkenyl, cycloalkyl, heterocyclic, or polycyclic; R^{b} is selected from hydrogen, alkyl, alkenyl, or cycloalkyl; Y is CR^{g}, N or N—O (N-oxide); R^{c}, R^{d}, R^{e} and R^{g} are each independently selected from hydrogen, deuterium, halide, alkyl, alkenyl, alkoxy, alkylthio, amino, sulfonyl, aminosulfonyl, aminocarbonyl, acyl, hydroxyl or nitro; R^{fa} and R^{fb} are each independently selected from hydrogen, deuterium, halide or alkyl; L^{a} and L^{b} are each independently selected from hydrogen, deuterium, alkyl or cycloalkyl, or together form a pi-bond; L^{c} and L^{d} are each independently selected from hydrogen, deuterium, alkyl or cycloalkyl; W is selected from O or —(C(L^{c})(L^{d}))ₛ-; s is 1, 2, 3, 4, 5 or 6; n is 0 or 1; and X is aryl, heterocyclic or heteroaryl.

Also with reference to this general formula, any or all of the following provisos may apply:

if W is CH₂ and L^{c} and L^{d} are both H, then X is not a benzopyran;

### if R is

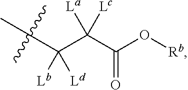

L^{c} and L^{d} are both H, and L^{a} and L^{b} are both H or together form a pi-bond, then X is not a benzopyran;
X is not substituted with —R^{x}-L^{x}-R^{x2}, where

R^{x} is selected from O, NR^{x3}, sulfonyl or S;

R^{x3} is selected from H, alkyl, alkenyl, alkynyl, cycloalkyl, cycloalkenyl, or aryl;

L^{x} is selected from a bond, alkylene, alkenylene, alkynylene, cycloalkyl, cycloalkenyl, heterocyclic, aryl, heteroaryl or CR^{x4}R^{x5};

R^{x4} and R^{x5} are each independently selected from H, D, halogen, alkyl, alkenyl, alkynyl, cycloalkyl, cycloalkenyl, —C(O)OR^{x6}, or —C(O)NR^{x6}R^{x7};

R^{x6} and R^{x7} are each independently selected from H, alkyl, alkenyl, alkynyl, cycloalkyl or cycloalkenyl;

R^{x2} is selected from —C(O)L^{x2}R^{x8} or a carboxyl bioisostere;

L^{x2} is a bond or NR^{x3};

R^{x8} is H, alkyl, alkenyl, alkynyl, cycloalkyl, cycloalkenyl, —OR^{x9}, N(R^{x9})₂, —C(O)R^{x9}, —S(O)₂R^{x9}, —C(O)OR^{x9}, —S(O)₂N(R^{x9})₂ or —C(O)N(R^{x9})₂; and

each R^{x9} is independently selected from H, alkyl, alkenyl, alkynyl, cycloalkyl or cycloalkenyl; and

### if R is

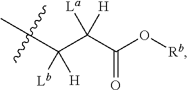

Y is CH, R^{c}, R^{d}, R^{e} and R^{fa} are all hydrogen, and L^{a} and L^{b} are both H or together form a pi-bond, then

if R^{a} is cyclohexyl, R^{b} is methyl, and R^{fb} is H then X is not phenyl, 4-biphenyl, 4-bromophenyl, 3-bromophenyl, 2-bromophenyl, 4-tert-butylphenyl, 3-methoxyphenyl, 3,5-dimethoxyphenyl, 3-(trifluoromethyl)phenyl, 4-(3,4-difluorophenyl)phenyl, 4-(3-acetylphenyl)phenyl, 4-(4-methylthiophenyl)phenyl, 4-(4-methoxyphenyl)phenyl, 4-(3-methoxyphenyl)phenyl, 4-(2-methoxyphenyl)phenyl, 4-(3,5-dichlorophenyl)phenyl, 4-(4-tert-butylphenyl)phenyl, 4-(3-ethoxyphenyl)phenyl, 4-(3-chlorophenyl)phenyl, 4-(3-methylphenyl)phenyl, 4-(4-methylphenyl)phenyl, 4-(2-methoxy-5-chlorophenyl)phenyl, 4-(3-chloro-4-fluorophenyl)phenyl, 4-(4-trifluoromethoxyphenyl)phenyl, 4-(3-trifluoromethoxyphenyl)phenyl, 4-(2,6-dimethoxyphenyl)phenyl, 4-(4-dimethylaminophenyl)phenyl,

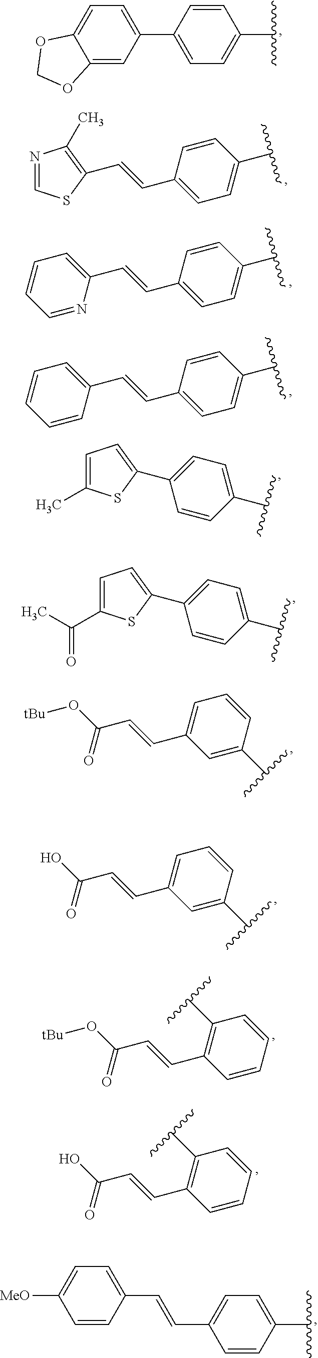

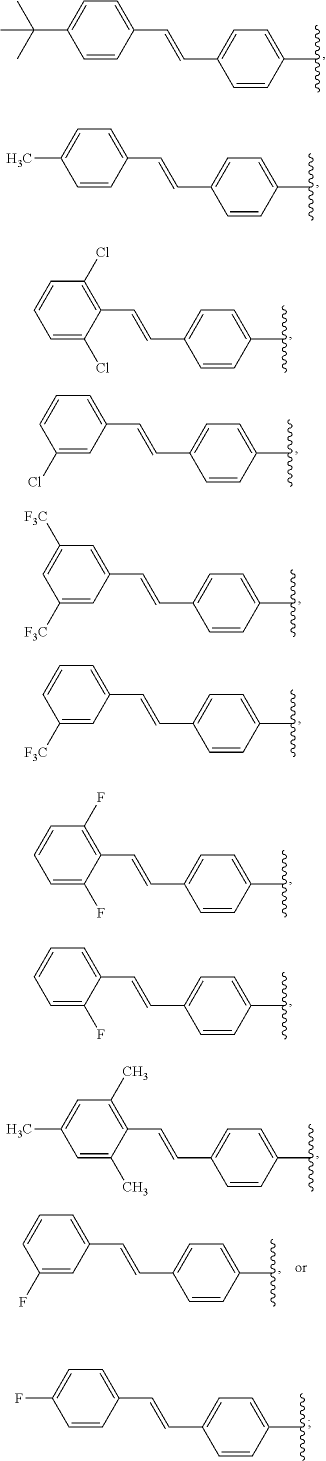

if R^{a} is cyclohexyl, R^{fb} is H and X is

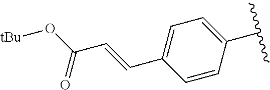 [SMILES: *c1ccc(/C=C/C(=O)OC(C)(C)C)cc1]

then R^{b} is not methyl, ethyl or tert-butyl;

if R^{b} is methyl, R^{fb} is H and X is

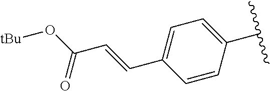 [SMILES: *c1ccc(/C=C/C(=O)OC(C)(C)C)cc1]

then R^{a} is not cyclopropyl, cyclobutyl, cyclopentyl, or cyclohexyl;

if R^{a} is cyclohexyl, R^{fb} is H and X is

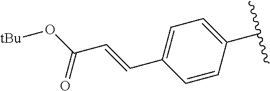 [SMILES: *c1ccc(/C=C/C(=O)OC(C)(C)C)cc1]

then R^{b} is not methyl or tert-butyl;

if R^{a} is cyclohexyl, R^{b} is methyl, R^{fb} is H and X is

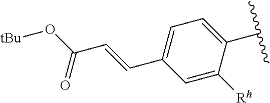

then R^{h} is not hydroxyl, (trimethylsilyl)ethoxymethyl-O, methoxy, O-benzyl, OCH₂CO₂Et, OC(O)CH₃, OC(O)Ph or OSO₂CH₃; and
if R^{a} is cyclohexyl, R^{b} is methyl, R is H and X is

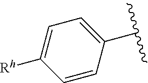

then R^{h} is not —CH═CHC(O)OMe, —CH═CHC(O)OEt, —CH═CHC(O)NMe₂, —CH═CHC(O)NH^{t}Bu, —CH═CHC(O)O^{t}Bu, —CH═CHC(O)OⁱPr, —CH═CHC(O)OCH₂Ph, —CH═CHC(O)OH, —CH═CHCH₂OMe, —CH═CHCH₂OEt or —CH═CHCH₂OPh.

In some embodiments, the compounds L^{c} and L^{d} are both H, and La and L^{b} together form a pi-bond.

Certain other disclosed compounds have the following general formula

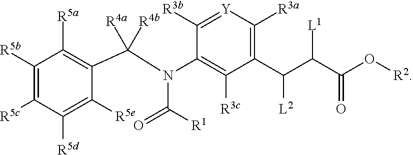

With reference to this general formula, R¹ is selected from aryl, heteroaryl, heterocyclic, alkyl, alkenyl, cycloalkyl, cycloalkenyl or polycyclic; R² is selected from alkyl, alkenyl, or cycloalkyl; Y is selected from N, N—O or CR^{3d}; R^{3a}, R^{3b}, R³ and R^{3d} are each independently selected from hydrogen, deuterium, halide, alkyl, alkenyl, alkoxy, alkylthio, amino, sulfonyl, aminosulfonyl, aminocarbonyl, acyl, hydroxyl or nitro; R^{4a} and R^{4b} are each independently selected from hydrogen, deuterium, halide or alkyl; L¹ and L² are independently selected from hydrogen, deuterium, alkyl, cycloalkyl, or together form a pi-bond; and R^{5a}, R^{5b}, R^{5c}, R^{5d} and R^{5e} are each independently selected from hydrogen, deuterium, halide, alkyl, alkenyl, alkoxy, alkylthio, amino, sulfonyl, aminosulfonyl, aminocarbonyl, acyl, aryl, heteroaryl, cycloalkyl, heterocyclyl, hydroxyl or nitro; or any two adjacent groups selected together form an aryl, heteroaryl, cycloalkyl or heterocyclic ring; and none of R^{5a}, R^{5b}, R^{5c}, R^{5d} or R^{5e} is —R^{x}-L^{x}-R^{x2} where R^{x} is selected from O, NR^{x3}, sulfonyl or S; R^{x3} is selected from H, alkyl, alkenyl, alkynyl, cycloalkyl, cycloalkenyl, or aryl; L^{x} is selected from a bond, alkylene, alkenylene, alkynylene, cycloalkyl, cycloalkenyl, heterocyclic, aryl, heteroaryl or CR^{x4}R^{x5}; R^{x4} and R^{x5} are each independently selected from H, D, halogen, alkyl, alkenyl, alkynyl, cycloalkyl, cycloalkenyl, —C(O)OR^{x6}, or —C(O)NR^{x6}R^{x7}; R^{x6} and R^{x7} are each independently selected from H, alkyl, alkenyl, alkynyl, cycloalkyl or cycloalkenyl; R^{x2} is selected from —C(O)L^{x2}R^{x8} or a carboxyl bioisostere; L^{x2} is a bond or NR^{x3}; R^{x8} is H, alkyl, alkenyl, alkynyl, cycloalkyl, cycloalkenyl, —OR^{x9}, N(R^{x9})₂, —C(O)R^{x9}, —S(O)₂R^{x9}, —C(O)OR⁹, —S(O)₂N(R^{x9})₂ or —C(O)N(R^{x9})₂; and each R^{x9} is independently selected from H, alkyl, alkenyl, alkynyl, cycloalkyl or cycloalkenyl. For certain embodiments where L¹ and L² are both hydrogen or together form a pi-bond, Y is N or C-halogen; or R¹ is polycyclic; or R⁴ is D, or R^{5a} is F, Cl or I; or R^{5d} and R^{5e} together form an aryl, heteroaryl, cycloalkyl or heterocyclic ring; or R^{5b} and R^{5c} together form an aryl, cycloalkyl, nitrogen-containing heterocyclic or nitrogen-containing heteroaryl ring; or any combination thereof.

In some embodiments, Y is C—R^{3d}, and R^{3d} or R^{5a} or both are halogen, and in certain examples the halogen is fluorine. In other embodiments, Y is N.

In certain embodiments, R¹ is polycyclic. Exemplary R¹ polycyclics are selected from

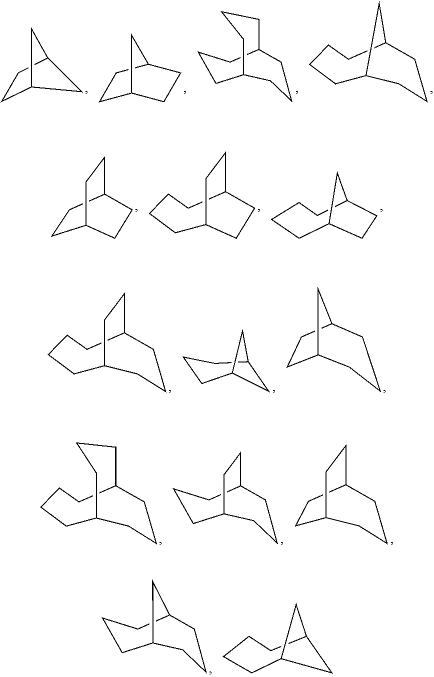

or adamantyl. In other examples, the polycyclic is selected from [2.1.1], [2.2.1], [3.3.3], [4.3.1], [2.2.2], [4.2.2], [4.2.1], [4.3.2], [3.1.1], [3.2.1], [4.3.3], [3.3.2], [3.2.2], [3.3.1], [4.1.1], or adamantyl. In certain working embodiments, the polycyclic is

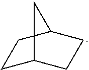

In some embodiments, R^{5c} is a nitrogen-containing heteroaryl ring, and the compound has a formula

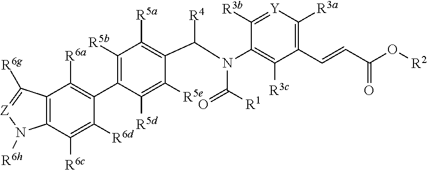

where Z is selected from N, CH, or C-alkyl; R^{6a}, R^{6c}, R^{6d} and R^{6g} each is independently selected from H, D, halogen or alkyl; and R^{6h} is selected from H, D, alkyl, cycloalkyl, aryl or heteroaryl. In some examples, Z is N, and/or R^{6a}, R^{6c}, R^{6d} and R^{6g} are all H. In particular embodiments, R^{6h} is methyl.

In other embodiments, R^{5c} comprises phenyl, leading to compounds having a formula

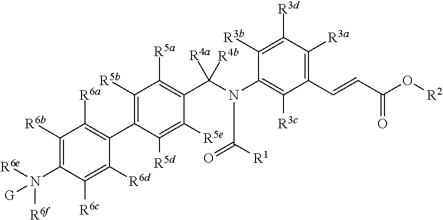

where R^{6a}, R^{6b}, R^{6c} and R^{6d} each is independently selected from H, D, halogen or alkyl; G is a lone pair of electrons, or an oxygen; R^{6e} and R^{6f} each is independently selected from alkyl, H or cycloalkyl; and
where R^{3d} or R^{5a} or both are halogen, or R⁴ is D, or R¹ is polycyclic, or any combination thereof. In some examples, R^{6e} and R^{6f} are both methyl.

In any of the above embodiments, R⁴ may be deuterium, and/or R² may be methyl. In certain embodiments, R¹ is cyclohexyl.

In particular embodiments, the compound is selected from

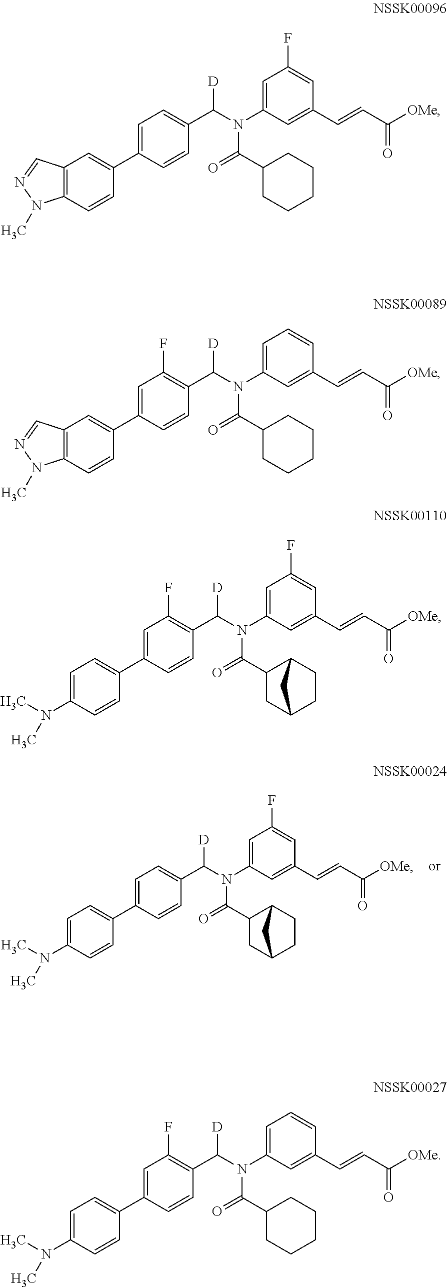 [SMILES: [2H]C(c1ccc(-c2ccc3c(cnn3C)c2)cc1)N(C(=O)C1CCCCC1)c1cc(F)cc(/C=C/C(=O)OC)c1 ; [2H]C(c1ccc(-c2ccc3c(cnn3C)c2)cc1F)N(C(=O)C1CCCCC1)c1cccc(/C=C/C(=O)OC)c1 ; [2H]C(c1ccc(-c2ccc(N(C)C)cc2)cc1F)N(C(=O)C1C[C@H]2CC[C@@H]1C2)c1cc(F)cc(/C=C/C(=O)OC)c1 ; [2H]C(c1ccc(-c2ccc(N(C)C)cc2)cc1)N(C(=O)C1C[C@H]2CC[C@@H]1C2)c1cc(F)cc(/C=C/C(=O)OC)c1 ; [2H]C(c1ccc(-c2ccc(N(C)C)cc2)cc1F)N(C(=O)C1CCCCC1)c1cccc(/C=C/C(=O)OC)c1]

Another disclosed embodiment concerns compounds having a formula

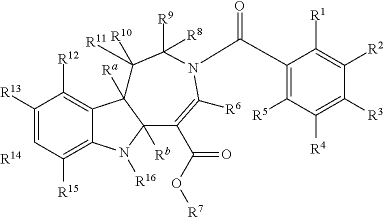

or a pharmaceutically acceptable salt, hydrate, N-oxide or solvate thereof. With reference to this formula: R¹-R¹⁵ independently are selected from hydrogen, deuterium, halogen, CF₃, NO₂, OH, amino, acyl, carboxyl, carboxyl ester, cyano, aminocarbonyl, aminosulfonyl, aliphatic, D-aliphatic, heteroaliphatic or D-heteroaliphatic; R¹⁶ is selected from hydrogen, aliphatic, D-aliphatic, heteroaliphatic or D-heteroaliphatic; R^{a} and R^{b} are independently hydrogen, deuterium, aliphatic or D-aliphatic, or together form a bond, such as a pi-bond; and if R^{a} and R^{b} together form a pi-bond then at least one of R¹-R¹⁶ is or comprises deuterium.

In some embodiments, disclosed compounds have a formula

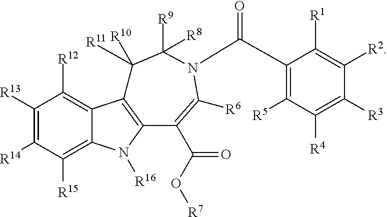

In other embodiments, disclosed compounds have a formula

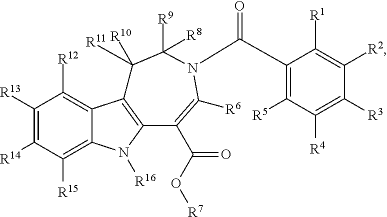

and at least one of R¹-R¹⁶ is or comprises deuterium.

In certain disclosed embodiments, R⁷ is alkyl or deuterated alkyl, such as isopropyl or a deuterated isopropyl group comprising from 1 to 7 deuterium atoms. In certain embodiments, at least one of R¹-R⁵ is a halogen, such as fluoro. For certain embodiments, R¹⁶ is hydrogen. In other disclosed embodiments, R¹⁰ and R¹¹ independently are alkyl or deuterated alkyl, such as methyl or deuterated methyl, wherein the deuterated alkyl group comprises from 1 to n halogen atoms where n is the total number of hydrogen atoms on the substituent, such as from 1 to 3 deuterium atoms for a methyl group. Exemplary compounds having this formula include

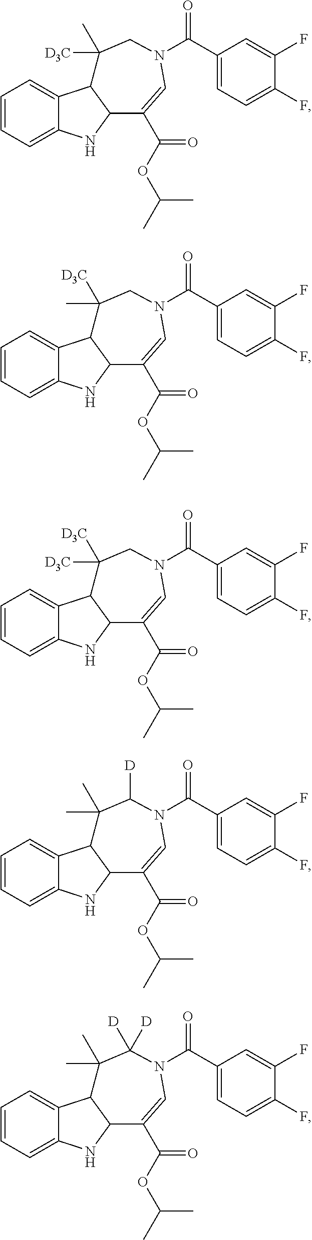

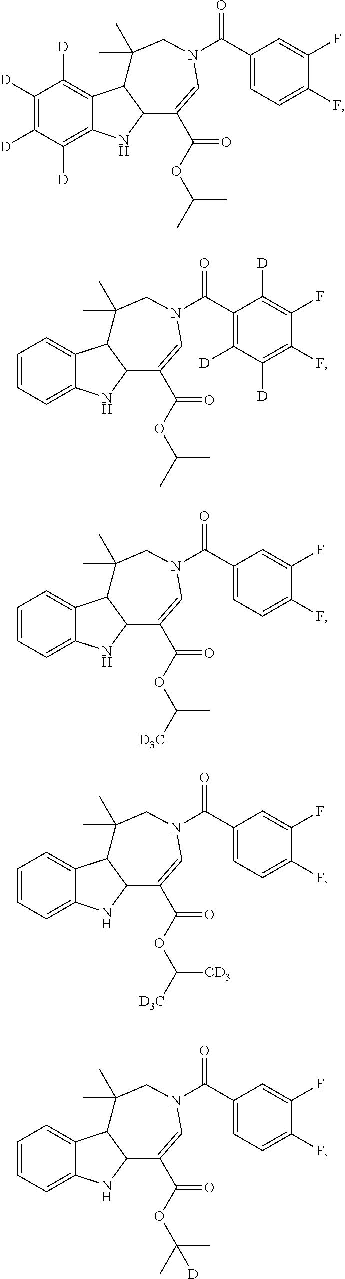

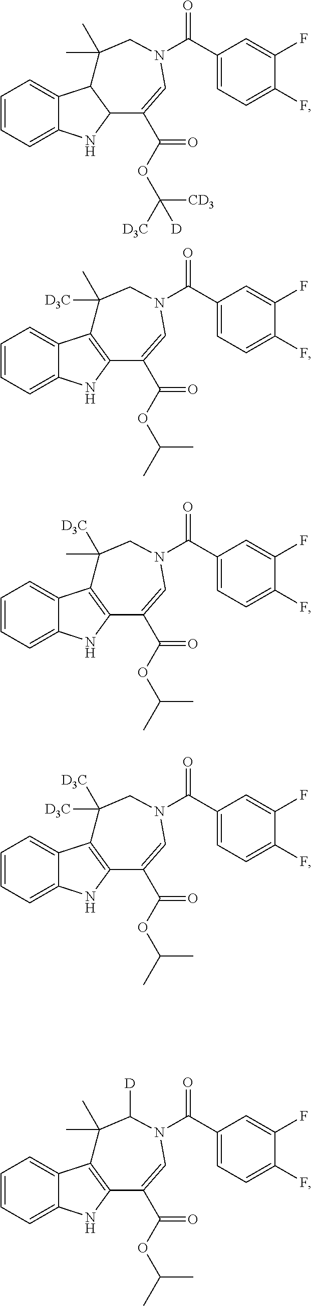

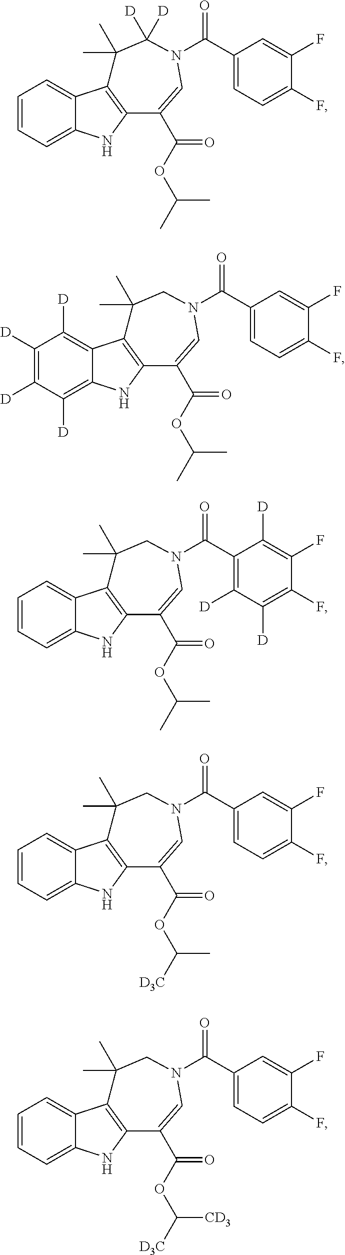

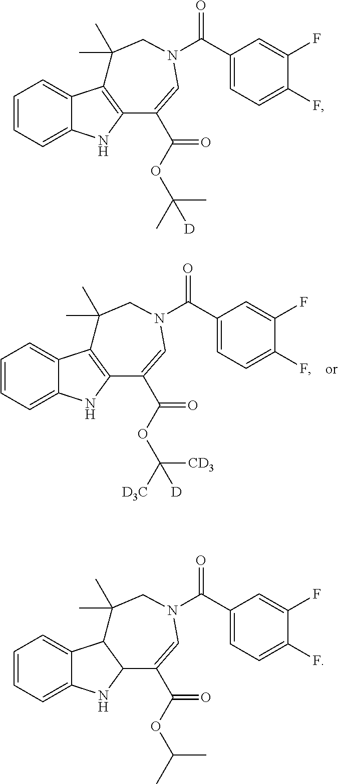

Another embodiment concerns compounds having a formula

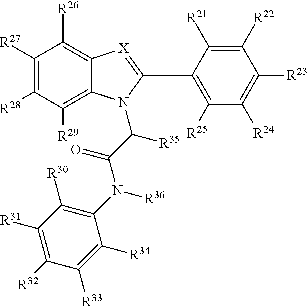

or a pharmaceutically acceptable salt, hydrate, N-oxide, or solvate thereof. With reference to this formula: R²¹-R³⁴ independently are selected from hydrogen, deuterium, halogen, CX₃, where X is a halogen, such as fluorine, with CF₃ being a particular example, NO₂, OH, amino, acyl, carboxyl, carboxyl ester, cyano, aminocarbonyl, aminosulfonyl, aliphatic, D-aliphatic, heteroaliphatic or D-heteroaliphatic; R³⁵ is aliphatic, D-aliphatic, heteroaliphatic or D-heteroaliphatic; R³⁶ is hydrogen, aliphatic, D-aliphatic, heteroaliphatic or D-heteroaliphatic; X is N or CR³⁷; and R³⁷ is hydrogen, deuterium, halogen, CF₃, NO₂, OH, amino, acyl, carboxyl, carboxyl ester, cyano, aminocarbonyl, aminosulfonyl, aliphatic, D-aliphatic, heteroaliphatic or D-heteroaliphatic; where if X is N, then at least one of R²¹-R³⁶ is or comprises deuterium.

In some embodiments, disclosed compounds have a formula

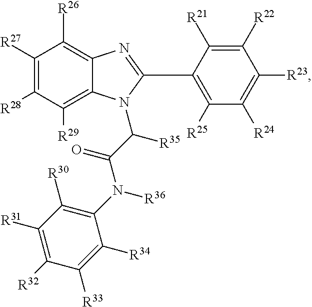

and in other embodiments, disclosed compounds have a formula

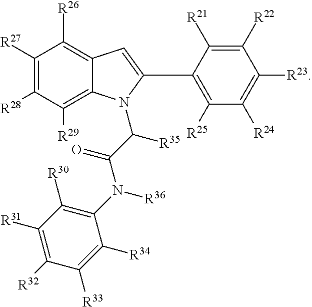

In particular embodiments, R³⁵ is alkyl, cycloalkyl, deuterated alkyl or deuterated cycloalkyl, such as cyclohexyl or deuterated cyclohexyl comprising 1 to 11 deuterium atoms. In particular embodiments, R³⁶ is hydrogen; R³⁴ is CF₃; and R²³ is halogen, such as fluorine or chlorine. Certain compounds are chiral, and all stereoisomers are included in this disclosure. For certain embodiments, the compound is the most biologically active stereoisomer, such as the S-stereoisomer. Exemplary compounds according to this formula include

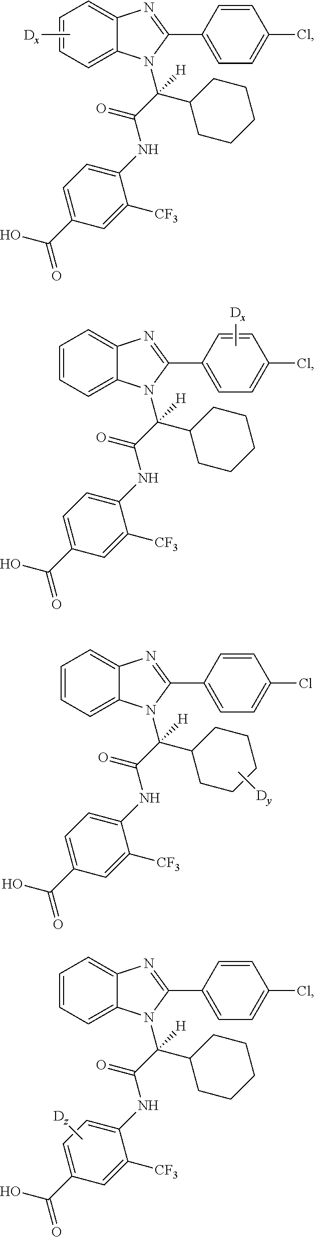

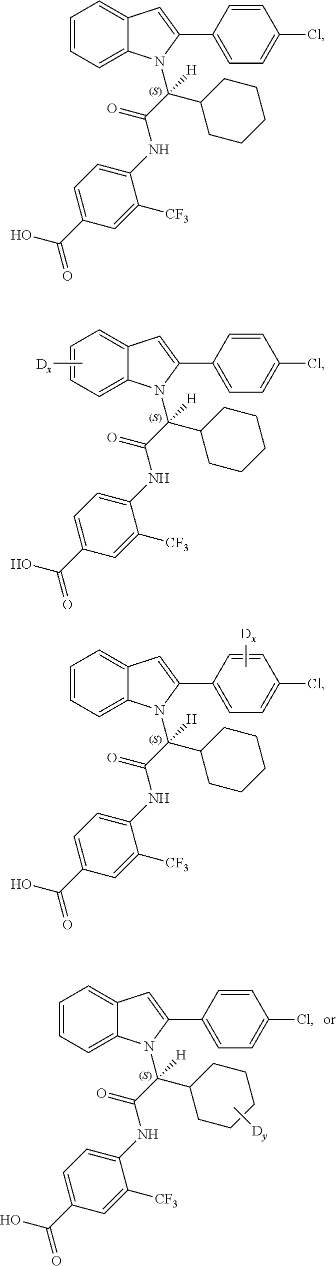

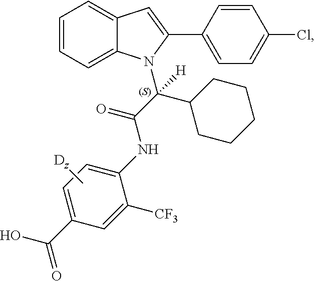

where x is 0 to 4, y is 0 to 11, and z is 0 to 3.

Another disclosed embodiment concerns compounds having a formula

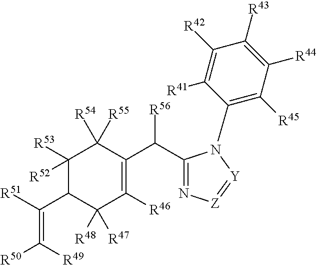

or a pharmaceutically acceptable salt, hydrate, N-oxide or solvate thereof, wherein: R⁴¹-R⁴⁸ and R⁵²-R⁵⁵ independently are selected from hydrogen, deuterium, halogen, CF₃, NO₂, OH, amino, acyl, carboxyl, carboxyl ester, cyano, aminocarbonyl, aminosulfonyl, aliphatic, D-aliphatic, heteroaliphatic or D-heteroaliphatic; R⁴⁹-R⁵¹ independently are selected from hydrogen, deuterium, aliphatic, D-aliphatic, heteroaliphatic or D-heteroaliphatic; R⁵⁶ is amino, cycloamino or substituted cycloamino; Y and Z are independently N or CR⁵⁷; and each R⁵⁷ independently is selected from deuterium, halogen, CF₃, NO₂, OH, amino, acyl, carboxyl, carboxyl ester, cyano, aminocarbonyl, aminosulfonyl, aliphatic, D-aliphatic, heteroaliphatic or D-heteroaliphatic.

In some embodiments, disclosed compounds have a formula selected from

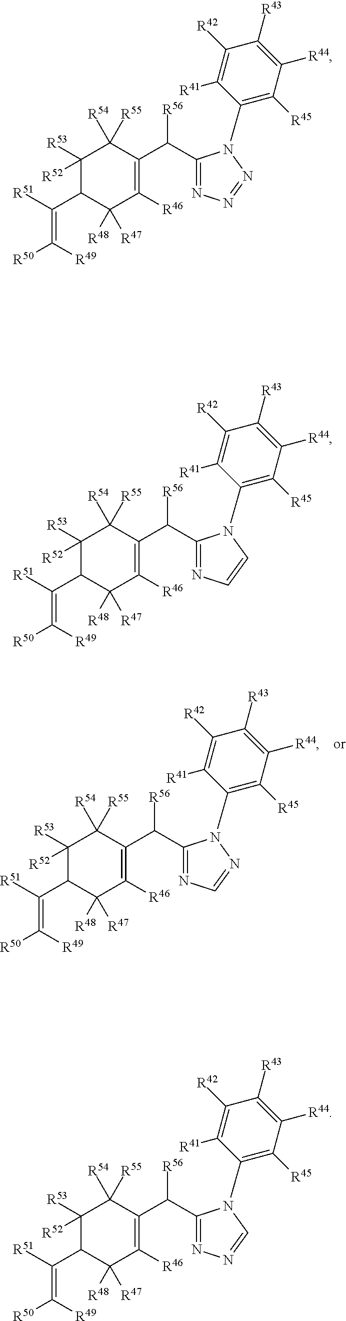

In some embodiments, at least one of R⁴¹-R⁵⁶ is or comprises deuterium. For certain disclosed embodiments, R⁵¹ is aliphatic or D-aliphatic, such as methyl or deuterated methyl having from 1 to 3 deuterium atoms. For certain disclosed embodiments, R⁴⁹ and R⁵⁰ independently are hydrogen or deuterium; and R⁴¹ and R⁴⁵ independently are aliphatic or D-aliphatic, such as methyl or deuterated methyl having from 1 to 3 deuterium atoms. For other embodiments, R⁵⁶ is a cycloamino or substituted cycloamino, such as pyrrolidine, 2-methylpyrrolidine, morpholine, 4-methylpiperazine, piperidine, or azepane. Exemplary compounds having this formula include

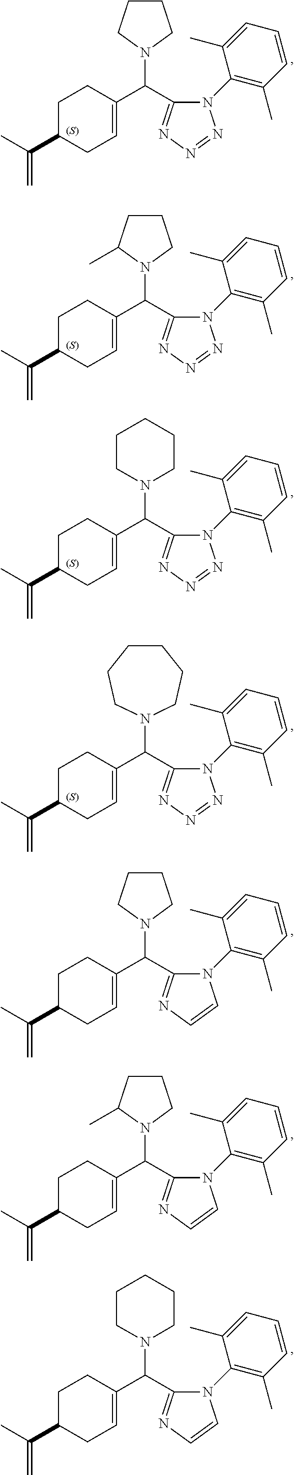

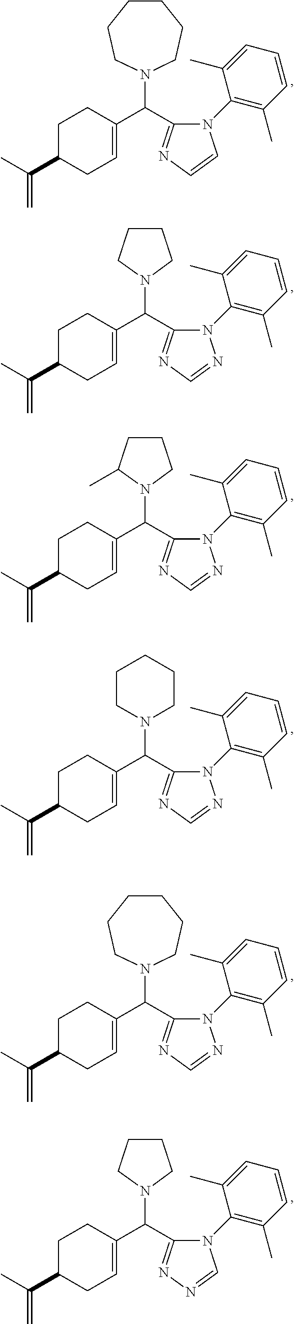

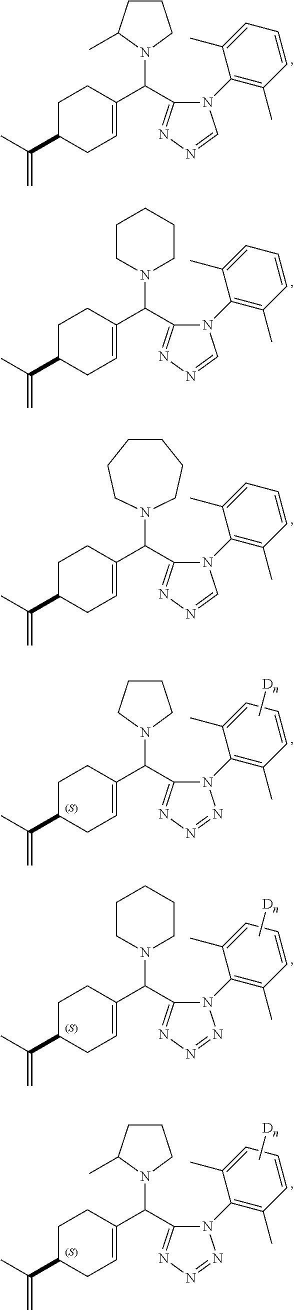

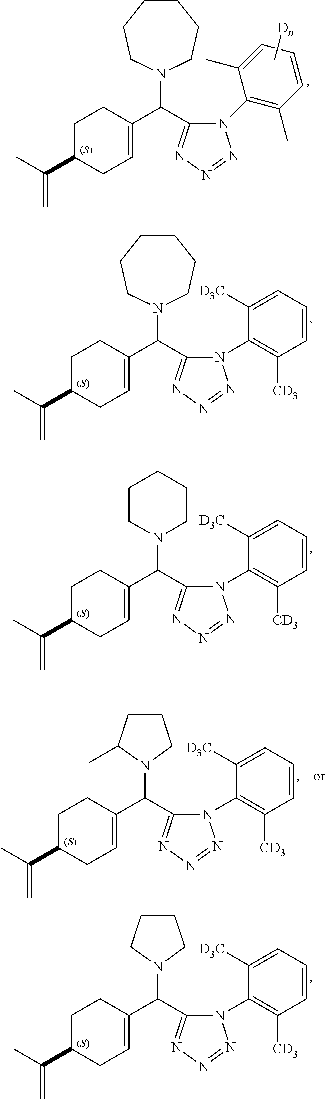

where n is 1 to 3.

Also, in any of the above embodiments, none of R¹-R⁵⁷ is —R-L^{x}-R^{x2}, where Rx is selected from O, NR^{x3}, sulfonyl or S; R^{x3} is selected from H, aliphatic, or aryl; L^{x} is selected from a bond, aliphatic, heteroaliphatic, aryl, heteroaryl or CR^{x4}R^{x5}; R^{x4} and R^{x5} are each independently selected from H, D, halogen, aliphatic, —C(O)OR^{x6}, or —C(O)NR^{x6}R^{x7}; R^{x6} and R^{x7} are each independently selected from H, aliphatic; R^{x2} is selected from —C(O)L^{x2}R^{x8} or a carboxyl bioisostere; L^{x2} is a bond or NR^{x3}; R^{x8} is H, aliphatic, —OR^{x9}, N(R^{x9})₂, —C(O)R^{x9}, —S(O)₂R^{x9}, —C(O)OR^{x9}, —S(O)₂N(R^{x9})₂ or —C(O)N(R^{x9})₂; and each R^{x9} is independently selected from H, aliphatic.

Compositions comprising the disclosed compounds also are provided. In some embodiments, the composition comprises a first disclosed compound, and an additional component, such as a pharmaceutically exceptable excipient, an additional therapeutic compound, or a combination thereof. In certain examples, the additional therapeutic compound is a second disclosed compound. In some embodiments, the composition has an enteric coating.

The foregoing and other objects and features of the disclosure will become more apparent from the following detailed description, which proceeds with reference to the accompanying figures.

### BRIEF DESCRIPTION OF THE DRAWINGS

FIGS. 1A-1D show that fexaramine (Fex) enhances thermogenesis in leptin-deficient ob/ob mice. Leptin-deficient ob/ob mice were treated daily with Vehicle or Fexaramine (100 mg/kg) via P.O. for 5 weeks. (A) Core temperature (B) QPCR of genes involved in thermogenesis in brown fat (C) Functional annotation of transcriptional altered genes (D) Heatmap showing that the expression of a large number of genes involved in oxidative phosphorylation in brown fat is increased with Fex treatment.

FIGS. 2A-2I show that Fex improves glucose homeostasis without body weight changes in leptin-deficient mice. Leptin-deficient ob/ob mice were treated daily with vehicle or Fex (100 mg/kg) via P.O. for 5 weeks. (A) Body weight curve (B) Body weight composition by MRI (C) Wet weight of inguinal (iWAT), gonadal (gWAT) white adipose tissue and liver (D) Serum glucose levels after 4 hour fast (E) Insulin levels after 4 hour fast (F) Glucose tolerance test (G) Insulin tolerance test (ITT) (H) Pyruvate tolerance test (PTT) (I) Progressive blood glucose levels with Fex treatment and after withdrawal of Fex. Data represent the mean±STD. Statistical analysis was performed with the Student's t test. *p<0.05, **p<0.01.

FIGS. 3A-3C show that Fex increases the M2-macrophage population to suppress inflammation in adipose tissues. Leptin-deficient ob/ob mice were treated daily with vehicle or Fex (100 mg/kg) via P.O. for 5 weeks. (A) Heatmap depicting that the expression of gene involved in cytokine signaling in gWAT are largely reduced by Fex treatment (B) Pathway analyses of gene expression changes in gWAT (C) Macrophage profile in gWAT. Data represent the mean±STD. Statistical analysis was performed with the Student's t test. *p<0.05, **p<0.01.

FIGS. 4A-4D show that Fex enhances insulin signaling in skeletal muscles. Leptin-deficient ob/ob mice were treated daily with vehicle or Fex (100 mg/kg) via P.O. for 5 weeks. (A) Pathway analyses of gene expression changes in skeletal muscles, including quadriceps and soleus (B) Heatmap depicting that the expression of gene involved in mitochrodial function and insulin signaling in quadriceps are largely increased upon Fex treatment (C) Heatmap depicting that the expression of gene involved in mitochrodial function and oxidation/reduction reactions in soleus are largely increased upon Fex treatment (D) qPCR of gene expression in quadriceps and soleus. Data represent the mean±STD. Statistical analysis was performed with the Student's t test. *p<0.05, **p<0.01

FIGS. 5A-5C show that Fex reduces gluconeogenesis/lipogenesis in the liver. Leptin-deficient ob/ob mice were treated daily with vehicle or Fex (100 mg/kg) via P.O. for 5 weeks. (A) Pathway analyses of gene expression changes in liver (B) Heatmap depicting that the expression of gene involved in gluconeogenesis, glycogen metabolism, and the insulin signaling pathway in the liver are largely suppressed upon Fex treatment. Data represent the mean±STD. Statistical analysis was performed with the Student's t test. *p<0.05, **p<0.01.

FIGS. 6A-6D show that Fex enhances glucose metabolism in leptin-deficient mice. Leptin-deficient ob/ob mice were treated daily with vehicle or Fex (100 mg/kg) via P.O. for 5 weeks. Mice were housed in metabolic cages and the following were measured: (A) Oxygen consumption (B) Carbon dioxide production (C) Respiratory Exchange Rate (D) Cumulative ambulatory counts. Data represent the mean±STD. Statistical analysis was performed with the Student's t test. *p<0.05, **p<0.01

FIGS. 7A-7G show that Fex enhances mitochondrial activity to increase glucagon-like peptide 1 (GLP-1) secretion in enteroendocrine L cells. (A-B) Leptin-deficient ob/ob mice were treated daily with vehicle or Fex (100 mg/kg) via P.O. for 5 weeks (A) Gene expression profile in ileum (B) Heatmap depicting that the expression of selected gene involved in mitochrondrial metabolism are increased upon Fex treatment (C-E) Human intestinal L cells (NCI-H716) were treated with vehicle or Fex (1 μM) for 24 hr. (C) Real-time oxygen consumption rate in vitro (D) ATP maintenance in vehicle or Fexa-treated L cells. ATP levels were measured upon treatment with Bethanechol chloride (uncoupler) (E) Glucose stimulated GLP-1 secretion from L cells after treatment with control (DMSO), Fex, or the TGR5 ligand INT-777, normalized to 0 mM glucose (F-G) Leptin-deficient ob/ob mice were treated daily with vehicle or fexaramine (100 mg/kg) via P.O. for 5 weeks. (F) Serum total GLP-1 levels (G) Glucose tolerance test performed after pre-treatment with the Dpp4 inhibitor, Sitagliptin (10 mg/kg). Data represent the mean±STD. Statistical analysis was performed with the Student's t test. *p<0.05, **p<0.01.

FIG. 8 shows that functional FXR is expressed in enteroendocrinal L cells. QPCR shows that the FXR target gene SHP is induced by Fex and Fex-D treatment, but not by the TGR5 ligand INT-777. Proglucagon expression is selectively induced by INT-777, while the expression of DPP4 is not affected by either FXR or TGR5 ligands.

FIGS. 9A-9B show that FEX enhances mitochondrial activity to maintain ATP levels in enteroendocrine L cells. Human intestinal L cells were treated with vehicle (DMSO), fexaramine (1 μM) or the FRX antagonist gugglusterone (10 μM) for 24 hr. (A) Oxygen consumption rate (OCR) measured in a Seahorse analyzer. (B) Total GLP-1 secretion at basal condition.

FIGS. 10A-10F show that Fex restores glucose-stimulated insulin secretion in pancreatic β cells. Leptin-deficient ob/ob mice were treated daily with vehicle or Fex (100 mg/kg) via P.O. for 5 weeks. (A) Insulin secretion in response to a glucose challenge. (B) Insulin content in islets (C) ex vivo glucose-stimulated insulin secretion from isolated pancreatic islets (D) Histological analysis (E) Heatmaps depicting that Fex treatment largely reduces the expression of genes involved in apoptosis, and increases the expression of genes involved in wound healing, cAMP signaling, insulin secretion, and Redox reactions in islets (F) Leptin-deficient ob/ob mice were treated daily with Fexaramine (100 mg/kg) via P.O. for 5 weeks followed by treatment with vehicle or the GLP-1 antagonist Ex-9 (100 mg/kg) via I.P for two weeks. Insulin secretion in response to a glucose challenge. Data represent the mean±STD. Statistical analysis was performed with the Student's t test. *p<0.05, **p<0.01.

FIG. 11 shows that FEX restores pancreatic beta cell physiology via GLP-1 signaling. Leptin-deficient ob/ob mice were treated daily with Fex (100 mg/kg) via P.O. for 6 weeks. During the last 2 weeks, Fex treatment was combined with vehicle or the GLP-1 antagonist Ex-9 (100 mg/kg) via I.P. Blood glucose levels during treatments. Data represent the mean±STD. Statistical analysis was performed with the Student's t test. *p<0.05, **p<0.01.

FIGS. 12A-12H show that structurally modified Fex analogues increase glucagon-like peptide 1 (GLP-1) secretion to restore glucose stimulated insulin secretion in leptin-deficient mice. Leptin-deficient ob/ob mice were treated daily with vehicle, Fex, or the analogue Fex-D (50 mg/kg) via P.O. for 14 days (A) Chemical structures of fexaramine and Fex-D (B) Schematic of the experimental design (C) Serum glucose levels (D) Glucose tolerance test (E) Insulin secretion in vivo (F) GLP-1 secretion in vivo (G) Heatmap showing hierarchal clustering of gene expression changes in islet induced by Fex (100 mg/kg, 4 wks) and FexD (50 mg/kg, 2 weeks) treatment in ob/ob mice (in vivo); by treatment of islets with XL335 (1 μM), Fex (1 μM), mFgf15 and hFgf19 (24 hours ex vivo); and mFgf15 (0.15 mg/kg, 2 weeks) and INT777 (Tgr5, 60 mg/kg, 4 weeks) treatment of ob/ob mice (in vivo). Gene expression was measured by RNA-Seq and changes are expressed as Z-score. (H) Ven diagram of changes in gene expression in pancreatic islets induced by FEX (100 mg/kg for 5 weeks) and FEX-D (50 mg/kg for 14 days). Pathway analysis of genes co-regulated by Fex and Fex-D. Heatmap comparing gene expression changes induced by Fex and Fex-D, revealing increased efficacy of Fex-D as well as altered specificity. Data represent the mean±STD. Statistical analysis was performed with the Student's t test. *p<0.05, **p<0.01.

FIGS. 13A-13D show that FEX-D enhances thermogenesis and oxygen consumption. (A-B) Leptin-deficient ob/ob mice were treated daily with vehicle, Fex, or Fex analogue Fex-D (50 mg/kg) via P.O. for 14 days. (A) Body weight curves and (B) Core body temperature. (C) Oxygen consumption rate (OCR) of human intestinal L cells treated with vehicle (DMSO), Fex (1 μM), FEX-D (1 μM) or XL335 (1 μM) for 24 hr. (D) Gene expression changes, as measured by QPCR, in the livers of ob/ob mice treated with vehicle or Fex-D, indicating that oral Fex-D delivery does not activate FXR target genes in liver. Data represent the mean±STD. Statistical analysis was performed with the Student's t test. *p<0.05, **p<0.01.

FIGS. 14A-14G show that Fex rescues β cells through enhanced redox and reduced apoptosis similar to TGR5 activation. Leptin-deficient ob/ob mice were treated daily with vehicle, Fex (100 mg/kg), or the TGR5 agonist, INT-777 (60 mg/kg) via P.O. for 5 weeks. (A) Body weight curve (B) MRI Body composition (C) Blood glucose levels (D) Glucose tolerance test, GTT (E) Insulin tolerance test, ITT (F) In vivo glucose stimulated insulin secretion (G) ex vivo glucose stimulated insulin secretion from islets. Data represent the mean±STD. Statistical analysis was performed with the Student's t test. *p<0.05, **p<0.01.

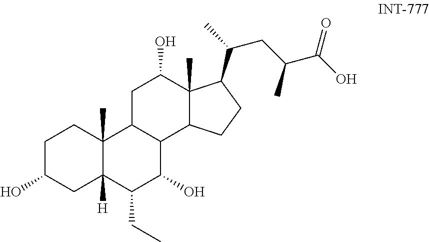 [SMILES: CC[C@H]1[C@@H](O)C2C3CC[C@H]([C@H](C)C[C@H](C)C(=O)O)[C@@]3(C)[C@@H](O)CC2[C@@]2(C)CC[C@@H](O)C[C@@H]12]

FIGS. 15A-15C show that Fex rescues β cells through enhanced redox and reduced apoptosis similar to TGR5 activation. Leptin-deficient ob/ob mice were treated daily with vehicle, Fex (100 mg/kg), or the TGR5 agonist, INT-777 (60 mg/kg) via P.O. for 5 weeks. (A) Ven diagrams showing overlap in gene expression changes induced in islet by Fex and INT-777 treatments (B) Pathway analyses of commonly regulated genes in islets (C) Heatmap of transcriptional changes in islets indicating that Fex and INT-777 treatments induce similar changes in genes involved in different physiological processes.

FIGS. 16A-16D show the effects of the FGF15 signaling pathway on glucose homeostasis. Leptin-deficient ob/ob mice were treated daily with vehicle or FGF-15 (0.15 mg/kg) via I.P. for 2 weeks. (A) Blood glucose levels (B) Glucose tolerance test (C) Insulin tolerance test (D) Glucose uptake into skeletal muscle (soleus). Data represent the mean±STD. Statistical analysis was performed with the Student's t test. *p<0.05, **p<0.01.

FIGS. 17A-17F show the transcriptional changes induced in pancreatic islets. Ob/ob mice were treated with Fex (100 mg/kg for 5 weeks), INT-777 (60 mg/kg for 5 weeks), or FGF-15 (0.15 mg/kg for 2 weeks). (A) Heatmap of expression changes in genes involved in sterol/cholesterol metabolic processes, showing similar levels of induction by Fex and INT-777, while FGF15 treatment largely suppressed these genes. (B) Heatmap of expression changes in genes involved in oxidation/reduction, showing similar levels of induction by Fex and INT-777, while FGF15 treatment largely suppressed these genes. (C) Heatmap of expression changes in genes involved in insulin secretion/cAMP pathway, showing similar levels of induction by Fex and INT-777, while FGF15 treatment had a minimal effect. (D) Heatmap of expression changes in genes involved in inflammation and immune responses, showing largely similar levels of induction by Fex and INT-777, while FGF15 treatment had a minimal effect. (E) Heatmap of expression changes in genes involved in wound healing responses, showing largely similar levels of induction by Fex, INT-777, and FGF15. (F) Heatmap of expression changes in genes involved in apoptosis, showing largely similar levels of induction by Fex and INT-777, while FGF15 treatment had a minimal effect.

FIG. 18 is a heatmap of expression changes of selected genes in pancreatic islets from ob/ob mice treated with fexaramine analogs, Fex, Fex-D, and Salk110, revealing similar expression changes with the three analogs.

### SEQUENCE LISTING

The amino acid sequences are shown using standard three letter code for amino acids, as defined in 37 C.F.R. 1.822.

SEQ ID NO. 1 is a protein sequence of GLP-1-(7-36).

SEQ ID NO. 2 is a protein sequence of GLP-2.

### DETAILED DESCRIPTION

### I. Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a FXR agonist” includes single or plural FXR agonists and is considered equivalent to the phrase “comprising at least one FXR agonist.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Dates of GenBank® Accession Nos. referred to herein are the sequences available at least as early as Mar. 13, 2015. All references, including patents and patent applications, and GenBank® Accession numbers cited herein are incorporated by reference.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

All groups herein are understood to include substituted groups unless specifically stated otherwise, or context indicates otherwise. A substituted group means that one or more hydrogen atoms of the specified group or radical is each, independently of one another, replaced with the same or different non-hydrogen substituent. Exemplary substituent groups are identified below.

Substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, deuterium, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻ M⁺, —SO₂OR⁷⁰, —OSO₂R⁷⁰, —OSO₂O M⁺, —OSO₂OR⁷⁰, —P(O)(O) 2(M⁺)₂, —P(O)(OR⁷⁰)O M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ or —NR⁷⁰C(NR⁷⁰)NR⁸⁰R⁸⁰, where R⁶⁰ is selected from alkyl, alkenyl, cycloalkyl, heteroalkyl, heterocycloalkylalkyl, cycloalkylalkyl, aryl, arylalkyl, heteroaryl or heteroarylalkyl, each of which maybe optionally further substituted; each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from O, N or S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as +N(R⁶⁰)₄; or an alkaline earth ion, such as [Ca²⁺]_{0.5}, [Mg²⁺]_{0.5}, or [Ba²⁺]_{0.5} (“subscript 0.5” means e.g. that one of the counter ions for such divalent alkali earth ions can be an ionized form of a compound of the invention and the other a typical counter ion such as chloride, or two ionized compounds of the invention can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl, N-morpholinyl and —N(alkyl)₂ such as, for example, —N(methyl)₂ or —N(methyl)(ethyl).

Substituent groups for hydrogens on unsaturated carbon atoms in “substituted” alkene, cycloalkene, alkyne, aryl and heteroaryl groups are, unless otherwise specified, —R⁶⁰, halo, deuterium, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻2(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ or —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

Substituent groups for replacing hydrogens on nitrogen atoms in “substituted” heterocyclic groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ or —NR⁷⁰C(NR⁷⁰)NR⁸⁰R⁸⁰, where R⁶⁰, R⁷⁰, R⁸⁰ and M⁺ are as previously defined.

In a preferred embodiment, a group that is substituted has 1 substituent, 1 or 2 substituents, 1, 2, or 3 substituents or 1, 2, 3 or 4 substituents.

Also, it is understood that the above definitions are not intended to include impermissible substitution patterns. Such impermissible substitution patterns are understood by a person having ordinary skill in the art.

Additionally, it is understood by a person of ordinary skill in the art that if an atom does not appear to have sufficient specific bonds to satisfy valence requirements, such as an apparent trivalent carbon, there are sufficient implicit hydrogens present to satisfy those valence requirements.

A wavy line “ [symbol] ” or “ [symbol] ” or an arrow “→” denoted a point of attachment of a group or moiety.

“Acyl” means, unless otherwise stated, —C(O)R where R is aliphatic, D-aliphatic, heteroaliphatic or D-heteroaliphatic.

“Aliphatic” refers to a substantially hydrocarbon-based compound, or a radical thereof (e.g., C₆H₁₃, for a hexane radical), including alkanes, alkenes, alkynes, including cyclic versions thereof, such as alkyl, alkenyl, alkynyl, cycloalkyl, cycloalkenyl, and cycloalkynyl, and further including straight- and branched-chain arrangements, and all stereo and position isomers as well. Unless expressly stated otherwise, an aliphatic group contains from one to at least twenty-five carbon atoms; for example, from one to fifteen, from one to ten, from one to six, or from one to four carbon atoms. The term “lower aliphatic” refers to an aliphatic group comprising from one to ten carbon atoms. An aliphatic chain may be substituted or unsubstituted. Unless expressly referred to as an “unsubstituted aliphatic,” an aliphatic group can either be unsubstituted or substituted. An aliphatic group can be substituted with one or more substituents (up to two substituents for each methylene [—CH₂-] carbon in an aliphatic chain, or up to one substituent for each carbon of a —C═C— double bond in an aliphatic chain, or up to one substituent for a carbon of a terminal methine group). Exemplary aliphatic substituents include, for instance, amino, amide, sulfonamide, halo, cyano, carboxy, hydroxyl, mercapto, trifluoromethyl, alkyl, alkoxy, alkylthio, thioalkoxy, arylalkyl, heteroaryl, alkylamino, dialkylamino, or other functionality.

“D-aliphatic” refers to an aliphatic group where at least one hydrogen has been substituted by deuterium.

“Alkyl” refers to a hydrocarbon group having a saturated carbon chain, which, unless otherwise specified, may optionally be substituted, particularly with substituents as described in the definition of “substituted.” The chain may be cyclic, branched or unbranched. The term “lower alkyl” means that the alkyl chain includes 1-10 carbon atoms e.g. methyl, ethyl, n-propyl, isopropyl, n-butyl, iso-butyl, sec-butyl, tert-butyl, n-pentyl, tert-pentyl, neopentyl, isopentyl, sec-pentyl, 3-pentyl, hexyl, heptyl, octyl, nonyl or decyl. Also, by way of example, a methyl group, an ethyl group, an n-propyl and an isopropyl group are all represented by the term C₁₋₃ alkyl. Likewise terms indicating larger numerical ranges of carbon atoms are representative of any linear or branched hydrocarbyl falling within the numerical range. This inclusiveness applies to other hydrocarbyl terms bearing such numerical ranges. The terms alkenyl and alkynyl refer to hydrocarbon groups having carbon chains containing one or more double or triple bonds, respectively.

“Alkylene” refers to divalent saturated aliphatic hydrocarbyl groups preferably having from 1 to 10 carbon atoms, more preferably 1 to 4 carbon atoms, that are either straight-chained or branched, which may optionally be substituted, particularly with substituents as described herein, unless otherwise specified. This term is exemplified by groups such as methylene (—CH₂—), ethylene (—CH₂CH₂—), n-propylene (—CH₂CH₂CH₂—), iso-propylene (—CH₂CH(CH₃)—) or (—CH(CH₃)CH₂—), and the like.

“Alkenylene” refers to divalent unsaturated aliphatic hydrocarbyl groups preferably having from 2 to 10 carbon atoms, more preferably 2 to 4 carbon atoms, that are either straight-chained or branched, and include at least one double bond. Unless otherwise specified, the group may be optionally be substituted, particularly with substituents as described herein. This term is exemplified by groups such as ethenylene (—CH═CH—) and propenylene (—CH═CHCH₂—) and the like.

“Alkynylene” refers to divalent unsaturated aliphatic hydrocarbyl groups preferably having from 2 to 10 carbon atoms, more preferably 2 to 4 carbon atoms, that are either straight-chained or branched and include at least one triple bond. Unless otherwise specified, the group may be optionally be substituted, particularly with substituents as described herein. This term is exemplified by groups such as ethynylene (—C≡C—) and n-propynylene (—C≡CCH₂—) and the like.

“Alkylthio” refers to the group —S-alkyl.

“Alkoxy” refers to the group —O-alkyl. Alkoxy includes, by way of example, methoxy, ethoxy, n-propoxy, isopropoxy, n-butoxy, t-butoxy, sec-butoxy, n-pentoxy, and the like.

“Acyl” refers to the groups H—C(O)—, alkyl-C(O)—, alkenyl-C(O)—, alkynyl-C(O)—, cycloalkyl-C(O)—, cycloalkenyl-C(O)—, aryl-C(O)—, heteroaryl-C(O)—, or heterocyclic-C(O)—. By way of example, “acyl” includes the “acetyl” group CH₃C(O)—.

“Amino” refers to the group —NR′R″, wherein R′ and R″ independently are selected from hydrogen, aliphatic, D-aliphatic, heteroaliphatic or D-heteroaliphatic, or where R′ and R″ are optionally joined together with the nitrogen bound thereto to form a cycloamino group such as a heterocyclic, deuterated heterocyclic, heteroaryl or deuterated heteroaryl group comprising at least one ring nitrogen. Exemplary cycloamino groups include, but are not limited to, pyrrolidine, pyrrole, imidazole, triazole, tetrazole, piperidine, triazinane, piperazine, morpholine, azepane, diazepane, azocane, diazocane, azonane or azecane.

“Aminocarbonyl” refers to a chemical functional group —C(═O)-amino, where amino is as defined herein. A primary aminocarbonyl is —CONH₂. “Aminocarbonyl” also refers to the group —C(O)NR′R, wherein R′ and R independently are selected from hydrogen, alkyl, alkenyl, alkynyl, aryl, cycloalkyl, cycloalkenyl, heteroaryl, or heterocyclic, or where R²¹ and R²² are optionally joined together with the nitrogen bound thereto to form a heterocyclic group.

“Aminosulfonyl” refers refers to a chemical function group —SO₂-amino. A primary aminosulfonyl is —SO₂NH₂. Certain embodiments refer to the group —SO₂NR′R where R and R″ are independently are selected from hydrogen, alkyl, alkenyl, alkynyl, aryl, cycloalkyl, cycloalkenyl, heteroaryl, or heterocyclic, or where R and R are optionally joined together with the nitrogen bound thereto to form a heterocyclic group.

“Aryl” or “Ar” refers to an aromatic moiety, such as a carbocyclic group of from 6 to 15 carbon atoms having a single ring (e.g., phenyl) or multiple condensed rings (e.g., naphthyl or anthryl) in which at least one of the condensed rings is aromatic (e.g., 2-benzoxazolinone, 2H-1,4-benzoxazin-3(4H)-one-7-yl, 9,10-dihydrophenanthrene, and the like), provided that the point of attachment is through an atom of the aromatic aryl group. Unless otherwise specified, the aryl group may be optionally be substituted, particularly with substituents as described herein. Preferred aryl groups include phenyl and naphthyl.

“Alkenyl” refers to straight chain or branched hydrocarbyl groups having from 2 to 6 carbon atoms and preferably 2 to 4 carbon atoms and having at least 1 double bond. Unless otherwise specified, the alkenyl group may be optionally substituted. Such groups are exemplified, for example, bi-vinyl, allyl, and but-3-en-1-yl. Included within this term are the cis and trans isomers or mixtures of these isomers, unless otherwise specified.

“Alkynyl” refers to straight chain or branched hydrocarbyl groups having from 2 to 6 carbon atoms, and preferably 2 to 4 carbon atoms, and having at least 1 site of triple bond unsaturation. Unless otherwise specified, the alkynyl group may be optionally substituted. Such groups are exemplified, for example, by ethynyl, 1-propynyl and 2-propynyl.

“Boronic acid” refers to the groups —B(OR)₂, where each R independently is selected from H, alkyl, cycloalkyl, aryl or where the R substituents form a ring, such as in a picolinate ester

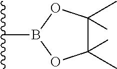 [SMILES: *B1OC(C)(C)C(C)(C)O1]

or a catechol ester

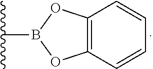 [SMILES: *B1Oc2ccccc2O1]

“Cyano” refers to the chemical functional group —CN.

“Carboxyl,” “carboxylic acid” or “carboxy” refers to the chemical functional group —CO₂H.

“Carboxyl ester,” “carboxylic acid ester,” or “carboxy ester” refers to the chemical functional group —CO₂R where R is aliphatic, D-aliphatic, heteroaliphatic or D-heteroaliphatic.

“Cycloalkyl” refers to a cyclic alkyl group of from 3 to 10 carbon atoms having a single ring, which, unless otherwise specified, may be optionally substituted. Examples of suitable cycloalkyl groups include, for instance, cyclopropyl, cyclobutyl, cyclopentyl, cyclohexyl, cycloheptyl, cyclooctyl and the like.

“Cycloalkenyl” refers to a cyclic alkenyl group of from 3 to 10 carbon atoms having a single ring, which, unless otherwise specified, may be optionally substituted. Examples of suitable cycloalkenyl groups include, for instance, cyclohexenyl, cyclopentenyl, and cyclobutenyl.

“Halo”, “halide” or “halogen” refers to fluoro, chloro, bromo, and iodo and is preferably fluoro or chloro.

“Hydroxy” or “hydroxyl” refers to the group —OH.

“Heteroaliphatic” refers to an aliphatic compound or group having at least one heteroatom, i.e., one or more carbon atoms has been replaced with an atom having at least one lone pair of electrons, typically nitrogen, oxygen, phosphorus, silicon, or sulfur. Heteroaliphatic compounds or groups may be substituted or unsubstituted, branched or unbranched, cyclic or acyclic, and include “heterocycle”, “heterocyclyl”, “heterocycloaliphatic”, or “heterocyclic” groups. Examples of heterocycles include morpholine and piperidine.

“D-heteroaliphatic” refers to a heteroaliphatic group where at least one hydrogen has been substituted by a deuterium.

“Heteroaryl” refers to an aromatic group having from 1 to 10 carbon atoms and at least one, and more typically 1 to 4, heteroatoms selected from oxygen, nitrogen or sulfur within the ring. Unless otherwise specified, the heteroaryl group may be optionally substituted. Such heteroaryl groups can have a single ring (e.g., pyridinyl, imidazolyl or furyl) or multiple condensed rings (e.g., indolizinyl, quinolinyl, benzimidazolyl, benzopyrazolyl or benzothienyl), wherein at least one of the condensed rings is aromatic and may or may not contain a heteroatom, provided that the point of attachment is through an atom of an aromatic ring. In one embodiment, the nitrogen and/or sulfur ring atom(s) of the heteroaryl group are optionally oxidized to provide N-oxide (N→O), sulfinyl, or sulfonyl moieties. Preferred heteroaryls include pyridinyl, pyrrolyl, indolyl, thiophenyl, benzopyrazolyl and furanyl.

“Heterocycle,” “heterocyclic,” “heterocycloalkyl,” and “heterocyclyl” refer to a saturated or unsaturated group having a single ring or multiple condensed rings, including fused, bridged and spiro ring systems, and having from 3 to 15 ring atoms, including at least one, and more typically 1 to 4, hetero atoms. The hetero atoms are selected from nitrogen, sulfur, or oxygen. Unless otherwise specified, the group may be optionally substituted. In fused ring systems, one or more of the rings can be cycloalkyl, aryl, or heteroaryl, provided that the point of attachment is through a non-aromatic ring. In one embodiment, the nitrogen and/or sulfur atom(s) of the heterocyclic group are optionally oxidized to provide for the N-oxide, —S(O)—, or —SO₂-moieties.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

“Nitro” refers to the group —NO₂.

“Polycyclic” refers to a saturated or unsaturated polycyclic ring system having from about 5 to about 25 carbon atoms and having two or more rings (e.g. 2, 3, 4, or 5 rings). The rings can be fused and/or bridged to form the polycyclic ring system, and unless otherwise specified, may be optionally substituted. For example, the term includes bicyclo [4,5], [5,5], [5,6] or [6,6] ring systems, as well as the following bridged ring systems:

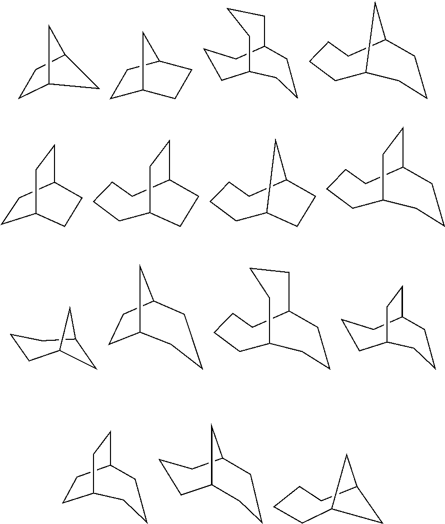

(i.e., [2.1.1], [2.2.1], [3.3.3], [4.3.1], [2.2.2], [4.2.2], [4.2.1], [4.3.2], [3.1.1], [3.2.1], [4.3.3], [3.3.2], [3.2.2], [3.3.1] and [4.1.1] polycyclic rings, respectively), and adamantyl. Polycyclic groups can be linked to the remainder of the compound through any synthetically feasible position. If a stereocenter is created then all possible stereocenters are contemplated. Like the other polycarbocycles, these representative bicyclo and fused ring systems can optionally comprise one or more double bonds in the ring system.

“Sulfonyl” refers to the group —SO₂—, and includes —SO₂-alkyl, —SO₂-alkenyl, —SO₂-cycloalkyl, —SO₂-cycloalkenyl, —SO₂-aryl, —SO₂-heteroaryl, or —SO₂-heterocyclic, wherein alkyl, alkenyl, cycloalkyl, cycloalkenyl, aryl, heteroaryl, and heterocyclic are as defined herein. Sulfonyl includes groups such as methyl-SO₂—, phenyl-SO₂—, and 4-methylphenyl-SO₂—.

“Carboxyl bioisosteric,” or “carboxyl bioisostere” refer to a group with similar physical or chemical properties to a carboxyl group that produce broadly similar biological properties, but which may reduce toxicity or modify the activity of the compound, and may alter the metabolism of the compound. Exemplary carboxyl bioisosteres include, but are not limited to,

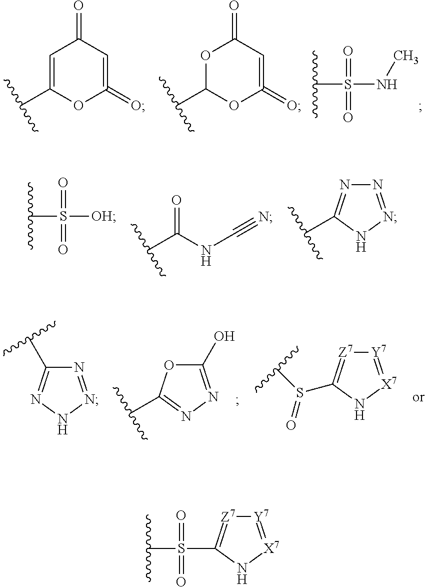

X⁷, Y⁷, and Z⁷ are each independently selected from N, CH₂ or CO;

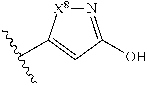

where X⁸ is selected from O, S or NMe;

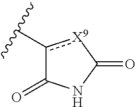

where X⁹ is selected from O, N, S, CH or CH₂;

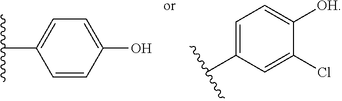 [SMILES: *c1ccc(O)cc1 ; *c1ccc(O)c(Cl)c1]

Additional carboxyl bioisosteric groups contemplated by the present disclosure include

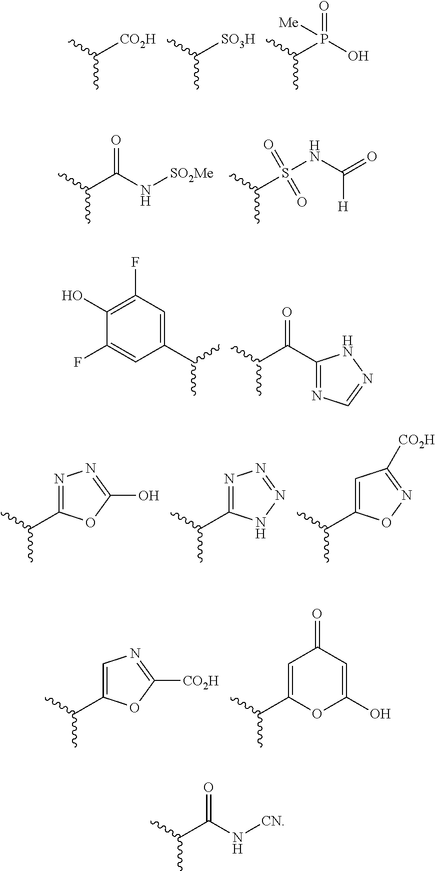

Particular examples of the presently disclosed compounds include one or more asymmetric centers; thus these compounds can exist in different stereoisomeric forms. Accordingly, compounds and compositions may be provided as individual pure enantiomers or as stereoisomeric mixtures, including racemic mixtures. In certain embodiments the compounds disclosed herein are synthesized in or are purified to be in substantially enantiopure form, such as in a 90% enantiomeric excess, a 95% enantiomeric excess, a 97% enantiomeric excess or even in greater than a 99% enantiomeric excess, such as in enantiopure form.

Prodrugs of the disclosed compounds also are contemplated herein. A prodrug is an active or inactive compound that is modified chemically through in vivo physiological action, such as hydrolysis, metabolism and the like, into an active compound following administration of the prodrug to a subject. The term “prodrug” as used throughout this text means the pharmacologically acceptable derivatives such as esters, amides and phosphates, such that the resulting in vivo biotransformation product of the derivative is the active drug as defined in the compounds described herein. Prodrugs preferably have excellent aqueous solubility, increased bioavailability and are readily metabolized into the active inhibitors in vivo. Prodrugs of a compounds described herein may be prepared by modifying functional groups present in the compound in such a way that the modifications are cleaved, either by routine manipulation or in vivo, to the parent compound. The suitability and techniques involved in making and using prodrugs are well known by those skilled in the art. For a general discussion of prodrugs involving esters see Svensson and Tunek, Drug Metabolism Reviews 165 (1988) and Bundgaard, Design of Prodrugs, Elsevier (1985).

“Prodrug” also is intended to include any covalently bonded carriers that release an active parent drug of the present invention in vivo when the prodrug is administered to a subject. Since prodrugs often have enhanced properties relative to the active agent pharmaceutical, such as, solubility and bioavailability, the compounds disclosed herein can be delivered in prodrug form. Thus, also contemplated are prodrugs of the presently disclosed compounds, methods of delivering prodrugs and compositions containing such prodrugs. Prodrugs of the disclosed compounds typically are prepared by modifying one or more functional groups present in the compound in such a way that the modifications are cleaved, either in routine manipulation or in vivo, to yield the parent compound. Prodrugs include compounds having a phosphonate and/or amino group functionalized with any group that is cleaved in vivo to yield the corresponding amino and/or phosphonate group, respectively. Examples of prodrugs include, without limitation, compounds having an acylated amino group, an ascorbate moiety, an ortho ester, an imidate group and/or a phosphonate ester or phosphonate amide group.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like. If the molecule contains a basic functionality, pharmaceutically acceptable salts include salts of organic or inorganic acids, such as hydrochloride, hydrobromide, tartrate, mesylate, acetate, maleate, oxalate, and the like.

“Pharmaceutically acceptable excipient” refers to a substantially physiologically inert substance that is used as an additive in a pharmaceutical composition. As used herein, an excipient may be incorporated within particles of a pharmaceutical composition, or it may be physically mixed with particles of a pharmaceutical composition. An excipient can be used, for example, as a carrier, flavoring, thickener, diluent, buffer, preservative, or surface active agent and/or to modify properties of a pharmaceutical composition. Examples of excipients include, but are not limited, to polyvinylpyrrolidone (PVP), tocopheryl polyethylene glycol 1000 succinate (also known as vitamin E TPGS, or TPGS), dipalmitoyl phosphatidyl choline (DPPC), trehalose, sodium bicarbonate, glycine, sodium citrate, and lactose.

“Enteric coating” refers to a coating such as may be applied to disclosed compounds or compositions comprising the compounds to help protect drugs from disintegration, digestion etc. in the stomach, such as by enzymes or the pH of the stomach. Typically, the coating helps prevent the drug from being digested in the stomach, and allows delivery of the medication to the intestine.

“Administer,” “administering”, “administration,” and the like, as used herein, refer to methods that may be used to enable delivery of agents or compositions to the desired site of biological action. These methods include, but are not limited to oral routes, intraduodenal routes and rectal administration. Administration techniques that are optionally employed with the agents and methods described herein are found in sources e.g., Goodman and Gilman, The Pharmacological Basis of Therapeutics, current ed.; Pergamon; and Remington's, Pharmaceutical Sciences (current edition), Mack Publishing Co., Easton, Pa. In certain embodiments, the agents and compositions described herein are administered orally.

“Calorie” refers to the amount of energy, e.g. heat, required to raise the temperature of 1 gram of water by 1° C. In various fields such as medicine, nutrition, and the exercise sciences, the term “calorie” is often used to describe a kilocalorie. A kilocalorie is the amount of energy needed to increase the temperature of 1 kilogram of water by 1° C. One kilocalorie equals 1000 calories. The kilocalorie is abbreviated as kc, kcal or Cal, whereas the calorie or gram calorie is abbreviated as cal. In some embodiments, food intake in the subject is measured in terms of overall calorie consumption. Likewise, in some embodiments, fat intake can be measured in terms of calories from fat.

“Co-administration,” “administered in combination with,” and their grammatical equivalents, are meant to encompass administration of the selected therapeutic agents to a subject, and are intended to include treatment regimens in which the agents are administered by the same or different route of administration or at the same (e.g., contemporaneously) or different times. In some embodiments the agents described herein will be co-administered with other agents. These terms encompass administration of two or more agents to the subject so that both agents and/or their metabolites are present in the subject at the same time. They include simultaneous administration in separate compositions, administration at different times in separate compositions, and/or administration in a composition in which both agents are present. Thus, in some embodiments, the agents described herein and the other agent(s) are administered in a single composition. In some embodiments, the agents described herein and the other agent(s) are admixed in the composition.

“effective amount,” “pharmaceutically effective amount” or “therapeutically effective amount,” refer to a sufficient amount of at least one agent being administered to achieve a desired result, e.g., to relieve to some extent one or more symptoms of a disease or condition being treated (e.g., LADA). In certain instances, the result is a reduction and/or alleviation of the signs, symptoms, or causes of a disease, or any other desired alteration of a biological system. In certain instances, an “effective amount” for therapeutic uses is the amount of the composition comprising an agent as set forth herein required to provide a clinically significant decrease in a disease. An appropriate “effective” amount in any individual case can be determined using any suitable technique, such as a dose escalation study.

“Enhancing enteroendocrine peptide secretion” refers to a sufficient increase in the level of the enteroendocrine peptide agent to, for example, decrease hunger in a subject, to curb appetite in a subject and/or decrease the food intake of a subject or individual and/or treat any disease or disorder described herein.

“FXR”: farnesoid X receptor (also known as nuclear receptor subfamily 1, group H, member 4 (NR¹H4)) (e.g., OMIM: 603826): This protein functions as a receptor for bile acids, and when bound to bile acids, regulates the expression of genes involved in bile acid synthesis and transport. FXR is expressed at high levels in the liver and intestine. Chenodeoxycholic acid and other bile acids are natural ligands for FXR. Similar to other nuclear receptors, when activated, FXR translocates to the cell nucleus, forms a dimer (in this case a heterodimer with RXR) and binds to hormone response elements on DNA, which up- or down-regulates the expression of certain genes. One of the primary functions of FXR activation is the suppression of cholesterol 7 alpha-hydroxylase (CYP7A1), the rate-limiting enzyme in bile acid synthesis from cholesterol. FXR does not directly bind to the CYP7A1 promoter. Rather, FXR induces expression of small heterodimer partner (SHP), which then functions to inhibit transcription of the CYP7A1 gene. In this way, a negative feedback pathway is established in which synthesis of bile acids is inhibited when cellular levels are already high. FXR sequences are publically available, for example from GenBank® sequence database (e.g., accession numbers NP_001193906 (human, protein) and NP_001156976 (mouse, protein), and NM_001206977 (human, nucleic acid) and NM_001163504 (mouse, nucleic acid)).

“Metabolic disorder” refers to any disorder that involves an alteration in the normal metabolism of carbohydrates, lipids, proteins, nucleic acids or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, but are not limited to, the endocrine (hormonal) control system (e.g., the insulin pathway, the enteroendocrine hormones including GLP-1, GLP-2, oxyntomodulin, PYY or the like), the neural control system (e.g., GLP-1 in the brain) or the like. Examples of metabolic disorders include and are not limited to diabetes, insulin resistance, dyslipidemia (such as an elevated serum lipids and/or triglycerides, such as a serum LDL of at least 100 mg/dL, such as at least 130 mg/dL, at least 160 mg/dL or at least 200 mg/dL, such as 100 to 129 mg/dL, 130 to 159 mg/dL, 160 to 199 mg/dL or greater than 200 mg/dL, and/or such as a serum triglyceride of at least of at least 151 mg/dL, such as at least 200 mg/dL, or at least 500 mg/dL, such as 151 to 199 mg/dL, 200 to 499 mg/dL or greater than 499 mg/dL), metabolic syndrome, or the like.

“Metabolic rate” refers to the rate at which the subject uses energy. This is also known as the rate of metabolism, or the rate of energy consumption, and reflects the overall activity of the individual's metabolism. The term basal metabolism refers to the minimum amount of energy required to maintain vital functions in an individual at complete rest, measured by the basal metabolic rate in a fasting individual who is awake and resting in a comfortably warm environment. The term “basal metabolic rate” refers to the rate at which energy is used by an individual at rest. Basal metabolic rate is measured in humans by the heat given off per unit time, and expressed as the calories released per kilogram of body weight or per square meter of body surface per hour. The heart beating, breathing, maintaining body temperature, and other basic bodily functions all contribute to basal metabolic rate. Basal metabolic rate can be determined to be the stable rate of energy metabolism measured in individuals under conditions of minimum environmental and physiological stress, or essentially at rest with no temperature change. The basal metabolic rate among individuals can vary widely. One example of an average value for basal metabolic rate is about 1 calorie per hour per kilogram of body weight.

“Non-systemic” or “minimally absorbed” as used herein refer to low systemic bioavailability and/or absorption of an administered compound. In some instances a non-systemic compound is a compound that is substantially not absorbed systemically. In some embodiments, FXR agonist compositions described herein deliver an FXR agonist to the distal ileum, colon, and/or rectum and not systemically (e.g., a substantial portion of the FXR agonist administered is not systemically absorbed). In some embodiments, the systemic absorption of a non-systemic compound is <0.1%, <0.3%, <0.5%, <0.6%, <0.7%, <0.8%, <0.9%, <1%, <1.5%, <2%, <3%, or <5% of the administered dose (wt. % or mol %). In some embodiments, the systemic absorption of a non-systemic compound is <15% of the administered dose. In some embodiments, the systemic absorption of a non-systemic compound is <25% of the administered dose. In an alternative approach, a non-systemic FXR agonist is a compound that has lower systemic bioavailability relative to the systemic bioavailability of a systemic FXR agonist. In some embodiments, the bioavailability of a non-systemic FXR agonist described herein is <30%, <40%, <50%, <60%, or <70% of the bioavailability of a systemic FXR agonist. In some embodiments, the serum concentration of the FXR agonist in the subject remains below the compound's EC₅₀ following administration (such as a level at least 20%, at least 30%, at least 40%, or even at least 50% below the compound's EC₅₀). In one specific example, if the compound's EC₅₀ is about 50 nM, following administration the serum concentration would be below 50 nM, such as less than 40 nM, less than 30 nM or less than 20 nM, for example 1 hour following administration.

“Prevent,” “preventing” or “prevention,” and other grammatical equivalents as used herein, include preventing additional symptoms, preventing the underlying metabolic causes of symptoms, inhibiting the disease or condition, e.g., arresting the development of the disease or condition and are intended to include prophylaxis. The terms further include achieving a prophylactic benefit. For prophylactic benefit, the compositions are optionally administered to a patient at risk of developing a particular disease (e.g., LADA), to a patient reporting one or more of the physiological symptoms of a disease (e.g., LADA), or to a patient at risk of reoccurrence of the disease (e.g., LADA).

“Subject”, “patient” or “individual” may be used interchangeably herein and refer to mammals and non-mammals, e.g., suffering from LADA. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, amphibians, and the like. In one embodiment of the methods and compositions provided herein, the mammal is a human.

“Treat,” “treating” or “treatment,” and other grammatical equivalents as used herein, include alleviating, inhibiting or reducing symptoms, reducing or inhibiting severity of, reducing incidence of, prophylactic treatment of, reducing or inhibiting recurrence of, preventing, delaying onset of, delaying recurrence of, abating or ameliorating a disease or condition symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, wherein the disease can be LADA. The terms further include achieving a therapeutic benefit. Therapeutic benefit means eradication or amelioration of the underlying disorder being treated, and/or the eradication or amelioration of one or more of the physiological symptoms associated with the underlying disorder, such that an improvement is observed in the patient.

### II. Overview

Disclosed herein are methods of using compounds that have activity as FXR agonists that are structurally distinct from bile acids, other synthetic FXR ligands, and other natural FXR ligands, to treat or prevent LADA. Such methods include administering a therapeutically effective amount of one or more FXR agonists to the GI tract of a subject, such as one or more of the FXR agonists disclosed herein. The absorption of these FXR agonists is substantially restricted to the intestinal lumen when delivered orally (e.g., serum levels of the compound are below the compound's EC₅₀ following administration of the compound, such as about 1 hour later). In various embodiments, administration of one or more FXR agonists results in activation of FXR transcriptional activity in the intestine, without substantially affecting other target tissues, such as liver or kidney.

Farnesoid X receptor (FXR) is a key component in regulating metabolic homeostasis including glucose and lipid metabolism as well as bile acid (BA) homeostasis. Fexaramine (Fex) is a gut-specific FXR agonist that can reduce diet-induced weight gain, body-wide inflammation and hepatic glucose production, while enhancing thermogenesis and browning of white adipose tissue. It is shown herein that Fex restores pancreatic β cell functions with robustly enhanced glucose-stimulated insulin secretion (GSIS) in diabetic mice without body weight changes. It is shown that Fex potentiates bioenergetics to enhance GLP-1 secretion in enteroendocrine L cells. Concomitantly, Fex increases gene expression of glucagon-like peptide-1 receptor (GLP-1R) in pancreatic β cells, resulting in restoration of GSIS in β cells to ameliorate hyperglycemia in ob/ob mice. Furthermore, Fex analogs, including Fex-D, are more effective at glucose lowering than Fex. Based on these observations, methods of activating enteroendocrinal FXR to treat LADA are provided.

### III. Compounds

Disclosed herein are farnesoid X receptor (FXR) agonist compounds, which can be used in disclosed embodiments of the method. Without limitation, these embodiments include compounds of Formula 1-35. Certain compounds are chiral, and all stereoisomers are included in this disclosure, as well as all geometric and structural isomers such as cis and trans isomers.

A. Farnesoid X Receptor (FXR) Agonist Compounds According to Formulas 1-18

Certain disclosed compound embodiments have the structure of formula 1

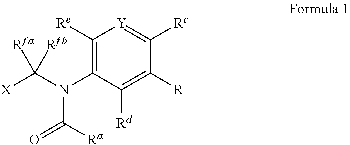

or a pharmaceutically acceptable salt thereof, wherein R is selected from

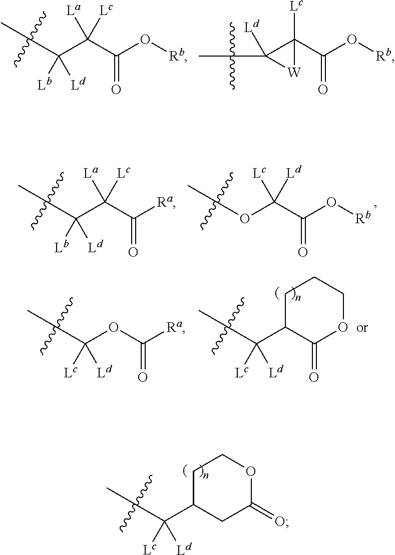

R^{a} is selected from aryl, heteroaryl, alkyl, alkenyl, cycloalkyl, heterocyclic, or polycyclic; R^{b} is selected from hydrogen, alkyl, alkenyl, or cycloalkyl; Y is CR^{g}, N or N—O (N-oxide); R^{c}, R^{d}, R^{e} and R^{g} are each independently selected from hydrogen, deuterium, halide, alkyl, alkenyl, alkoxy, alkylthio, amino, sulfonyl, aminosulfonyl, aminocarbonyl, acyl, hydroxyl or nitro; R^{fa} and R^{fb} are each independently selected from hydrogen, deuterium, halide or alkyl; L^{a} and L^{b} are each independently selected from hydrogen, deuterium, alkyl or cycloalkyl, or together form a pi-bond; L^{c} and L^{d} are each independently selected from hydrogen, deuterium, alkyl or cycloalkyl; W is selected from O or —(C(L^{c})(L^{d}))ₛ-; s is 1, 2, 3, 4, 5 or 6; n is 0 or 1; and X is aryl, heterocyclic or heteroaryl. In some embodiments when R^{b} is hydrogen, the compounds have activity as FXR agonists. In other embodiments when R^{b} is hydrogen, the compounds may have reduced or substantially no activity as FXR agonists.

Also with reference to formula 1, any or all of the following provisos may apply:

if W is CH₂ and L^{c} and L^{d} are both H, then X is not a benzopyran;

### if R is

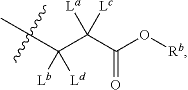

L^{c} and L^{d} are both H, and L^{a} and L^{b} are both H or together form a pi-bond, then X is not a benzopyran;

X is not substituted with —R^{x}-L^{x}-R^{x2}, where R^{x} is selected from O, NR^{x3}, sulfonyl or S; R^{x3} is selected from H, alkyl, alkenyl, alkynyl, cycloalkyl, cycloalkenyl, or aryl; L^{x} is selected from a bond, alkylene, alkenylene, alkynylene, cycloalkyl, cycloalkenyl, heterocyclic, aryl, heteroaryl or CR^{x4}R^{x5}; R^{x4} and R^{x5} are each independently selected from H, D, halogen, alkyl, alkenyl, alkynyl, cycloalkyl, cycloalkenyl, —C(O)OR^{x6}, or —C(O)NR^{x6}R^{x7}; R^{x6} and R^{x7} are each independently selected from H, alkyl, alkenyl, alkynyl, cycloalkyl or cycloalkenyl; R^{x2} is selected from —C(O)L^{x2}R^{x8} or a carboxyl bioisostere; L^{x2} is a bond or NR^{x3}; R^{x8} is H, alkyl, alkenyl, alkynyl, cycloalkyl, cycloalkenyl, —OR^{x9}, N(R^{x9})₂, —C(O)R^{x9}, —S(O)₂R^{x9}, —C(O)OR^{x9}, —S(O)₂N(R^{x9})₂ or —C(O)N(R⁹)₂; and each R^{x9} is independently selected from H, alkyl, alkenyl, alkynyl, cycloalkyl or cycloalkenyl; and

when R is

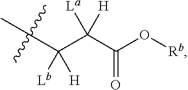

R^{c}, R^{d}, R^{e} and R^{fa} are all hydrogen, Y is CH and L^{a} and L^{b} are both H or together form a pi-bond, then
if R^{a} is cyclohexyl, R^{b} is methyl, and R^{fb} is H then X is not phenyl, 4-biphenyl, 4-bromophenyl, 3-bromophenyl, 2-bromophenyl, 4-tert-butylphenyl, 3-methoxyphenyl, 3,5-dimethoxyphenyl, 3-(trifluoromethyl)phenyl, 4-(3,4-difluorophenyl)phenyl, 4-(3-acetylphenyl)phenyl, 4-(4-methylthiophenyl)phenyl, 4-(4-methoxyphenyl)phenyl, 4-(3-methoxyphenyl)phenyl, 4-(2-methoxyphenyl)phenyl, 4-(3,5-dichlorophenyl)phenyl, 4-(4-tert-butylphenyl)phenyl, 4-(3-ethoxyphenyl)phenyl, 4-(3-chlorophenyl)phenyl, 4-(3-methylphenyl)phenyl, 4-(4-methylphenyl)phenyl, 4-(2-methoxy-5-chlorophenyl)phenyl, 4-(3-chloro-4-fluorophenyl)phenyl, 4-(4-trifluoromethoxyphenyl)phenyl, 4-(3-trifluoromethoxyphenyl)phenyl, 4-(2,6-dimethoxyphenyl)phenyl, 4-(4-dimethylaminophenyl)phenyl,

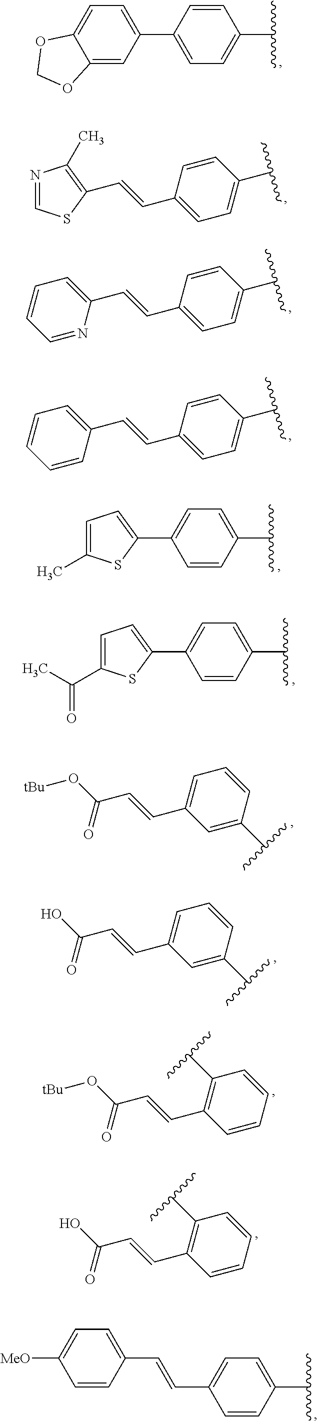

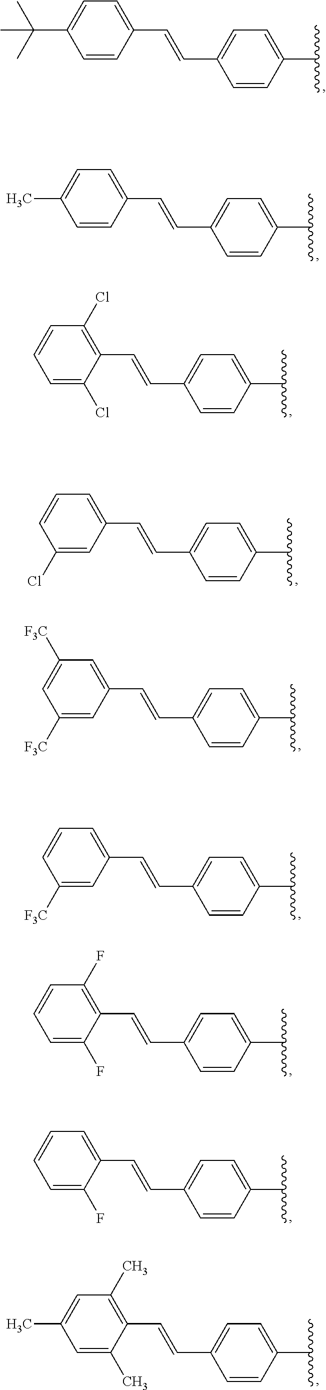

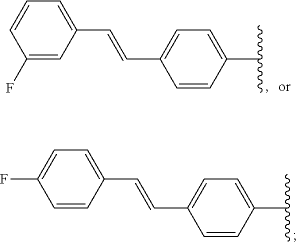 [SMILES: *c1ccc(/C=C/c2cccc(F)c2)cc1 ; *c1ccc(/C=C/c2ccc(F)cc2)cc1]

if R^{a} is cyclohexyl, R^{fb} is H and X is

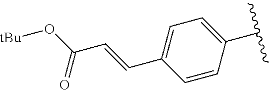 [SMILES: *c1ccc(/C=C/C(=O)OC(C)(C)C)cc1]

then R^{b} is not methyl, ethyl or tert-butyl;
if R^{b} is methyl, R^{fb} is H and X is

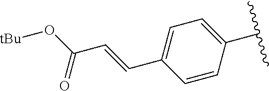 [SMILES: *c1ccc(/C=C/C(=O)OC(C)(C)C)cc1]

then R^{a} is not cyclopropyl, cyclobutyl, cyclopentyl, or cyclohexyl;
if R^{a} is cyclohexyl, R^{fb} is H and X is

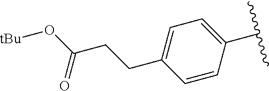 [SMILES: *c1ccc(CCC(=O)OC(C)(C)C)cc1]

then R^{b} is not methyl or tert-butyl;
if R^{a} is cyclohexyl, R^{b} is methyl, R^{fb} is H and X is

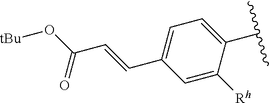

R^{h} then R^{h} is not hydroxyl, (trimethylsilyl)ethoxymethyl-O, methoxy, O-benzyl, OCH₂CO₂Et, OC(O)CH₃, OC(O)Ph or OSO₂CH₃; and
if R^{a} is cyclohexyl, R^{b} is methyl, R^{b} is H and X is

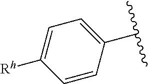

then R^{h} is not —CH═CHC(O)OMe, —CH═CHC(O)OEt, —CH═CHC(O)NMe₂, —CH═CHC(O)NH^{t}Bu, —CH═CHC(O)O^{t}Bu, —CH═CHC(O)OⁱPr, —CH═CHC(O)OCH₂Ph, —CH═CHC(O)OH, —CH═CHCH₂OMe, —CH═CHCH₂OEt or —CH═CHCH₂OPh.

In some embodiments of formula 1, R^{b} is substituted with substituents that improve the compounds water solubility. In certain embodiments, R^{b} is selected from alkyl, alkenyl, or cycloalkyl, each substituted with one or more hydroxyl groups.

In some embodiments, R^{a} is substituted with one or more hydroxyl groups, or a lower PEG group, such as PEG 2, PEG 3, PEG 4, PEG 5, PEG 6, PEG 8, PEG 10.

In some embodiments, X is not a benzopyran.

In particular embodiments, R is

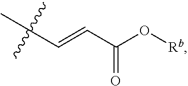

leading to compounds having the structure of formula 2

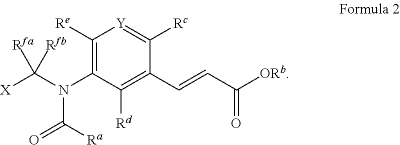

In some disclosed embodiments, the compounds having activity as FXR agonists have the structure of formula 3

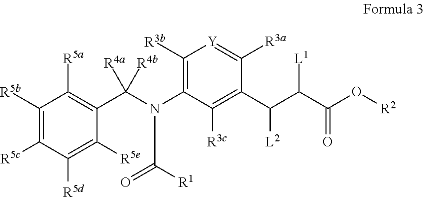

or a pharmaceutically acceptable salt thereof. With reference to formula 3, R¹ is selected from aryl, heteroaryl, heterocyclic, alkyl, alkenyl, cycloalkyl, cycloalkenyl or polycyclic; R² is selected from hydrogen, alkyl, alkenyl, or cycloalkyl; Y is selected from N, N—O (N-oxide) or C—R^{3d}; R^{3a}, R^{3b}, R^{3c} and R^{3d} are each independently selected from hydrogen, deuterium, halide, alkyl, alkenyl, alkoxy, alkylthio, amino, sulfonyl, aminosulfonyl, aminocarbonyl, acyl, hydroxyl or nitro; R^{4a} and R^{4b} are each independently selected from hydrogen (H), deuterium (D), halide or alkyl; L¹ and L² are independently selected from hydrogen, deuterium, alkyl, cycloalkyl, or together form a pi-bond; and R^{5a}, R^{5b}, R^{5c}, R^{5d} and R^{5e} are each independently selected from hydrogen, deuterium, halide, alkyl, alkenyl, alkoxy, alkylthio, amino, sulfonyl, aminosulfonyl, aminocarbonyl, acyl, aryl, heteroaryl, cycloalkyl, heterocyclyl, hydroxyl or nitro, or any two adjacent groups selected together form an aryl, heteroaryl, cycloalkyl or heterocyclic ring. In some embodiments when R² is hydrogen, the compounds have activity as FXR agonists. In other embodiments when R² is hydrogen, the compounds may have reduced or substantially no activity as FXR agonists.

Also with reference to formula 2, in some embodiments, none of R^{5a}, R^{5b}, R^{5c}, R^{5d} or R^{5e} is —R^{x}-L^{x}-R^{x2}, where R^{x} is selected from O, NR^{x3}, sulfonyl or S; R^{x3} is selected from H, alkyl, alkenyl, alkynyl, cycloalkyl, cycloalkenyl, or aryl; L^{x} is selected from a bond, alkylene, alkenylene, alkynylene, cycloalkyl, cycloalkenyl, heterocyclic, aryl, heteroaryl or CR^{x4}R^{x5}; R^{x4} and R^{x5} are each independently selected from H, D, halogen, alkyl, alkenyl, alkynyl, cycloalkyl, cycloalkenyl, —C(O)OR^{x6}, or —C(O)NR^{x6}R^{x7}; R^{x6} and R^{x7} are each independently selected from H, alkyl, alkenyl, alkynyl, cycloalkyl or cycloalkenyl; R^{x2} is selected from —C(O)L^{x2}R^{x8} or a carboxyl bioisostere; L^{x2} is a bond or NR^{x3}; R^{x8} is H, alkyl, alkenyl, alkynyl, cycloalkyl, cycloalkenyl, —OR^{x9}, N(R^{x9})₂, —C(O)R^{x9}, —S(O)₂R^{x9}, —C(O)OR^{x9}, —S(O)₂N(R^{x9})₂ or —C(O)N(R^{x9})₂; and each R^{x9} is independently selected from H, alkyl, alkenyl, alkynyl, cycloalkyl or cycloalkenyl; and

if L¹ and L² are both hydrogen or together form a pi-bond then at least one of the following conditions applies: Y is N or C-halogen; or R¹ is polycyclic; or R^{4a} is D; or R^{5a} is F, Cl, I; or R^{5d} and R^{5e} together form an aryl, heteroaryl, cycloalkyl or heterocyclic ring; or R^{5b} and R^{5c} together form an aryl, cycloalkyl, nitrogen-containing heterocyclic or nitrogen-containing heteroaryl ring, or any combination thereof.

In some embodiments, R² is substituted with one or more groups that improve the compounds water solubility. In certain embodiments, R² is substituted with one or more hydroxyl groups.

In some embodiments, one or more of R^{5a}, R^{5b}, R^{5c}, R^{5d} or R^{5e} is selected from

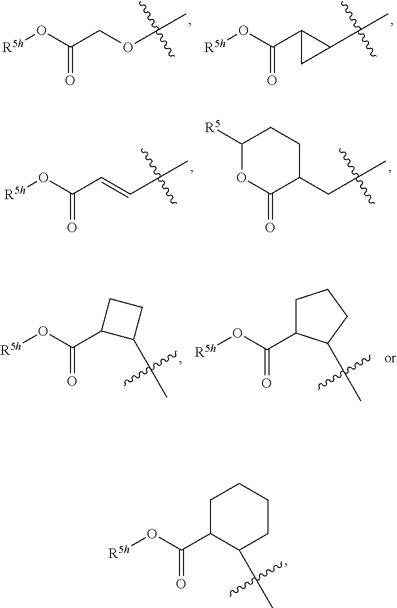

where R^{5h} is alkyl, alkenyl, hydrogen, cycloalkyl, or heterocyclic.

In particular embodiments, L¹ and L² together form a pi-bond, leading to compounds having the structure of formula 4

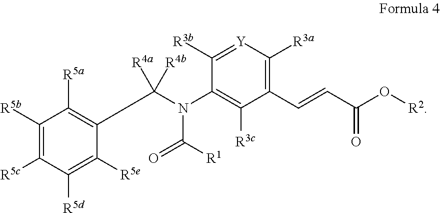

In some embodiments of general formula 4, Y is CR^{3d}, leading to compounds having the structure of formula 5

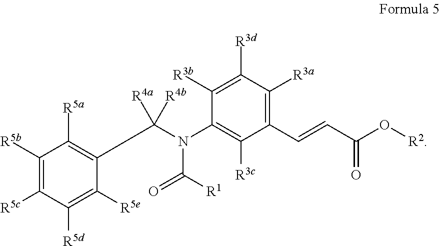

With reference to formula 5, R^{3d} or R^{5a} or both are halogen, such as F, Cl, Br or I, and R¹, R², R^{3a}, R^{3b}, R^{3c}, R^{4a}, R^{4b}, R^{5b}, R^{5c}, R^{5d} and R^{5e} are defined as for formula 3, above. In some working embodiments, R^{3d} or R^{5a} or both are F.

In other embodiments of formula 4, Y is N, resulting in compounds having the structure of formula 6

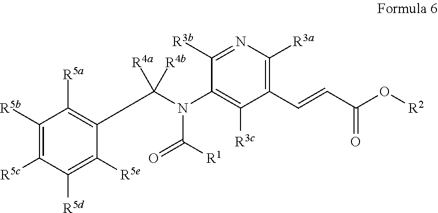

where R¹, R², R^{3a}, R^{3b}, R^{3c}, R^{4a}, R^{4b}, R^{5a}, R^{5b}, R^{5c}, R^{5d} and R^{5e} are defined as for formula 3.

In certain embodiments of formula 4, R¹ is polycyclic. This leads to compounds having the structure of formula 7

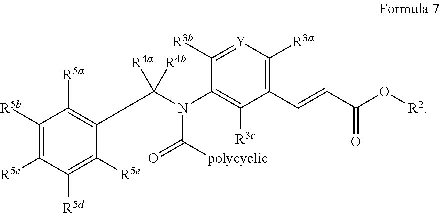

With reference to formula 7, R², R^{3a}, R^{3b}, R^{3c}, R^{4a}, R^{4b}, R^{5a}, R^{5b}, R^{5c}, R^{5d}, R^{5e} and Y are defined as for formula 3, above. In some examples, the polycyclic is selected from

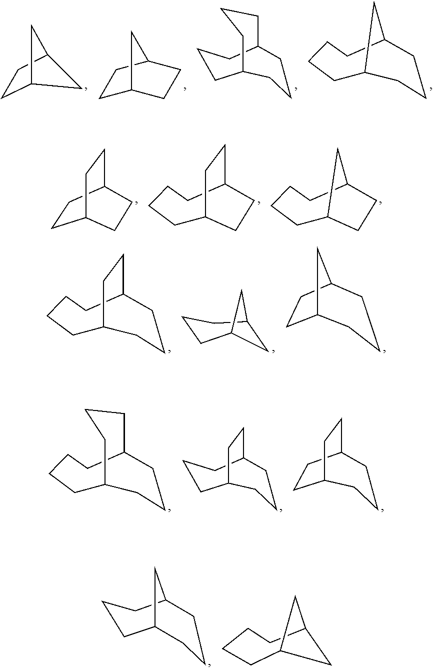

or adamantyl. In other examples, the polycyclic is selected from [2.1.1], [2.2.1], [3.3.3], [4.3.1], [2.2.2], [4.2.2], [4.2.1], [4.3.2], [3.1.1], [3.2.1], [4.3.3], [3.3.2], [3.2.2], [3.3.1], [4.1.1], or adamantyl. In certain working embodiments the polycyclic is

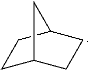

In certain embodiments of general formula 3, R^{5c} is a nitrogen-containing heteroaryl ring. Exemplary nitrogen-containing heteroaryl rings include, but are not limited to, pyridine, pyrazole, pyrrole, imidazole, oxazole, isoxazole, thiazole, isothiazole, triazole, pyrimidine, pyrazine, triazine, benzopyrazole, benzimidazole, indole, quinoline, indazole, purine, quinoxaline, and acridine. In particular embodiments, the compounds have the structure of formula 8

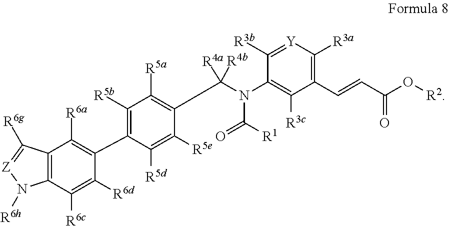

With reference to formula 8, R¹, R², R^{3a}, R^{3b}, R^{3c}, R^{4a}, R^{4b}, R^{5a}, R^{5b}, R^{5d}, R^{5e} and Y are defined as for formula 3, R^{6a}, R^{6c}, R^{6d} and R^{6g} are each independently selected from H, D, halogen or alkyl, R^{6h} is selected from H, D, alkyl, cycloalkyl, aryl or heteroaryl, and Z is selected from N, CH or C-alkyl. In certain working embodiments, Z is N and/or R^{6h} is methyl. In some examples R^{6a}, R^{6c}, R^{6d} and R^{6g} are all hydrogen. In particular examples, Y is C—R^{3d} and at least one of R^{3d} and R^{5a} is F.

In certain embodiments of general formula 5, R^{5c} is a 4-aminophenyl, leading to compounds having the structure of formula 9

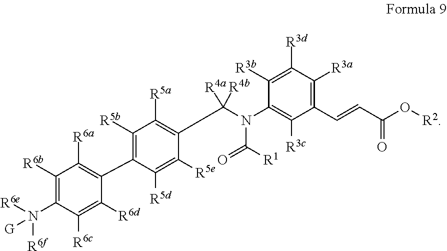

With reference to formula 9, R¹, R², R^{3a}, R^{3b}, R^{3c}, R^{3d}, R⁴, R^{5a}, R^{5b}, R^{5d} and R^{e} are defined as for formula 5, R^{6a}, R^{6b}, R^{6c} and R^{6d} are each independently selected from H, D, halogen or alkyl, G is a lone pair of electrons or an oxygen, and R^{6e} and R^{6f} are each independently selected from alkyl, H or cycloalkyl, with the provisos that R^{3d} or R^{5a} or both are halogen, or R⁴ is D, or R¹ is polycyclic, or any combination thereof. In working embodiments, R^{6e} and R^{6f} are both methyl.

In certain embodiments, compounds having formula 9 are N-oxides, leading to compounds having the structure of formula 10

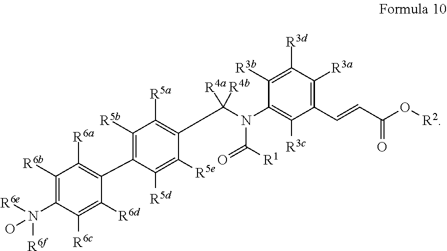

In particular examples of any of the above embodiments R^{4a} is D, R^{4b} is H, and/or R² is methyl. In other examples, both R^{4a} and R^{4b} are D. And in particular embodiments of formulas 3, 4, 5, 6, 7, 9 or 10, R¹ is cyclohexyl.

In some disclosed embodiments, compounds having activity as FXR agonists have the structure of formula 11

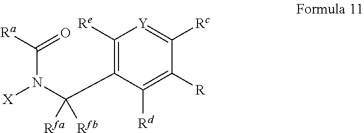

or a pharmaceutically acceptable salt thereof, wherein R^{a} is selected from aryl, heteroaryl, alkyl, alkenyl, cycloalkyl, heterocyclic, or polycyclic; R^{b} is selected from alkyl, alkenyl, or cycloalkyl; Y is CR^{g}, N or N—O (N-oxide); R^{c}, R^{d}, R^{e} and R^{g} are each independently selected from hydrogen, deuterium, halide, alkyl, alkenyl, alkoxy, alkylthio, amino, sulfonyl, aminosulfonyl, aminocarbonyl, cycloalkyl, heterocyclic, acyl, hydroxyl or nitro; R^{fa} and R^{fb} are each independently selected from hydrogen, deuterium, halide or alkyl; X is aryl, heterocyclic or heteroaryl; R is selected from

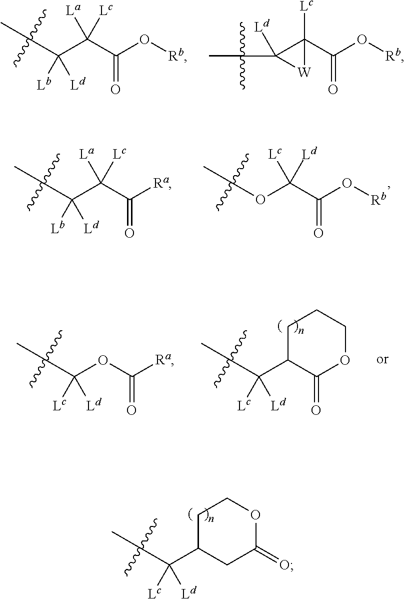

L^{a} and L^{b} are each independently selected from hydrogen, deuterium, alkyl or cycloalkyl, or together form a pi-bond; L^{c} and L^{d} are each independently selected from hydrogen, deuterium, alkyl or cycloalkyl; W is selected from O or —(C(L^{c})(L^{d}))ₛ-; s is 1, 2, 3, 4, 5 or 6; n is 0 or 1; and X is not substituted with —R^{x}-L^{x}-R^{x2}, where R^{x} is selected from O, NR^{x3}, sulfonyl or S; R^{x3} is selected from H, alkyl, alkenyl, alkynyl, cycloalkyl, cycloalkenyl, or aryl; L^{x} is selected from a bond, alkylene, alkenylene, alkynylene, cycloalkyl, cycloalkenyl, heterocyclic, aryl, heteroaryl or CR^{x4}R^{x5}; R^{x4} and R^{x5} are each independently selected from H, D, halogen, alkyl, alkenyl, alkynyl, cycloalkyl, cycloalkenyl, —C(O)OR^{x6}, or —C(O)NR⁶R⁷; R^{x6} and R^{x7} are each independently selected from H, alkyl, alkenyl, alkynyl, cycloalkyl or cycloalkenyl; R^{x2} is selected from —C(O)L^{x2}R^{x8} or a carboxyl bioisostere; L^{x2} is a bond or NR^{x3}; R^{x8} is H, alkyl, alkenyl, alkynyl, cycloalkyl, cycloalkenyl, —OR^{x9}, N(R^{x9})₂, —C(O)R^{x9}, —S(O)₂R^{x9}, —C(O)OR^{x9}, —S(O)₂N(R^{x9})₂ or —C(O)N(R^{x9})₂; and each R^{x9} is independently selected from H, alkyl, alkenyl, alkynyl, cycloalkyl or cycloalkenyl.

In some disclosed embodiments, the compounds having activity as FXR agonists have the structure of formula 12

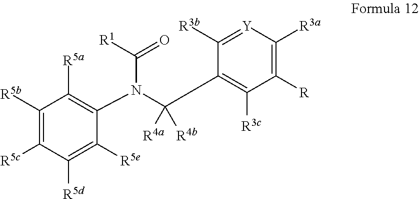

or a pharmaceutically acceptable salt thereof. With reference to formula 12, R is selected from

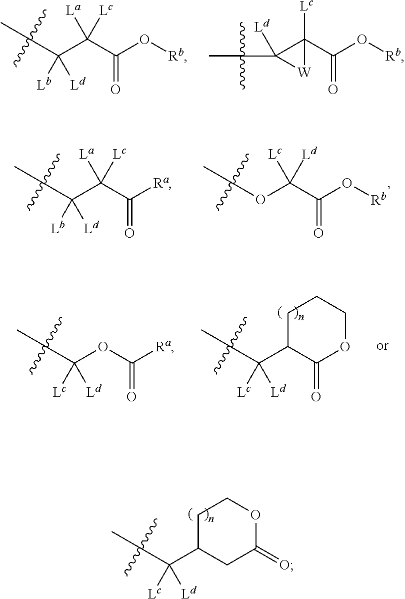

L^{a} and L^{b} are each independently selected from hydrogen, deuterium, alkyl or cycloalkyl, or together form a pi-bond; L^{c} and L^{d} are each independently selected from hydrogen, deuterium, alkyl or cycloalkyl; W is selected from O or —(C(L^{c})(L^{d}))ₛ-; s is 1, 2, 3, 4, 5 or 6; n is 0 or 1; R¹ is selected from aryl, heteroaryl, heterocyclic, alkyl, alkenyl, cycloalkyl, cycloalkenyl or polycyclic; R² is selected from alkyl, alkenyl, or cycloalkyl; Y is selected from N, N—O (N-oxide) or C—R^{3d}; R^{3a}, R^{3b}, R^{3c} and R^{3d} are each independently selected from hydrogen, deuterium, halide, alkyl, alkenyl, alkoxy, alkylthio, amino, sulfonyl, aminosulfonyl, aminocarbonyl, acyl, hydroxyl or nitro; R^{4a} and R^{4b} are each independently selected from hydrogen, deuterium, halide or alkyl; R^{5a}, R^{5b}, R^{5c}, R^{5d} and R^{5e} are each independently selected from hydrogen, deuterium, halide, alkyl, alkenyl, alkoxy, alkylthio, amino, sulfonyl, aminosulfonyl, aminocarbonyl, acyl, aryl, heteroaryl, cycloalkyl, heterocyclyl, hydroxyl or nitro, or any two adjacent groups selected together form an aryl, heteroaryl, cycloalkyl or heterocyclic ring; and none of R^{5a}, R^{5b}, R^{5c}, R^{5d} or R^{5e} is —R^{x}-L^{x}-R^{x2}, where R^{x} is selected from O, NR^{x3}, sulfonyl or S; R^{x3} is selected from H, alkyl, alkenyl, alkynyl, cycloalkyl, cycloalkenyl, or aryl; L^{x} is selected from a bond, alkylene, alkenylene, alkynylene, cycloalkyl, cycloalkenyl, heterocyclic, aryl, heteroaryl or CR^{x4}R^{x5}; R^{x4} and R^{x5} are each independently selected from H, D, halogen, alkyl, alkenyl, alkynyl, cycloalkyl, cycloalkenyl, —C(O)OR^{x6}, or —C(O)NR⁶R⁷; R^{x6} and R^{x7} are each independently selected from H, alkyl, alkenyl, alkynyl, cycloalkyl or cycloalkenyl; R^{x2} is selected from —C(O)L^{x2}R^{x8} or a carboxyl bioisostere; L^{x2} is a bond or NR^{x3}; R^{x8} is H, alkyl, alkenyl, alkynyl, cycloalkyl, cycloalkenyl, —OR^{x9}, N(R^{x9})₂, —C(O)R^{x9}, —S(O)₂R^{x9}, —C(O)OR⁹, —S(O)₂N(R^{x9})₂ or —C(O)N(R^{x9})₂; and each R^{x9} is independently selected from H, alkyl, alkenyl, alkynyl, cycloalkyl or cycloalkenyl.

In some other embodiments, the compounds having activity as FXR agonists have the structure of formula 13

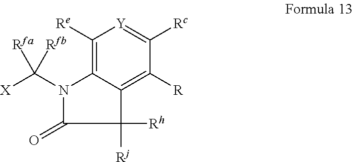

or a pharmaceutically acceptable salt thereof, wherein R is selected from

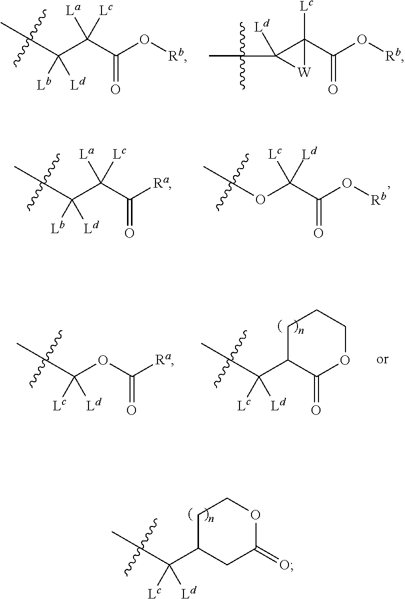

L^{a} and L^{b} are each independently selected from hydrogen, deuterium, alkyl or cycloalkyl, or together form a pi-bond; L^{c} and L^{d} are each independently selected from hydrogen, deuterium, alkyl or cycloalkyl; W is selected from O or —(C(L^{c})(L^{d}))ₛ-; s is 1, 2, 3, 4, 5 or 6; n is 0 or 1; R^{b} is selected from alkyl, alkenyl, or cycloalkyl; Y is CR^{g}, N or N—O (N-oxide); R^{c}, R^{e} and R^{g} are each independently selected from hydrogen, deuterium, halide, alkyl, alkenyl, alkoxy, alkylthio, amino, sulfonyl, aminosulfonyl, aminocarbonyl, cycloalkyl, heterocyclic, acyl, hydroxyl or nitro; R^{fa} and R^{fb} are each independently selected from hydrogen, deuterium, halide or alkyl; R^{h} and R^{j} are each independently selected from hydrogen, deuterium, halide, alkyl, cycloalkyl, heterocycloalkyl, alkenyl, aryl or heteroaryl; X is aryl, heterocyclic or heteroaryl; and X is not substituted with —R^{x}-L^{x}-R^{x2}, where R^{x} is selected from O, NR^{x3}, sulfonyl or S; R^{x3} is selected from H, alkyl, alkenyl, alkynyl, cycloalkyl, cycloalkenyl, or aryl; L^{x} is selected from a bond, alkylene, alkenylene, alkynylene, cycloalkyl, cycloalkenyl, heterocyclic, aryl, heteroaryl or CR^{x4}R^{x5}; R^{x4} and R^{x5} are each independently selected from H, D, halogen, alkyl, alkenyl, alkynyl, cycloalkyl, cycloalkenyl, —C(O)OR^{x6}, or —C(O)NR⁶R⁷; R^{x6} and R^{x7} are each independently selected from H, alkyl, alkenyl, alkynyl, cycloalkyl or cycloalkenyl; R^{x2} is selected from —C(O)L^{x2}R^{x8} or a carboxyl bioisostere; L^{x2} is a bond or NR^{x3}; R^{x8} is H, alkyl, alkenyl, alkynyl, cycloalkyl, cycloalkenyl, —OR^{x9}, N(R^{x9})₂, —C(O)R^{x9}, —S(O)₂R^{x9}, —C(O)OR^{x9}, —S(O)₂N(R⁹)₂ or —C(O)N(R^{x9})₂; and each R^{x9} is independently selected from H, alkyl, alkenyl, alkynyl, cycloalkyl or cycloalkenyl.

In some embodiments, prodrugs of compounds having activity as FXR agonists have the structure of formula 14

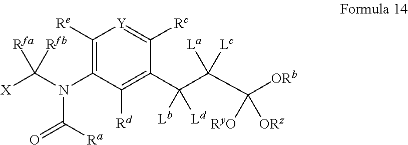

or a pharmaceutically acceptable salt thereof, wherein R^{a} is selected from aryl, heteroaryl, alkyl, alkenyl, cycloalkyl, heterocyclic, or polycyclic; R^{b} is selected from alkyl, alkenyl, or cycloalkyl; Y is CR^{g}, N or N—O (N-oxide); R^{c}, R^{d}, R^{e} and R^{g} are each independently selected from hydrogen, deuterium, halide, alkyl, alkenyl, alkoxy, alkylthio, amino, sulfonyl, aminosulfonyl, aminocarbonyl, acyl, hydroxyl or nitro; R^{fa} and R^{fb} are each independently selected from hydrogen, deuterium, halide or alkyl; X is aryl, heterocyclic or heteroaryl; R^{y} and R^{z} are selected from alkyl, cycloalkyl, heterocyclic alkyl, aryl, or heteroaryl, or R^{y} and R^{z} may together form a cycloheteroalkyl ring; L^{a} and L^{b} are independently H, D or alkyl or together form a π-bond, a cyclopropyl or an epoxide ring; L^{c} and L^{d} are independently H, D or alkyl; and X is not substituted with —R^{x}-L^{x}-R^{x2}, where R^{x} is selected from O, NR^{x3}, sulfonyl or S; R^{x3} is selected from H, alkyl, alkenyl, alkynyl, cycloalkyl, cycloalkenyl, or aryl; L^{x} is selected from a bond, alkylene, alkenylene, alkynylene, cycloalkyl, cycloalkenyl, heterocyclic, aryl, heteroaryl or CR^{x4}R^{x5}; R^{x4} and R^{x5} are each independently selected from H, D, halogen, alkyl, alkenyl, alkynyl, cycloalkyl, cycloalkenyl, —C(O)OR^{x6}, or —C(O)NR^{x6}R^{x7}; R^{x6} and R^{x7} are each independently selected from H, alkyl, alkenyl, alkynyl, cycloalkyl or cycloalkenyl; R^{x2} is selected from —C(O)L^{x2}R^{x8} or a carboxyl bioisostere; L^{x2} is a bond or NR^{x3}; R^{x8} is H, alkyl, alkenyl, alkynyl, cycloalkyl, cycloalkenyl, —OR^{x9}, N(R^{x9})₂, —C(O)R^{x9}, —S(O)₂R⁹, —C(O)OR⁹, —S(O)₂N(R^{x9})₂ or —C(O)N(R^{x9})₂; and each R^{x9} is independently selected from H, alkyl, alkenyl, alkynyl, cycloalkyl or cycloalkenyl.

In particular embodiments of formula 14, R^{y} and R^{z} together form a 5-membered heteroalkyl ring substituted with an ascorbate moiety, leading to compounds having the structure of formula 15

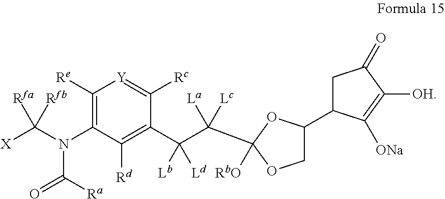

In other embodiments, prodrugs of compounds having activity as FXR agonists have the structure of formula 16

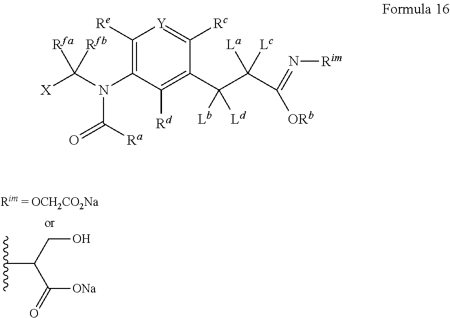

or a pharmaceutically acceptable salt thereof, wherein R^{a} is selected from aryl, heteroaryl, alkyl, alkenyl, cycloalkyl, heterocyclic, or polycyclic; R^{b} is selected from alkyl, alkenyl, or cycloalkyl; Y is CR^{g}, N or N—O (N-oxide); R^{c}, R^{d}, R^{e} and R^{g} are each independently selected from hydrogen, deuterium, halide, alkyl, alkenyl, alkoxy, alkylthio, amino, sulfonyl, aminosulfonyl, aminocarbonyl, acyl, hydroxyl or nitro; R^{fa} and R^{fb} are each independently selected from hydrogen, deuterium, halide or alkyl; X is aryl, heterocyclic or heteroaryl, L^{a} and L^{b} are independently H, D or alkyl or together form a π-bond, a cyclopropyl or an epoxide ring; L^{c} and L^{d} are independently H, D or alkyl; and X is not substituted with —R^{x}-L^{x}-R^{x2}, where R^{x} is selected from O, NR^{x3}, sulfonyl or S; R^{x3} is selected from H, alkyl, alkenyl, alkynyl, cycloalkyl, cycloalkenyl, or aryl; L^{x} is selected from a bond, alkylene, alkenylene, alkynylene, cycloalkyl, cycloalkenyl, heterocyclic, aryl, heteroaryl or CR^{x4}R^{x5}; R^{x4} and R^{x5} are each independently selected from H, D, halogen, alkyl, alkenyl, alkynyl, cycloalkyl, cycloalkenyl, —C(O)OR^{x6}, or —C(O)NR^{x6}R^{x7}; R^{x6} and R^{x7} are each independently selected from H, alkyl, alkenyl, alkynyl, cycloalkyl or cycloalkenyl; R^{x2} is selected from —C(O)L^{x2}R^{x8} or a carboxyl bioisostere; L^{x2} is a bond or NR^{x3}; R^{x8} is H, alkyl, alkenyl, alkynyl, cycloalkyl, cycloalkenyl, —OR^{x9}, N(R^{x9})₂, —C(O)R^{x9}, —S(O)₂R^{x9}, —C(O)OR^{x9}, —S(O)₂N(R^{x9})₂ or —C(O)N(R^{x9})₂; and each R^{x9} is independently selected from H, alkyl, alkenyl, alkynyl, cycloalkyl or cycloalkenyl.

In still further embodiments, prodrugs of compounds having activity as FXR agonists have the structure of formula 17

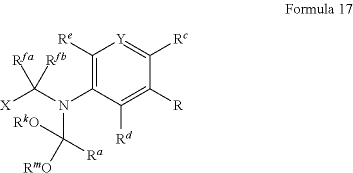

or a pharmaceutically acceptable salt thereof, wherein R is selected from

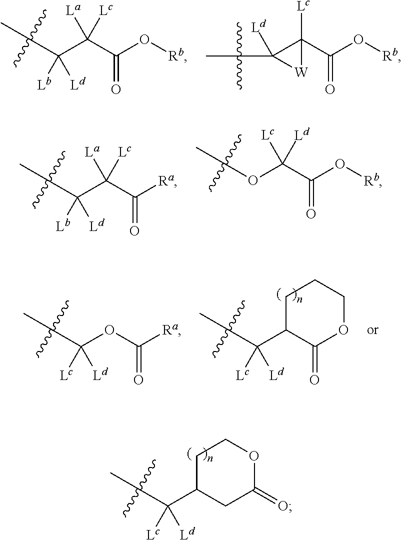

L^{a} and L^{b} are each independently selected from hydrogen, deuterium, alkyl or cycloalkyl, or together form a pi-bond; L^{c} and L^{d} are each independently selected from hydrogen, deuterium, alkyl or cycloalkyl; W is selected from O or —(C(L^{c})(L^{d}))ₛ-; s is 1, 2, 3, 4, 5 or 6; n is 0 or 1; R^{a} is selected from aryl, heteroaryl, alkyl, alkenyl, cycloalkyl, heterocyclic, or polycyclic; R^{b} is selected from alkyl, alkenyl, or cycloalkyl; Y is CR^{g}, N or N—O (N-oxide); R^{c}, R^{d}, R^{e} and R^{g} are each independently selected from hydrogen, deuterium, halide, alkyl, alkenyl, alkoxy, alkylthio, amino, sulfonyl, aminosulfonyl, aminocarbonyl, cycloalkyl, heterocyclic, acyl, hydroxyl or nitro; R^{fa} and R^{fb} are each independently selected from hydrogen, deuterium, halide or alkyl; R^{k} and R^{m} are independently selected from H, alkyl, aryl, cycloalkyl, heterocycloalkyl, heteroaryl, or together R^{k} and R^{m} form a cycloalkyl or heterocycloalkyl ring; X is aryl, heterocyclic or heteroaryl; and X is not substituted with —R^{x}-L^{x}-R^{x2}, where R^{x} is selected from O, NR^{x3}, sulfonyl or S; R^{x3} is selected from H, alkyl, alkenyl, alkynyl, cycloalkyl, cycloalkenyl, or aryl; L^{x} is selected from a bond, alkylene, alkenylene, alkynylene, cycloalkyl, cycloalkenyl, heterocyclic, aryl, heteroaryl or CR^{x4}R^{x5}; R^{x4} and R^{x5} are each independently selected from H, D, halogen, alkyl, alkenyl, alkynyl, cycloalkyl, cycloalkenyl, —C(O)OR^{x6}, or —C(O)NR^{x6}R^{x7}; R^{x6} and R^{x7} are each independently selected from H, alkyl, alkenyl, alkynyl, cycloalkyl or cycloalkenyl; R^{x2} is selected from —C(O)L^{x2}R^{x8} or a carboxyl bioisostere; L^{x2} is a bond or NR^{x3}; R^{x8} is H, alkyl, alkenyl, alkynyl, cycloalkyl, cycloalkenyl, —OR^{x9}, N(R^{x9})₂, —C(O)R^{x9}, —S(O)₂R^{x9}, —C(O)OR^{x9}, —S(O)₂N(R^{x9})₂ or —C(O)N(R^{x9})₂; and each R^{x9} is independently selected from H, alkyl, alkenyl, alkynyl, cycloalkyl or cycloalkenyl.

In some embodiments of formula 17 R^{k} and R^{m} together form a 5-membered ring, leading to compounds having a structure of formula 18

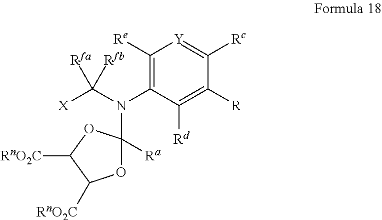

or pharmaceutically acceptable salt thereof, wherein each Rⁿ is independently selected from H, alkyl, or a metal salt such as Na, K, or Li.

In any of the above embodiments of formulas 1, 9 or 13-18, X is heteroaryl or heterocyclic, and in particular embodiments, X is pyridine or piperidine.

In other embodiments of formulas 1, 10, 11, or 13-18, X is a phenyl substituted with an aryl or heteroaryl group. In certain embodiments, X is a phenyl substituted with the aryl or heteroaryl group selected from benzoxazine, dihydrobenzoxazine, quinoxaline, tetrahydroquinoxaline, benzodioxane, benzothiazine, dihydrobenzothiazine, dihydrobenzothiazine-1,1-dioxide, benzodithiine, benzodithiine-1,1,4,4-tetraoxide, benzofuran, benzothiophene, indole, benzisoxazole, indazole, benzotriazole, benzimidazole, benzoxazole, benzthiazole or benzisothiazole. In particular embodiments, X is selected from

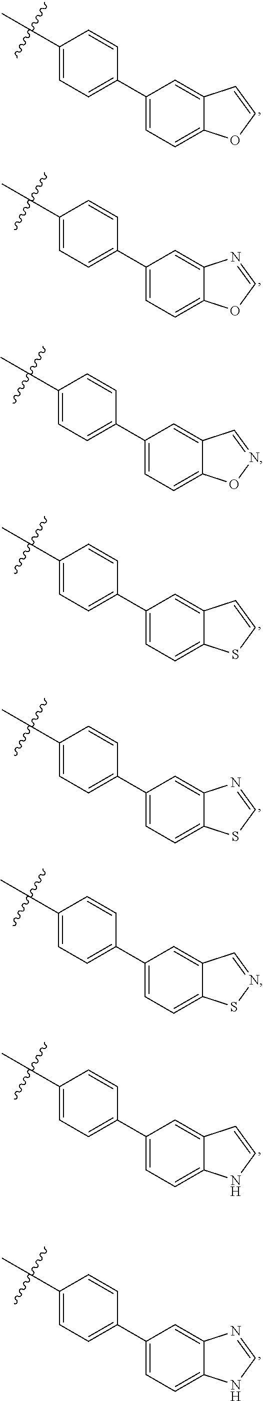

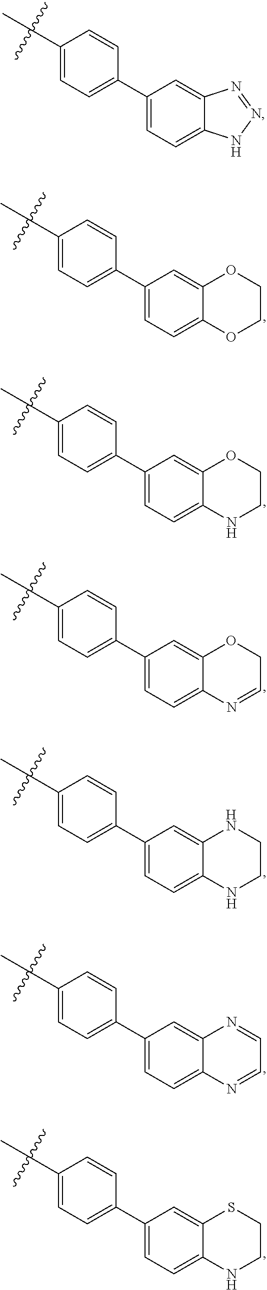

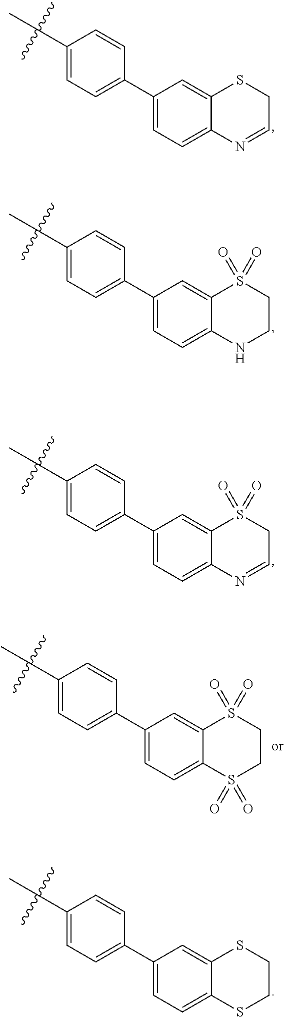 [SMILES: *c1ccc(-c2ccc3c(c2)SCC=N3)cc1 ; *c1ccc(-c2ccc3c(c2)S(=O)(=O)CCN3)cc1 ; *c1ccc(-c2ccc3c(c2)S(=O)(=O)CC=N3)cc1 ; *c1ccc(-c2ccc3c(c2)S(=O)(=O)CCS3(=O)=O)cc1 ; *c1ccc(-c2ccc3c(c2)SCCS3)cc1]

A person of ordinary skill in the art will appreciate that compounds of any of the above embodiments may have one or more stereocenter, and that each stereocenter independently may have an R or S configuration.

A person of ordinary skill in the art will appreciate that prodrug compounds satisfying one of formulas 14-18 may also have intrinsic activity as FXR agonists, as well as acting as a prodrug for a compound having FXR activity.

Exemplary compounds having activity as FXR agonists and satisfying one or more of the general formulas 1-18 are provided below.

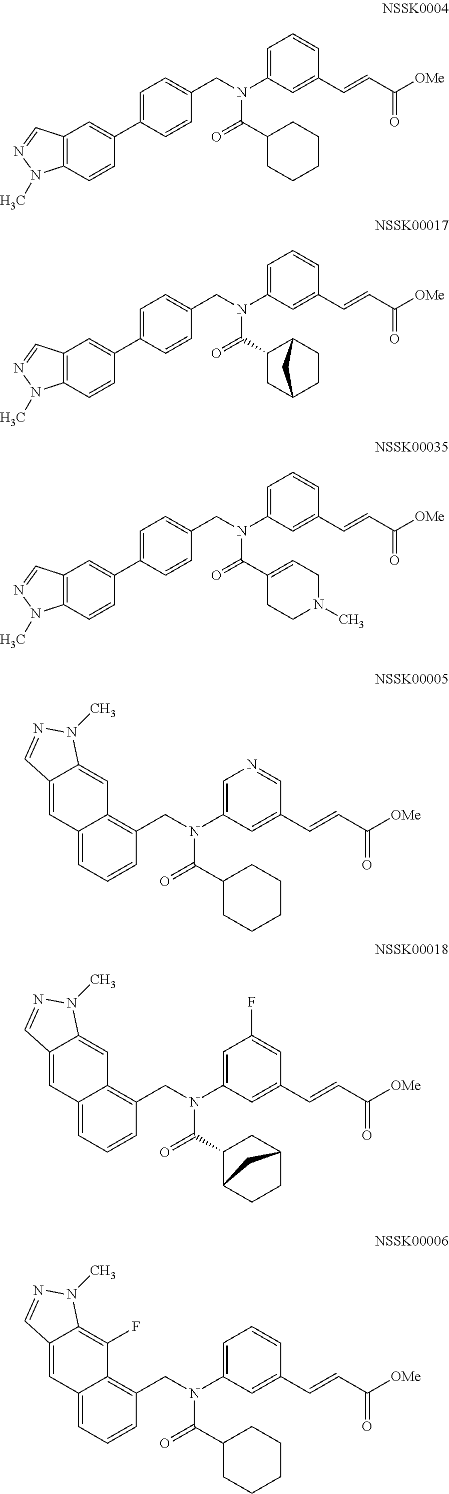

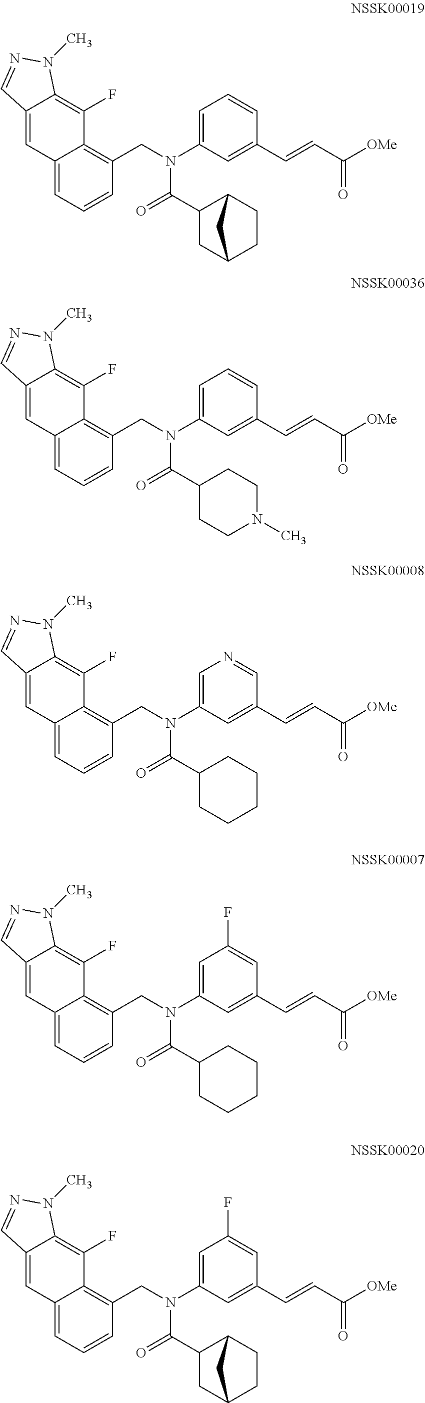

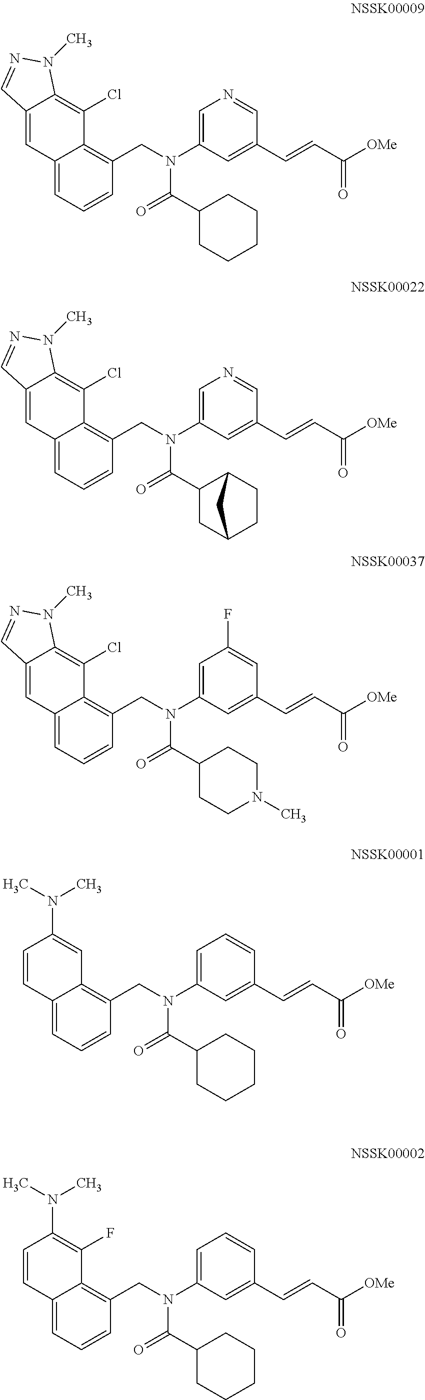

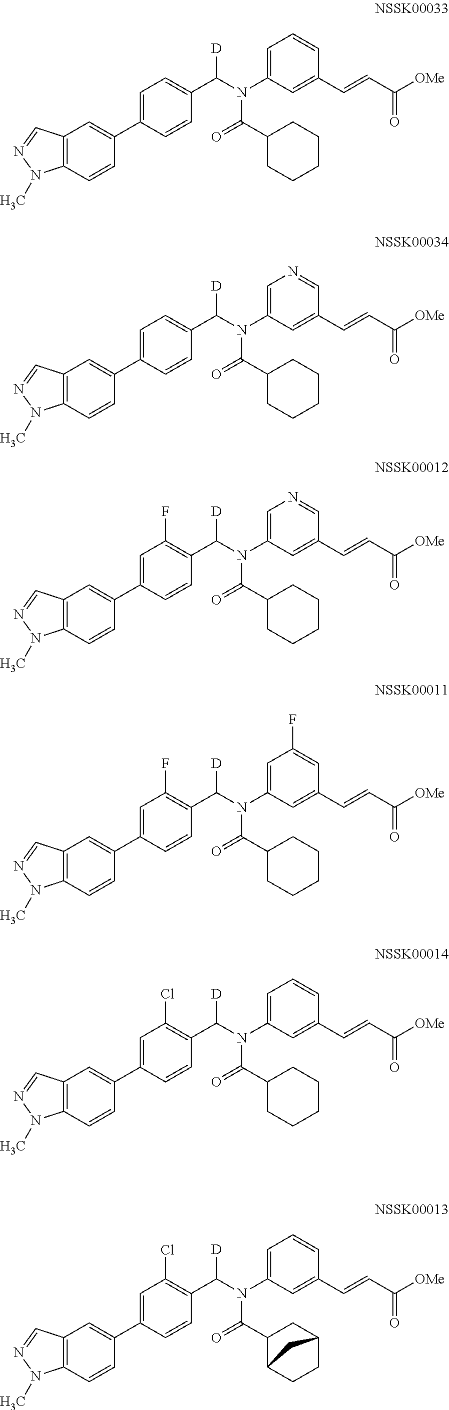

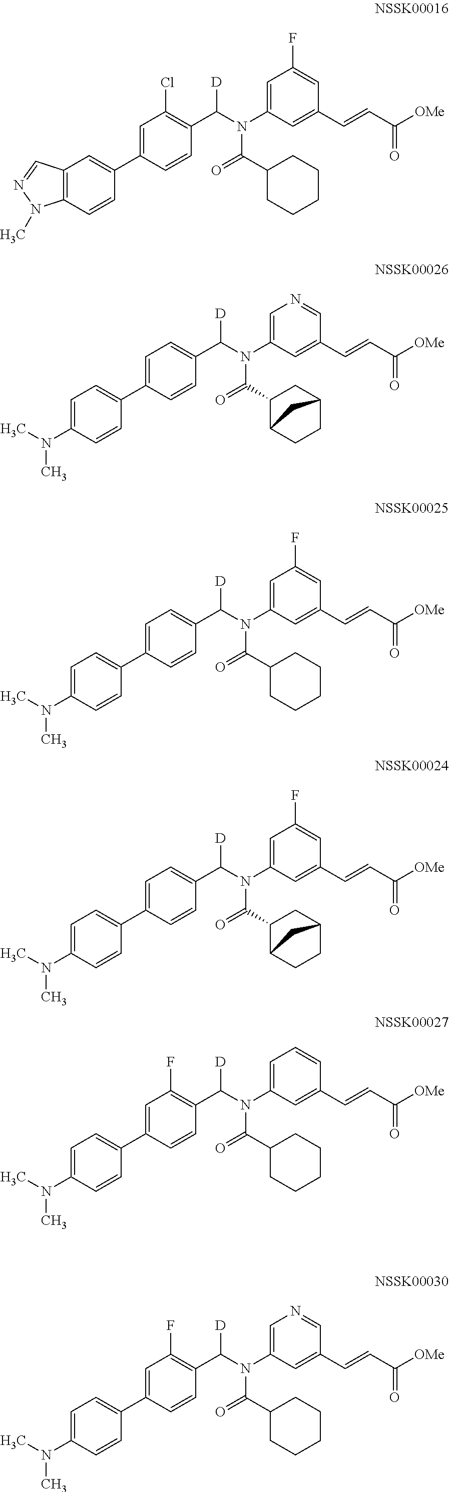

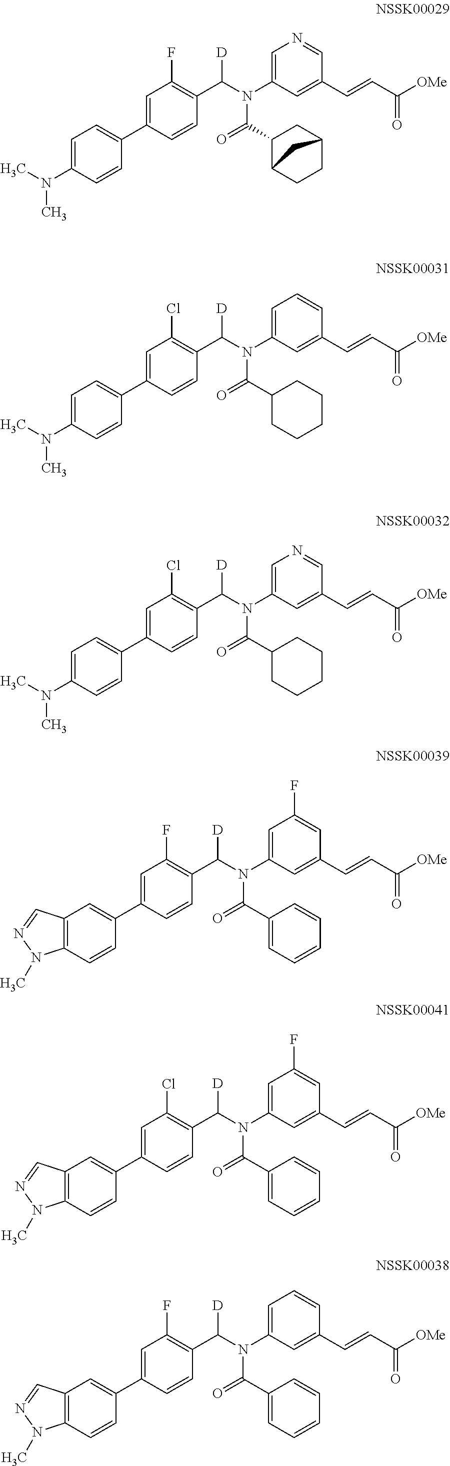

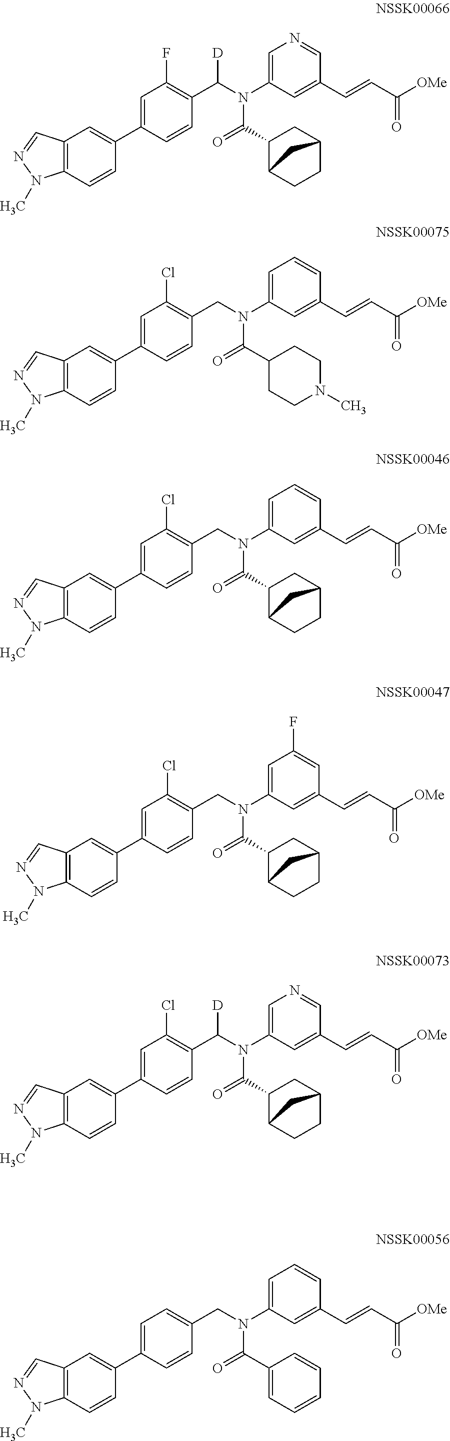

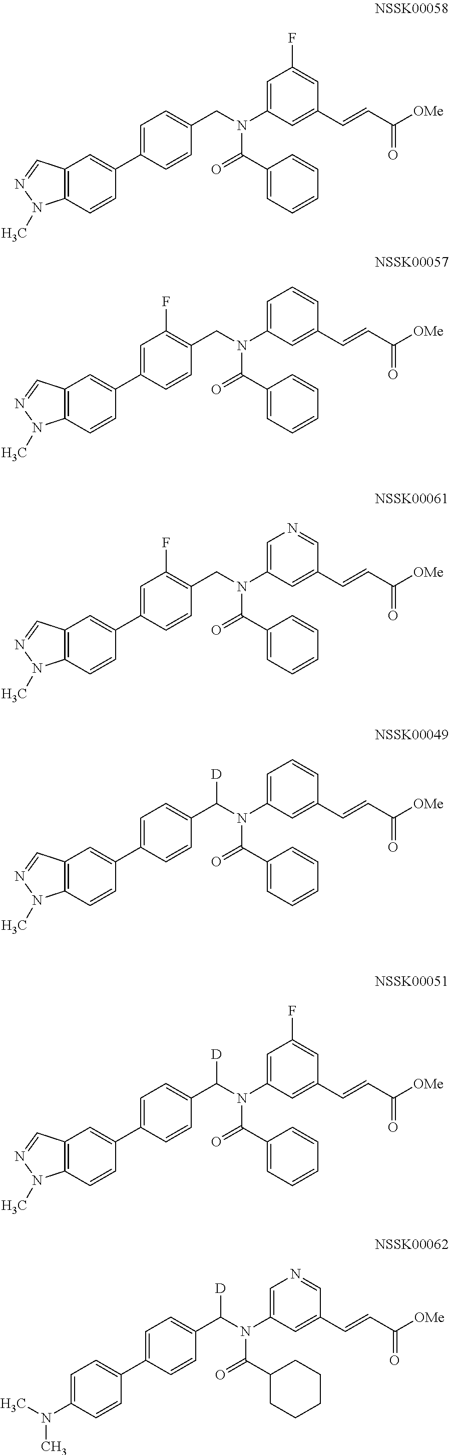

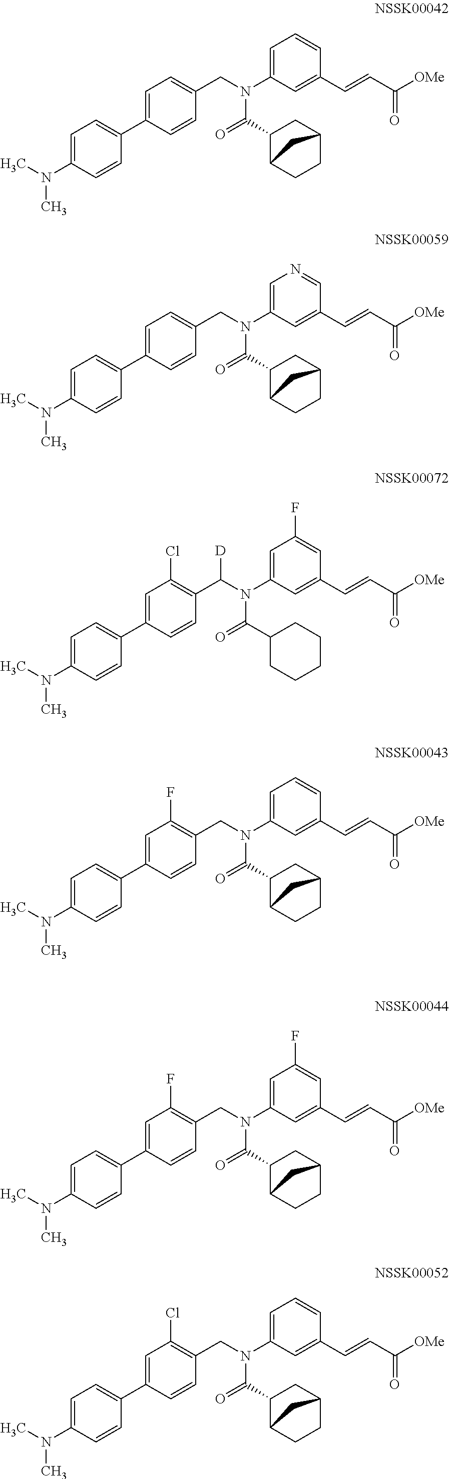

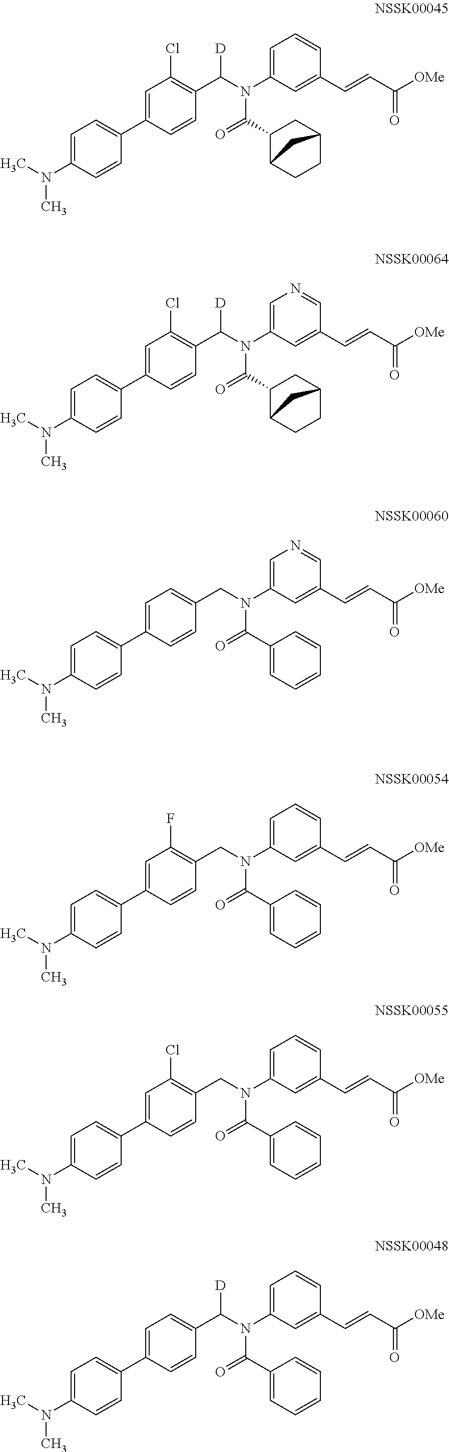

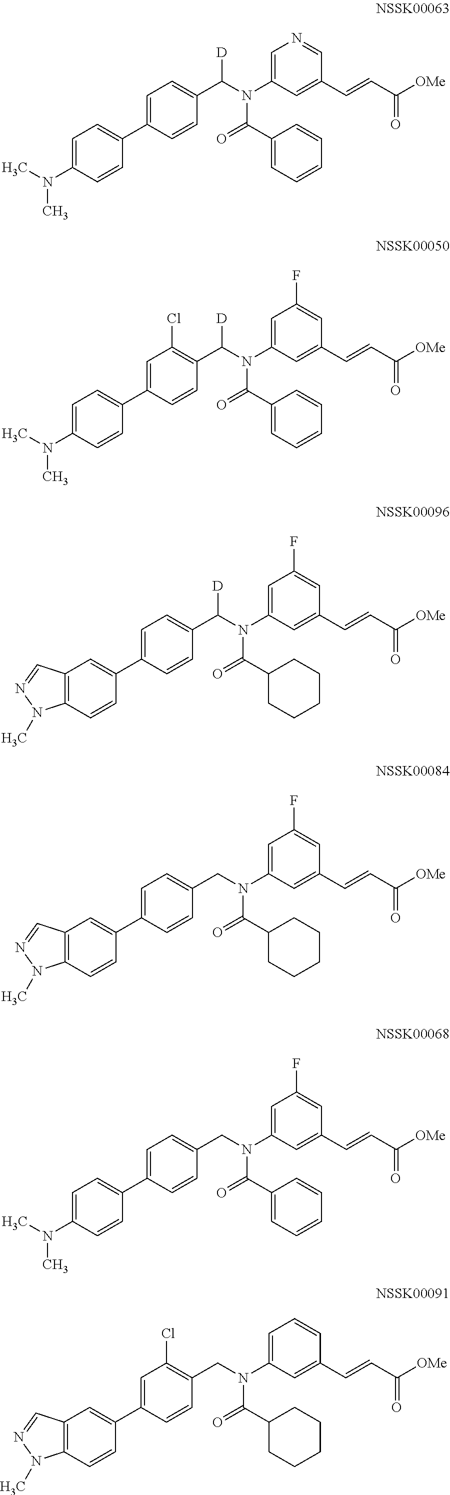

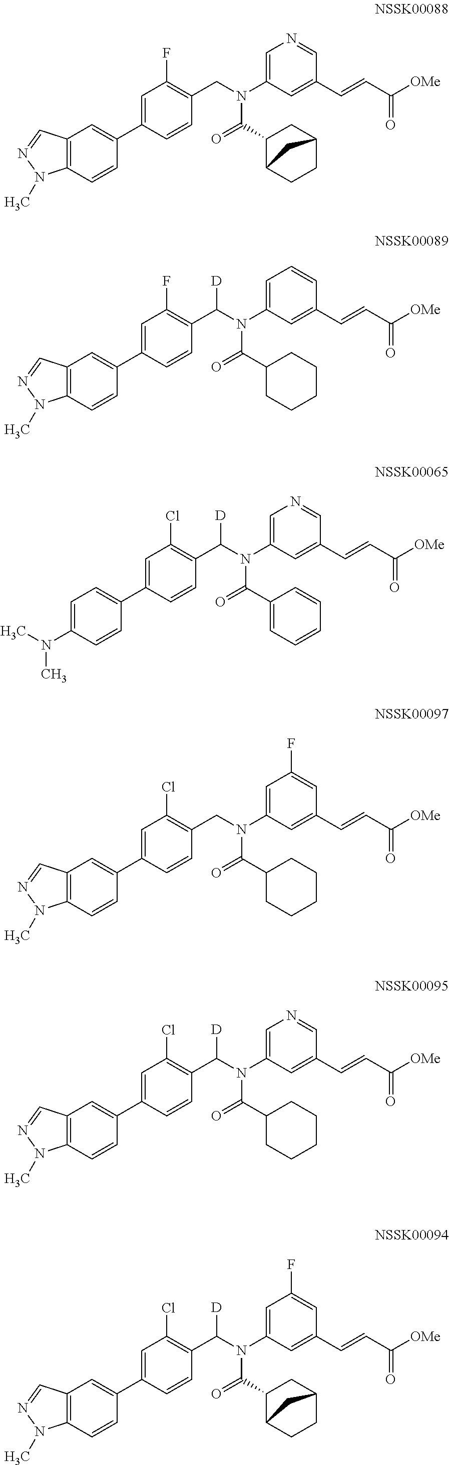

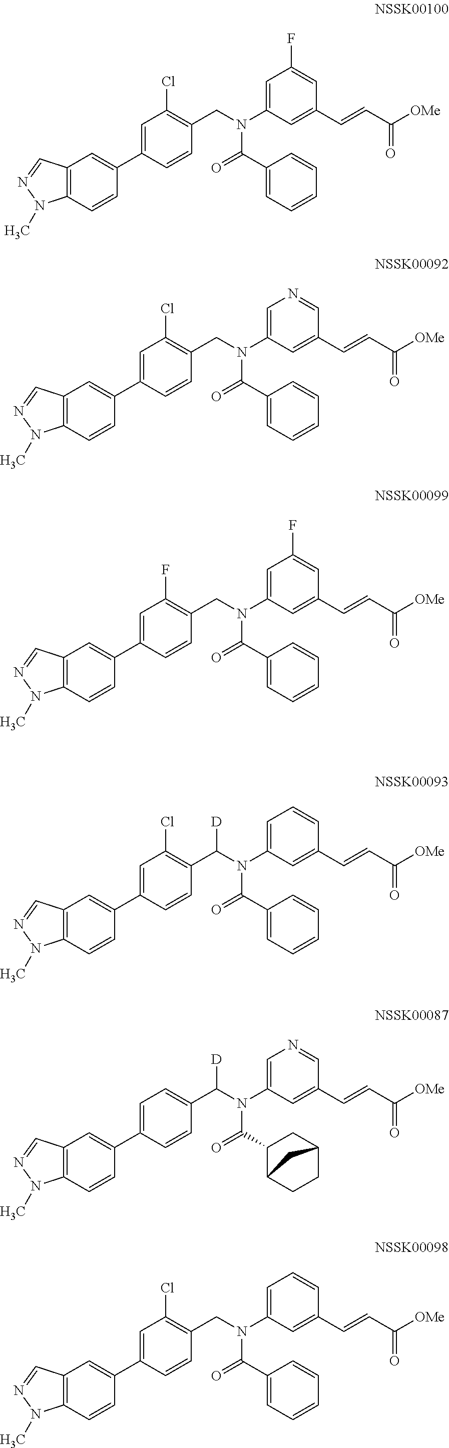

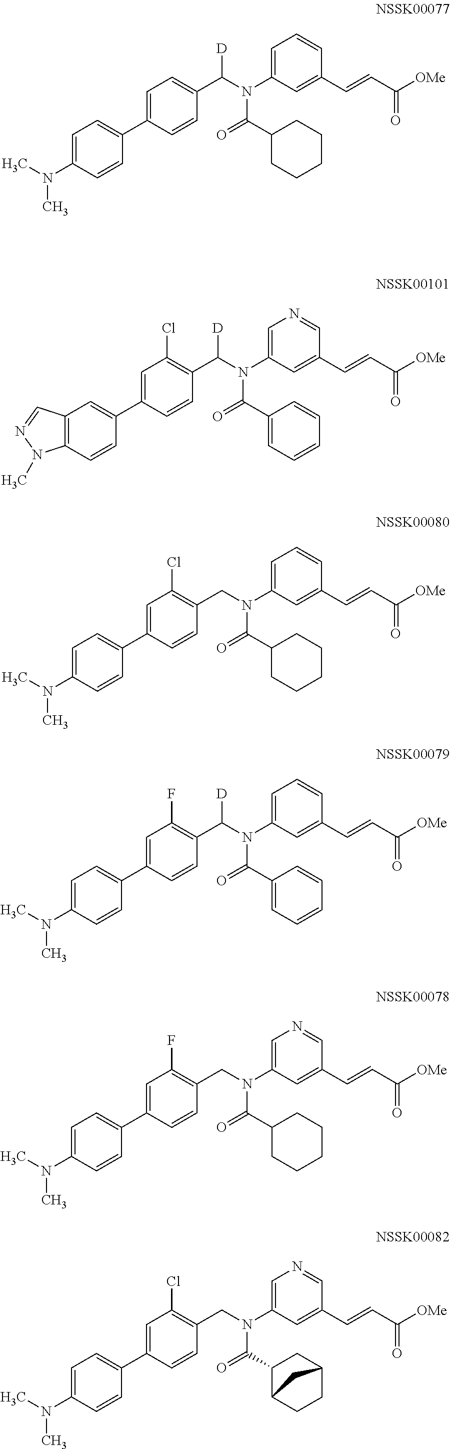

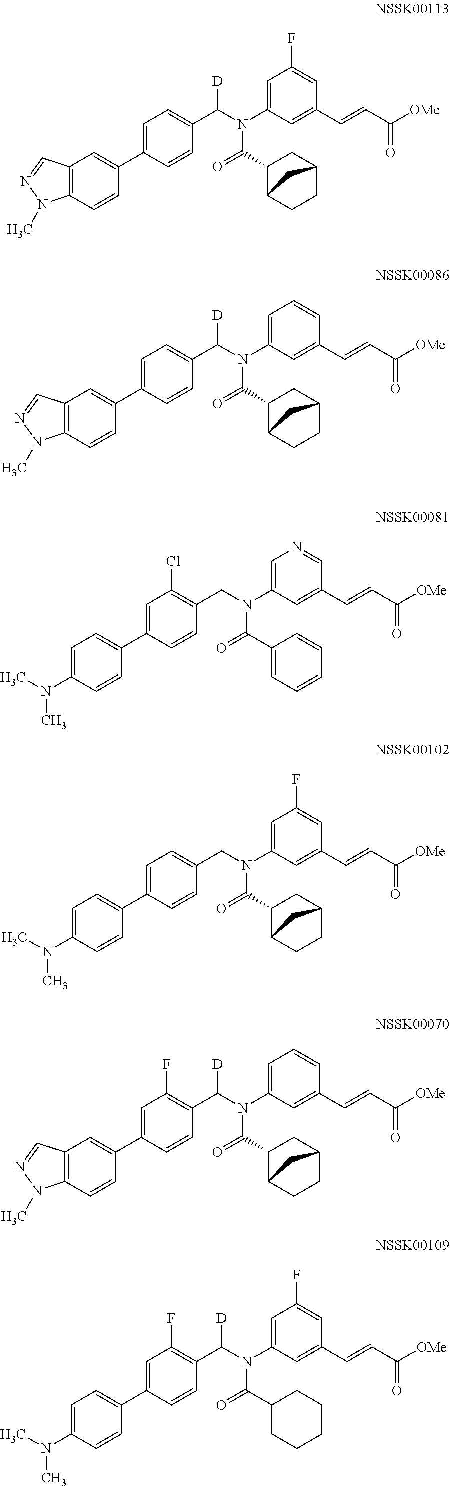

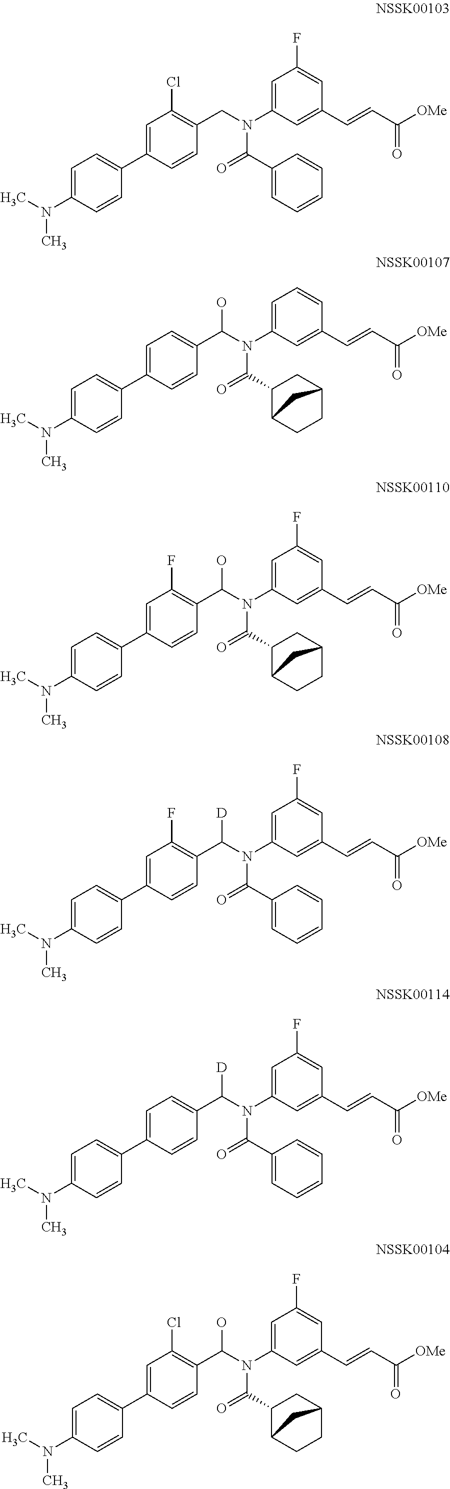

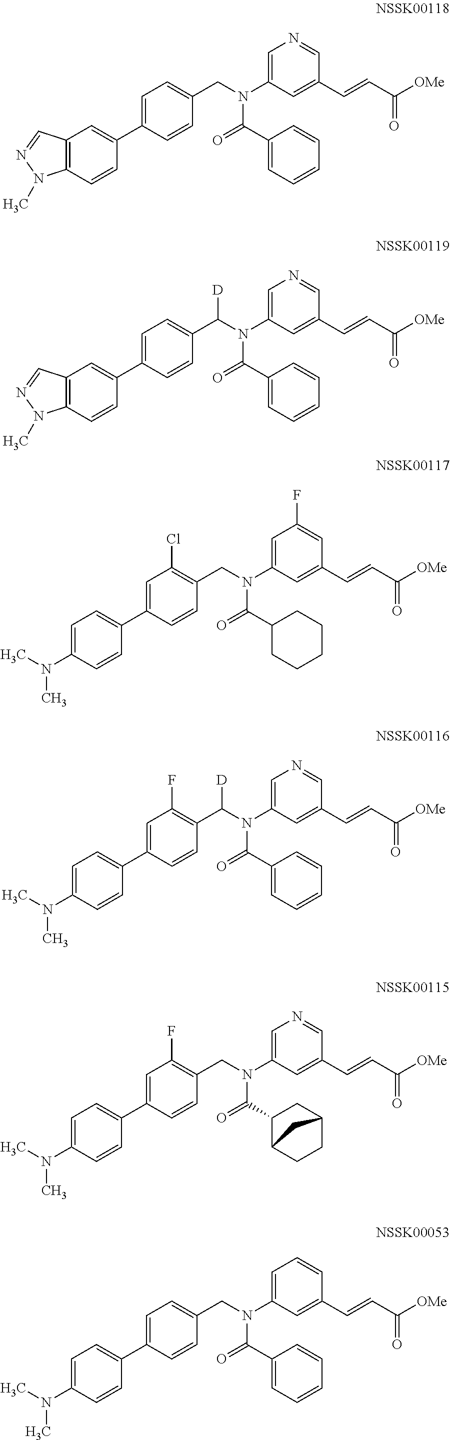

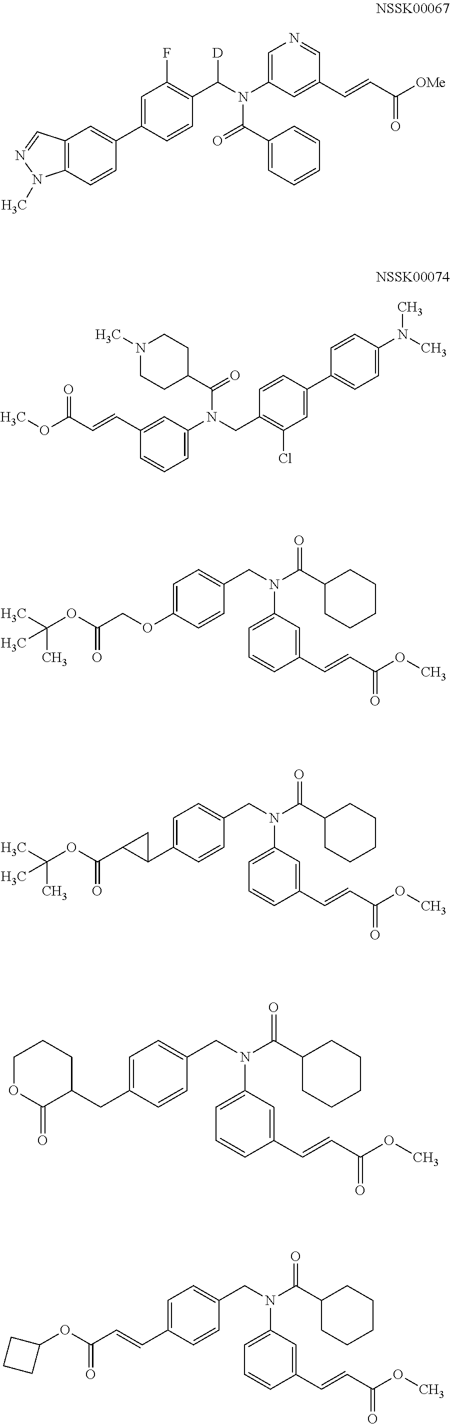

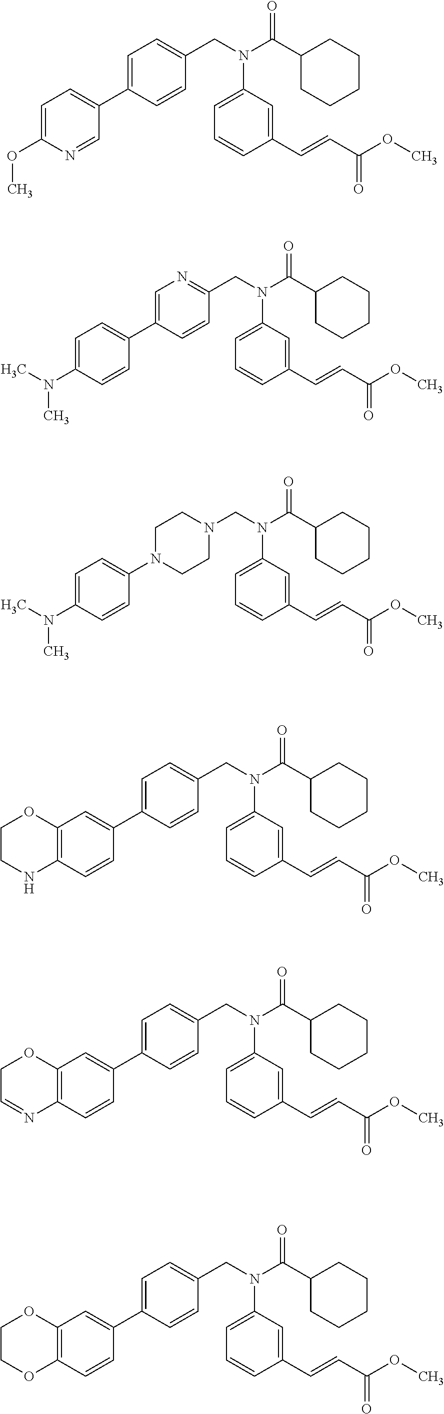

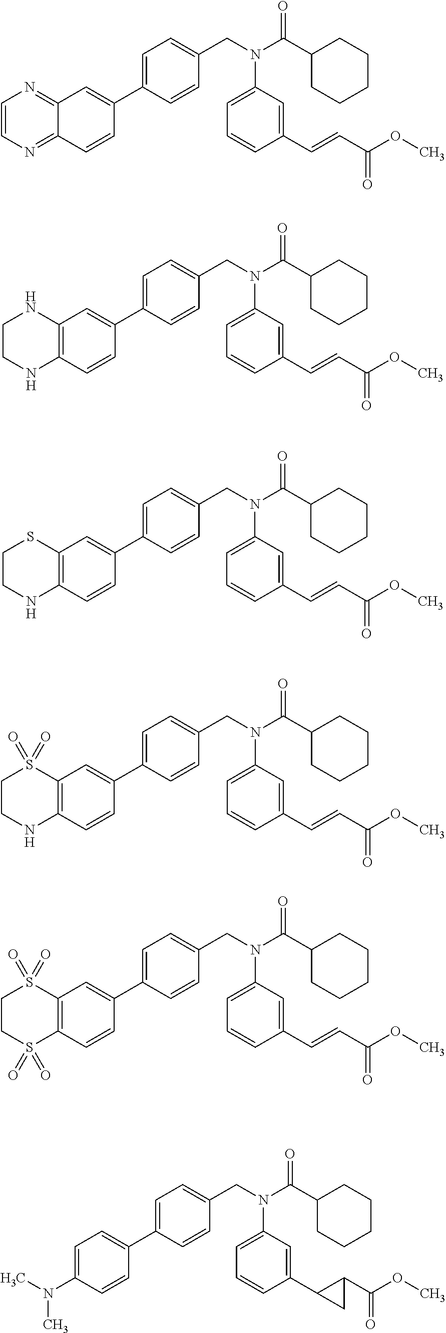

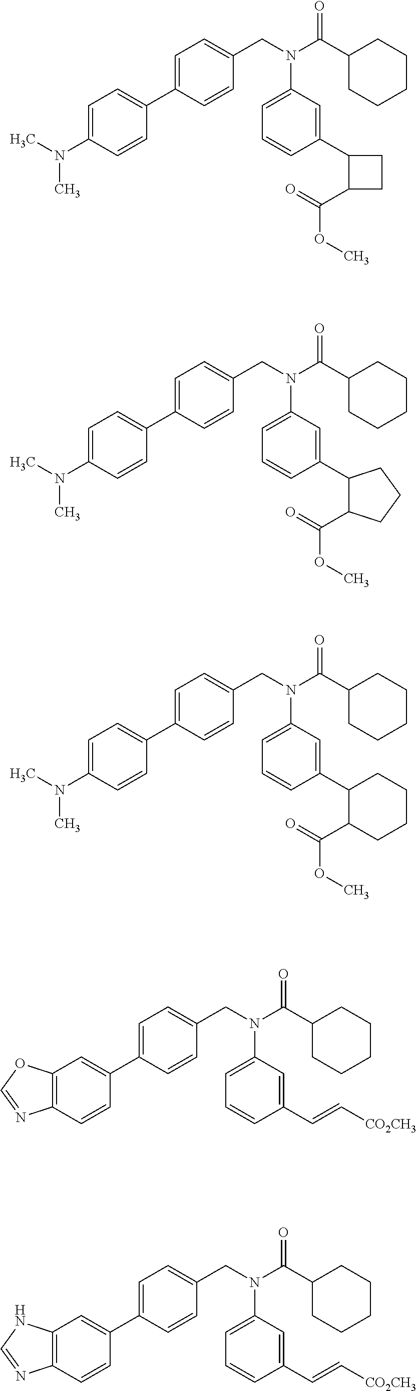

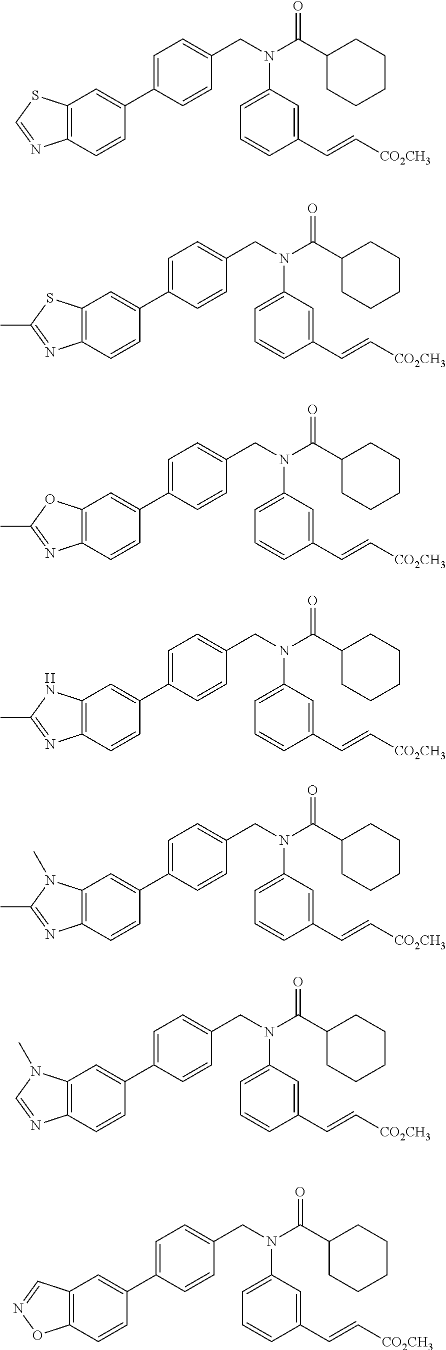

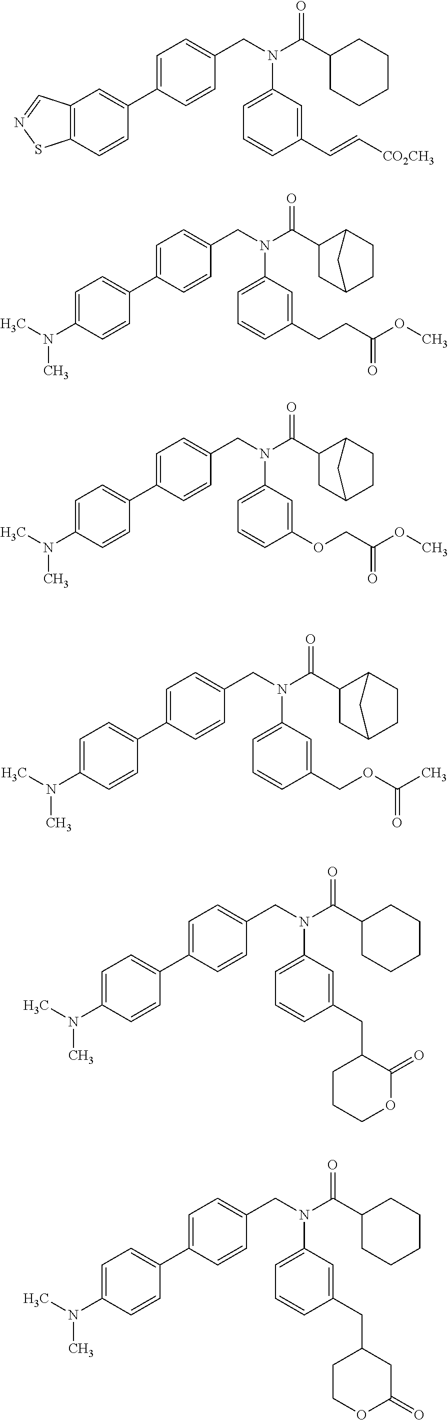

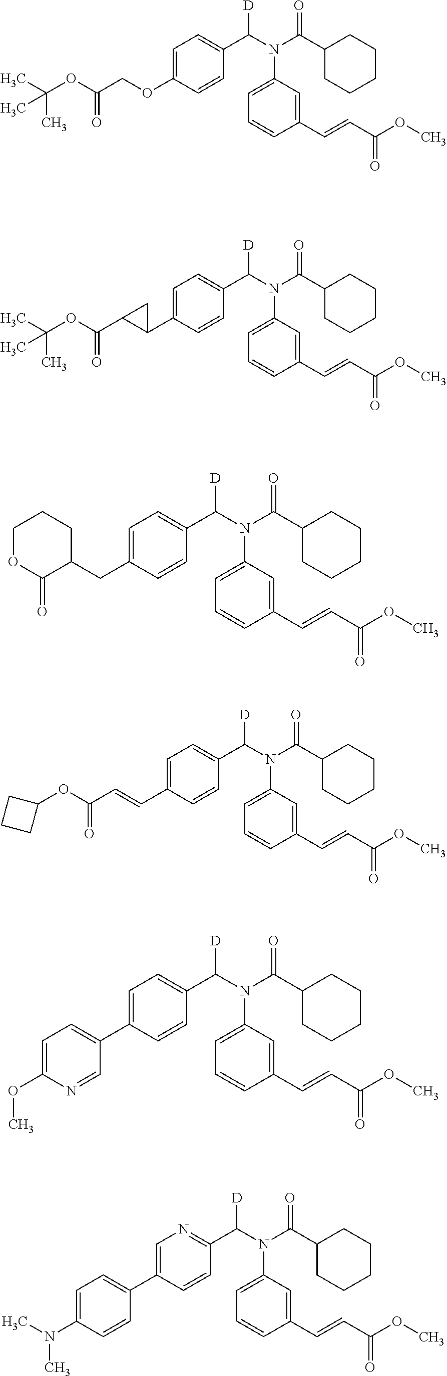

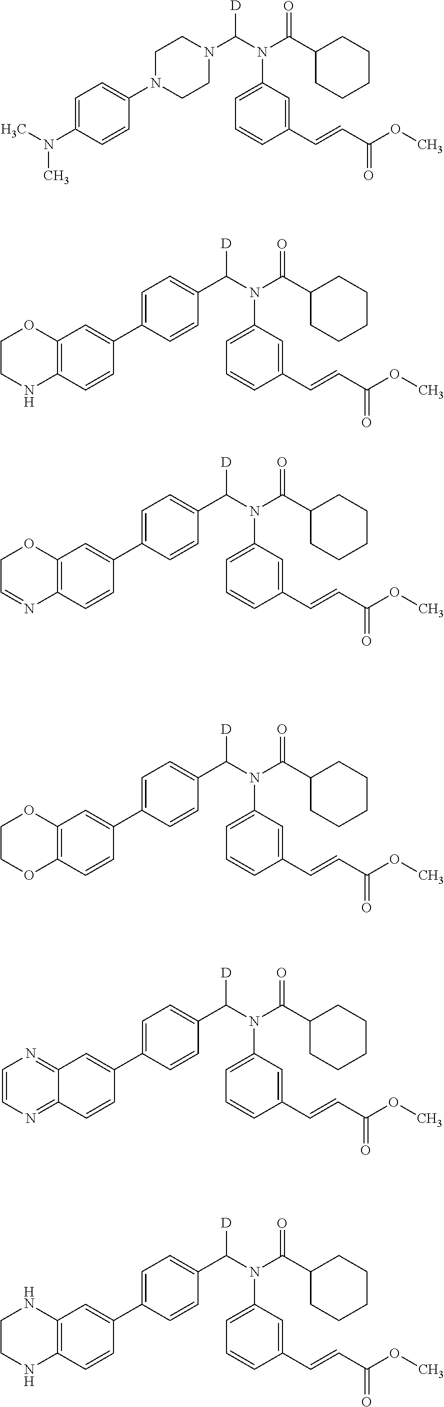

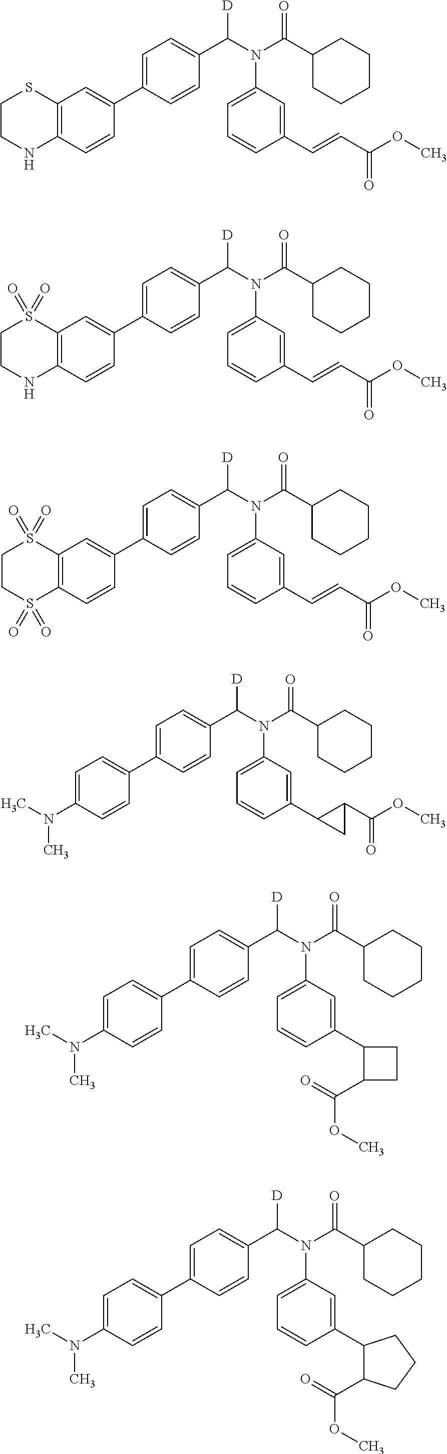

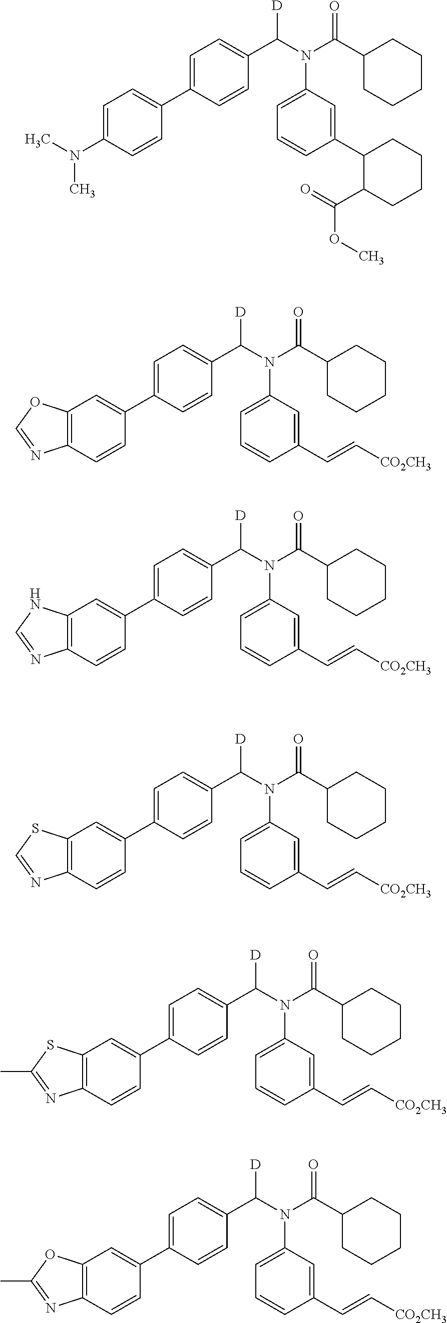

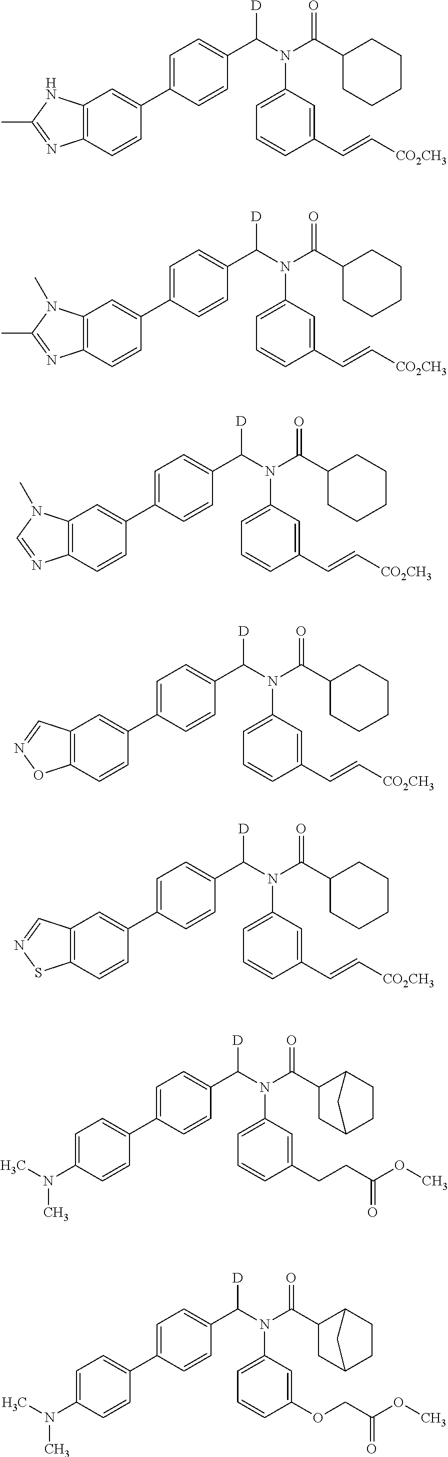

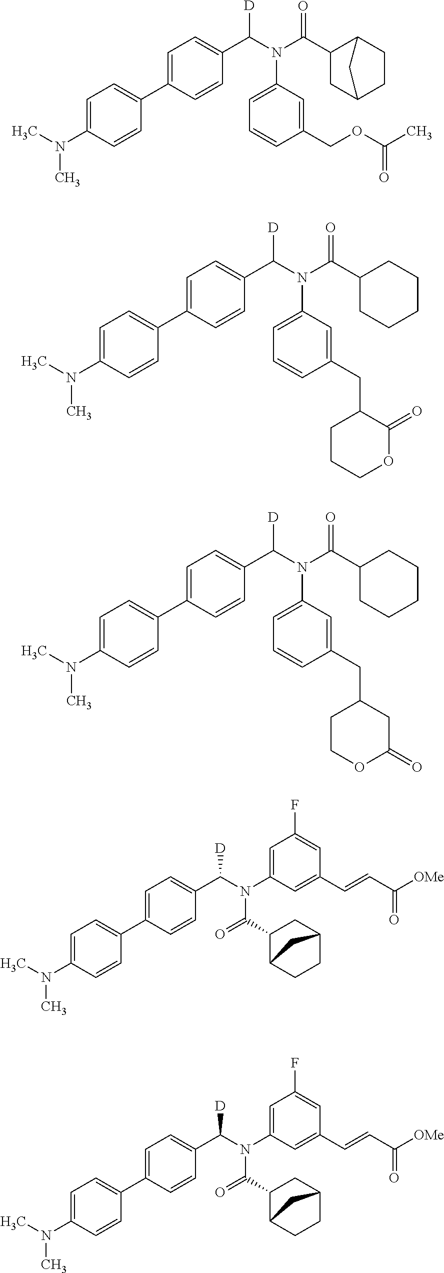

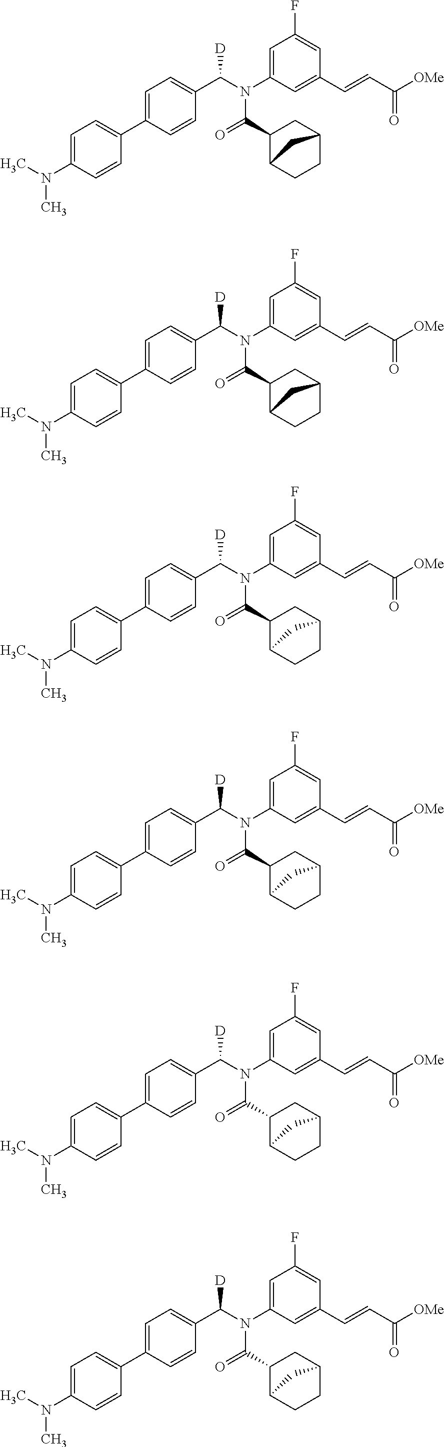

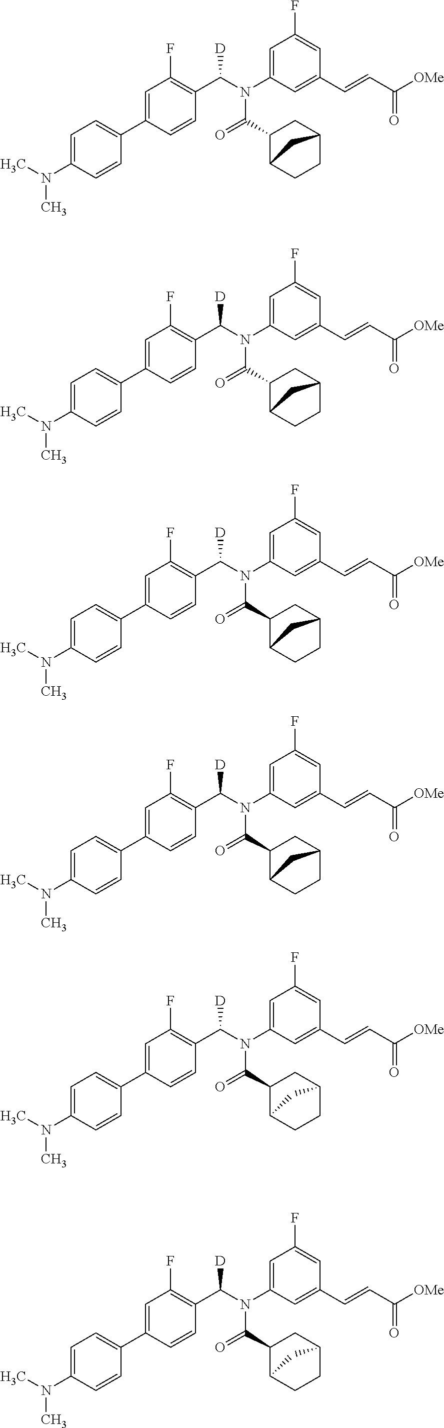

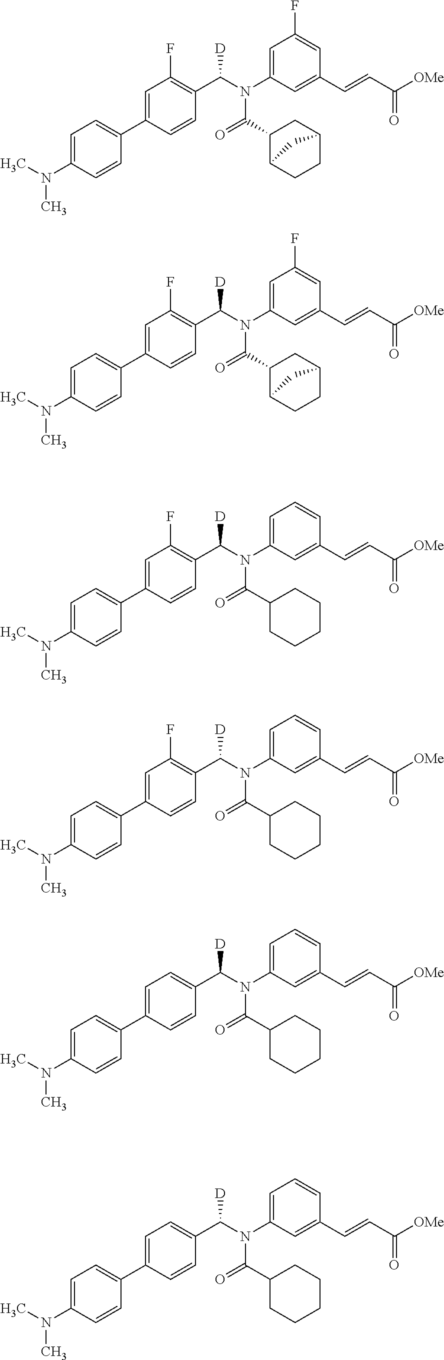

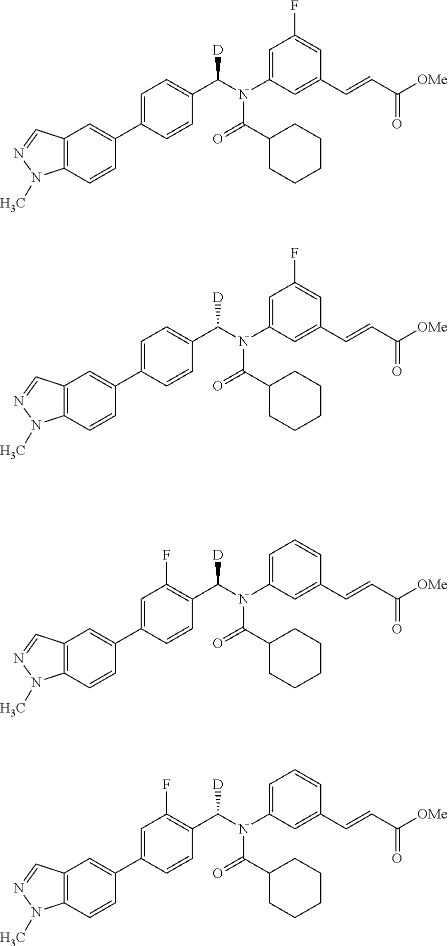

Other exemplary working embodiments include:
methyl (E)-3-(3-(N-(4-(1-methyl-1H-indazol-5-yl)benzyl)cyclohexanecarboxamido)phenyl)acrylate,
methyl (E)-3-(3-((1R,2S,4S)—N-(4-(1-methyl-1H-indazol-5-yl)benzyl)bicyclo[2.2.1]heptane-2-carboxamido)phenyl)acrylate,
methyl (E)-3-(3-(1-methyl-N-(4-(1-methyl-1H-indazol-5-yl)benzyl)piperidine-4-carboxamido)phenyl)acrylate,
methyl (E)-3-(5-(N-((1-methyl-1H-benzo[f]indazol-8-yl)methyl)cyclohexanecarboxamido)pyridin-3-yl)acrylate,
methyl (E)-3-(3-fluoro-5-((1S,2R,4R)—N-((1-methyl-1H-benzo[f]indazol-8-yl)methyl)bicyclo[2.2.1]heptane-2-carboxamido)phenyl)acrylate,
methyl (E)-3-(3-(N-((9-fluoro-1-methyl-1H-benzo[f]indazol-8-yl)methyl)cyclohexanecarboxamido)phenyl)acrylate,
methyl (E)-3-(3-((1R,4S)—N-((9-fluoro-1-methyl-1H-benzo[f]indazol-8-yl)methyl)bicyclo[2.2.1]heptane-2-carboxamido)phenyl)acrylate,
methyl (E)-3-(3-(N-((9-fluoro-1-methyl-1H-benzo[f]indazol-8-yl)methyl)-1-methylpiperidine-4-carboxamido)phenyl)acrylate,
methyl (E)-3-(5-(N-((9-fluoro-1-methyl-1H-benzo[f]indazol-8-yl)methyl)cyclohexanecarboxamido)pyridin-3-yl)acrylate,
methyl (E)-3-(3-fluoro-5-(N-((9-fluoro-1-methyl-1H-benzo[f]indazol-8-yl)methyl)cyclohexanecarboxamido)phenyl)acrylate,
methyl (E)-3-(3-fluoro-5-((1R,4S)—N-((9-fluoro-1-methyl-1H-benzo[f]indazol-8-yl)methyl)bicyclo [2.2.1]heptane-2-carboxamido)phenyl)acrylate,
methyl (E)-3-(5-(N-((9-chloro-1-methyl-1H-benzo[f]indazol-8-yl)methyl)cyclohexanecarboxamido)pyridin-3-yl)acrylate,
methyl (E)-3-(5-((1R,4S)—N-((9-chloro-1-methyl-1H-benzo[f]indazol-8-yl)methyl)bicyclo[2.2.1]heptane-2-carboxamido)pyridin-3-yl)acrylate,
methyl (E)-3-(3-(N-((9-chloro-1-methyl-1H-benzo[f]indazol-8-yl)methyl)-1-methylpiperidine-4-carboxamido)-5-fluorophenyl)acrylate,
methyl (E)-3-(3-(N-((7-(dimethylamino)naphthalen-1-yl)methyl)cyclohexanecarboxamido)phenyl)acrylate,
methyl (E)-3-(3-(N-((7-(dimethylamino)-8-fluoronaphthalen-1-yl)methyl)cyclohexanecarboxamido)phenyl)acrylate,
methyl (E)-3-(3-(N-((4-(1-methyl-1H-indazol-5-yl)phenyl)methyl-d)cyclohexanecarboxamido)phenyl)acrylate,
methyl (E)-3-(5-(N-((4-(1-methyl-1H-indazol-5-yl)phenyl)methyl-d)cyclohexanecarboxamido)pyridin-3-yl)acrylate,
methyl (E)-3-(5-(N-((2-fluoro-4-(1-methyl-1H-indazol-5-yl)phenyl)methyl-d)cyclohexanecarboxamido)pyridin-3-yl)acrylate,
methyl (E)-3-(3-fluoro-5-(N-((2-fluoro-4-(1-methyl-1H-indazol-5-yl)phenyl)methyl-d)cyclohexanecarboxamido)phenyl)acrylate,
methyl (E)-3-(3-(N-((2-chloro-4-(1-methyl-1H-indazol-5-yl)phenyl)methyl-d)cyclohexanecarboxamido)phenyl)acrylate,
methyl (E)-3-(3-((1S,2R,4R)—N-((2-chloro-4-(1-methyl-1H-indazol-5-yl)phenyl)methyl-d)bicyclo[2.2.1]heptane-2-carboxamido)phenyl)acrylate,
methyl (E)-3-(3-(N-((2-chloro-4-(1-methyl-1H-indazol-5-yl)phenyl)methyl-d)cyclohexanecarboxamido)-5-fluorophenyl)acrylate,
methyl (E)-3-(5-((1S,2R,4R)—N-((4′-(dimethylamino)-[1,1′-biphenyl]-4-yl)methyl-d)bicyclo[2.2.1]heptane-2-carboxamido)pyridin-3-yl)acrylate,
methyl (E)-3-(3-(N-((4′-(dimethylamino)-[1,1′-biphenyl]-4-yl)methyl-d)cyclohexanecarboxamido)-5-fluorophenyl)acrylate,
methyl (E)-3-(3-((1S,2R,4R)—N-((4′-(dimethylamino)-[1,1′-biphenyl]-4-yl)methyl-d)bicyclo[2.2.1]heptane-2-carboxamido)-5-fluorophenyl)acrylate,
methyl (E)-3-(3-(N-((4′-(dimethylamino)-3-fluoro-[1,1′-biphenyl]-4-yl)methyl-d)cyclohexanecarboxamido)phenyl)acrylate,
methyl (E)-3-(5-(N-((4′-(dimethylamino)-3-fluoro-[1,1′-biphenyl]-4-yl)methyl-d)cyclohexanecarboxamido)pyridin-3-yl)acrylate,
methyl (E)-3-(5-((1S,2R,4R)—N-((4′-(dimethylamino)-3-fluoro-[1,1′-biphenyl]-4-yl)methyl-d)bicyclo[2.2.1]heptane-2-carboxamido)pyridin-3-yl)acrylate,
methyl (E)-3-(3-(N-((3-chloro-4′-(dimethylamino)-[1,1′-biphenyl]-4-yl)methyl-d)cyclohexanecarboxamido)phenyl)acrylate,
methyl (E)-3-(5-(N-((3-chloro-4′-(dimethylamino)-[1,1′-biphenyl]-4-yl)methyl-d)cyclohexanecarboxamido)pyridin-3-yl)acrylate,
methyl (E)-3-(3-fluoro-5-(N-((2-fluoro-4-(1-methyl-1H-indazol-5-yl)phenyl)methyl-d)benzamido)phenyl)acrylate,
methyl (E)-3-(3-(N-((2-chloro-4-(1-methyl-1H-indazol-5-yl)phenyl)methyl-d)benzamido)-5-fluorophenyl)acrylate,
methyl (E)-3-(3-(N-((2-fluoro-4-(1-methyl-1H-indazol-5-yl)phenyl)methyl-d)benzamido)phenyl)acrylate,
methyl (E)-3-(5-((1S,2R,4R)—N-((2-fluoro-4-(1-methyl-1H-indazol-5-yl)phenyl)methyl-d)bicyclo[2.2.1]heptane-2-carboxamido)pyridin-3-yl)acrylate,
methyl (E)-3-(3-(N-(2-chloro-4-(1-methyl-1H-indazol-5-yl)benzyl)-1-methylpiperidine-4-carboxamido)phenyl)acrylate,
methyl (E)-3-(3-((1S,2R,4R)—N-(2-chloro-4-(1-methyl-1H-indazol-5-yl)benzyl)bicyclo[2.2.1]heptane-2-carboxamido)phenyl)acrylate,
methyl (E)-3-(3-((1S,2R,4R)—N-(2-chloro-4-(1-methyl-1H-indazol-5-yl)benzyl)bicyclo[2.2.1]heptane-2-carboxamido)-5-fluorophenyl)acrylate,
methyl (E)-3-(5-((1S,2R,4R)—N-((2-chloro-4-(1-methyl-1H-indazol-5-yl)phenyl)methyl-d)bicyclo[2.2.1]heptane-2-carboxamido)pyridin-3-yl)acrylate,
methyl (E)-3-(3-(N-(4-(1-methyl-1H-indazol-5-yl)benzyl)benzamido)phenyl)acrylate,
methyl (E)-3-(3-fluoro-5-(N-(4-(1-methyl-1H-indazol-5-yl)benzyl)benzamido)phenyl)acrylate,
methyl (E)-3-(3-(N-(2-fluoro-4-(1-methyl-1H-indazol-5-yl)benzyl)benzamido)phenyl)acrylate,
methyl (E)-3-(5-(N-(2-fluoro-4-(1-methyl-1H-indazol-5-yl)benzyl)benzamido)pyridin-3-yl)acrylate,
methyl (E)-3-(3-(N-((4-(1-methyl-1H-indazol-5-yl)phenyl)methyl-d)benzamido)phenyl)acrylate,
methyl (E)-3-(3-fluoro-5-(N-((4-(1-methyl-1H-indazol-5-yl)phenyl)methyl-d)benzamido)phenyl)acrylate,
methyl (E)-3-(5-(N-((4′-(dimethylamino)-[1,1′-biphenyl]-4-yl)methyl-d)cyclohexanecarboxamido)pyridin-3-yl)acrylate,
methyl (E)-3-(3-((1S,2R,4R)—N-((4′-(dimethylamino)-[1,1′-biphenyl]-4-yl)methyl)bicyclo[2.2.1]heptane-2-carboxamido)phenyl)acrylate,
methyl (E)-3-(5-((1S,2R,4R)—N-((4′-(dimethylamino)-[1,1′-biphenyl]-4-yl)methyl)bicyclo[2.2.1]heptane-2-carboxamido)pyridin-3-yl)acrylate,
methyl (E)-3-(3-(N-((3-chloro-4′-(dimethylamino)-[1,1′-biphenyl]-4-yl)methyl-d)cyclohexanecarboxamido)-5-fluorophenyl)acrylate,
methyl (E)-3-(3-((1S,2R,4R)—N-((4′-(dimethylamino)-3-fluoro-[1,1′-biphenyl]-4-yl)methyl)bicyclo[2.2.1]heptane-2-carboxamido)phenyl)acrylate,
methyl (E)-3-(3-((1S,2R,4R)—N-((4′-(dimethylamino)-3-fluoro-[1,1′-biphenyl]-4-yl)methyl)bicyclo[2.2.1]heptane-2-carboxamido)-5-fluorophenyl)acrylate,
methyl (E)-3-(3-((1S,2R,4R)—N-((3-chloro-4′-(dimethylamino)-[1,1′-biphenyl]-4-yl)methyl)bicyclo[2.2.1]heptane-2-carboxamido)phenyl)acrylate,
methyl (E)-3-(3-((1S,2R,4R)—N-((3-chloro-4′-(dimethylamino)-[1,1′-biphenyl]-4-yl)methyl-d)bicyclo [2.2.1]heptane-2-carboxamido)phenyl)acrylate,
methyl (E)-3-(5-((1S,2R,4R)—N-((3-chloro-4′-(dimethylamino)-[1,1′-biphenyl]-4-yl)methyl-d)bicyclo[2.2.1]heptane-2-carboxamido)pyridin-3-yl)acrylate,
methyl (E)-3-(5-(N-((4′-(dimethylamino)-[1,1′-biphenyl]-4-yl)methyl)benzamido)pyridin-3-yl)acrylate,
methyl (E)-3-(3-(N-((4′-(dimethylamino)-3-fluoro-[1,1′-biphenyl]-4-yl)methyl)benzamido)phenyl)acrylate,
methyl (E)-3-(3-(N-((3-chloro-4′-(dimethylamino)-[1,1′-biphenyl]-4-yl)methyl)benzamido)phenyl)acrylate,
methyl (E)-3-(3-(N-((4′-(dimethylamino)-[1,1′-biphenyl]-4-yl)methyl-d)benzamido)phenyl)acrylate,
methyl (E)-3-(5-(N-((4′-(dimethylamino)-[1,1′-biphenyl]-4-yl)methyl-d)benzamido)pyridin-3-yl)acrylate,
methyl (E)-3-(3-(N-((3-chloro-4′-(dimethylamino)-[1,1′-biphenyl]-4-yl)methyl-d)benzamido)-5-fluorophenyl)acrylate,
methyl (E)-3-(3-fluoro-5-(N-((4-(1-methyl-1H-indazol-5-yl)phenyl)methyl-d)cyclohexanecarboxamido)phenyl)acrylate,
methyl (E)-3-(3-fluoro-5-(N-(4-(1-methyl-1H-indazol-5-yl)benzyl)cyclohexanecarboxamido)phenyl)acrylate,
methyl (E)-3-(3-(N-((4′-(dimethylamino)-[1,1′-biphenyl]-4-yl)methyl)benzamido)-5-fluorophenyl)acrylate,
methyl (E)-3-(3-(N-(2-chloro-4-(1-methyl-1H-indazol-5-yl)benzyl)cyclohexanecarboxamido)phenyl)acrylate,
methyl (E)-3-(5-((1S,2R,4R)—N-(2-fluoro-4-(1-methyl-1H-indazol-5-yl)benzyl)bicyclo[2.2.1]heptane-2-carboxamido)pyridin-3-yl)acrylate,
methyl (E)-3-(3-(N-((2-fluoro-4-(1-methyl-1H-indazol-5-yl)phenyl)methyl-d)cyclohexanecarboxamido)phenyl)acrylate,
methyl (E)-3-(5-(N-((3-chloro-4′-(dimethylamino)-[1,1′-biphenyl]-4-yl)methyl-d)benzamido)pyridin-3-yl)acrylate,
methyl (E)-3-(3-(N-(2-chloro-4-(1-methyl-1H-indazol-5-yl)benzyl)cyclohexanecarboxamido)-5-fluorophenyl)acrylate,
methyl (E)-3-(5-(N-((2-chloro-4-(1-methyl-1H-indazol-5-yl)phenyl)methyl-d)cyclohexanecarboxamido)pyridin-3-yl)acrylate,
methyl (E)-3-(3-((1S,2R,4R)—N-((2-chloro-4-(1-methyl-1H-indazol-5-yl)phenyl)methyl-d)bicyclo[2.2.1]heptane-2-carboxamido)-5-fluorophenyl)acrylate,
methyl (E)-3-(3-(N-(2-chloro-4-(1-methyl-1H-indazol-5-yl)benzyl)benzamido)-5-fluorophenyl)acrylate,
methyl (E)-3-(5-(N-(2-chloro-4-(1-methyl-1H-indazol-5-yl)benzyl)benzamido)pyridin-3-yl)acrylate,
methyl (E)-3-(3-fluoro-5-(N-(2-fluoro-4-(1-methyl-1H-indazol-5-yl)benzyl)benzamido)phenyl)acrylate,
methyl (E)-3-(3-(N-((2-chloro-4-(1-methyl-1H-indazol-5-yl)phenyl)methyl-d)benzamido)phenyl)acrylate,
methyl (E)-3-(5-((1S,2R,4R)—N-((4-(1-methyl-1H-indazol-5-yl)phenyl)methyl-d)bicyclo[2.2.1]heptane-2-carboxamido)pyridin-3-yl)acrylate,
methyl (E)-3-(3-(N-(2-chloro-4-(1-methyl-1H-indazol-5-yl)benzyl)benzamido)phenyl)acrylate,
methyl (E)-3-(3-(N-((4′-(dimethylamino)-[1,1′-biphenyl]-4-yl)methyl-d)cyclohexanecarboxamido)phenyl)acrylate,
methyl (E)-3-(5-(N-((2-chloro-4-(1-methyl-1H-indazol-5-yl)phenyl)methyl-d)benzamido)pyridin-3-yl)acrylate,
methyl (E)-3-(3-(N-((3-chloro-4′-(dimethylamino)-[1,1′-biphenyl]-4-yl)methyl)cyclohexanecarboxamido)phenyl)acrylate,
methyl (E)-3-(3-(N-((4′-(dimethylamino)-3-fluoro-[1,1′-biphenyl]-4-yl)methyl-d)benzamido)phenyl)acrylate,
methyl (E)-3-(5-(N-((4′-(dimethylamino)-3-fluoro-[1,1′-biphenyl]-4-yl)methyl)cyclohexanecarboxamido)pyridin-3-yl)acrylate,
methyl (E)-3-(5-((1S,2R,4R)—N-((3-chloro-4′-(dimethylamino)-[1,1′-biphenyl]-4-yl)methyl)bicyclo[2.2.1]heptane-2-carboxamido)pyridin-3-yl)acrylate,
methyl (E)-3-(3-fluoro-5-((1S,2R,4R)—N-((4-(1-methyl-1H-indazol-5-yl)phenyl)methyl-d)bicyclo[2.2.1]heptane-2-carboxamido)phenyl)acrylate,
methyl (E)-3-(3-((1S,2R,4R)—N-((4-(1-methyl-1H-indazol-5-yl)phenyl)methyl-d)bicyclo[2.2.1]heptane-2-carboxamido)phenyl)acrylate,
methyl (E)-3-(5-(N-((3-chloro-4′-(dimethylamino)-[1,1′-biphenyl]-4-yl)methyl)benzamido)pyridin-3-yl)acrylate,
methyl (E)-3-(3-((1S,2R,4R)—N-((4′-(dimethylamino)-[1,1′-biphenyl]-4-yl)methyl)bicyclo[2.2.1]heptane-2-carboxamido)-5-fluorophenyl)acrylate,
methyl (E)-3-(3-((1S,2R,4R)—N-((2-fluoro-4-(1-methyl-1H-indazol-5-yl)phenyl)methyl-d)bicyclo[2.2.1]heptane-2-carboxamido)phenyl)acrylate,
methyl (E)-3-(3-(N-((4′-(dimethylamino)-3-fluoro-[1,1′-biphenyl]-4-yl)methyl-d)cyclohexanecarboxamido)-5-fluorophenyl)acrylate,
methyl (E)-3-(3-(N-((3-chloro-4′-(dimethylamino)-[1,1′-biphenyl]-4-yl)methyl)benzamido)-5-fluorophenyl)acrylate,
methyl (E)-3-(3-((1S,2R,4R)—N-((4′-(dimethylamino)-[1,1′-biphenyl]-4-yl)methyl-d)bicyclo[2.2.1]heptane-2-carboxamido)phenyl)acrylate,
methyl (E)-3-(3-((1S,2R,4R)—N-((4′-(dimethylamino)-3-fluoro-[1,1′-biphenyl]-4-yl)methyl-d)bicyclo[2.2.1]heptane-2-carboxamido)-5-fluorophenyl)acrylate,
methyl (E)-3-(3-(N-((4′-(dimethylamino)-3-fluoro-[1,1′-biphenyl]-4-yl)methyl-d)benzamido)-5-fluorophenyl)acrylate,
methyl (E)-3-(3-(N-((4′-(dimethylamino)-[1,1′-biphenyl]-4-yl)methyl-d)benzamido)-5-fluorophenyl)acrylate,
methyl (E)-3-(3-((1S,2R,4R)—N-((3-chloro-4′-(dimethylamino)-[1,1′-biphenyl]-4-yl)methyl-d)bicyclo[2.2.1]heptane-2-carboxamido)-5-fluorophenyl)acrylate,
methyl (E)-3-(5-(N-(4-(1-methyl-1H-indazol-5-yl)benzyl)benzamido)pyridin-3-yl)acrylate,
methyl (E)-3-(5-(N-((4-(1-methyl-1H-indazol-5-yl)phenyl)methyl-d)benzamido)pyridin-3-yl)acrylate,
methyl (E)-3-(3-(N-((3-chloro-4′-(dimethylamino)-[1,1′-biphenyl]-4-yl)methyl)cyclohexanecarboxamido)-5-fluorophenyl)acrylate,
methyl (E)-3-(5-(N-((4′-(dimethylamino)-3-fluoro-[1,1′-biphenyl]-4-yl)methyl-d)benzamido)pyridin-3-yl)acrylate,
methyl (E)-3-(5-((1S,2R,4R)—N-((4′-(dimethylamino)-3-fluoro-[1,1′-biphenyl]-4-yl)methyl)bicyclo[2.2.1]heptane-2-carboxamido)pyridin-3-yl)acrylate,
methyl (E)-3-(3-(N-(4-(2-(tert-butoxy)-2-oxoethoxy)benzyl)cyclohexanecarboxamido)phenyl)acrylate,
tert-butyl (E)-2-(4-((N-(3-(3-methoxy-3-oxoprop-1-en-1-yl)phenyl)cyclohexanecarboxamido)methyl)phenyl)cyclopropane-1-carboxylate,
methyl (E)-3-(3-(N-(4-((2-oxotetrahydro-2H-pyran-3-yl)methyl)benzyl)cyclohexanecarboxamido)phenyl)acrylate,
cyclobutyl (E)-3-(4-((N-(3-((E)-3-methoxy-3-oxoprop-1-en-1-yl)phenyl)cyclohexanecarboxamido)methyl)phenyl)acrylate,
methyl (E)-3-(3-(N-(4-(6-methoxypyridin-3-yl)benzyl)cyclohexanecarboxamido)phenyl)acrylate,
methyl (E)-3-(3-(N-((5-(4-(dimethylamino)phenyl)pyridin-2-yl)methyl)cyclohexanecarboxamido)phenyl)acrylate,
methyl (E)-3-(3-(N-((4-(4-(dimethylamino)phenyl)piperazin-1-yl)methyl)cyclohexanecarboxamido)phenyl)acrylate,
methyl (E)-3-(3-(N-(4-(3,4-dihydro-2H-benzo[b][1,4]oxazin-7-yl)benzyl)cyclohexanecarboxamido)phenyl)acrylate,
methyl (E)-3-(3-(N-(4-(2H-benzo[b][1,4]oxazin-7-yl)benzyl)cyclohexanecarboxamido)phenyl)acrylate,
methyl (E)-3-(3-(N-(4-(2,3-dihydrobenzo[b] [l 1,4]dioxin-6-yl)benzyl)cyclohexanecarboxamido)phenyl)acrylate,
methyl (E)-3-(3-(N-(4-(quinoxalin-6-yl)benzyl)cyclohexanecarboxamido)phenyl)acrylate,
methyl (E)-3-(3-(N-(4-(1,2,3,4-tetrahydroquinoxalin-6-yl)benzyl)cyclohexanecarboxamido)phenyl)acrylate,
methyl (E)-3-(3-(N-(4-(3,4-dihydro-2H-benzo[b][1,4]thiazin-7-yl)benzyl)cyclohexanecarboxamido)phenyl)acrylate,
methyl (E)-3-(3-(N-(4-(1,1-dioxido-3,4-dihydro-2H-benzo[b][1,4]thiazin-7-yl)benzyl)cyclohexanecarboxamido)phenyl)acrylate,
methyl (E)-3-(3-(N-(4-(1,1,4,4-tetraoxido-2,3-dihydrobenzo[b][1,4]dithiin-6-yl)benzyl)cyclohexanecarboxamido)phenyl)acrylate,
methyl 2-(3-(N-((4′-(dimethylamino)-[1,1′-biphenyl]-4-yl)methyl)cyclohexanecarboxamido)phenyl)cyclopropane-1-carboxylate,
methyl 2-(3-(N-((4′-(dimethylamino)-[1,1′-biphenyl]-4-yl)methyl)cyclohexanecarboxamido)phenyl)cyclobutane-1-carboxylate,
methyl 2-(3-(N-((4′-(dimethylamino)-[1,1′-biphenyl]-4-yl)methyl)cyclohexanecarboxamido)phenyl)cyclopentane-1-carboxylate,
methyl 2-(3-(N-((4′-(dimethylamino)-[1,1′-biphenyl]-4-yl)methyl)cyclohexanecarboxamido)phenyl)cyclohexane-1-carboxylate,
methyl (E)-3-(3-(N-(4-(benzo[d]oxazol-6-yl)benzyl)cyclohexanecarboxamido)phenyl)acrylate,
methyl (E)-3-(3-(N-(4-(1H-benzo[d]imidazol-6-yl)benzyl)cyclohexanecarboxamido)phenyl)acrylate,
methyl (E)-3-(3-(N-(4-(benzo[d]thiazol-6-yl)benzyl)cyclohexanecarboxamido)phenyl)acrylate,
methyl (E)-3-(3-(N-(4-(2-methylbenzo[d]thiazol-6-yl)benzyl)cyclohexanecarboxamido)phenyl)acrylate,
methyl (E)-3-(3-(N-(4-(2-methylbenzo[d]oxazol-6-yl)benzyl)cyclohexanecarboxamido)phenyl)acrylate,
methyl (E)-3-(3-(N-(4-(2-methyl-1H-benzo[d]imidazol-6-yl)benzyl)cyclohexanecarboxamido)phenyl)acrylate,
methyl (E)-3-(3-(N-(4-(1,2-dimethyl-1H-benzo[d]imidazol-6-yl)benzyl)cyclohexanecarboxamido)phenyl)acrylate,
methyl (E)-3-(3-(N-(4-(1-methyl-1H-benzo[d]imidazol-6-yl)benzyl)cyclohexanecarboxamido)phenyl)acrylate,
methyl (E)-3-(3-(N-(4-(benzo[d]isoxazol-5-yl)benzyl)cyclohexanecarboxamido)phenyl)acrylate,
methyl (E)-3-(3-(N-(4-(benzo[d]isothiazol-5-yl)benzyl)cyclohexanecarboxamido)phenyl)acrylate,
methyl 3-(3-(N-((4′-(dimethylamino)-[1,1′-biphenyl]-4-yl)methyl)bicyclo[2.2.1]heptane-2-carboxamido)phenyl)propanoate,
methyl 2-(3-(N-((4′-(dimethylamino)-[1,1′-biphenyl]-4-yl)methyl)bicyclo[2.2.1]heptane-2-carboxamido)phenoxy)acetate,
3-(N-((4′-(dimethylamino)-[1,1′-biphenyl]-4-yl)methyl)bicyclo[2.2.1]heptane-2-carboxamido)benzyl acetate,
N-((4′-(dimethylamino)-[1,1′-biphenyl]-4-yl)methyl)-N-(3-((2-oxotetrahydro-2H-pyran-3-yl)methyl)phenyl)cyclohexanecarboxamide,
N-((4′-(dimethylamino)-[1,1′-biphenyl]-4-yl)methyl)-N-(3-((2-oxotetrahydro-2H-pyran-4-yl)methyl)phenyl)cyclohexanecarboxamide,
methyl (E)-3-(3-(N-((4-(2-(tert-butoxy)-2-oxoethoxy)phenyl)methyl-d)cyclohexanecarboxamido)phenyl)acrylate,
tert-butyl (E)-2-(4-((N-(3-(3-methoxy-3-oxoprop-1-en-1-yl)phenyl)cyclohexanecarboxamido)methyl-d)phenyl)cyclopropane-1-carboxylate,
methyl (E)-3-(3-(N-((4-((2-oxotetrahydro-2H-pyran-3-yl)methyl)phenyl)methyl-d)cyclohexanecarboxamido)phenyl)acrylate,
cyclobutyl (E)-3-(4-((N-(3-((E)-3-methoxy-3-oxoprop-1-en-1-yl)phenyl)cyclohexanecarboxamido)methyl-d)phenyl)acrylate,
methyl (E)-3-(3-(N-((4-(6-methoxypyridin-3-yl)phenyl)methyl-d)cyclohexanecarboxamido)phenyl)acrylate,
methyl (E)-3-(3-(N-((5-(4-(dimethylamino)phenyl)pyridin-2-yl)methyl-d)cyclohexanecarboxamido)phenyl)acrylate,
methyl (E)-3-(3-(N-((4-(4-(dimethylamino)phenyl)piperazin-1-yl)methyl-d)cyclohexanecarboxamido)phenyl)acrylate,
methyl (E)-3-(3-(N-((4-(3,4-dihydro-2H-benzo [b][1,4]oxazin-7-yl)phenyl)methyl-d)cyclohexanecarboxamido)phenyl)acrylate,
methyl (E)-3-(3-(N-((4-(2H-benzo[b][1,4]oxazin-7-yl)phenyl)methyl-d)cyclohexanecarboxamido)phenyl)acrylate,
methyl (E)-3-(3-(N-((4-(2,3-dihydrobenzo[b][1,4]dioxin-6-yl)phenyl)methyl-d)cyclohexanecarboxamido)phenyl)acrylate,
methyl (E)-3-(3-(N-((4-(quinoxalin-6-yl)phenyl)methyl-d)cyclohexanecarboxamido)phenyl)acrylate,
methyl (E)-3-(3-(N-((4-(1,2,3,4-tetrahydroquinoxalin-6-yl)phenyl)methyl-d)cyclohexanecarboxamido)phenyl)acrylate,
methyl (E)-3-(3-(N-((4-(3,4-dihydro-2H-benzo [b][1,4]thiazin-7-yl)phenyl)methyl-d)cyclohexanecarboxamido)phenyl)acrylate,
methyl (E)-3-(3-(N-((4-(1,1-dioxido-3,4-dihydro-2H-benzo[b][1,4]thiazin-7-yl)phenyl)methyl-d)cyclohexanecarboxamido)phenyl)acrylate,
methyl (E)-3-(3-(N-((4-(1,1,4,4-tetraoxido-2,3-dihydrobenzo[b][1,4]dithiin-6-yl)phenyl)methyl-d)cyclohexanecarboxamido)phenyl)acrylate,
methyl 2-(3-(N-((4′-(dimethylamino)-[1,1′-biphenyl]-4-yl)methyl-d)cyclohexanecarboxamido)phenyl)cyclopropane-1-carboxylate,
methyl 2-(3-(N-((4′-(dimethylamino)-[1,1′-biphenyl]-4-yl)methyl-d)cyclohexanecarboxamido)phenyl)cyclobutane-1-carboxylate,
methyl 2-(3-(N-((4′-(dimethylamino)-[1,1′-biphenyl]-4-yl)methyl-d)cyclohexanecarboxamido)phenyl)cyclopentane-1-carboxylate,
methyl 2-(3-(N-((4′-(dimethylamino)-[1,1′-biphenyl]-4-yl)methyl-d)cyclohexanecarboxamido)phenyl)cyclohexane-1-carboxylate,
methyl (E)-3-(3-(N-((4-(benzo[d]oxazol-6-yl)phenyl)methyl-d)cyclohexanecarboxamido)phenyl)acrylate,
methyl (E)-3-(3-(N-((4-(1H-benzo[d]imidazol-6-yl)phenyl)methyl-d)cyclohexanecarboxamido)phenyl)acrylate,
methyl (E)-3-(3-(N-((4-(benzo[d]thiazol-6-yl)phenyl)methyl-d)cyclohexanecarboxamido)phenyl)acrylate,
methyl (E)-3-(3-(N-((4-(2-methylbenzo[d]thiazol-6-yl)phenyl)methyl-d)cyclohexanecarboxamido)phenyl)acrylate,
methyl (E)-3-(3-(N-((4-(2-methylbenzo[d]oxazol-6-yl)phenyl)methyl-d)cyclohexanecarboxamido)phenyl)acrylate,
methyl (E)-3-(3-(N-((4-(2-methyl-1H-benzo[d]imidazol-6-yl)phenyl)methyl-d)cyclohexanecarboxamido)phenyl)acrylate,
methyl (E)-3-(3-(N-((4-(1,2-dimethyl-1H-benzo[d]imidazol-6-yl)phenyl)methyl-d)cyclohexanecarboxamido)phenyl)acrylate,
methyl (E)-3-(3-(N-((4-(1-methyl-1H-benzo[d]imidazol-6-yl)phenyl)methyl-d)cyclohexanecarboxamido)phenyl)acrylate,
methyl (E)-3-(3-(N-((4-(benzo[d]isoxazol-5-yl)phenyl)methyl-d)cyclohexanecarboxamido)phenyl)acrylate,
methyl (E)-3-(3-(N-((4-(benzo[d]isothiazol-5-yl)phenyl)methyl-d)cyclohexanecarboxamido)phenyl)acrylate,
methyl 3-(3-(N-((4′-(dimethylamino)-[1,1′-biphenyl]-4-yl)methyl-d)bicyclo[2.2.1]heptane-2-carboxamido)phenyl)propanoate,
methyl 2-(3-(N-((4′-(dimethylamino)-[1,1′-biphenyl]-4-yl)methyl-d)bicyclo[2.2.1]heptane-2-carboxamido)phenoxy)acetate,
3-(N-((4′-(dimethylamino)-[1,1′-biphenyl]-4-yl)methyl-d)bicyclo[2.2.1]heptane-2-carboxamido)benzyl acetate,
N-((4′-(dimethylamino)-[1,1′-biphenyl]-4-yl)methyl-d)-N-(3-((2-oxotetrahydro-2H-pyran-3-yl)methyl)phenyl)cyclohexanecarboxamide,
N-((4′-(dimethylamino)-[1,1′-biphenyl]-4-yl)methyl-d)-N-(3-((2-oxotetrahydro-2H-pyran-4-yl)methyl)phenyl)cyclohexanecarboxamide,
methyl (E)-3-(3-((1S,2R,4R)—N—((S)-(4′-(dimethylamino)-[1,1′-biphenyl]-4-yl)methyl-d)bicyclo[2.2.1]heptane-2-carboxamido)-5-fluorophenyl)acrylate,
methyl (E)-3-(3-((1S,2R,4R)—N—((R)-(4′-(dimethylamino)-[1,1′-biphenyl]-4-yl)methyl-d)bicyclo[2.2.1]heptane-2-carboxamido)-5-fluorophenyl)acrylate,
methyl (E)-3-(3-((1S,2S,4R)—N—((S)-(4′-(dimethylamino)-[1,1′-biphenyl]-4-yl)methyl-d)bicyclo[2.2.1]heptane-2-carboxamido)-5-fluorophenyl)acrylate,
methyl (E)-3-(3-((1S,2S,4R)—N—((R)-(4′-(dimethylamino)-[1,1′-biphenyl]-4-yl)methyl-d)bicyclo[2.2.1]heptane-2-carboxamido)-5-fluorophenyl)acrylate,
methyl (E)-3-(3-((1R,2S,4S)—N—((S)-(4′-(dimethylamino)-[1,1′-biphenyl]-4-yl)methyl-d)bicyclo[2.2.1]heptane-2-carboxamido)-5-fluorophenyl)acrylate,
methyl (E)-3-(3-((1R,2S,4S)—N—((R)-(4′-(dimethylamino)-[1,1′-biphenyl]-4-yl)methyl-d)bicyclo[2.2.1]heptane-2-carboxamido)-5-fluorophenyl)acrylate,
methyl (E)-3-(3-((1R,2R,4S)—N—((S)-(4′-(dimethylamino)-[1, 1′-biphenyl]-4-yl)methyl-d)bicyclo[2.2.1]heptane-2-carboxamido)-5-fluorophenyl)acrylate,
methyl (E)-3-(3-((1R,2R,4S)—N—((R)-(4′-(dimethylamino)-[1,1′-biphenyl]-4-yl)methyl-d)bicyclo[2.2.1]heptane-2-carboxamido)-5-fluorophenyl)acrylate,
methyl (E)-3-(3-((1S,2R,4R)—N—((S)-(4′-(dimethylamino)-3-fluoro-[1,1′-biphenyl]-4-yl)methyl-d)bicyclo[2.2.1]heptane-2-carboxamido)-5-fluorophenyl)acrylate,
methyl (E)-3-(3-((1S,2R,4R)—N—((R)-(4′-(dimethylamino)-3-fluoro-[1,1′-biphenyl]-4-yl)methyl-d)bicyclo[2.2.1]heptane-2-carboxamido)-5-fluorophenyl)acrylate,
methyl (E)-3-(3-((1S,2S,4R)—N—((S)-(4′-(dimethylamino)-3-fluoro-[1,1′-biphenyl]-4-yl)methyl-d)bicyclo[2.2.1]heptane-2-carboxamido)-5-fluorophenyl)acrylate,
methyl (E)-3-(3-((1S,2S,4R)—N—((R)-(4′-(dimethylamino)-3-fluoro-[1,1′-biphenyl]-4-yl)methyl-d)bicyclo[2.2.1]heptane-2-carboxamido)-5-fluorophenyl)acrylate,
methyl (E)-3-(3-((1R,2S,4S)—N—((S)-(4′-(dimethylamino)-3-fluoro-[1,1′-biphenyl]-4-yl)methyl-d)bicyclo[2.2.1]heptane-2-carboxamido)-5-fluorophenyl)acrylate,
methyl (E)-3-(3-((1R,2S,4S)—N—((R)-(4′-(dimethylamino)-3-fluoro-[1,1′-biphenyl]-4-yl)methyl-d)bicyclo[2.2.1]heptane-2-carboxamido)-5-fluorophenyl)acrylate,
methyl (E)-3-(3-((1R,2R,4S)—N—((S)-(4′-(dimethylamino)-3-fluoro-[1,1′-biphenyl]-4-yl)methyl-d)bicyclo[2.2.1]heptane-2-carboxamido)-5-fluorophenyl)acrylate,
methyl (E)-3-(3-((1R,2R,4S)—N—((R)-(4′-(dimethylamino)-3-fluoro-[1,1′-biphenyl]-4-yl)methyl-d)bicyclo[2.2.1]heptane-2-carboxamido)-5-fluorophenyl)acrylate,
methyl (R,E)-3-(3-(N—((4′-(dimethylamino)-3-fluoro-[1,1′-biphenyl]-4-yl)methyl-d)cyclohexanecarboxamido)phenyl)acrylate,
methyl (S,E)-3-(3-(N—((4′-(dimethylamino)-3-fluoro-[1,1′-biphenyl]-4-yl)methyl-d)cyclohexanecarboxamido)phenyl)acrylate,
methyl (R,E)-3-(3-(N—((4′-(dimethylamino)-[1,1′-biphenyl]-4-yl)methyl-d)cyclohexanecarboxamido)phenyl)acrylate,
methyl (S,E)-3-(3-(N—((4′-(dimethylamino)-[1,1′-biphenyl]-4-yl)methyl-d)cyclohexanecarboxamido)phenyl)acrylate,
methyl (R,E)-3-(3-fluoro-5-(N—((4-(1-methyl-1H-indazol-5-yl)phenyl)methyl-d)cyclohexanecarboxamido)phenyl)acrylate,
methyl (S,E)-3-(3-fluoro-5-(N—((4-(1-methyl-1H-indazol-5-yl)phenyl)methyl-d)cyclohexanecarboxamido)phenyl)acrylate,
methyl (R,E)-3-(3-(N—((2-fluoro-4-(1-methyl-1H-indazol-5-yl)phenyl)methyl-d)cyclohexanecarboxamido)phenyl)acrylate, or
methyl (S,E)-3-(3-(N—((2-fluoro-4-(1-methyl-1H-indazol-5-yl)phenyl)methyl-d)cyclohexanecarboxamido)phenyl)acrylate.

In some embodiments, the compound is selected from

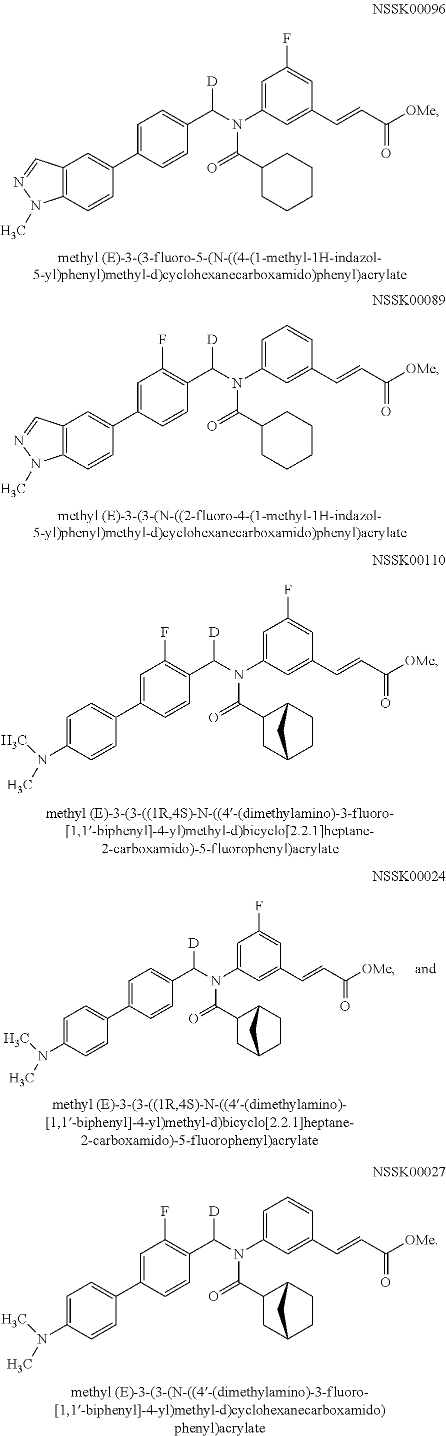 [SMILES: [2H]C(c1ccc(-c2ccc3c(cnn3C)c2)cc1)N(C(=O)C1CCCCC1)c1cc(F)cc(/C=C/C(=O)OC)c1 ; [2H]C(c1ccc(-c2ccc3c(cnn3C)c2)cc1F)N(C(=O)C1CCCCC1)c1cccc(/C=C/C(=O)OC)c1 ; [2H]C(c1ccc(-c2ccc(N(C)C)cc2)cc1F)N(C(=O)C1C[C@H]2CC[C@@H]1C2)c1cc(F)cc(/C=C/C(=O)OC)c1 ; [2H]C(c1ccc(-c2ccc(N(C)C)cc2)cc1)N(C(=O)C1C[C@H]2CC[C@@H]1C2)c1cc(F)cc(/C=C/C(=O)OC)c1 ; [2H]C(c1ccc(-c2ccc(N(C)C)cc2)cc1F)N(C(=O)C1C[C@H]2CC[C@@H]1C2)c1cccc(/C=C/C(=O)OC)c1]

In other embodiments, the compound is
methyl (E)-3-(3-((1S,2R,4R)—N—((S)-(4′-(dimethylamino)-[1,1′-biphenyl]-4-yl)methyl-d)bicyclo[2.2.1]heptane-2-carboxamido)-5-fluorophenyl)acrylate,
methyl (E)-3-(3-((1S,2R,4R)—N—((R)-(4′-(dimethylamino)-[1,1′-biphenyl]-4-yl)methyl-d)bicyclo[2.2.1]heptane-2-carboxamido)-5-fluorophenyl)acrylate,
methyl (E)-3-(3-((1S,2S,4R)—N—((S)-(4′-(dimethylamino)-[1,1′-biphenyl]-4-yl)methyl-d)bicyclo[2.2.1]heptane-2-carboxamido)-5-fluorophenyl)acrylate,
methyl (E)-3-(3-((1S,2S,4R)—N—((R)-(4′-(dimethylamino)-[1,1′-biphenyl]-4-yl)methyl-d)bicyclo[2.2.1]heptane-2-carboxamido)-5-fluorophenyl)acrylate,
methyl (E)-3-(3-((1R,2S,4S)—N—((S)-(4′-(dimethylamino)-[1,1′-biphenyl]-4-yl)methyl-d)bicyclo[2.2.1]heptane-2-carboxamido)-5-fluorophenyl)acrylate,
methyl (E)-3-(3-((1R,2S,4S)—N—((R)-(4′-(dimethylamino)-[1,1′-biphenyl]-4-yl)methyl-d)bicyclo[2.2.1]heptane-2-carboxamido)-5-fluorophenyl)acrylate,
methyl (E)-3-(3-((1R,2R,4S)—N—((S)-(4′-(dimethylamino)-[1,1′-biphenyl]-4-yl)methyl-d)bicyclo[2.2.1]heptane-2-carboxamido)-5-fluorophenyl)acrylate,
methyl (E)-3-(3-((1R,2R,4S)—N—((R)-(4′-(dimethylamino)-[1,1′-biphenyl]-4-yl)methyl-d)bicyclo[2.2.1]heptane-2-carboxamido)-5-fluorophenyl)acrylate,
methyl (E)-3-(3-((1S,2R,4R)—N—((S)-(4′-(dimethylamino)-3-fluoro-[1,1′-biphenyl]-4-yl)methyl-d)bicyclo[2.2.1]heptane-2-carboxamido)-5-fluorophenyl)acrylate,
methyl (E)-3-(3-((1S,2R,4R)—N—((R)-(4′-(dimethylamino)-3-fluoro-[1,1′-biphenyl]-4-yl)methyl-d)bicyclo[2.2.1]heptane-2-carboxamido)-5-fluorophenyl)acrylate,
methyl (E)-3-(3-((1S,2S,4R)—N—((S)-(4′-(dimethylamino)-3-fluoro-[1,1′-biphenyl]-4-yl)methyl-d)bicyclo[2.2.1]heptane-2-carboxamido)-5-fluorophenyl)acrylate,
methyl (E)-3-(3-((1S,2S,4R)—N—((R)-(4′-(dimethylamino)-3-fluoro-[1,1′-biphenyl]-4-yl)methyl-d)bicyclo[2.2.1]heptane-2-carboxamido)-5-fluorophenyl)acrylate,
methyl (E)-3-(3-((1R,2S,4S)—N—((S)-(4′-(dimethylamino)-3-fluoro-[1,1′-biphenyl]-4-yl)methyl-d)bicyclo[2.2.1]heptane-2-carboxamido)-5-fluorophenyl)acrylate,
methyl (E)-3-(3-((1R,2S,4S)—N—((R)-(4′-(dimethylamino)-3-fluoro-[1,1′-biphenyl]-4-yl)methyl-d)bicyclo[2.2.1]heptane-2-carboxamido)-5-fluorophenyl)acrylate,
methyl (E)-3-(3-((1R,2R,4S)—N—((S)-(4′-(dimethylamino)-3-fluoro-[1,1′-biphenyl]-4-yl)methyl-d)bicyclo[2.2.1]heptane-2-carboxamido)-5-fluorophenyl)acrylate,
methyl (E)-3-(3-((1R,2R,4S)—N—((R)-(4′-(dimethylamino)-3-fluoro-[1,1′-biphenyl]-4-yl)methyl-d)bicyclo[2.2.1]heptane-2-carboxamido)-5-fluorophenyl)acrylate,
methyl (R,E)-3-(3-(N—((4′-(dimethylamino)-3-fluoro-[1,1′-biphenyl]-4-yl)methyl-d)cyclohexanecarboxamido)phenyl)acrylate,
methyl (S,E)-3-(3-(N—((4′-(dimethylamino)-3-fluoro-[1,1′-biphenyl]-4-yl)methyl-d)cyclohexanecarboxamido)phenyl)acrylate,
methyl (R,E)-3-(3-(N—((4′-(dimethylamino)-[1,1′-biphenyl]-4-yl)methyl-d)cyclohexanecarboxamido)phenyl)acrylate,
methyl (S,E)-3-(3-(N—((4′-(dimethylamino)-[1,1′-biphenyl]-4-yl)methyl-d)cyclohexanecarboxamido)phenyl)acrylate,
methyl (R,E)-3-(3-fluoro-5-(N—((4-(1-methyl-1H-indazol-5-yl)phenyl)methyl-d)cyclohexanecarboxamido)phenyl)acrylate,
methyl (S,E)-3-(3-fluoro-5-(N—((4-(1-methyl-1H-indazol-5-yl)phenyl)methyl-d)cyclohexanecarboxamido)phenyl)acrylate,
methyl (R,E)-3-(3-(N—((2-fluoro-4-(1-methyl-1H-indazol-5-yl)phenyl)methyl-d)cyclohexanecarboxamido)phenyl)acrylate, or
methyl (S,E)-3-(3-(N—((2-fluoro-4-(1-methyl-1H-indazol-5-yl)phenyl)methyl-d)cyclohexanecarboxamido)phenyl)acrylate.

Also provided herein are kits that include any FXR agonist (or composition containing such an agonist) described herein and a device for localized delivery within a region of the intestines, such as the ileum or colon. In certain embodiments, the device is a syringe, bag, or a pressurized container.

### B. Farnesoid X Receptor (FXR) Agonist Compounds According to Formulas 19-35

Additional embodiments of farnesoid X receptor (FXR) agonist compounds also are disclosed according to formulas 19-35. Certain disclosed FXR agonist compounds have the structure of formula 19

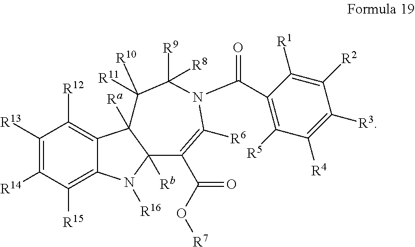

With reference to formula 19, R¹-R¹⁵ independently are selected from hydrogen, deuterium, halogen, CF₃, NO₂, OH, amino, acyl, carboxyl, carboxyl ester, cyano, aminocarbonyl, aminosulfonyl, aliphatic, D-aliphatic, heteroaliphatic, D-heteroaliphatic, or —(CH₂)ₙ₁—R¹⁵⁰—(CH₂)ₙ₂—R¹⁵¹, wherein n1 and n2 are independently selected from the group consisting of 0, 1, 2, 3, and 4, R¹⁵⁰ is O, NR¹⁶, or absent, and R¹⁵¹ is carboxyl ester or amino; R¹⁶ is selected from hydrogen, aliphatic, D-aliphatic, heteroaliphatic or D-heteroaliphatic; R^{a} and R^{b} are independently hydrogen, deuterium, aliphatic or D-aliphatic, or together form a pi-bond.

Also with reference to formula 19, in some embodiments, none of R¹-R¹⁶ is —R^{x}-L^{x}-R^{x2}, where R^{x} is selected from O, NR^{x3}, sulfonyl or S; R^{x3} is selected from H, aliphatic, or aryl; L^{x} is selected from a bond, aliphatic, heteroaliphatic, aryl, heteroaryl or CR^{x4}R^{x5}; R^{x4} and R^{x5} are each independently selected from H, D, halogen, aliphatic, —C(O)OR^{x6}, or —C(O)NR⁶R⁷; R^{x6} and R^{x7} are each independently selected from H, aliphatic; R^{x2} is selected from —C(O)L^{x2}R^{x8} or a carboxyl bioisostere; L^{x2} is a bond or NR^{x3}; R^{x8} is H, aliphatic, —OR^{x9}, N(R^{x9})₂, —C(O)R^{x9}, —S(O)₂R^{x9}, —C(O)OR^{x9}, —S(O)₂N(R⁹)₂ or —C(O)N(R^{x9})₂; and each R^{x9} is independently selected from H, aliphatic.

In some embodiments, at least one of R¹-R¹⁶ is or comprises deuterium.

R⁷ may be H, aliphatic, D-aliphatic, heteroaliphatic or D-heteroaliphatic. In some embodiments, R⁷ is alkyl or deuterated alkyl, and in certain embodiments, R⁷ is isopropyl or deuterated isopropyl, having from 1 to 7 deuterium atoms.

In some embodiments, at least one of R¹-R⁵ is a halogen. In certain examples, R² and R³ are both fluoro.

In some embodiments, R¹⁶ is hydrogen.

In some examples, R¹⁰ and R¹¹ independently are alkyl or deuterated alkyl, and in certain examples, R¹⁰ and R¹¹ independently are methyl or deuterated methyl, having from 1 to 3 deuterium atoms.

In some embodiments, R^{a} and R^{b} together form a pi-bond, leading to compounds have the structure of formula 20

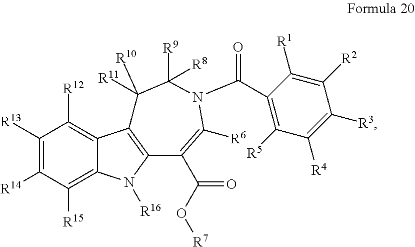

where R¹-R¹⁶ are as defined above with respect to formula 19, and at least one of R¹-R¹⁵ is or comprises deuterium.

In other embodiments, R^{a} and R^{b} are both hydrogen, leading to compounds having a structure of formula 21

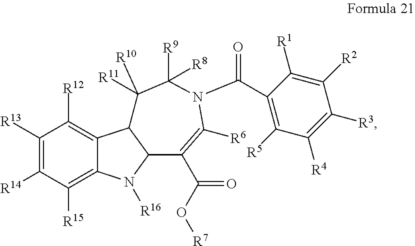

where R¹-R¹⁶ are as defined above with respect to formula 19.

Exemplary compounds having the structure of formula 19 include:

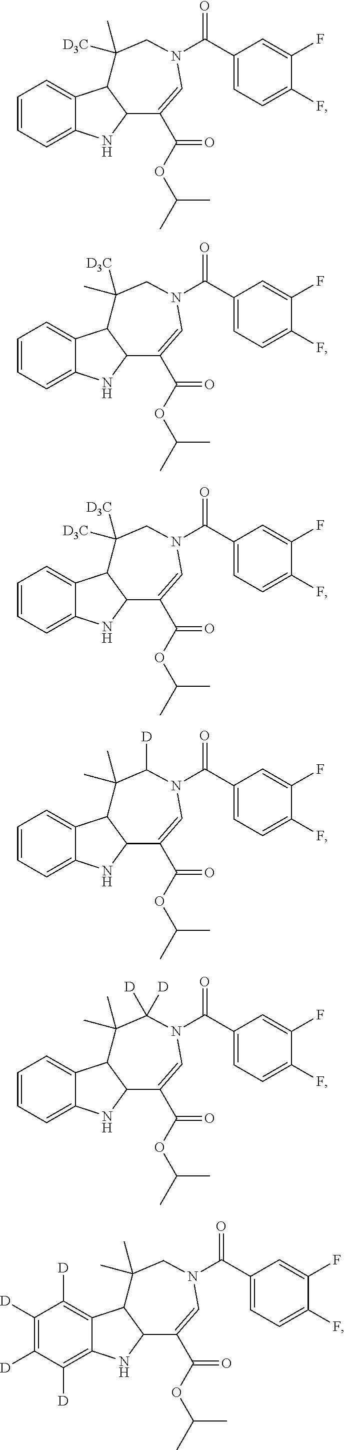

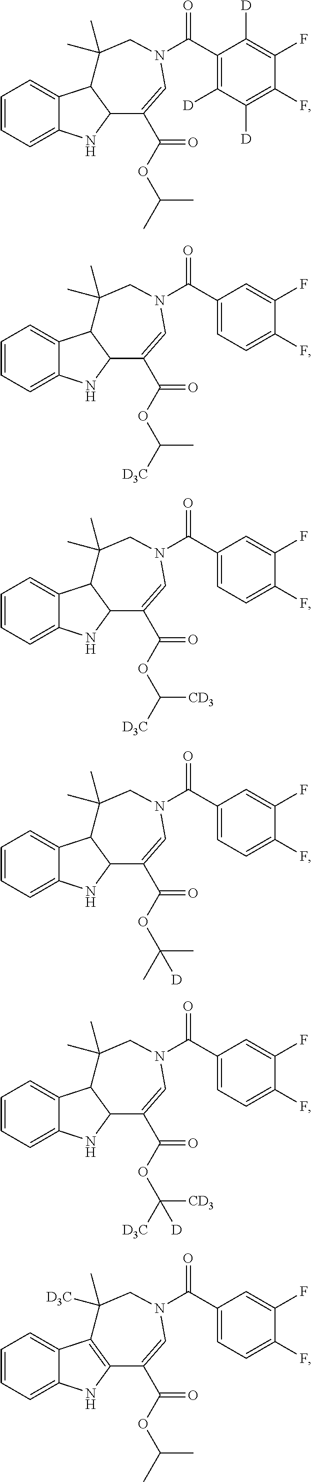

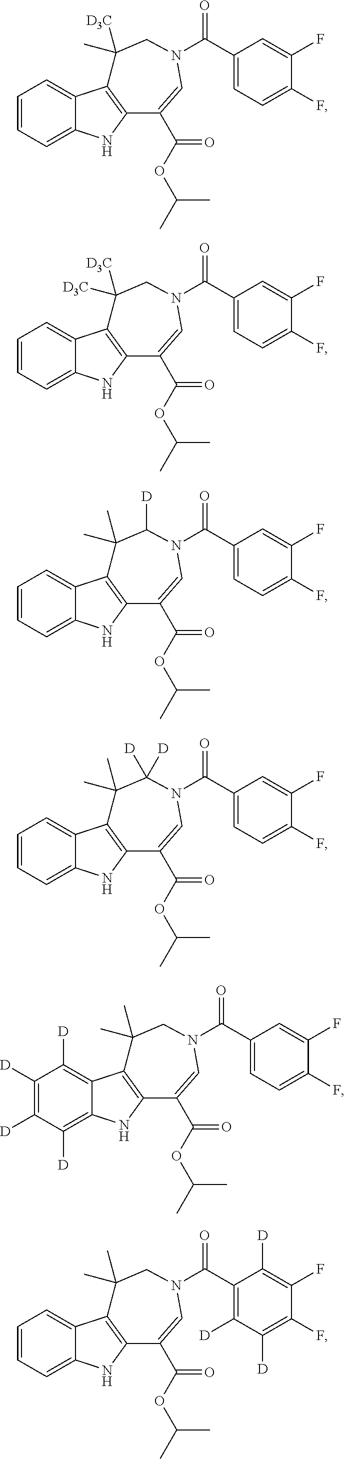

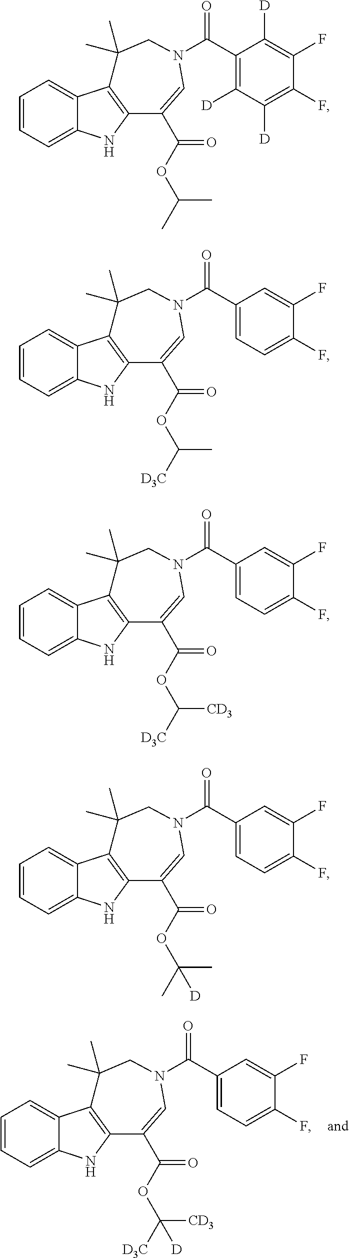

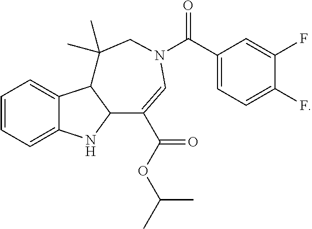 [SMILES: CC(C)OC(=O)C1=CN(C(=O)c2ccc(F)c(F)c2)CC(C)(C)C2c3ccccc3NC12]

Also disclosed herein are compounds having the structure of formula 22

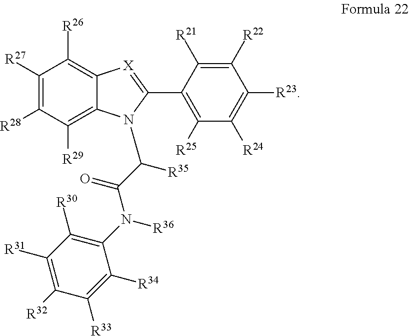

With reference to formula 22, X is N or CR³⁷; R²¹-R³⁴ independently are selected from hydrogen, deuterium, halogen, CF₃, NO₂, OH, amino, acyl, carboxyl, carboxyl ester, cyano, aminocarbonyl, aminosulfonyl, aliphatic, D-aliphatic, heteroaliphatic or D-heteroaliphatic; R³⁵ is aliphatic, D-aliphatic, heteroaliphatic or D-heteroaliphatic; R³⁶ is hydrogen, aliphatic, D-aliphatic, heteroaliphatic or D-heteroaliphatic; and R³⁷ is hydrogen, deuterium, halogen, CF₃, NO₂, OH, amino, acyl, carboxyl, carboxyl ester, cyano, aminocarbonyl, aminosulfonyl, aliphatic, D-aliphatic, heteroaliphatic or D-heteroaliphatic. In some embodiments, at least one of R²¹-R³⁵ and R³⁷ is or comprises deuterium, and in certain embodiments, at least one of R²¹-R³⁵ is or comprises deuterium.

Also with reference to formula 22, in some embodiments, none of R²¹-R³⁷ is —R^{x}-L^{x}-R^{x2}, where R^{x} is selected from O, NR^{x3}, sulfonyl or S; R^{x3} is selected from H, aliphatic, or aryl; L^{x} is selected from a bond, aliphatic, heteroaliphatic, aryl, heteroaryl or CR^{x4}R^{x5}; R^{x4} and R^{x5} are each independently selected from H, D, halogen, aliphatic, —C(O)OR^{x6}, or —C(O)NR^{x6}R^{x7}; R^{x6} and R^{x7} are each independently selected from H, aliphatic; R^{x2} is selected from —C(O)L^{x2}R^{x8} or a carboxyl bioisostere; L^{x2} is a bond or NR^{x3}; R^{x8} is H, aliphatic, —OR^{x9}, N(R^{x9})₂, —C(O)R^{x9}, —S(O)₂R⁹, —C(O)OR^{x9}, —S(O)₂N(R^{x9})₂ or —C(O)N(R^{x9})₂; and each R^{x9} is independently selected from H, aliphatic.

In some embodiments, R³⁵ is alkyl, cycloalkyl, deuterated alkyl or deuterated cycloalkyl. In certain disclosed embodiments, R³⁵ is cycloalkyl or deuterated cycloalkyl, typically cyclohexyl or deuterated cyclohexyl, having from 1 to 11 deuterium atoms.

In some examples, R³⁶ is hydrogen.

In some embodiments, R³² is carboxyl and/or R³⁴ is CF₃.

In some embodiments, R²³ is halogen, and in certain embodiments R²³ is chloro.

In some embodiments, the compound is chiral, and in certain embodiments, the compound is the S-stereoisomer.

In some embodiments, X is N, leading to compounds having a structure of formula 23

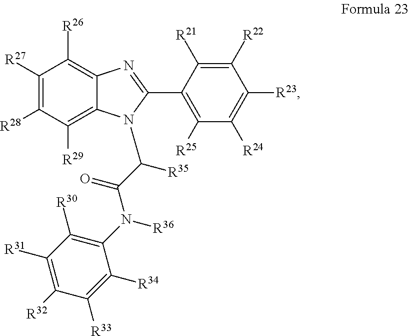

where R²¹-R³⁶ is as defined above with respect to formula 22, and at least one of R²¹-R³⁶ is or comprises deuterium.

In other embodiments, X is CH, leading to compounds having the structure of formula 24

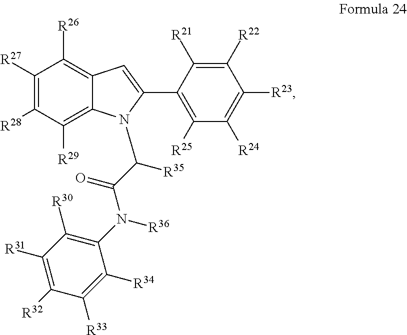

where R²¹-R³⁶ is as defined above with respect to formula 22.

Exemplary compounds having the structure of formula 22 include:

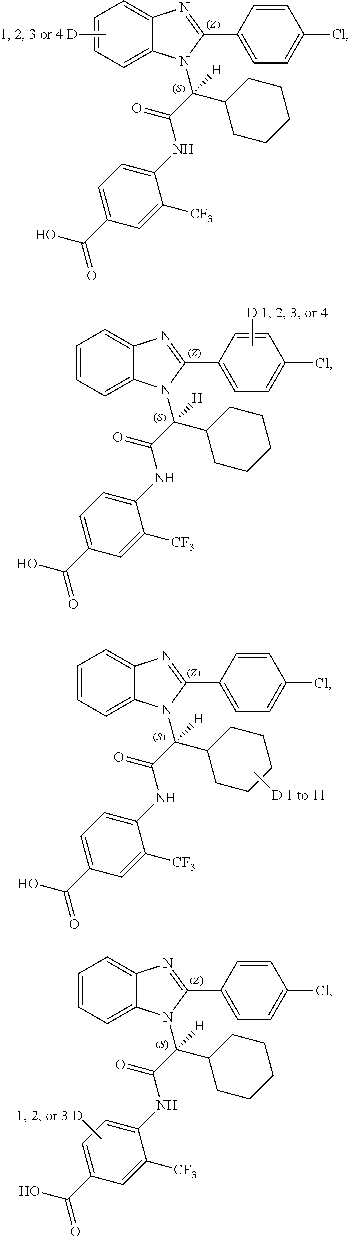

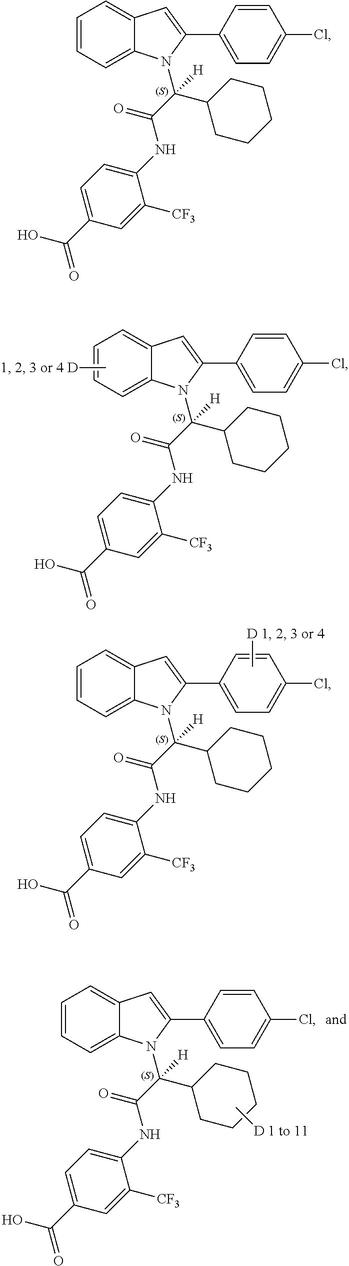

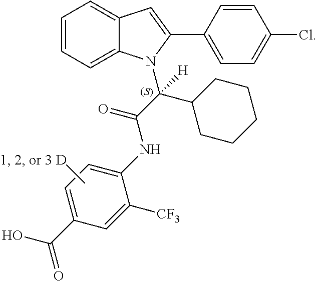

Also disclosed herein are compounds having the structure of formula 25

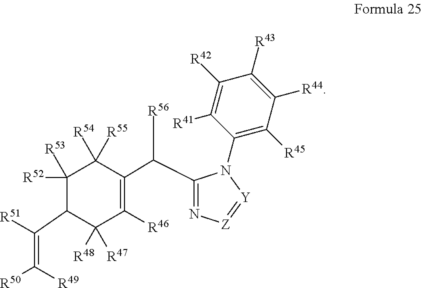

With reference to formula 25, R⁴¹-R⁴⁸ and R⁵²-R⁵⁵ independently are selected from hydrogen, deuterium, halogen, CF₃, NO₂, OH, amino, acyl, carboxyl, carboxyl ester, cyano, aminocarbonyl, aminosulfonyl, aliphatic, D-aliphatic, heteroaliphatic or D-heteroaliphatic; R⁴⁹-R⁵¹ independently are selected from hydrogen, deuterium, aliphatic, D-aliphatic, heteroaliphatic or D-heteroaliphatic; R⁵⁶ is amino, cycloamino or substituted cycloamino, such as 5-, 6-, or 7-membered cycloamino; Y and Z are independently N or CR⁵⁷; and each R⁵⁷ independently is selected from deuterium, halogen, CF₃, NO₂, OH, amino, acyl, carboxyl, carboxyl ester, cyano, aminocarbonyl, aminosulfonyl, aliphatic, D-aliphatic, heteroaliphatic or D-heteroaliphatic.

Also with reference to formula 25, in some embodiments, none of R⁴¹-R⁵⁷ is —R^{x}-L^{x}-R^{x2}, where R^{x} is selected from O, NR^{x3}, sulfonyl or S; R^{x3} is selected from H, aliphatic, or aryl; L^{x} is selected from a bond, aliphatic, heteroaliphatic, aryl, heteroaryl or CR^{x4}R^{x5}; R^{x4} and R^{x5} are each independently selected from H, D, halogen, aliphatic, —C(O)OR^{x6}, or —C(O)NR^{x6}R^{x7}; R^{x6} and R^{x7} are each independently selected from H, aliphatic; R^{x2} is selected from —C(O)L^{x2}R^{x8} or a carboxyl bioisostere; L^{x2} is a bond or NR^{x3}; R^{x8} is H, aliphatic, —OR^{x9}, N(R^{x9})₂, —C(O)R^{x9}, —S(O)₂R^{x9}, —C(O)OR^{x9}, —S(O)₂N(R⁹)₂ or —C(O)N(R^{x9})₂; and each R^{x9} is independently selected from H, aliphatic.

In some embodiments, at least one of R⁴¹-R⁵⁶ is or comprises deuterium.

In some embodiments, R⁵¹ is an aliphatic or D-aliphatic, and in certain embodiments, R⁵¹ is a methyl or deuterated methyl, having from 1 to 3 deuterium atoms.

In some embodiments, R⁴⁹ and R⁵⁰ independently are hydrogen or deuterium.

In some embodiments, R⁴¹ and R⁴⁵ independently are aliphatic or D-aliphatic, and in particular embodiments, R⁴¹ and R⁴⁵ are methyl or deuterated methyl, having from 1 to 3 deuterium atoms.

In some embodiments, R⁵⁶ is a cycloamino or substituted cycloamino, such as pyrrolidine, 2-methylpyrrolidine, morpholine, 4-methylpiperazine, piperidine, or azepane (homopiperidine).

In some embodiments, Y is N and Z is N leading to compounds having a structure of formula 26

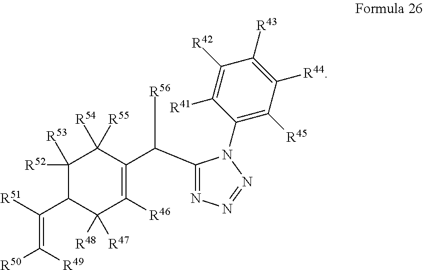

In other embodiments, Y is CH and Z is CH leading to compounds having a structure of formula 27

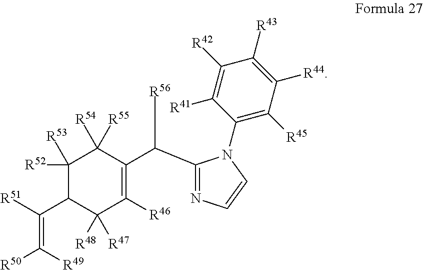

In other examples, Y is N and Z is CH leading to compounds having a structure of formula 28

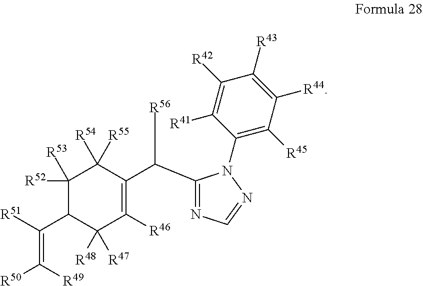

And in other examples Y is CH and Z is N leading to compounds having a structure of formula 29

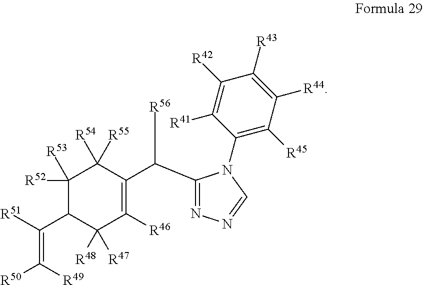

With respect to formulas 26-29, R⁴¹-R⁵⁶ are as defined for formula 25.

Exemplary compounds having the structure of formula 25 include:

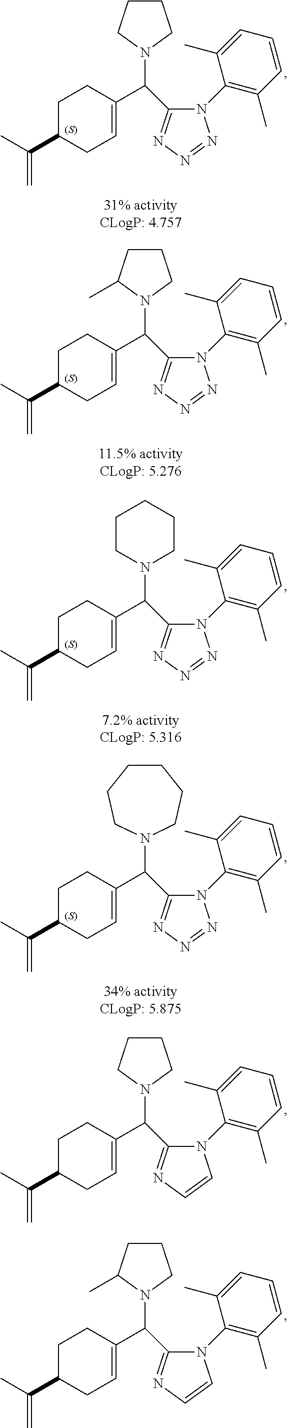

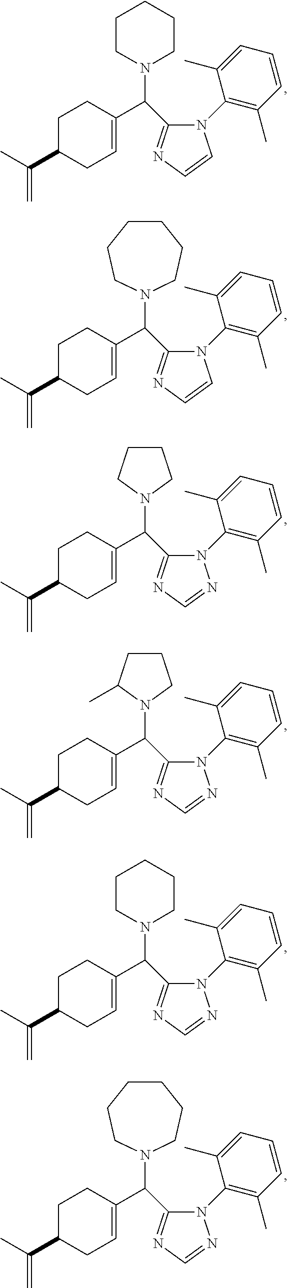

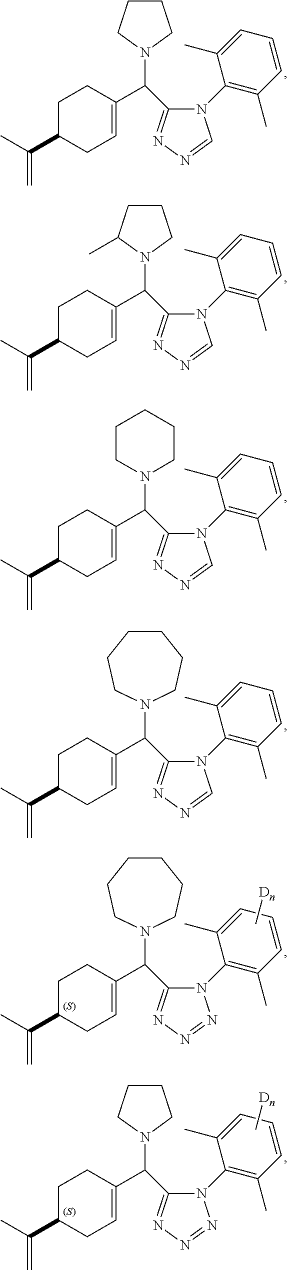

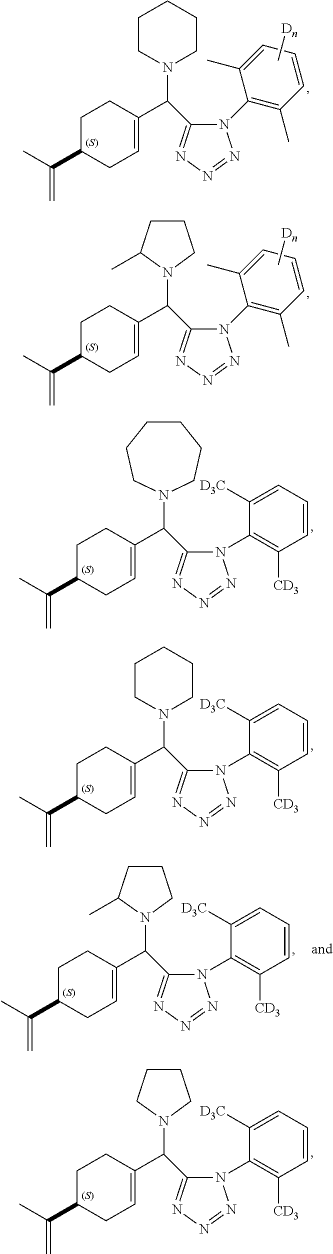

wherein n is from 1 to 3.

Also disclosed herein are compounds having the structure of formula 30,

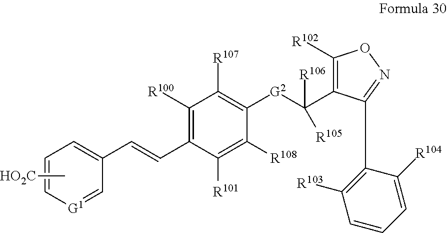

wherein G¹ is CH or N; G² is O or NH; R¹⁰⁰ and R¹⁰¹ are independently H, D, halogen, aliphatic, D-aliphatic, heteroaliphatic or D-heteroaliphatic; R¹⁰² is aliphatic, heteropaliphatic, D-aliphatic or D-heteroaliphatic; R¹⁰³ and R¹⁰⁴ are independently H, D, halogen, OH, alkoxy, O-polyhaloalkyl, aliphatic, D-aliphatic, heteroaliphatic or D-heteroaliphatic; R¹⁰⁵ and R¹⁰⁶ are each independently H, D, halogen, aliphatic or D-aliphatic; R¹⁰⁷ and R¹⁰⁸ are each independently H, D, aliphatic, D-aliphatic or halogen. In some embodiments, R¹⁰⁰ and R¹⁰¹ are independently H, D, lower alkyl, halogen, or CF₃; R¹⁰² is lower alkyl; R¹⁰³ and R¹⁰⁴ are independently H, D, lower alkyl, halogen, CF₃, OH, O-alkyl, or O-polyhaloalkyl; R¹⁰⁵ and R¹⁰⁶ are each independently H, D, halogen, alkyl or deuterated alkyl; R¹⁰⁷ and R¹⁰⁸ are each independently H, D, alkyl, deuterated alkyl or halogen. In some embodiments, at least one of R¹⁰⁰, R¹⁰¹, R¹⁰², R¹⁰³, R¹⁰⁴, R¹⁰⁵, R¹⁰⁶, R¹⁰⁷ and R¹⁰⁸ is or comprises deuterium. In some embodiments, at least one of R¹⁰⁵, R¹⁰⁶, R¹⁰⁷ and R¹⁰⁸ is or comprises deuterium. In other embodiments, at least one of R¹⁰⁷ and R¹⁰⁸ is halogen, and may be fluoro.

In certain embodiments, the compound has a structure of formula 31

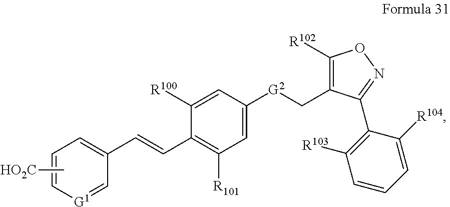

wherein G¹ is CH or N; G² is O or NH; R¹⁰⁰ and R¹⁰¹ are independently H, lower alkyl, halogen, or CF₃; R¹⁰² is lower alkyl; R¹⁰³ and R¹⁰⁴ are independently H, lower alkyl, halogen, CF₃, OH, O-alkyl, or O-polyhaloalkyl.

Exemplary compounds having a structure of formula 30 or formula 31 include

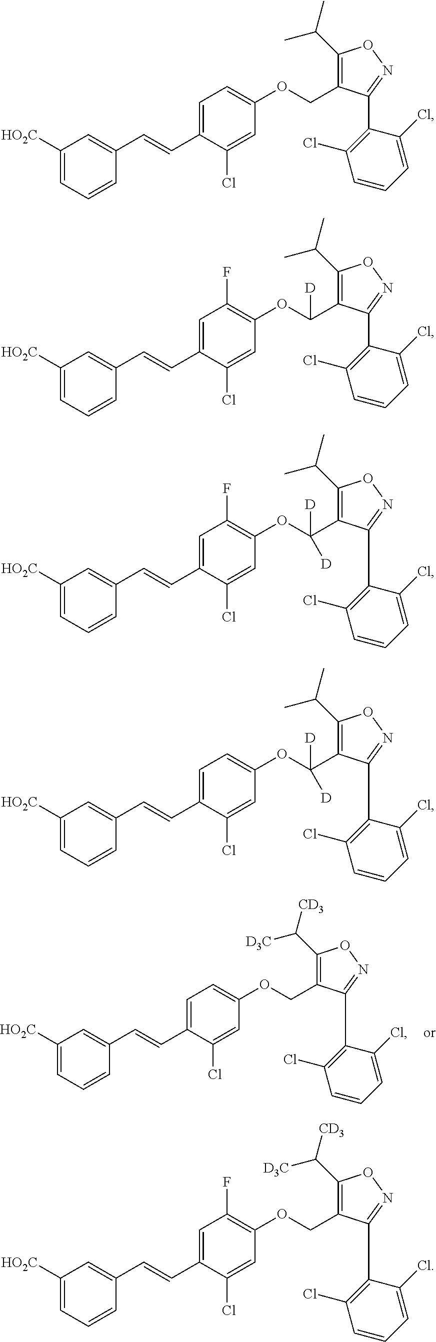

Other exemplary compounds having the structure of formula 30 or formula 31 include
(E)-3-(2-chloro-4-((3-(2,6-dichlorophenyl)-5-isopropylisoxazol-4-yl)methoxy)styryl)benzoic acid;
(E)-3-(2-chloro-4-((3-(2,6-dichlorophenyl)-5-isopropylisoxazol-4-yl)methoxy-d)-5-fluorostyryl-d)benzoic acid;
(E)-3-(2-chloro-4-((3-(2,6-dichlorophenyl)-5-isopropylisoxazol-4-yl)methoxy-d2)-5-fluorostyryl-d2)benzoic acid;
(E)-3-(2-chloro-4-((3-(2,6-dichlorophenyl)-5-isopropylisoxazol-4-yl)methoxy-d2)styryl-d2)benzoic acid;
(E)-3-(2-chloro-4-((3-(2,6-dichlorophenyl)-5-(propan-2-yl-1,1,1,3,3,3-d6)isoxazol-4-yl)methoxy)styryl-d6)benzoic acid; or
(E)-3-(2-chloro-4-((3-(2,6-dichlorophenyl)-5-(propan-2-yl-1,1,1,3,3,3-d6)isoxazol-4-yl)methoxy)-5-fluorostyryl-d6)benzoic acid.

Also disclosed herein are compounds having the structure of formula 32,

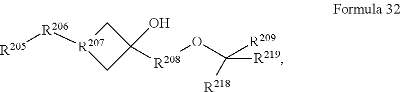

wherein R²⁰⁵ is selected from the group consisting of COOR²¹⁰, CONR²¹¹R²¹², tetrazolyl, SO₂NR²¹¹R²¹², C₁₋₆ alkyl, SO₂—C₁₋₆ alkyl and H, with R²¹⁰ independently selected from the group consisting of H or C₁₋₆ alkyl, and R²¹¹ and R²¹² independently from each other selected from the group consisting of H, C₁₋₆ alkyl, halo-C₁₋₆ alkyl, C₁₋₆ alkylene-R²¹³, SO₂—C₁₋₆ alkyl, wherein R²¹³ is selected from the group consisting of COOH, OH and SO₃H;

R²⁰⁶ is selected from the group consisting of phenyl, pyridyl, pyrimidyl, pyrazolyl, indolyl, thienyl, benzothienyl, indazolyl, benzisoxazolyl, benzofuranyl, benzotriazolyl, furanyl, benzothiazolyl, thiazolyl, oxadiazolyl, each optionally substituted with one or two groups independently selected from the group consisting of OH, O-C₁₋₆ alkyl, O-halo-C₁₋₆ alkyl, C₁₋₆ alkyl, halo-C₁₋₆ alkyl, C₃₋₆ cycloalkyl, D and halogen;

R²⁰⁷ is selected from N or CH;

R²⁰⁸ is selected from the group consisting of phenyl, pyridyl, thiazolyl, thiophenyl, pyrimidyl, each optionally substituted with one or two groups independently selected from the group consisting of D, C₁₋₆ alkyl, halo-C₁₋₆ alkyl, halogen and CF₃;

R²⁰⁹ is selected from

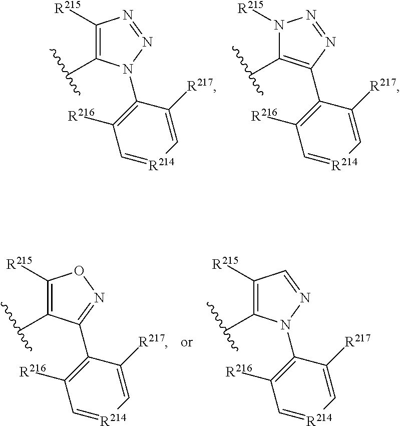

wherein

R²¹⁴═CH, N, NO, CD;

R²¹⁵ is selected from the group consisting of hydrogen, C₁₋₃ alkyl, C₃₋₆ cycloalkyl, C₄₋₅ alkylcycloalkyl, wherein C₁₋₃ alkyl is optionally substituted with 1 to 3 substituents independently selected from halogen, hydroxy or C₁₋₆ alkoxy;

R²¹⁶ and R²¹⁷ are independently selected from the group consisting of hydrogen, D, C₁₋₃ alkyl, C₁₋₃ haloalkyl, C₁₋₃ alkoxy, C₁₋₃ haloalkoxy, D-aliphatic and halogen.

R²¹⁸ and R²¹⁹ are each independently H or D. In some embodiments, R²¹⁸ and R²¹⁹ are both H. In other embodiments, at least one of R²¹⁸ and R²¹⁹ is D.

In some embodiments, the compound comprises at least one deuterium. In some embodiments, R²⁰⁶ and/or R²⁰⁸ comprise at least one deuterium. In other embodiments, R²¹⁴ is CD. In certain embodiments, at least one of R²¹⁶ and R²¹⁷ is or comprises deuterium.

In some embodiments for compounds having the structure of formula 32, R²⁰⁵, —R²⁰⁶ is selected

from

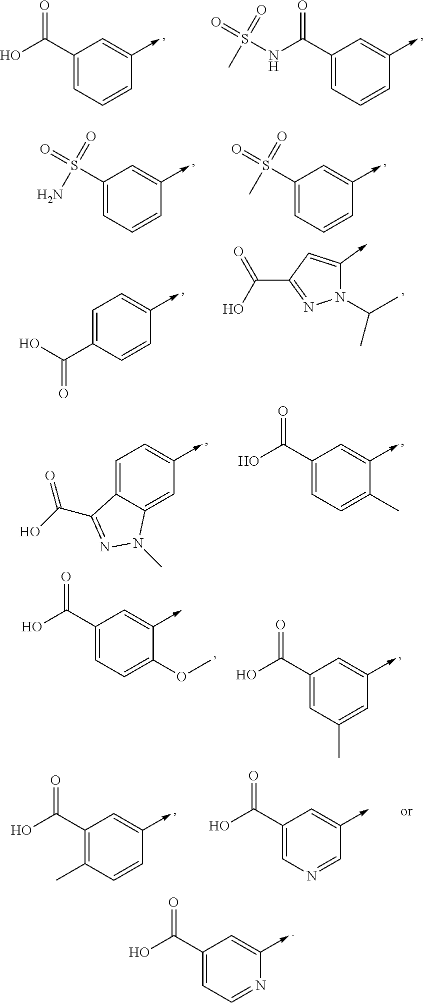

In some embodiments for compounds having the structure of formula 32, R²⁰⁸ is

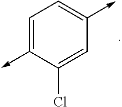

In some embodiments for compounds having the structure of formula 32,

### R²⁰⁹ is

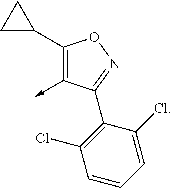

Exemplary compounds having the structure of formula 32 include:

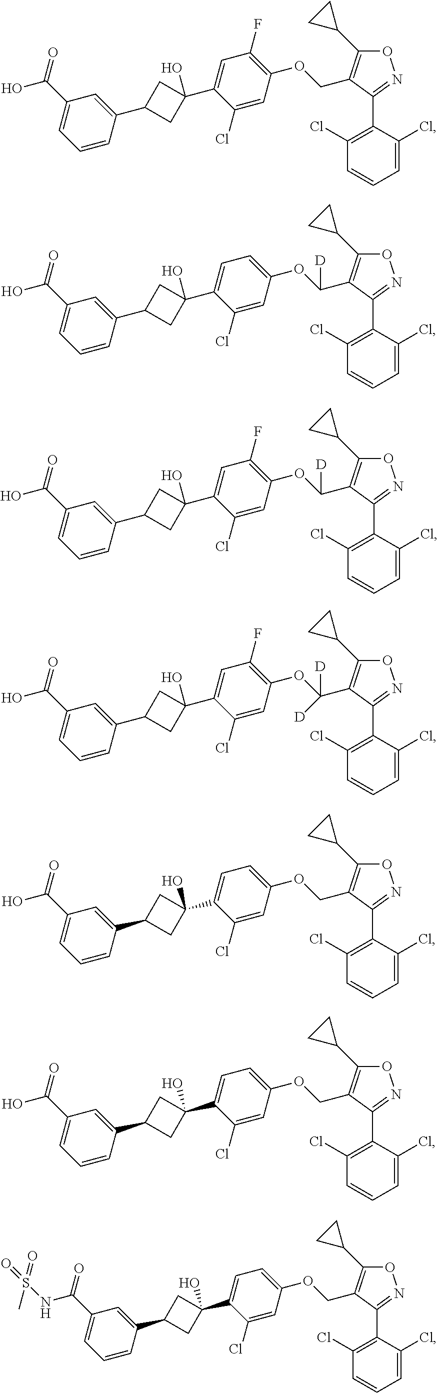

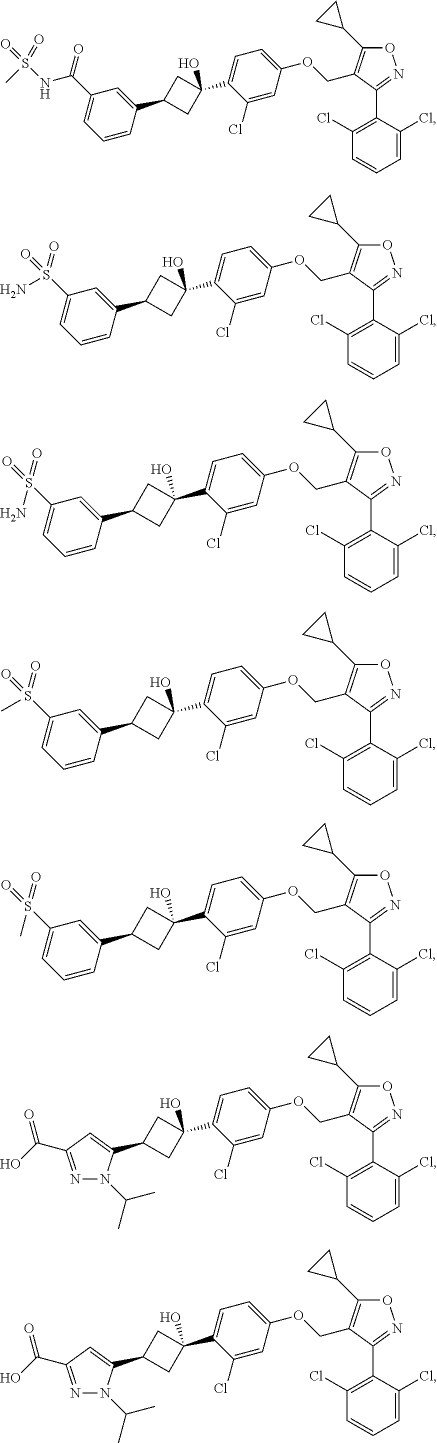

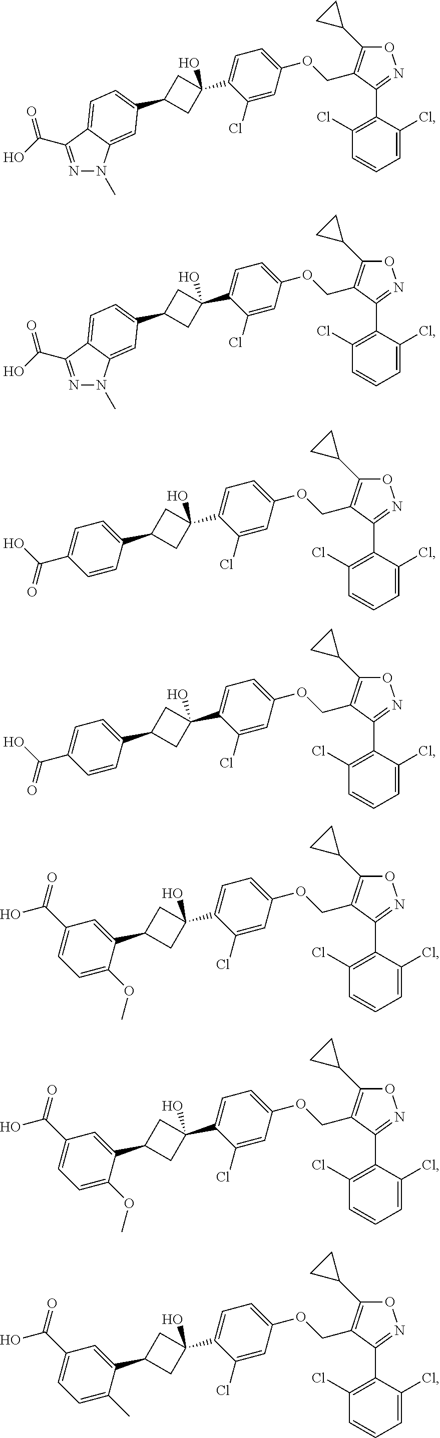

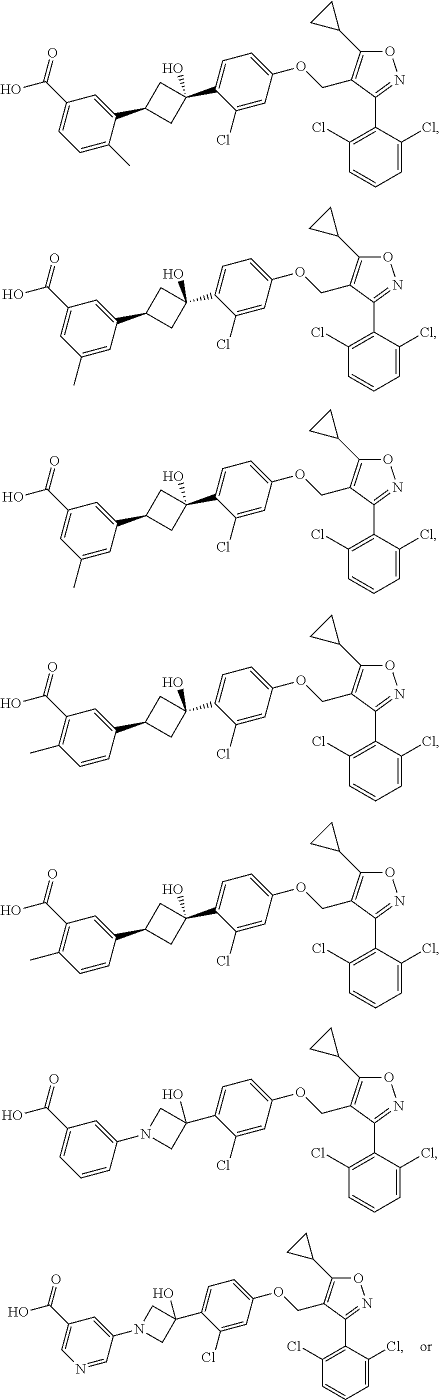

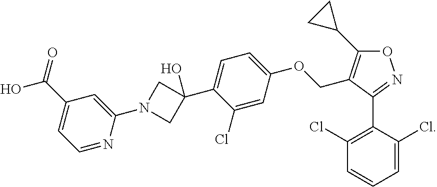 [SMILES: O=C(O)c1ccnc(N2CC(O)(c3ccc(OCc4c(-c5c(Cl)cccc5Cl)noc4C4CC4)cc3Cl)C2)c1]

Other Exemplary compounds having the structure of formula 32 include
3-(3-(2-chloro-4-((5-cyclopropyl-3-(2,6-dichlorophenyl)isoxazol-4-yl)methoxy)-5-fluorophenyl)-3-hydroxycyclobutyl)benzoic acid;
3-(3-(2-chloro-4-((5-cyclopropyl-3-(2,6-dichlorophenyl)isoxazol-4-yl)methoxy-d)phenyl)-3-hydroxycyclobutyl)benzoic acid;
3-(3-(2-chloro-4-((5-cyclopropyl-3-(2,6-dichlorophenyl)isoxazol-4-yl)methoxy-d)-5-fluorophenyl)-3-hydroxycyclobutyl)benzoic acid;
3-(3-(2-chloro-4-((5-cyclopropyl-3-(2,6-dichlorophenyl)isoxazol-4-yl)methoxy-d2)-5-fluorophenyl)-3-hydroxycyclobutyl)benzoic acid;
3-((1s,3s)-3-(2-chloro-4-((5-cyclopropyl-3-(2,6-dichlorophenyl)isoxazol-4-yl)methoxy)phenyl)-3-hydroxycyclobutyl)benzoic acid;
3-((1r,3r)-3-(2-chloro-4-((5-cyclopropyl-3-(2,6-dichlorophenyl)isoxazol-4-yl)methoxy)phenyl)-3-hydroxycyclobutyl)benzoic acid;
3-((1r,3r)-3-(2-chloro-4-((5-cyclopropyl-3-(2,6-dichlorophenyl)isoxazol-4-yl)methoxy)phenyl)-3-hydroxycyclobutyl)-N—(methylsulfonyl)benzamide;
3-((1s,3s)-3-(2-chloro-4-((5-cyclopropyl-3-(2,6-dichlorophenyl)isoxazol-4-yl)methoxy)phenyl)-3-hydroxycyclobutyl)-N—(methylsulfonyl)benzamide;
3-((1s,3s)-3-(2-chloro-4-((5-cyclopropyl-3-(2,6-dichlorophenyl)isoxazol-4-yl)methoxy)phenyl)-3-hydroxycyclobutyl)benzenesulfonamide;
3-((1r,3r)-3-(2-chloro-4-((5-cyclopropyl-3-(2,6-dichlorophenyl)isoxazol-4-yl)methoxy)phenyl)-3-hydroxycyclobutyl)benzenesulfonamide;
(1s,3 s)-1-(2-chloro-4-((5-cyclopropyl-3-(2,6-dichlorophenyl)isoxazol-4-yl)methoxy)phenyl)-3-(3-(methylsulfonyl)phenyl)cyclobutan-1-ol;
(1r,3r)-1-(2-chloro-4-((5-cyclopropyl-3-(2,6-dichlorophenyl)isoxazol-4-yl)methoxy)phenyl)-3-(3-(methylsulfonyl)phenyl)cyclobutan-1-ol;
5-((1s,3s)-3-(2-chloro-4-((5-cyclopropyl-3-(2,6-dichlorophenyl)isoxazol-4-yl)methoxy)phenyl)-3-hydroxycyclobutyl)-1-isopropyl-1H-pyrazole-3-carboxylic acid;
5-((1r,3r)-3-(2-chloro-4-((5-cyclopropyl-3-(2,6-dichlorophenyl)isoxazol-4-yl)methoxy)phenyl)-3-hydroxycyclobutyl)-1-isopropyl-1H-pyrazole-3-carboxylic acid;
6-((1s,3 s)-3-(2-chloro-4-((5-cyclopropyl-3-(2,6-dichlorophenyl)isoxazol-4-yl)methoxy)phenyl)-3-hydroxycyclobutyl)-1-methyl-1H-indazole-3-carboxylic acid;
6-((1r,3r)-3-(2-chloro-4-((5-cyclopropyl-3-(2,6-dichlorophenyl)isoxazol-4-yl)methoxy)phenyl)-3-hydroxycyclobutyl)-1-methyl-1H-indazole-3-carboxylic acid;
4-((1s,3s)-3-(2-chloro-4-((5-cyclopropyl-3-(2,6-dichlorophenyl)isoxazol-4-yl)methoxy)phenyl)-3-hydroxycyclobutyl)benzoic acid;
4-((1r,3r)-3-(2-chloro-4-((5-cyclopropyl-3-(2,6-dichlorophenyl)isoxazol-4-yl)methoxy)phenyl)-3-hydroxycyclobutyl)benzoic acid;
3-((1s,3 s)-3-(2-chloro-4-((5-cyclopropyl-3-(2,6-dichlorophenyl)isoxazol-4-yl)methoxy)phenyl)-3-hydroxycyclobutyl)-4-methoxybenzoic acid;
3-((1r,3r)-3-(2-chloro-4-((5-cyclopropyl-3-(2,6-dichlorophenyl)isoxazol-4-yl)methoxy)phenyl)-3-hydroxycyclobutyl)-4-methoxybenzoic acid;
3-((1s,3s)-3-(2-chloro-4-((5-cyclopropyl-3-(2,6-dichlorophenyl)isoxazol-4-yl)methoxy)phenyl)-3-hydroxycyclobutyl)-4-methylbenzoic acid;
3-((1r,3r)-3-(2-chloro-4-((5-cyclopropyl-3-(2,6-dichlorophenyl)isoxazol-4-yl)methoxy)phenyl)-3-hydroxycyclobutyl)-4-methylbenzoic acid;
3-((1s,3s)-3-(2-chloro-4-((5-cyclopropyl-3-(2,6-dichlorophenyl)isoxazol-4-yl)methoxy)phenyl)-3-hydroxycyclobutyl)-5-methylbenzoic acid;
3-((1r,3r)-3-(2-chloro-4-((5-cyclopropyl-3-(2,6-dichlorophenyl)isoxazol-4-yl)methoxy)phenyl)-3-hydroxycyclobutyl)-5-methylbenzoic acid;
5-((1s,3 s)-3-(2-chloro-4-((5-cyclopropyl-3-(2,6-dichlorophenyl)isoxazol-4-yl)methoxy)phenyl)-3-hydroxycyclobutyl)-2-methylbenzoic acid;
5-((1r,3r)-3-(2-chloro-4-((5-cyclopropyl-3-(2,6-dichlorophenyl)isoxazol-4-yl)methoxy)phenyl)-3-hydroxycyclobutyl)-2-methylbenzoic acid;
3-(3-(2-chloro-4-((5-cyclopropyl-3-(2,6-dichlorophenyl)isoxazol-4-yl)methoxy)phenyl)-3-hydroxyazetidin-1-yl)benzoic acid;
5-(3-(2-chloro-4-((5-cyclopropyl-3-(2,6-dichlorophenyl)isoxazol-4-yl)methoxy)phenyl)-3-hydroxyazetidin-1-yl)nicotinic acid; or
2-(3-(2-chloro-4-((5-cyclopropyl-3-(2,6-dichlorophenyl)isoxazol-4-yl)methoxy)phenyl)-3-hydroxyazetidin-1-yl)isonicotinic acid.

Also disclosed herein are compounds having the structure of formula 33,

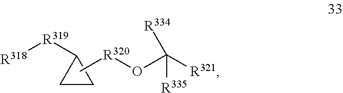

wherein

R³¹⁸ is selected from the group consisting of COOR³²², CONR³²³R³²⁴, tetrazolyl or H, with R³²² independently selected from the group consisting of H, or lower alkyl, and R³²³ and R³²⁴ independently from each other selected from the group consisting of H, lower alkyl, C₁₋₆ haloalkyl, C₁₋₆ alkylene-R³²⁵, SO₂—C₁₋₆ alkyl wherein R³²⁵ is selected from the group consisting of COOH, OH, or SO₃H;

R³¹⁹ is selected from the group consisting of phenyl, pyridyl, pyrazolyl, indolyl, thienyl, benzothienyl, indazolyl, benzisoxazolyl, benzofuranyl, benzotriazolyl, furanyl, benzothiazolyl, thiazolyl, each optionally substituted with one or two groups independently selected from the group consisting of OH, lower alkyl, lower cycloalkyl, or halogen;

R³²⁰ is selected from the group consisting of phenyl, pyridyl, thiazolyl, thiophenyl, pyrimidyl, each optionally substituted with one or two groups independently selected from the group consisting of lower alkyl, halogen, D or CF₃;

R³²¹ is

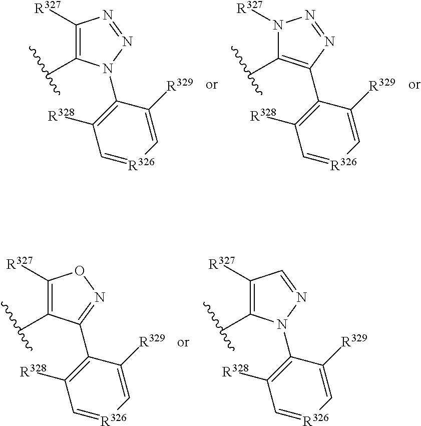

wherein R³²⁶ is CH, N, NO;

R₃₂₇ is selected from the group consisting of hydrogen, C₁-C₃ alkyl, C₃-C₆ cycloalkyl, C₄-C₅ alkylcycloalkyl, wherein C₁₋₃ alkyl is optionally substituted with 1 to 3 substituents independently selected from halogen, hydroxy or C₁₋₆ alkoxy,

R³²⁸ and R³²⁹ are independently selected from the group consisting of hydrogen, C₁-C₃ alkyl, C₁-C₃ haloalkyl, C₁-C₃ alkoxy, C₁-C₃ haloalkoxy and halogen.

R³³⁴ and R³³⁵ are each independently H or D. In some embodiments, at least one of R³³⁴ and R³³⁵ are D.

In some embodiments, R³²⁰ is substituted with at least one halogen or deuterium.

In some embodiments for compounds having formula 33, R³¹⁸ is selected from the group consisting of COOR³²², CONR³²³R³²⁴, tetrazolyl or H, with R³²², R³²³ and R³²⁴ independently selected from the group consisting of H, lower alkyl;

R³¹⁹ is selected from the group consisting of phenyl, pyridyl, indolyl, thienyl, benzothienyl, indazolyl, benzisoxazolyl, benzofuranyl, benzotriazolyl, furanyl, benzothiazolyl, thiazolyl, each optionally substituted with one or two groups independently selected from the group consisting of OH, lower alkyl, lower cycloalkyl;

R³²⁰ is selected from the group consisting of phenyl, pyridyl, thiazolyl, thiophenyl, pyrimidyl, each optionally substituted with one or two groups independently selected from the group consisting of lower alkyl, halogen, D or CF₃;

R³²¹ is

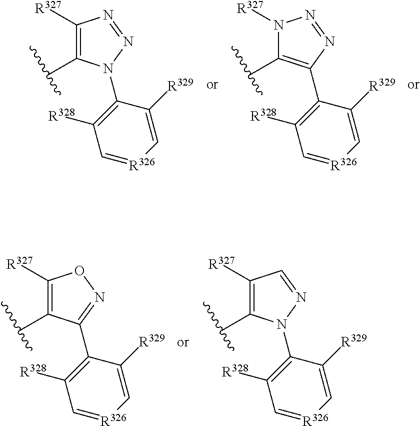

wherein R³²⁶ is CH, N, NO;

R³²⁷ is selected from the group consisting of hydrogen, C₁-C₃ alkyl, C₁-C₃ haloalkyl, C₃-C₆ cycloalkyl, C₄-C₅ alkylcycloalkyl;

R³²⁸ and R³²⁹ are independently selected from the group consisting of hydrogen, C₁-C₃ alkyl, C₁-C₃ haloalkyl, C₁-C₃ alkoxy, C₁-C₃ haloalkoxy and halogen.

In some embodiments, compounds having the structure of formula 33 have the structure of formula 34

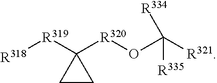

In other embodiments, compounds having structure of formula 33, have the structure of formula 35,

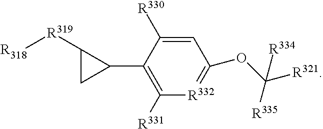

wherein R³³² is CH, CD or N;

R³³⁰ and R³³¹ are independently selected from the group consisting of H, D, lower alkyl, halogen and CF₃;

R³¹⁸-R³¹⁹ is selected from

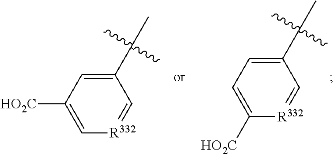

R³²⁷ is selected from the group consisting of isopropyl, t-butyl and cyclopropyl;

R³²⁸ and R³²⁹ are independently selected from the group consisting of halogen, C₁-C₃ alkyl, methoxy and trifluoromethoxy;

R³³⁴ and R³³⁵ are each independently H or D. In some embodiments, at least one of R³³⁴ and R³³⁵ are D.

In other embodiments for compounds having the formulas 33, 34 or 35, wherein R³¹⁹ is phenyl;

R³²⁰ is optionally substituted phenyl, preferably substituted with one substituent, preferably halogen, or two substituents, preferably both halogen or one halogen one deuterium;

R³²⁶ is CH;

R³²⁷ is cycloalkyl; and

R³²⁸ and R³²⁹ each are halogen.

Exemplary compounds having the structure of formulas 33, 34 or 35 include:

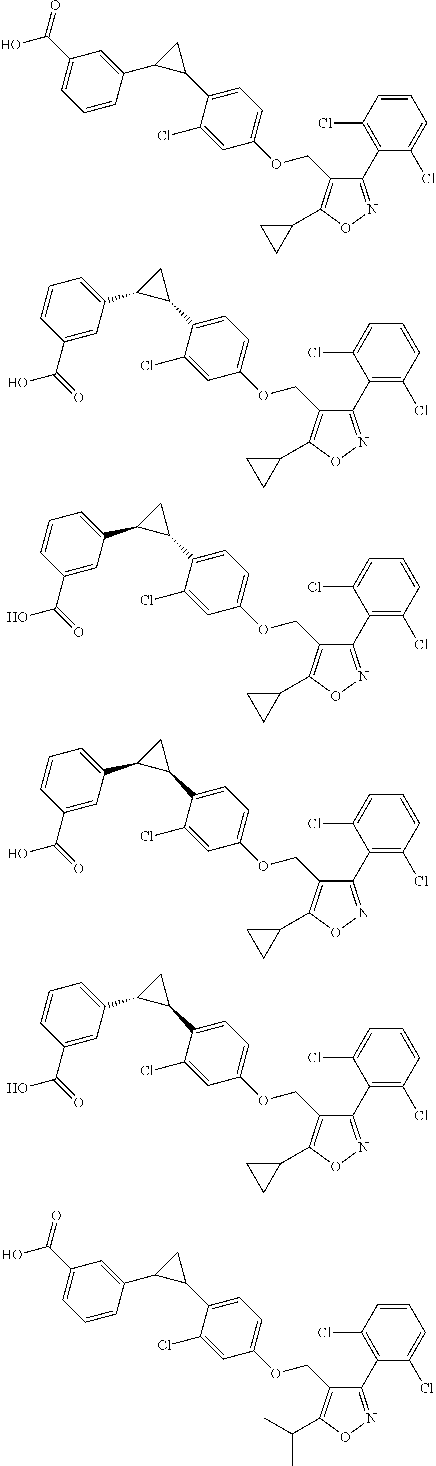 [SMILES: O=C(O)c1cccc(C2CC2c2ccc(OCc3c(-c4c(Cl)cccc4Cl)noc3C3CC3)cc2Cl)c1 ; O=C(O)c1cccc([C@@H]2C[C@@H]2c2ccc(OCc3c(-c4c(Cl)cccc4Cl)noc3C3CC3)cc2Cl)c1 ; O=C(O)c1cccc([C@H]2C[C@@H]2c2ccc(OCc3c(-c4c(Cl)cccc4Cl)noc3C3CC3)cc2Cl)c1 ; O=C(O)c1cccc([C@H]2C[C@H]2c2ccc(OCc3c(-c4c(Cl)cccc4Cl)noc3C3CC3)cc2Cl)c1 ; O=C(O)c1cccc([C@@H]2C[C@H]2c2ccc(OCc3c(-c4c(Cl)cccc4Cl)noc3C3CC3)cc2Cl)c1 ; CC(C)c1onc(-c2c(Cl)cccc2Cl)c1COc1ccc(C2CC2c2cccc(C(=O)O)c2)c(Cl)c1]

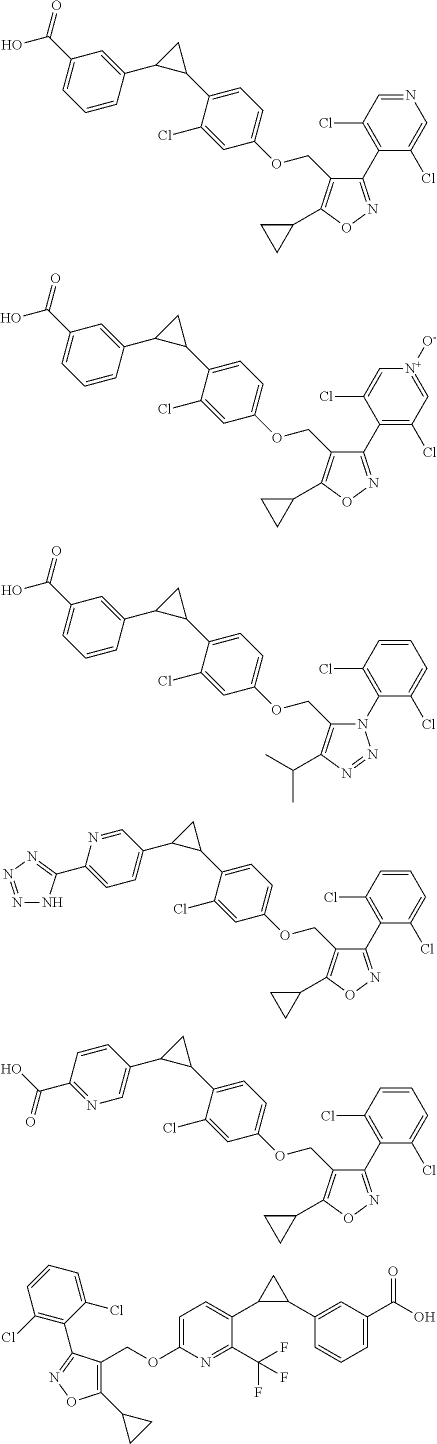

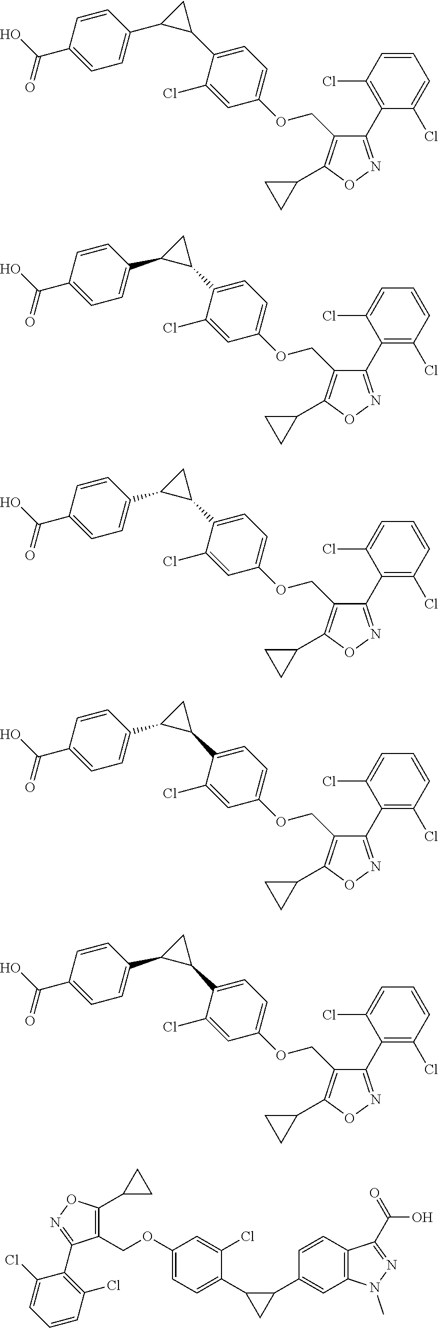 [SMILES: O=C(O)c1ccc(C2CC2c2ccc(OCc3c(-c4c(Cl)cccc4Cl)noc3C3CC3)cc2Cl)cc1 ; O=C(O)c1ccc([C@H]2C[C@@H]2c2ccc(OCc3c(-c4c(Cl)cccc4Cl)noc3C3CC3)cc2Cl)cc1 ; O=C(O)c1ccc([C@@H]2C[C@@H]2c2ccc(OCc3c(-c4c(Cl)cccc4Cl)noc3C3CC3)cc2Cl)cc1 ; O=C(O)c1ccc([C@@H]2C[C@H]2c2ccc(OCc3c(-c4c(Cl)cccc4Cl)noc3C3CC3)cc2Cl)cc1 ; O=C(O)c1ccc([C@H]2C[C@H]2c2ccc(OCc3c(-c4c(Cl)cccc4Cl)noc3C3CC3)cc2Cl)cc1 ; Cn1nc(C(=O)O)c2ccc(C3CC3c3ccc(OCc4c(-c5c(Cl)cccc5Cl)noc4C4CC4)cc3Cl)cc21]

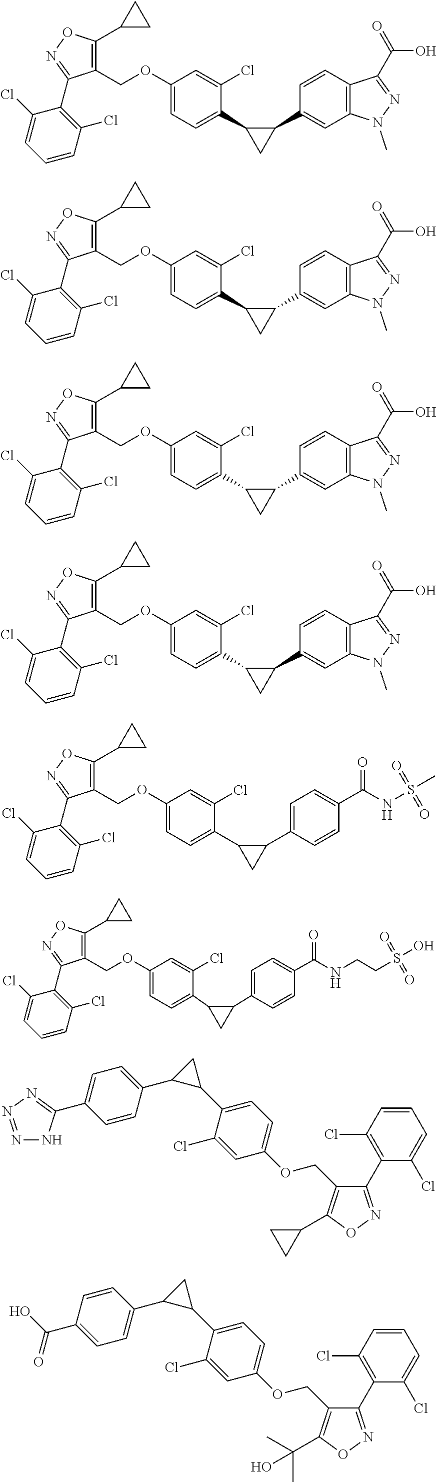

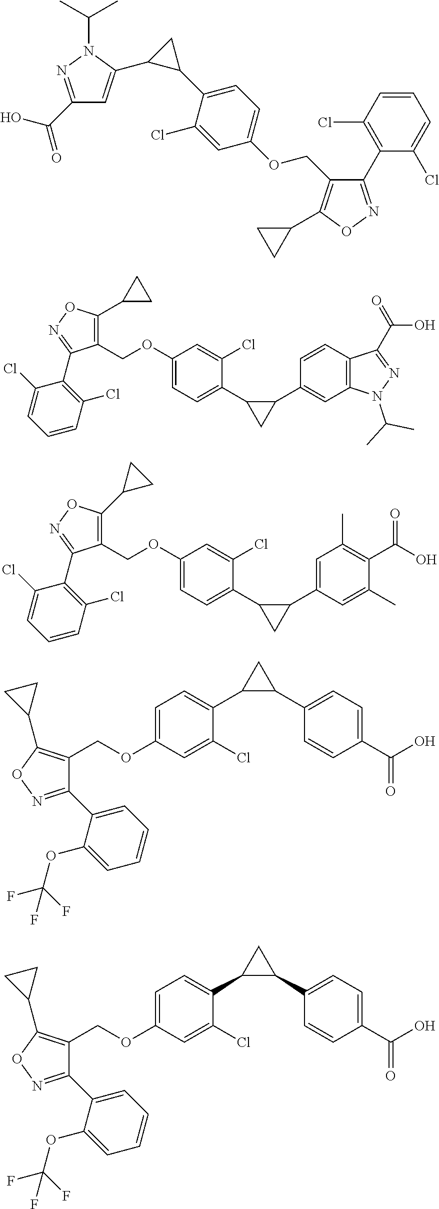

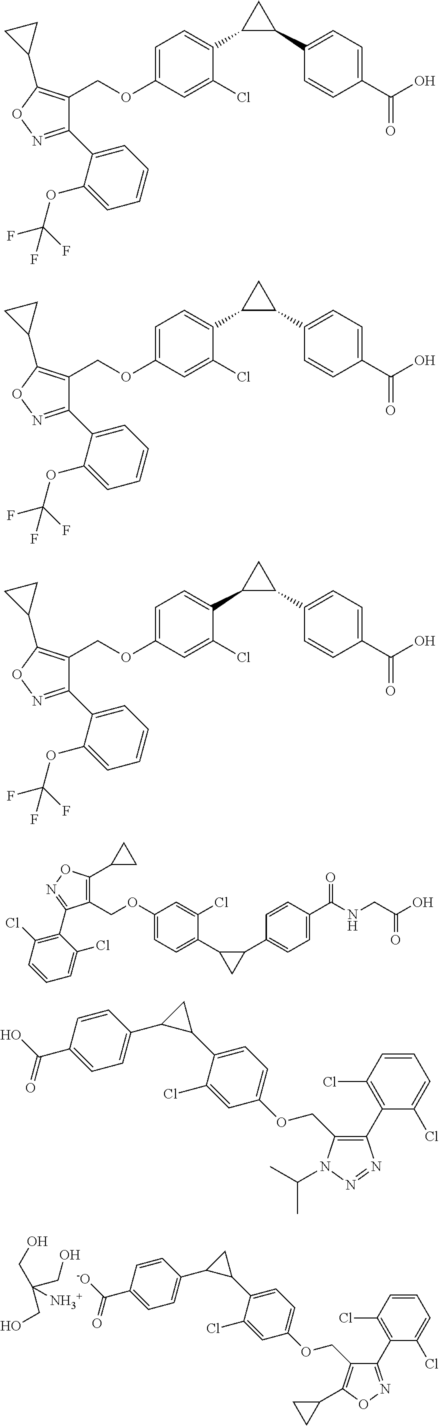

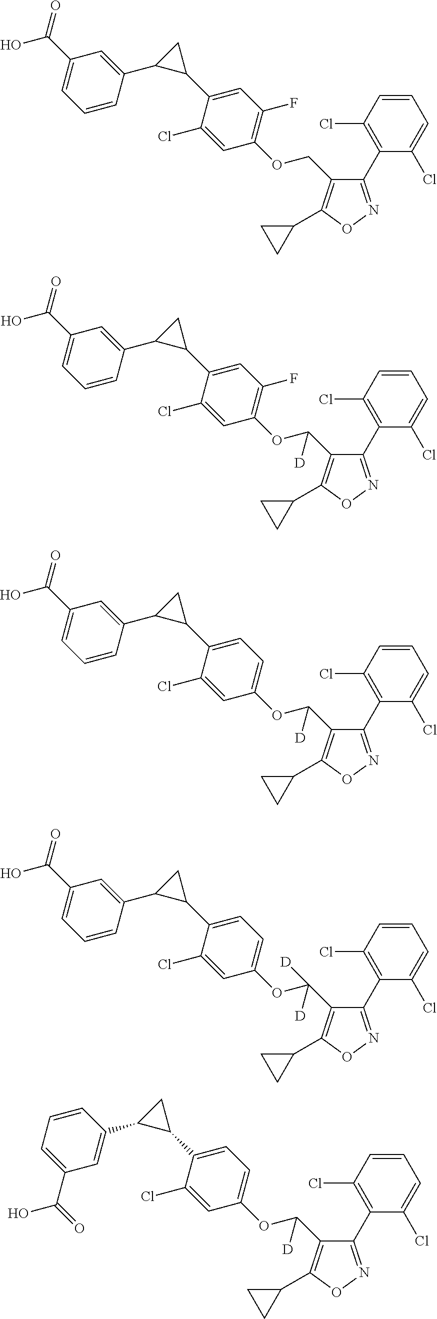 [SMILES: O=C(O)c1cccc(C2CC2c2cc(F)c(OCc3c(-c4c(Cl)cccc4Cl)noc3C3CC3)cc2Cl)c1 ; [2H]C(Oc1cc(Cl)c(C2CC2c2cccc(C(=O)O)c2)cc1F)c1c(-c2c(Cl)cccc2Cl)noc1C1CC1 ; [2H]C(Oc1ccc(C2CC2c2cccc(C(=O)O)c2)c(Cl)c1)c1c(-c2c(Cl)cccc2Cl)noc1C1CC1 ; [2H]C([2H])(Oc1ccc(C2CC2c2cccc(C(=O)O)c2)c(Cl)c1)c1c(-c2c(Cl)cccc2Cl)noc1C1CC1 ; [2H]C(Oc1ccc([C@H]2C[C@H]2c2cccc(C(=O)O)c2)c(Cl)c1)c1c(-c2c(Cl)cccc2Cl)noc1C1CC1]

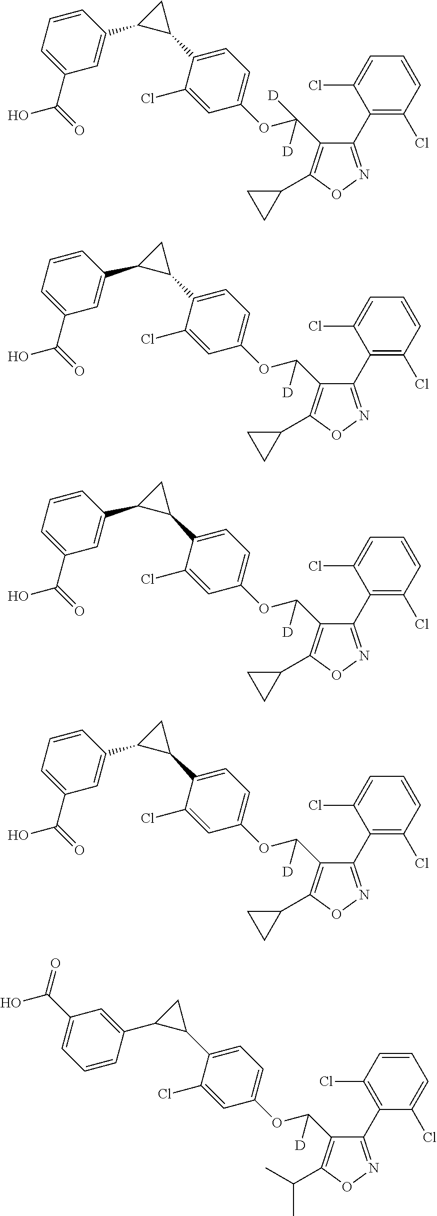 [SMILES: [2H]C([2H])(Oc1ccc([C@H]2C[C@H]2c2cccc(C(=O)O)c2)c(Cl)c1)c1c(-c2c(Cl)cccc2Cl)noc1C1CC1 ; [2H]C(Oc1ccc([C@H]2C[C@@H]2c2cccc(C(=O)O)c2)c(Cl)c1)c1c(-c2c(Cl)cccc2Cl)noc1C1CC1 ; [2H]C(Oc1ccc([C@@H]2C[C@@H]2c2cccc(C(=O)O)c2)c(Cl)c1)c1c(-c2c(Cl)cccc2Cl)noc1C1CC1 ; [2H]C(Oc1ccc([C@@H]2C[C@H]2c2cccc(C(=O)O)c2)c(Cl)c1)c1c(-c2c(Cl)cccc2Cl)noc1C1CC1 ; [2H]C(Oc1ccc(C2CC2c2cccc(C(=O)O)c2)c(Cl)c1)c1c(-c2c(Cl)cccc2Cl)noc1C(C)C]

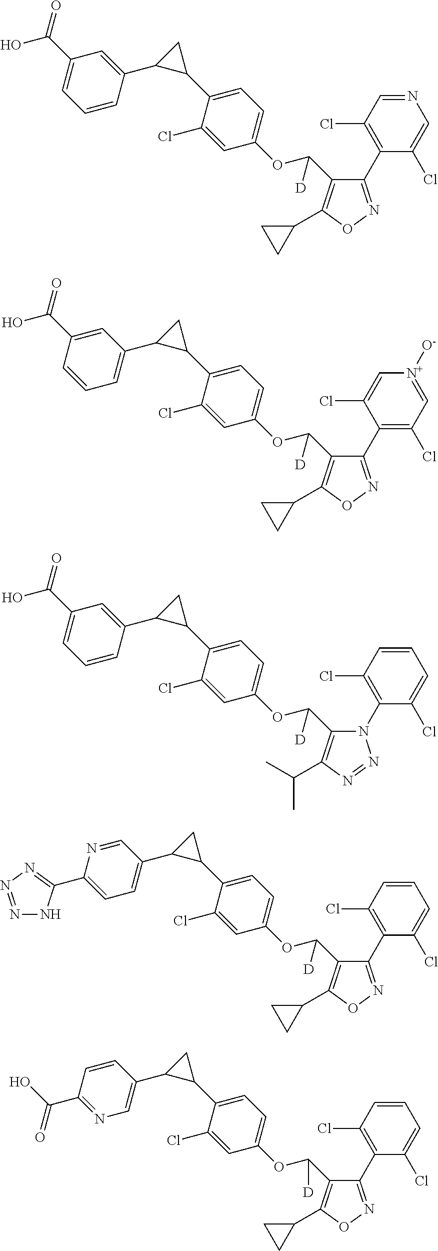 [SMILES: [2H]C(Oc1ccc(C2CC2c2cccc(C(=O)O)c2)c(Cl)c1)c1c(-c2c(Cl)cncc2Cl)noc1C1CC1 ; [2H]C(Oc1ccc(C2CC2c2cccc(C(=O)O)c2)c(Cl)c1)c1c(-c2c(Cl)c[n+]([O-])cc2Cl)noc1C1CC1 ; [2H]C(Oc1ccc(C2CC2c2cccc(C(=O)O)c2)c(Cl)c1)c1c(C(C)C)nnn1-c1c(Cl)cccc1Cl ; [2H]C(Oc1ccc(C2CC2c2ccc(-c3nnn[nH]3)nc2)c(Cl)c1)c1c(-c2c(Cl)cccc2Cl)noc1C1CC1 ; [2H]C(Oc1ccc(C2CC2c2ccc(C(=O)O)nc2)c(Cl)c1)c1c(-c2c(Cl)cccc2Cl)noc1C1CC1]

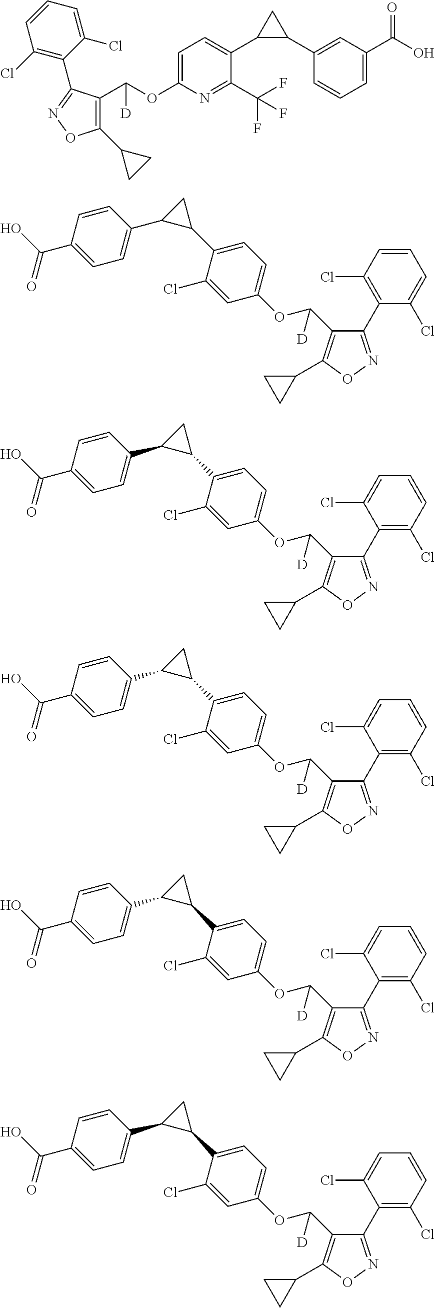

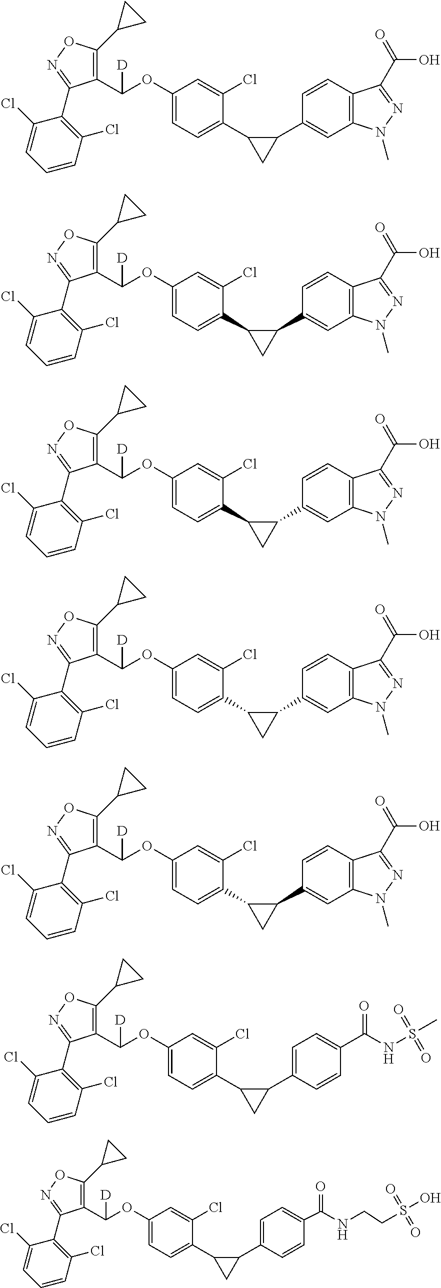

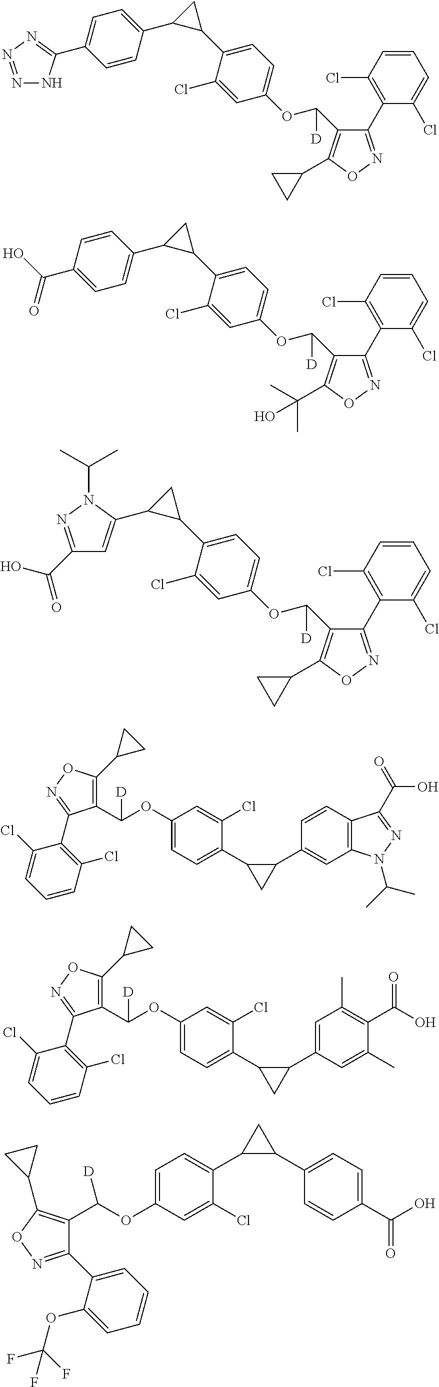

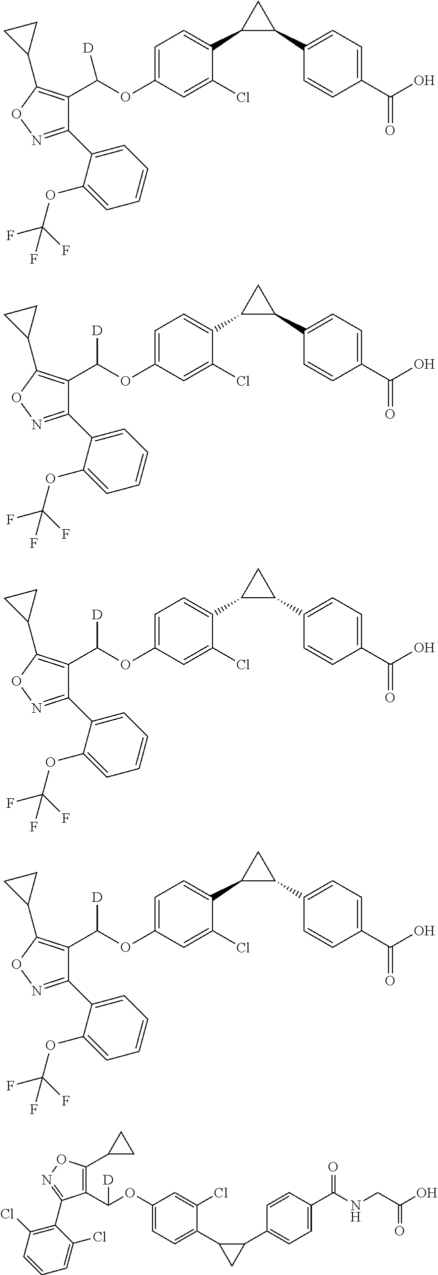

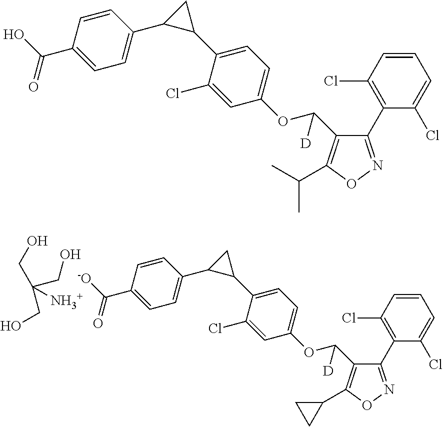

3-(2-(2-chloro-4-((5-cyclopropyl-3-(2,6-dichlorophenyl)isoxazol-4-yl)methoxy)phenyl)cyclopropyl)benzoic acid,
(−)-3-(2-(2-chloro-4-((5-cyclopropyl-3-(2,6-dichlorophenyl)isoxazol-4-yl)methoxy)phenyl)cyclopropyl)benzoic acid,
3-((1R,2R)-2-(2-chloro-4-((5-cyclopropyl-3-(2,6-dichlorophenyl)isoxazol-4-yl)methoxy)phenyl)cyclopropyl)benzoic acid,
3-((1S,2R)-2-(2-chloro-4-((5-cyclopropyl-3-(2,6-dichlorophenyl)isoxazol-4-yl)methoxy)phenyl)cyclopropyl)benzoic acid,
3-((1S,2S)-2-(2-chloro-4-((5-cyclopropyl-3-(2,6-dichlorophenyl)isoxazol-4-yl)methoxy)phenyl)cyclopropyl)benzoic acid,
3-((1R,2S)-2-(2-chloro-4-((5-cyclopropyl-3-(2,6-dichlorophenyl)isoxazol-4-yl)methoxy)phenyl)cyclopropyl)benzoic acid,
(+)-3-(2-(2-chloro-4-((5-cyclopropyl-3-(2,6-dichlorophenyl)isoxazol-4-yl)methoxy)phenyl)cyclopropyl)benzoic acid,
3-(2-(2-chloro-4-((3-(2,6-dichlorophenyl)-5-isopropylisoxazol-4-yl)methoxy)phenyl)cyclopropyl)benzoic acid,
3-(2-(2-chloro-4-((5-cyclopropyl-3-(3,5-dichloropyridin-4-yl)isoxazol-4-yl)methoxy)phenyl)cyclopropyl)benzoic acid,
4-(4-((4-(2-(3-carboxyphenyl)cyclopropyl)-3-chlorophenoxy)methyl)-5-cyclopropylisoxazol-3-yl)-3,5-dichloropyridine 1-oxide,
3-(2-(2-chloro-4-((1-(2,6-dichlorophenyl)-4-isopropyl-1H-1,2,3-triazol-5-yl)methoxy)phenyl)cyclopropyl)benzoic acid,
4-((4-(2-(6-(1H-tetrazol-5-yl)pyridin-3-yl)cyclopropyl)-3-chlorophenoxy)methyl)-5-cyclopropyl-3-(2,6-dichlorophenyl)isoxazole,
5-(2-(2-chloro-4-((5-cyclopropyl-3-(2,6-dichlorophenyl)isoxazol-4-yl)methoxy)phenyl)cyclopropyl)picolinic acid.
3-(2-(6-((5-cyclopropyl-3-(2,6-dichlorophenyl)isoxazol-4-yl)methoxy)-2-(trifluoromethy)pyridin-3-yl)cyclopropyl)benzoic acid,
4-(2-(2-chloro-4-((5-cyclopropyl-3-(2,6-dichlorophenyl)isoxazol-4-yl)methoxy)phenyl)cyclopropyl)benzoic acid,
1,3-dihydroxy-2-(hydroxymethyl)propan-2-aminium 4-(2-(2-chloro-4-((5-cyclopropyl-3-(2,6-dichlorophenyl)isoxazol-4-yl)methoxy)phenyl)cyclopropyl)benzoate,
(+)-4-(2-(2-chloro-4-((5-cyclopropyl-3-(2,6-dichlorophenyl)isoxazol-4-yl)methoxy)phenyl)cyclopropyl)benzoic acid,
4-((1S,2R)-2-(2-chloro-4-((5-cyclopropyl-3-(2,6-dichlorophenyl)isoxazol-4-yl)methoxy)phenyl)cyclopropyl)benzoic acid,
4-((1R,2R)-2-(2-chloro-4-((5-cyclopropyl-3-(2,6-dichlorophenyl)isoxazol-4-yl)methoxy)phenyl)cyclopropyl)benzoic acid,
4-((1R,2S)-2-(2-chloro-4-((5-cyclopropyl-3-(2,6-dichlorophenyl)isoxazol-4-yl)methoxy)phenyl)cyclopropyl)benzoic acid,
4-((1S,2S)-2-(2-chloro-4-((5-cyclopropyl-3-(2,6-dichlorophenyl)isoxazol-4-yl)methoxy)phenyl)cyclopropyl)benzoic acid,
(−)-4-(2-(2-chloro-4-((5-cyclopropyl-3-(2,6-dichlorophenyl)isoxazol-4-yl)methoxy)phenyl)cyclopropyl)benzoic acid,
6-(2-(2-chloro-4-((5-cyclopropyl-3-(2,6-dichlorophenyl)isoxazol-4-yl)methoxy)phenyl)cyclopropyl)-1-methyl-1H-indazole-3-carboxylic acid,
(+)-6-(2-(2-chloro-4-((5-cyclopropyl-3-(2,6-dichlorophenyl)isoxazol-4-yl)methoxy)phenyl)cyclopropyl)-1-methyl-1H-indazole-3-carboxylic acid,
6-((1S,2R)-2-(2-chloro-4-((5-cyclopropyl-3-(2,6-dichlorophenyl)isoxazol-4-yl)methoxy)phenyl)cyclopropyl)-1-methyl-1H-indazole-3-carboxylic acid,
6-((1R,2R)-2-(2-chloro-4-((5-cyclopropyl-3-(2,6-dichlorophenyl)isoxazol-4-yl)methoxy)phenyl)cyclopropyl)-1-methyl-1H-indazole-3-carboxylic acid,
6-((1R,2S)-2-(2-chloro-4-((5-cyclopropyl-3-(2,6-dichlorophenyl)isoxazol-4-yl)methoxy)phenyl)cyclopropyl)-1-methyl-1H-indazole-3-carboxylic acid,
6-((1S,2S)-2-(2-chloro-4-((5-cyclopropyl-3-(2,6-dichlorophenyl)isoxazol-4-yl)methoxy)phenyl)cyclopropyl)-1-methyl-1H-indazole-3-carboxylic acid,
(−)-6-(2-(2-chloro-4-((5-cyclopropyl-3-(2,6-dichlorophenyl)isoxazol-4-yl)methoxy)phenyl)cyclopropyl)-1-methyl-1H-indazole-3-carboxylic acid,
4-(2-(2-chloro-4-((5-cyclopropyl-3-(2,6-dichlorophenyl)isoxazol-4-yl)methoxy)phenyl)cyclopropyl)-N—(methylsulfonyl)benzamide,
2-(4-(2-(2-chloro-4-((5-cyclopropyl-3-(2,6-dichlorophenyl)isoxazol-4-yl)methoxy)phenyl)cyclopropyl)benzamido)ethanesulfonic acid,
4-((4-(2-(4-(1H-tetrazol-5-yl)phenyl)cyclopropyl)-3-chlorophenoxy)methyl)-5-cyclopropyl-3-(2,6-dichlorophenyl)isoxazole,
4-(2-(2-chloro-4-((3-(2,6-dichlorophenyl)-5-(2-hydroxypropan-2-yl)isoxazol-4-yl)methoxy)phenyl)cyclopropyl)benzoic acid,
5-(2-(2-chloro-4-((5-cyclopropyl-3-(2,6-dichlorophenyl)isoxazol-4-yl)methoxy)phenyl)cyclopropyl)-1-isopropyl-1H-pyrazole-3-carboxylic acid,
6-(2-(2-chloro-4-((5-cyclopropyl-3-(2,6-dichlorophenyl)isoxazol-4-yl)methoxy)phenyl)cyclopropyl)-1-isopropyl-1H-indazole-3-carboxylic acid,
4-(2-(2-chloro-4-((5-cyclopropyl-3-(2,6-dichlorophenyl)isoxazol-4-yl)methoxy)phenyl)cyclopropyl)-2,6-dimethylbenzoic acid,
4-(2-(2-chloro-4-((5-cyclopropyl-3-(2-(trifluoromethoxy)phenyl)isoxazol-4-yl)methoxy)phenyl)cyclopropyl)benzoic acid,
(+)-2-(4-(2-(2-chloro-4-((5-cyclopropyl-3-(2,6-dichlorophenyl)isoxazol-4-yl)methoxy)phenyl)cyclopropyl)benzamido)ethanesulfonic acid,
4-((1R,2S)-2-(2-chloro-4-((5-cyclopropyl-3-(2-(trifluoromethoxy)phenyl)isoxazol-4-yl)methoxy)phenyl)cyclopropyl)benzoic acid,
4-((1R,2R)-2-(2-chloro-4-((5-cyclopropyl-3-(2-(trifluoromethoxy)phenyl)isoxazol-4-yl)methoxy)phenyl)cyclopropyl)benzoic acid,
4-((1S,2R)-2-(2-chloro-4-((5-cyclopropyl-3-(2-(trifluoromethoxy)phenyl)isoxazol-4-yl)methoxy)phenyl)cyclopropyl)benzoic acid,
4-((1S,2S)-2-(2-chloro-4-((5-cyclopropyl-3-(2-(trifluoromethoxy)phenyl)isoxazol-4-yl)methoxy)phenyl)cyclopropyl)benzoic acid,
(−)-2-(4-(2-(2-chloro-4-((5-cyclopropyl-3-(2,6-dichlorophenyl)isoxazol-4-yl)methoxy)phenyl)cyclopropyl)benzamido)ethanesulfonic acid,
2-(4-(2-(2-chloro-4-((5-cyclopropyl-3-(2,6-dichlorophenyl)isoxazol-4-yl)methoxy)phenyl)cyclopropyl)benzamido)acetic acid,
4-(2-(2-chloro-4-((4-(2,6-dichlorophenyl)-1-isopropyl-1H-1,2,3-triazol-5-yl)methoxy)phenyl)cyclopropyl)benzoic acid,
3-(2-(2-chloro-4-((5-cyclopropyl-3-(2,6-dichlorophenyl)isoxazol-4-yl)methoxy)-5-fluorophenyl)cyclopropyl)benzoic acid,
3-(2-(2-chloro-4-((5-cyclopropyl-3-(2,6-dichlorophenyl)isoxazol-4-yl)methoxy-d)-5-fluorophenyl)cyclopropyl)benzoic acid,
3-(2-(2-chloro-4-((5-cyclopropyl-3-(2,6-dichlorophenyl)isoxazol-4-yl)methoxy-d)phenyl)cyclopropyl)benzoic acid,
3-(2-(2-chloro-4-((5-cyclopropyl-3-(2,6-dichlorophenyl)isoxazol-4-yl)methoxy-d2)phenyl)cyclopropyl)benzoic acid,
3-((1R,2S)-2-(2-chloro-4-((5-cyclopropyl-3-(2,6-dichlorophenyl)isoxazol-4-yl)methoxy-d)phenyl)cyclopropyl)benzoic acid,
3-((1R,2S)-2-(2-chloro-4-((5-cyclopropyl-3-(2,6-dichlorophenyl)isoxazol-4-yl)methoxy-d2)phenyl)cyclopropyl)benzoic acid,
3-((1S,2S)-2-(2-chloro-4-((5-cyclopropyl-3-(2,6-dichlorophenyl)isoxazol-4-yl)methoxy-d)phenyl)cyclopropyl)benzoic acid,
3-((1S,2R)-2-(2-chloro-4-((5-cyclopropyl-3-(2,6-dichlorophenyl)isoxazol-4-yl)methoxy-d)phenyl)cyclopropyl)benzoic acid,
3-((1R,2R)-2-(2-chloro-4-((5-cyclopropyl-3-(2,6-dichlorophenyl)isoxazol-4-yl)methoxy-d)phenyl)cyclopropyl)benzoic acid,
3-(2-(2-chloro-4-((3-(2,6-dichlorophenyl)-5-isopropylisoxazol-4-yl)methoxy-d)phenyl)cyclopropyl)benzoic acid,
5-(2-(2-chloro-4-((5-cyclopropyl-3-(2,6-dichlorophenyl)isoxazol-4-yl)methoxy-d)phenyl)cyclopropyl)picolinic acid,
3-(2-(6-((5-cyclopropyl-3-(2,6-dichlorophenyl)isoxazol-4-yl)methoxy-d)-2-(trifluoromethyl)pyridin-3-yl)cyclopropyl)benzoic acid,
4-(2-(2-chloro-4-((5-cyclopropyl-3-(2,6-dichlorophenyl)isoxazol-4-yl)methoxy-d)phenyl)cyclopropyl)benzoic acid,
4-((1S,2S)-2-(2-chloro-4-((5-cyclopropyl-3-(2,6-dichlorophenyl)isoxazol-4-yl)methoxy-d)phenyl)cyclopropyl)benzoic acid,
4-((1R,2S)-2-(2-chloro-4-((5-cyclopropyl-3-(2,6-dichlorophenyl)isoxazol-4-yl)methoxy-d)phenyl)cyclopropyl)benzoic acid,
4-((1R,2R)-2-(2-chloro-4-((5-cyclopropyl-3-(2,6-dichlorophenyl)isoxazol-4-yl)methoxy-d)phenyl)cyclopropyl)benzoic acid,
4-((1S,2R)-2-(2-chloro-4-((5-cyclopropyl-3-(2,6-dichlorophenyl)isoxazol-4-yl)methoxy-d)phenyl)cyclopropyl)benzoic acid,
6-(2-(2-chloro-4-((5-cyclopropyl-3-(2,6-dichlorophenyl)isoxazol-4-yl)methoxy-d)phenyl)cyclopropyl)-1-methyl-1H-indazole-3-carboxylic acid,
6-((1S,2R)-2-(2-chloro-4-((5-cyclopropyl-3-(2,6-dichlorophenyl)isoxazol-4-yl)methoxy-d)phenyl)cyclopropyl)-1-methyl-1H-indazole-3-carboxylic acid,
6-((1R,2R)-2-(2-chloro-4-((5-cyclopropyl-3-(2,6-dichlorophenyl)isoxazol-4-yl)methoxy-d)phenyl)cyclopropyl)-1-methyl-1H-indazole-3-carboxylic acid,
6-((1R,2S)-2-(2-chloro-4-((5-cyclopropyl-3-(2,6-dichlorophenyl)isoxazol-4-yl)methoxy-d)phenyl)cyclopropyl)-1-methyl-1H-indazole-3-carboxylic acid,
6-((1S,2S)-2-(2-chloro-4-((5-cyclopropyl-3-(2,6-dichlorophenyl)isoxazol-4-yl)methoxy-d)phenyl)cyclopropyl)-1-methyl-1H-indazole-3-carboxylic acid,
4-(2-(2-chloro-4-((5-cyclopropyl-3-(2,6-dichlorophenyl)isoxazol-4-yl)methoxy-d)phenyl)cyclopropyl)-N—(methylsulfonyl)benzamide,
2-(4-(2-(2-chloro-4-((5-cyclopropyl-3-(2,6-dichlorophenyl)isoxazol-4-yl)methoxy-d)phenyl)cyclopropyl)benzamido)ethane-1-sulfonic acid,
4-((4-(2-(4-(1H-tetrazol-5-yl)phenyl)cyclopropyl)-3-chlorophenoxy)methyl-d)-5-cyclopropyl-3-(2,6-dichlorophenyl)isoxazole,
4-(2-(2-chloro-4-((3-(2,6-dichlorophenyl)-5-(2-hydroxypropan-2-yl)isoxazol-4-yl)methoxy-d)phenyl)cyclopropyl)benzoic acid,
5-(2-(2-chloro-4-((5-cyclopropyl-3-(2,6-dichlorophenyl)isoxazol-4-yl)methoxy-d)phenyl)cyclopropyl)-1-isopropyl-1H-pyrazole-3-carboxylic acid,
6-(2-(2-chloro-4-((5-cyclopropyl-3-(2,6-dichlorophenyl)isoxazol-4-yl)methoxy-d)phenyl)cyclopropyl)-1-isopropyl-1H-indazole-3-carboxylic acid,
4-(2-(2-chloro-4-((5-cyclopropyl-3-(2,6-dichlorophenyl)isoxazol-4-yl)methoxy-d)phenyl)cyclopropyl)-2,6-dimethylbenzoic acid,
4-(2-(2-chloro-4-((5-cyclopropyl-3-(2-(trifluoromethoxy)phenyl)isoxazol-4-yl)methoxy-d)phenyl)cyclopropyl)benzoic acid,
4-((1R,2S)-2-(2-chloro-4-((5-cyclopropyl-3-(2-(trifluoromethoxy)phenyl)isoxazol-4-yl)methoxy-d)phenyl)cyclopropyl)benzoic acid,
4-((1R,2R)-2-(2-chloro-4-((5-cyclopropyl-3-(2-(trifluoromethoxy)phenyl)isoxazol-4-yl)methoxy-d)phenyl)cyclopropyl)benzoic acid,
4-((1S,2R)-2-(2-chloro-4-((5-cyclopropyl-3-(2-(trifluoromethoxy)phenyl)isoxazol-4-yl)methoxy-d)phenyl)cyclopropyl)benzoic acid,
4-((1S,2S)-2-(2-chloro-4-((5-cyclopropyl-3-(2-(trifluoromethoxy)phenyl)isoxazol-4-yl)methoxy-d)phenyl)cyclopropyl)benzoic acid,
(4-(2-(2-chloro-4-((5-cyclopropyl-3-(2,6-dichlorophenyl)isoxazol-4-yl)methoxy-d)phenyl)cyclopropyl)benzoyl)glycine,
4-(2-(2-chloro-4-((4-(2,6-dichlorophenyl)-1-isopropyl-1H-1,2,3-triazol-5-yl)methoxy-d)phenyl)cyclopropyl)benzoic acid,
1,3-dihydroxy-2-(hydroxymethyl)propan-2-aminium 4-(2-(2-chloro-4-((5-cyclopropyl-3-(2,6-dichlorophenyl)isoxazol-4-yl)methoxy-d)phenyl)cyclopropyl)benzoate.

Also provided herein are kits that include any FXR agonist (or composition containing such an agonist) described herein and a device for localized delivery within a region of the intestines, such as the ileum or colon. In certain embodiments, the device is a syringe, bag, or a pressurized container.

### IV. Compositions

Also disclosed herein are pharmaceutical compositions comprising at least one compound according to any of formulas 1-35, which can be used with embodiments of the method provided herein. Remington's Pharmaceutical Sciences, by E. W. Martin, Mack Publishing Co., Easton, Pa., 15th Edition, 1975, incorporated herein by reference, describes exemplary formulations (and components thereof) suitable for pharmaceutical delivery of the disclosed compounds. Pharmaceutical compositions comprising at least one of the disclosed compounds can be formulated for use in human or veterinary medicine. Particular formulations of a disclosed pharmaceutical composition may depend, for example, on the mode of administration (e.g., oral). In some embodiments, disclosed pharmaceutical compositions include a pharmaceutically acceptable carrier in addition to at least one or two or more active ingredients, such as a compound or compounds disclosed herein. In other embodiments, other medicinal or pharmaceutical agents, for example, with similar, related or complementary effects on the affliction being treated (such as LADA), can also be included as active ingredients in a pharmaceutical composition. For example, one or more of the disclosed compounds can be formulated with one or more of (such as 1, 2, 3, 4, or 5 of) a statin, an insulin sensitizing drug, alpha-glucosidase inhibitor, amylin agonist, dipeptidyl-peptidase 4 (DPP-4) inhibitor (such as sitagliptin, vildagliptin, saxagliptin, linagliptin, anaglptin, teneligliptin, alogliptin, gemiglptin, or dutoglpitin), meglitinide, sulfonylurea, peroxisome proliferator-activated receptor (PPAR)-gamma agonist (e.g., a thiazolidinedione (TZD) [such as ioglitazone, rosiglitazone, rivoglitazone, or troglitazone], aleglitazar, farglitazar, muraglitazar, or tesaglitazar), a glucagon-like peptide (GLP) agonist, anti-inflammatory agent (e.g., oral corticosteroid), nicotinamide ribonucleoside and analogs thereof that promote NAD+ production of which is a substrate for many enzymatic reactions such as p450s which are a target of FXR (for examples see Yang et al., J. Med. Chem., 50:6458-61, 2007, herein incorporated by reference) and the like.

Pharmaceutically acceptable carriers useful for the disclosed methods and compositions can depend on the particular mode of administration being employed. For example, for solid compositions (e.g., powder, pill, tablet, or capsule forms), conventional non-toxic solid carriers can include, without limitation, pharmaceutical grades of sugars, such as mannitol or lactose, polysaccharides, such as starch, or salts of organic acids, such as magnesium stearate. In addition to biologically neutral carriers, pharmaceutical compositions can optionally contain amounts of auxiliary substances (e.g., excipients), such as wetting or emulsifying agents, preservatives, and pH buffering agents and the like; for example, sodium acetate or sorbitan monolaurate. Other non-limiting excipients include nonionic solubilizers, such as cremophor, or proteins, such as human serum albumin or plasma preparations. In some embodiments, the pharmaceutical composition includes a sufficient amount of a disclosed compound to have a desired therapeutic effect. Typically, the disclosed compound constitutes greater than 0% to less than 100% of the pharmaceutical composition, such as 10% or less, 20% or less, 30% or less, 40% or less, 50% or less, 60% or less, 70% or less, 80% or less, 90% or less, or 90% to less than 100% of the pharmaceutical composition.

The disclosed pharmaceutical compositions may be formulated as a pharmaceutically acceptable salt, solvate, hydrate, N-oxide or combination thereof, of a disclosed compound. Additionally, the pharmaceutical composition may comprise one or more polymorph of the disclosed compound. Pharmaceutically acceptable salts are salts of a free base form of a compound that possesses the desired pharmacological activity of the free base. These salts may be derived from inorganic or organic acids. Non-limiting examples of suitable inorganic acids include hydrochloric acid, nitric acid, hydrobromic acid, sulfuric acid, hydriodic acid, and phosphoric acid. Non-limiting examples of suitable organic acids include acetic acid, propionic acid, glycolic acid, lactic acid, pyruvic acid, malonic acid, succinic acid, malic acid, maleic acid, fumaric acid, tartaric acid, citric acid, benzoic acid, cinnamic acid, mandelic acid, methanesulfonic acid, ethanesulfonic acid, p-toluenesulfonic acid, methyl sulfonic acid, salicylic acid, formic acid, trichloroacetic acid, trifluoroacetic acid, gluconic acid, asparagic acid, aspartic acid, benzenesulfonic acid, p-toluenesulfonic acid, naphthalenesulfonic acid, and the like. Examples of other suitable pharmaceutically acceptable salts are found in Remington's Pharmaceutical Sciences, 17th Edition, Mack Publishing Company, Easton, Pa., 1985.

In some embodiments, compounds disclosed herein according to formulas 1-35 are formulated to have a suitable particle size. A suitable particle size may be one which reduces or substantially precludes separation of the components of the composition, e.g., no separation between the drug and any other components of the composition, such as a second drug, a pharmaceutically acceptable excipient, a corticosteroid, an antibiotic or any combination thereof. Additionally, the particle size may be selected to ensure the composition is suitable for delivery, such as oral delivery.

In certain embodiments, the composition further includes an enteric coating. Typically, an enteric coating is a polymer barrier applied to an oral medication to help protect the drug from the acidity and/or enzymes of the stomach, esophagus and/or mouth. In some embodiments, this coating can reduce or substantially prevent systemic delivery of the disclosed compound, thereby allowing substantially selective delivery to the intestines. In some embodiments, the enteric coating will not dissolve in the acid environment of the stomach, which has an acidic, pH of about 3, but will dissolve in the alkaline environments of the small intestine, with, for example, a pH of about 7 to 9. Materials used for enteric coating include, but are not limited to, fatty acids, waxes, shellac, plastics and plant fibers. In some embodiments, the coating may comprise methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), methyl methacrylate-methacrylic acid copolymers, shellac, cellulose acetate trimellitate, sodium alginate, or any combination thereof.

### V. Methods of Making the Compounds

A. Methods of Making Compounds Having the Structure of Formulas 1-18

Embodiments of a method of making compounds that have the structure of formulas 1-18 are also disclosed herein. A general method of making the compounds comprises reacting an aldehyde with a first amine to form an imine, reacting the imine with a reducing agent to form a second amine, and reacting the second amine with an activated carboxylic acid derivative or a carboxylic acid to form an amide.

Other embodiments further comprise contacting the aldehyde with a boronic acid, contacting the amide with a vinyl ester, contacting the first amine with a vinyl ester, contacting the amide with a boronic acid, or any combination thereof. In certain embodiments the reducing agent is a deuterated reducing agent, and the compound comprises deuterium.

One exemplary embodiment of the general method is shown in Scheme 1.

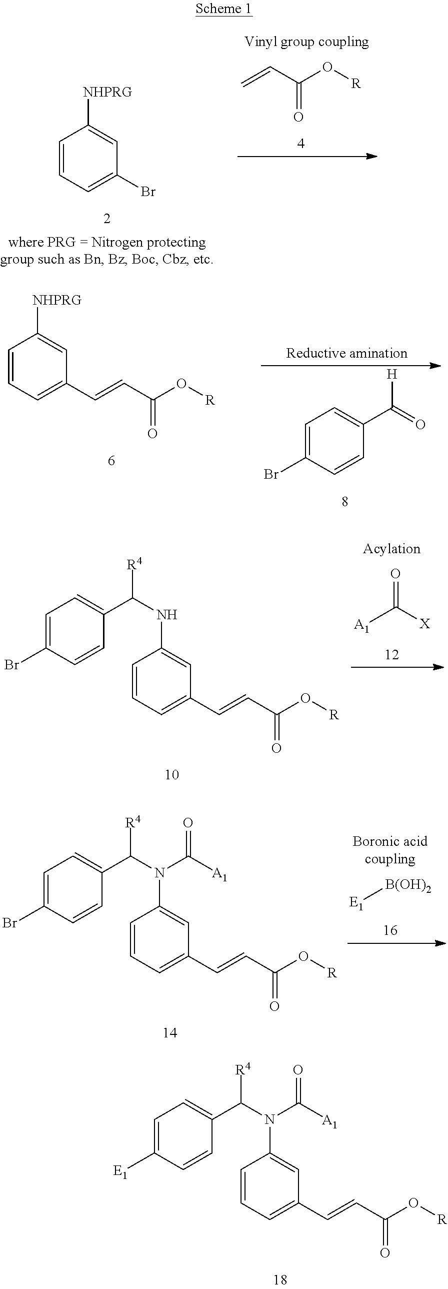

A. Vinyl Group Coupling

With reference to scheme 1, a protected aromatic amine 2 was coupled to a vinyl ester 4 by a suitable coupling technique to form compound 6. The amine of the aromatic amine 2 was protected by a suitable protecting group, as will be understood by a person of ordinary skill in the art. Additional information concerning protecting groups is provided by Greene and Wuts, Protective Groups in Organic Synthesis; 3rd Ed.; John Wiley & Sons, New York, 1999, which is incorporated herein by reference. Exemplary amine protecting groups include, but are not limited to, tert-butyloxycarbonyl (Boc), benzyl, benzoyl, or benzoyloxycarbonyl (Cbz). In some embodiments the technique is a Stille coupling. In certain working embodiments, coupling comprised treating the protected aromatic amine with a vinyl group in the presence of a suitable catalyst, such as a palladium catalyst, and optionally, a suitable phosphine compound. Suitable palladium catalysts include, but are not limited to, Bis(dibenzylideneacetone)palladium (Pd₂(dba)₃) or palladium acetate (Pd(OAc)₃). In certain working examples Pd₂(dba)₃ was used as a catalyst with tri(o-tolyl)phosphine (P(o-tol)₃) as the phosphine. The coupling reaction is conducted in any suitable solvent, such as dimethylformamide, at a temperature effective to facilitate a reaction. In some embodiments the effective temperature is from greater than 0° C. to at least 130 OC, such as from about 20° C. to about 110° C., from about 80° C. to about 100° C. In certain working embodiments the temperature was about 95° C.

B. Reductive Amination

The amine protecting group of compound 6 was removed by treatment with a suitable reagent. Suitable de-protection reagents and conditions for a specific protecting group can be selected by a person of ordinary skill in the art, and is further disclosed by consulting Greene and Wuts. In certain working embodiments, trifluoroacetic acid (TFA) was used to remove a Boc protecting group. In certain disclosed embodiments, the de-protected amine (not shown) was then treated with an aldehyde, such as aldehyde 8, in the presence of a reducing agent. In other embodiments, the amine was treated with an aldehyde, and subsequently treated by a reducing agent. The reducing agent is selected to place a desired R⁴ group into the molecule. In some embodiments R⁴ is hydrogen; in others it is deuterium. Suitable reducing agents include, but are not limited to, sodium triacetoxyborohydride, sodium triacetoxyborodeuteride, sodium cyanoborohydride, sodium cyanoborodeuteride, sodium borohydride, lithium borohydride, sodium borodeuteride or lithium borodeuteride. Suitable solvents for the reduction include, but are not limited to, toluene, halogenated solvents, THF, hexanes, cyclohexane, acetic acid, deuterated acetic acid, alcohols such as methanol, ethanol propanol, isopropanol, or deuterated alcohols such as methanol-d₄. Typically, the reducing agent was NaBH(OAc)₃, NaBD(OAc)₃, NaBD₃CN, NaBH₄ or NaBD₄ and the solvent was THF, CD₃OD, acetic acid or deuterated acetic acid.

C. Acylation

Subsequent to the reductive amination, compound 10 was acylated with acylating agent 12 under suitable conditions, such as by treatment with a carboxylic acid or an activated carboxylic acid derivative, such as an acid chloride, an acid bromide, or an anhydride. A person of ordinary skill in the art will understand which activated carboxylic acid derivatives are suitable for a particular carboxylic acid. Alternatively, a carboxylic acid may be coupled to the amine using a suitable coupling reagent known to a person of ordinary skill in the art. Exemplary coupling reagents include, but are not limited to, HATU, dicyclohexylcarbodiimide (DCCI, DCC) or 1-ethyl-3-(3-dimethylaminopropyl)carbodiimide (EDC, EDCI, EDAC). In working embodiments the carboxylic acid was activated by forming an acid chloride. The acylation reactions proceed in a suitable solvent, typically an aprotic solvent, such as pyridine, dichloromethane, chloroform, dioxane, toluene, DMF, THF or acetonitrile. Optionally, the reaction with a carboxylic acid or a carboxylic acid derivative may proceed in the presence of one or more additional compounds, such as potassium carbonate, triethylamine, diisopropylethylamine, sodium carbonate, 4-(dimethylamino)pyridine (DMAP) or pyridine. The reactions are performed at a temperature effective to facilitate the reaction, such as from greater than about −10° C. to greater than about 120° C., typically from about 5° C. to about 90° C., more typically from about 25° C. to about 65° C.

D. Boronic Acid Coupling

After the acylation reaction, compound 14 was treated with a boronic acid 16, in a Suzuki-type coupling. In some embodiments, the coupling was performed in the presence of a catalyst effective to facilitate the coupling reaction, and optionally in the presence of one or more additional compounds. Typical catalysts for a Suzuki coupling are palladium or nickel catalysts, including but not limited to, NiCl₂(dppf), NiCl₂(dppp), Pd(PPh₃)₄, Pd(OAC)₂ or PdCl₂(PPh₃)₄. In working embodiments the catalyst was Pd(PPh₃)₄. Typical additional compounds include, but are not limited to, triphenylphosphine (PPh₃), and/or bases such as potassium carbonate, sodium carbonate, cesium carbonate, sodium hydroxide, potassium hydroxide, triethylamine, sodium ethoxide, sodium methoxide, tripotassium phosphate or any combination thereof. In certain working embodiments, the additional compound was sodium carbonate. The coupling reaction is performed in any suitable solvent, such as DMF, ethanol, methanol, isopropanol, propanol, benzene, toluene, THF, dioxane, water or any combination thereof. In certain working embodiments, DMF-ethanol-water was used as the solvent.

A person of ordinary skill in the art will recognize that the various steps described above with reference to Scheme 1 do not necessarily have to be performed in the particular order depicted. The reactions can be performed in any order suitable to result in making the desired compound 18. For example, in certain embodiments, the sequence of reactions followed the order described in Scheme 2.

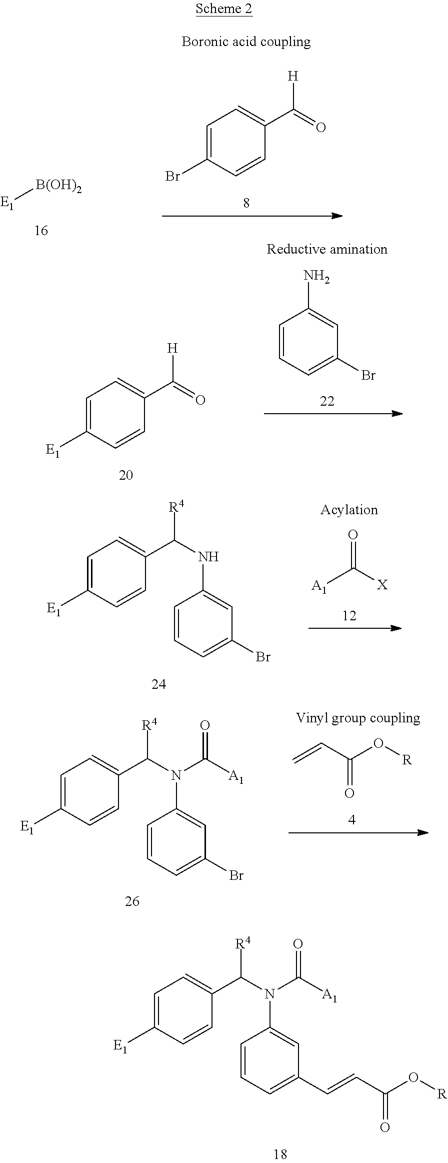

With reference to Scheme 2, boronic acid 16 was first coupled to aldehyde 8. The resulting product 20 was then treated with an amine compound 22 in a reductive amination step to form compound 24. Compound 24 was then acylated using acylating reagent 12 to form compound 26, which was then coupled to vinyl ester 4 to form compound 18.

Another variation of Scheme 1 is shown in Scheme 3. In this scheme, the compounds are made using a solid-phase synthetic method as used for the synthesis of fexaramine in U.S. Pat. No. 7,647,217, which is incorporated herein by reference. Thus, protected amine 6, where R is hydrogen, is immobilized onto a solid support 34, such as a bead or resin, typically Merrifield resin, through the action of a suitable base, for example, cesium carbonate, sodium carbonate or potassium carbonate, to make conjugate 36. The reductive amination, acylation and boronic acid coupling steps then proceed on the immobilized compound as described for Scheme 1, making conjugates 38, 40 and 42 respectively. Conjugate 42 is then treated with an alkoxide salt, such as sodium methoxide, to release the desired compound 18 from the solid support.

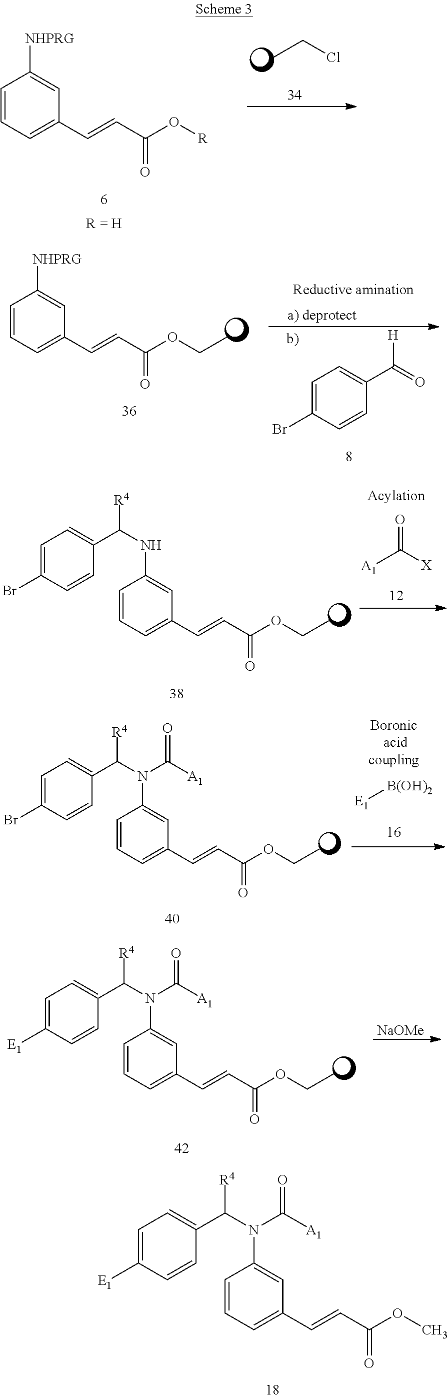

In certain embodiments, an alternative reaction pathway was followed. Scheme 4 outlines an exemplary alternative route. First, a halogenated nitrobenzene 28 was coupled to vinyl ester 4 in the presence of a suitable catalyst and an additional compound (not shown) to form compound 30. In working embodiments the catalyst was Pd(OAc)₂, and the additional compound was sodium acetate. The nitro group of compound 30 was then reduced to an amine by a suitable reagent to form amine 32. Suitable reagents include, but are not limited to, tin chloride, iron powder in an acid medium, zinc powder or catalytic hydrogenation using a transition metal catalyst comprising palladium, platinium, nickel, rhodium or ruthenium. In working embodiments tin chloride (SnCl₂) was used as the reducing agent.

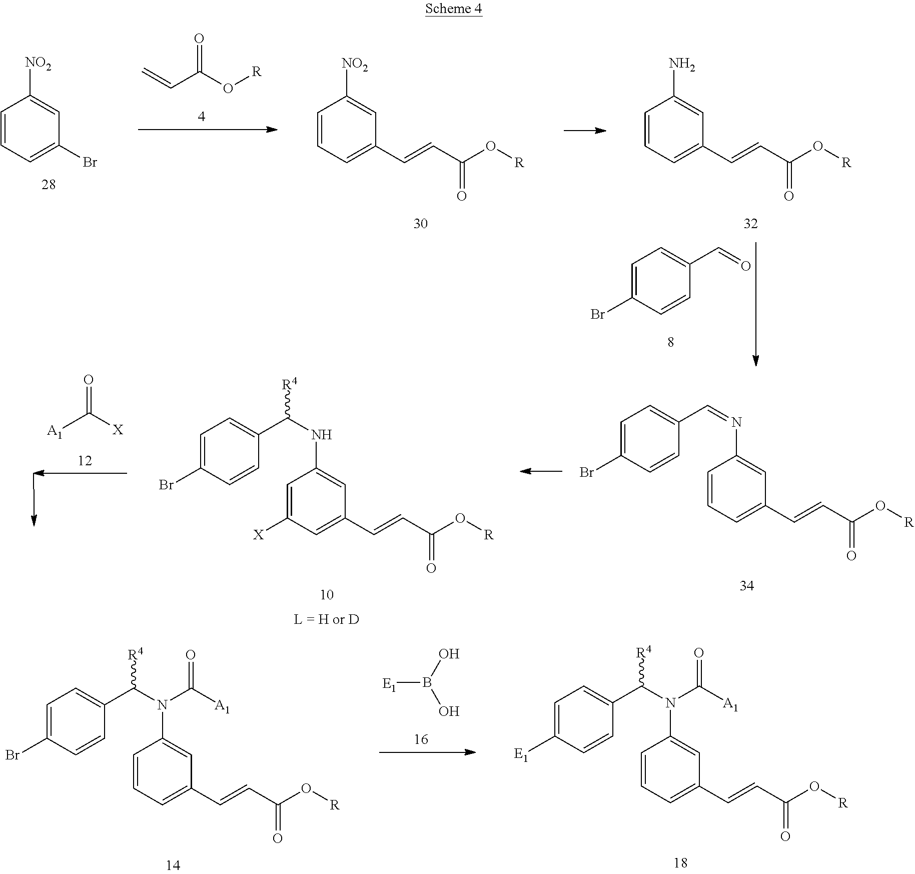

Amine 32 was then treated with an aldehyde 8 in a de-hydration reaction. Suitable dehydrating agents include, but are not limited to, an acid catalyst such as para-toluene sulfonic acid, a base such as triethylamine, malononitrile, molecular sieve, magnesium sulfate, sodium sulfate, or any combination thereof. Suitable solvents for the de-hydration reaction include toluene, xylenes, DMSO, DMF, THF, alcohols such as methanol or any combination thereof. Resulting imine compound 34 was then treated with a suitable reducing agent to form amine 10. In working embodiments, a deuterated reducing agent was used. In some embodiments sodium cyanodeuteroborohydride was used, and in others sodium deuteroborohydride was used. Any suitable, non-protonated solvent can be used, and in some working embodiments the solvent was methanol-d4 and in others it was THF.

Amine 10 was then treated with acylating reagent 12, as described above with reference to Scheme 1, to form compound 14. In working embodiments the amine was treated with carboxylic acids in the presence of HATU and diisopropylethylamine in DMF. In other working embodiments the amine was treated with carboxylic acid chlorides in dichloromethane in the presence of triethylamine.

Compound 14 was then treated with boronic acid 16 as described above with reference to Scheme 1. In certain working embodiments, compound 14 was treated with boronic acid 16 in a DME-ethanol-water solvent system, in the presence of Pd(PPh₃)₄ and sodium carbonate. In other working embodiments dioxane-water was used as the solvent, Pd(dppf)C₁₂ was the catalyst and potassium carbonate was used as a base.

One exemplary method of making compounds having the structure of formula 13 is shown in Scheme 5. This method is a modification of the method of Lee and Hartwig, J. Org. Chem. 2001, 66, 3402-3415.

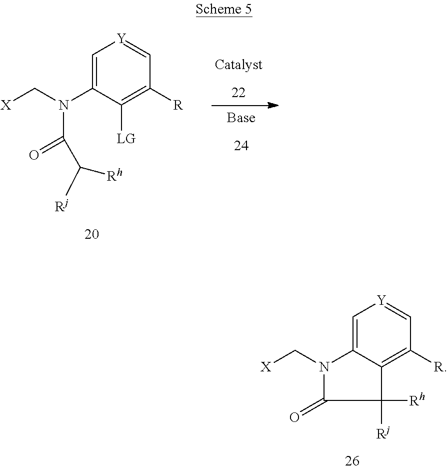

With reference to Scheme 5, compound 20 is reacted with a catalyst 22 and a base 24 in a suitable solvent, to form compound 26. Leaving group LG on compound 20 is any suitable leaving group, such as a halide, mesylate, tosylate or trifluoromethylsulfonate. Catalyst 22 is any catalyst that facilitates the formation of compound 26. Suitable catalysts include, but are not limited to, palladium catalysts such as Pd(OAc)₂, and may also comprise one or more ligands, such as PCy₃, or sterically hindered N-heterocyclic carbine ligands. The amount of the catalyst used is any suitable amount to catalyze the reaction at a suitable rate, such as from about 1 mol % to greater than about 20 mol %, preferably from about 5 mol % to about 10 mol %. Base 24 is any suitable base that facilitates the reaction. In some embodiments an excess of the base is used, such as from greater than 1 equivalent to greater than about 5 equivalents, preferably from about 1.1 equivalents to about 2 equivalents. Suitable bases include, but are not limited to, tert-butoxide salts, such as sodium, lithium or potassium tert-butoxide. The solvent can be any solvent suitable to facilitate a reaction. In some embodiments the solvent is 1, 4-dioxane.

Embodiments of a method of making prodrugs of compounds having formulas 1-13 are also disclosed herein. One general method of making prodrugs is disclosed by Poon, et al. Bioorg. med. Chem. Lett. 2005, 15: 2259-2263, and is shown in Scheme 6. Briefly, the method comprises making the thioester of the compound, and forming the ortho ester or imidate.

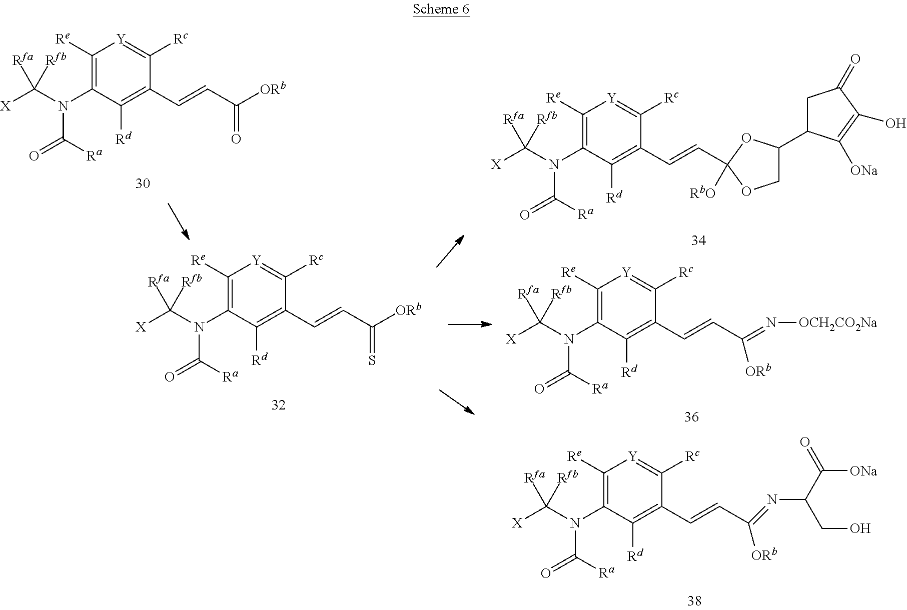

With reference to Scheme 6, ester compound 30 is reacted with reagent suitable to form thioester compound 32. Suitable reagents include, but are not limited to, Lawesson's reagent or P₂S₅. The reaction is performed in a suitable solvent, usually an aprotic solvent such as toluene, acetonitrile, cyclohexane, dichloromethane, or chloroform. The reaction may also be heated, such as to reflux.

The thioester compound 32 is then reacted with reagents suitable to form the desired prodrug, in the presence of a metal salt and a base. The metal salt is any metal salt suitable to mediate the desulfurization-condensation reaction between the thioester compound 32 and the alcohol or amine. Suitable metal salts include, but are not limited to, silver salts such as AgoTf. Suitable bases include, but are not limited to organic bases such as triethylamine or diisopropylethylamine. The reactions are performed in solvent suitable to facilitate the reaction, such as an aprotic solvent. Suitable solvents include, but are not limited to, acetonitrile, DMF, dimethylacetyl, N-methyl-2-pyrrolidone.

To form compound 34, thioester 32 is reacted with dibenzylascorbate, AgOTf and triethylamine in acetonitrile. An intermediate compound is formed initially (not shown) which is then reacted with hydrogen in the presence of a palladium catalyst in alcohol to form compound 34. Compound 36 is formed by reacting compound 32 with hydroxylamine, in the presence of AgOTf and triethylamine in acetonitrile. The intermediate compound (not shown) is then reacted with 2-bromoacetic acid and sodium hydroxide, to form compound 36. Compound 38 is made by reacting compound 32 with serine-OMe in the presence of AgOTf and triethylamine, in acetonitrile. The intermediate compound formed (not shown) is then reacted with Sodium trimethylsilanolate (NaOTMS) in THF to form compound 38.

A method of making compounds having formulas 17 and 18 is shown in Scheme 7, and is a modification of a method disclosed by Ates and Curran, J. Am. Chem. Soc. 2001, 123: 5130-5131.

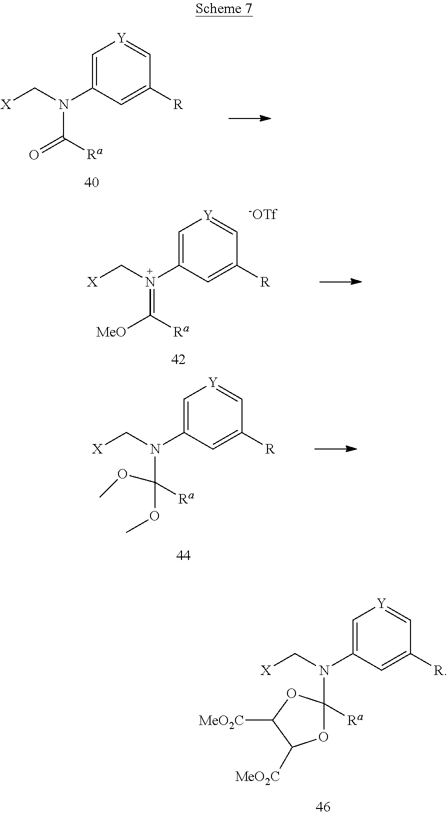

With reference to Scheme 7, compound 40 is reacted with a methylating agent, such as methyl trifluoromethanesulfonate, in a suitable solvent to make compound 42. Suitable solvents include, but are not limited to, halogenated solvents such as dichloromethane and chloroform. Compound 42 is reacted with a metal alkoxide solution, such as sodium methoxide in methanol, to form compound 44, an exemplary compound satisfying formula 17. Compound 44 is further reacted with dimethyl tartrate in a vacuum to form compound 46, an exemplary compound satisfying formula 18.

B. Methods of Making Compounds According to Formulas 19-35

A person of ordinary skill in the art will understand how to make the compounds of formulas 19-35. Additional information concerning the methods for making the disclosed compounds can be found in PCT application publication Nos. WO2003090745, WO2013007387 and WO2011020615, and in the Schemes below.

One exemplary embodiment of a general method of making a compound having the structure of formula 19 is shown in Scheme 8. This method is a modification of the method of Flatt, B. et al., J. Med. Chem. 2009, 52, 904-907, which is incorporated herein in its entirety. A person of ordinary skill in the art will appreciate that other suitable methods for making compounds having the structure of formula 19 can be determined.

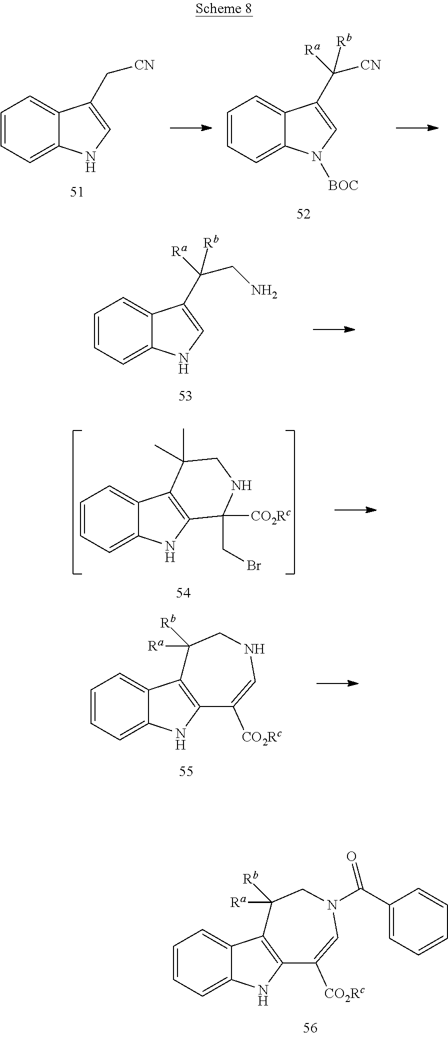

With reference to Scheme 8, an indole acetonitrile 51 is treated with a suitable protecting group. Scheme 8 illustrates using di-tert-butyl dicarbonate, in the presence of a base and in a suitable solvent, to form a BOC-protected indole (not shown). Suitable solvents include, but are not limited to, aprotic solvents, such as dichloromethane, dichloroethane, THF, chloroform, or combinations thereof. Suitable bases include, but are not limited to, triethylamine, 4-dimethylaminopyridine (DMAP), diiospropylethylamine, or combinations thereof. The BOC-protected indole is further reacted with lithium bis(trimethylsilyl)amide (LiHMDS) in a suitable, aprotic solvent such as THF or ether, and at a temperature effective to facilitate a reaction, to form compound 52. In some embodiments, the effective temperature is from about −100° C. to about −50° C., such as from about −80° C. to about −60° C. A suitable alkyl halide is then added to the reaction mixture, and the reaction mixture is warmed, or allowed to warm, to room temperature, such as to from about 20° C. to 25° C. A person of ordinary skill in the art will appreciate that the alkyl portion of the alkyl halide will correspond to the desired R^{a} and/or R^{b} group. For example, if R^{a} and/or R^{b} is methyl, a suitable alkyl halide may be methyl iodide. A person of ordinary skill in the art will also appreciate that if both R^{a} and R^{b} are alkyl, then an excess of LiHMDS and alkyl halide are used in the reaction, such as about 2.5 equivalents. However, if only one of R^{a} or R^{b} is alkyl, and the other is hydrogen, then only 1 equivalent of LiHMDS and alkyl halide is used.

Compound 52 is then deprotected, such as by removal of the BOC group, to form the deprotected indole compound (not shown). Suitable deprotection methods are known to persons of ordinary skill in the art and typically include reacting with an acid or acidic solution, including, but not limited to, trifluoroacetic acid or hydrochloric acid. The cyano group on the deprotected indole compound is then reduced by a suitable reducing agent, such as lithium aluminum hydride (LAH, LiAlH₄), at a temperature effective to facilitate a reaction, to form compound 53. Suitable solvents for the reduction reaction include any aprotic solvent that will not react with the reducing agent, such as THF and ethers. In some embodiments, the effective temperature is from about 20° C. to greater than 100° C., such as from about 40° C. to about 80° C.

Compound 53 is then reacted with a halopyruvate, such as R^{c}-bromopyruvate, where R^{c} is the desired ester. The reaction is conducted in the presence of an acid, and in a suitable solvent and at an effective temperature, to form compound 54. Exemplary bromopyruvates include ethyl bromopyruvate and isopropyl bromopyruvate. Suitable acids include aqueous acid such as hydrochloric acid. Suitable solvents include protic solvents, such as alcohols. In some embodiments, ethanol is used as the solvent. Typically, the effective temperature is from about 20° C. to greater than 100° C., such as from about 50° C. to about 80° C.

Compound 54 is then reacted with a base at a temperature effective to form compound 55. Suitable bases include, but are not limited to, triethylamine, diisopropylethylamine, pyridine or combinations thereof. In some embodiments, the effective temperature is from about 20° C. to greater than 120° C., such as from about 50° C. to about 110° C.

Compound 55 is then reacted with a suitable acid or activated acid derivative, such as an acid chloride, to form the desired compound 56. The reaction is conducted in a suitable solvent, and in the presence of a suitable base. Suitable solvents include, but are not limited to, halogenated solvents such as chloroform, dichloroethane and dichloromethane, aprotic solvents such as DMF, DMSO, THF, acetonitrile, pyridine, toluene, or combinations thereof. Suitable bases include, but are not limited to, triethylamine, diisopropylethylamine, pyridine, potassium carbonate, sodium carbonate or sodium hydrogen carbonate. The reaction is conducted at a temperature effective to facilitate a reaction. In some embodiments, the effective temperature is from greater than 20° C. to greater than 120° C., such as from about 50° C. to about 100° C.

Another exemplary embodiment of a general method of making a compound having the structure of formula 19 is shown in Scheme 9. This method is a modification of the method disclosed by Wang, et al. Tetrahedron Letters, 2011, 52, 3295-3297, which is incorporated herein in its entirety.

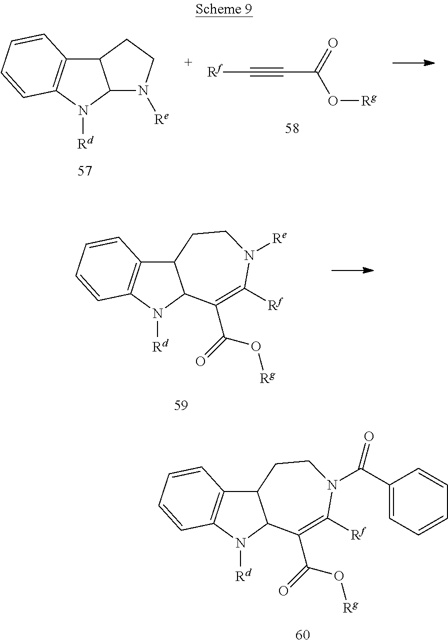

With reference to Scheme 9, a pyrroloindoline 57 is reacted with an acetylene ester 58 in a suitable solvent, and at a temperature effective to facilitate a reaction, to form compound 59. In some embodiments, the reaction is performed under an inert atmosphere, such as nitrogen or argon. Suitable solvents include, but are not limited to, polar, aprotic solvents such as DMF, DMSO or acetonitrile. In some embodiments, the effective temperature is from greater than 0° C. to greater than about 100° C., such as from about 10° C. to about 50° C., or about 20° C. to about 30° C. In some embodiments, the reaction proceeds in the presence of a catalyst. Suitable catalysts include, but are not limited to, copper halides, such as copper iodide, copper bromide, or copper chloride, salts of vitamin C such as sodium salt, potassium salt or lithium salt, or combinations thereof.

With reference to compound 59, R^{e} can be hydrogen or methyl. In embodiments where R^{e} is methyl, compound 59 is demethylated prior to acylation (not shown). The demethylation can be performed by any suitable method such as by reacting the tertiary amine with 1-chloroethylchloroformate in a suitable solvent. Solvents suitable for the demethylation include, but are not limited to, halogenated solvents such as dichloromethane, dichloroethane and chloroform, or THF. The reaction mixture is evaporated and then heated with an alcohol such as methanol for a time effective to form the secondary amine. The effective time is from greater than 1 minute to greater than 1 hour, such as from about 10 minutes to about 30 minutes.

Compound 59, or the demethylated compound 59, is then reacted with a suitable acid or activated acid derivative, such as an acid chloride, to form the desired compound 60. The reaction is conducted in a suitable solvent, and in the presence of a suitable base. Suitable solvents include, but are not limited to, halogenated solvents such as chloroform, dichloroethane and dichloromethane, aprotic solvents such as DMF, DMSO, THF, acetonitrile, pyridine, toluene, or combinations thereof. Suitable bases include, but are not limited to, triethylamine, diisopropylethylamine, pyridine, potassium carbonate, sodium carbonate or sodium hydrogen carbonate. The reaction is conducted at a temperature effective to facilitate a reaction. In some embodiments, the effective temperature is from greater than 20° C. to greater than 120° C., such as from about 50° C. to about 100° C.

One exemplary embodiment of a method of making a compound having formula 22 is shown in Scheme 10. A person of ordinary skill in the art will appreciate that other suitable methods for making compounds having the structure of formula 22 can be determined.

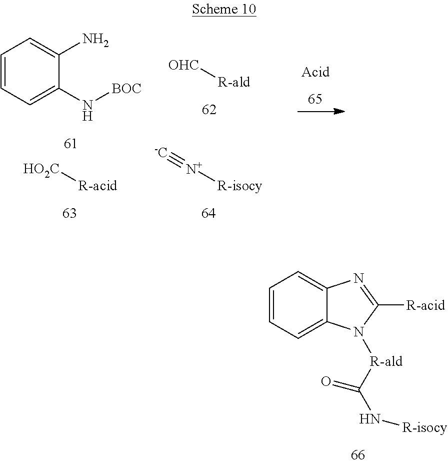

With reference to Scheme 10, a protected diamine 61, such as a BOC-protected diamine, is reacted with an aldehyde 62 in a suitable solvent for from about 10 minutes to greater than 60 minutes, such as from about 20 minutes to about 40 minutes. Suitable solvents include, but are not limited to, alcohols, such as methanol or ethanol, water or polar, aprotic solvents such as DMF or DMSO, or combinations thereof. Acid 63 and isocyanide 64 are then added. After an amount of time effective to allow the reaction to proceed, the resulting product is deprotected, such as by adding a suitable acid 65 for removing the BOC protecting group. The effective amount of time is from about 30 minutes to greater than 12 hours, such as from about 1 hour to about 4 hours. Suitable acids are those known to a person of ordinary skill in the art to remove the protecting group, and include, but are not limited to, hydrochloric acid and trifluoroacetic acid. After the addition of the acid, the reaction mixture is left for an amount of time effective to facilitate a reaction to form compound 66, such as from about 6 hours to greater than 24 hours, such as from about 12 hours to about 20 hours.

Typically, the reaction mixture is agitated, such as by stirring or shaking, for at least some of the reaction time, and in some embodiments, for substantially all of the reaction time. The reaction is conducted at a temperature effective to facilitate a reaction, such as from about 10° C. to greater than about 50° C., typically from about 20° C. to about 40° C.

Another exemplary method of making a compound having the structure of formula 22 is shown in Scheme 11. The method is a modification of the method disclosed in WO2004087714, which is incorporated herein in its entirety.

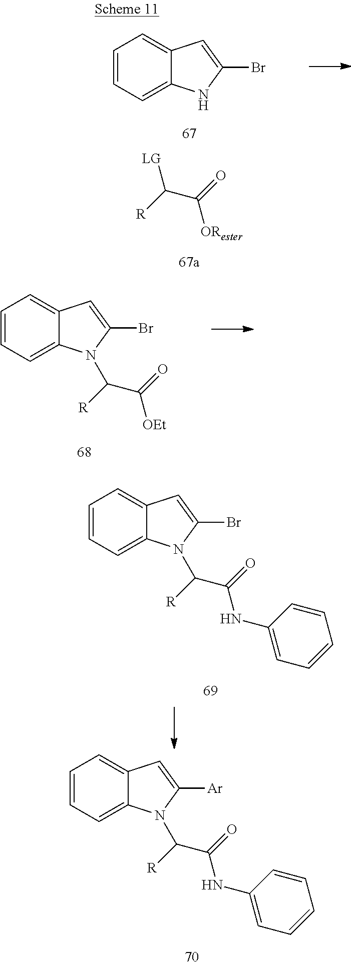

With reference to Scheme 11, a haloindole 67, such as a bromo indole, is reacted with an ester compound 67a, which comprises a desired R group and a leaving group LG, to form compound 68. The leaving group can be any suitable leaving group, such as a halide, triflate, mesalate or tosylate. The reaction is performed in the presence of a base, such as sodium hydride, and in a suitable solvent, such as DMF or THF.

Compound 68 is typically saponified to an acid (not shown) by any suitable method known to a person of ordinary skill in the art, such as by reacting the acid with a hydroxide base, or by treatment with an aqueous acid, such as hydrochloric acid. The acid is then typically activated, such as by forming an acid chloride, and then reacted with aniline to form compound 69. The reaction is conducted in a suitable solvent, and in the presence of a suitable base. Suitable solvents include, but are not limited to, halogenated solvents such as chloroform, dichloroethane and dichloromethane, aprotic solvents such as DMF, DMSO, THF, acetonitrile, pyridine, toluene, or combinations thereof. Suitable bases include, but are not limited to, triethylamine, diisopropylethylamine, pyridine, potassium carbonate, sodium carbonate or sodium hydrogen carbonate. The reaction is conducted at a temperature effective to facilitate a reaction. In some embodiments, the effective temperature is from greater than 20° C. to greater than 120° C., such as from about 50° C. to about 100° C.

Compound 69 is then reacted with a boronic acid (not shown) in a Suzuki-type coupling to form compound 70. In some embodiments, the boronic acid is an aromatic boronic acid. In some embodiments, the coupling is performed in the presence of a catalyst effective to facilitate the coupling reaction, and optionally in the presence of one or more additional compounds. Typical catalysts for a Suzuki coupling are palladium or nickel catalysts, including but not limited to, NiCl₂(dppf), NiCl₂(dppp), Pd(PPh₃)₄, Pd(OAC)₂ or PdCl₂(PPh₃)₄. Typical additional compounds include, but are not limited to, triphenylphosphine (PPh₃), and/or bases such as potassium carbonate, sodium carbonate, cesium carbonate, sodium hydroxide, potassium hydroxide, triethylamine, sodium ethoxide, sodium methoxide, tripotassium phosphate or any combination thereof. The coupling reaction is performed in any suitable solvent, such as DMF, ethanol, methanol, isopropanol, propanol, benzene, toluene, THF, dioxane, water or any combination thereof.

One exemplary embodiment of a method of making a compound having the structure of formula 25 is shown in Scheme 12. A person of ordinary skill in the art will appreciate that other suitable methods for making compounds having the structure of formula 25 can be determined.

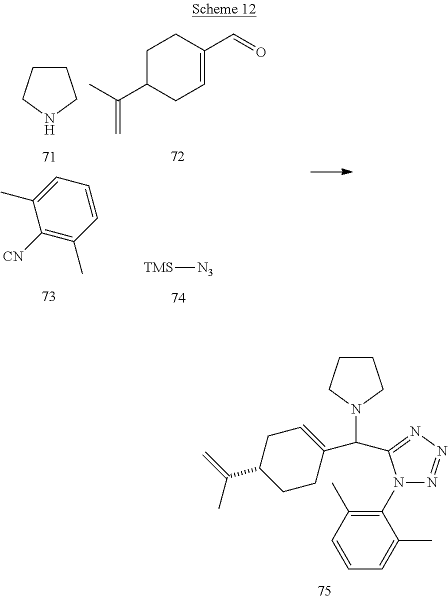

With reference to Scheme 12, an amine 71 is reacted with an aldehyde 72. The reaction typically is conducted in a suitable solvent, such as an alcohol, such as methanol or ethanol, water, or polar, aprotic solvents such as DMF or DMSO, or combinations thereof, for from about 10 minutes to greater than 60 minutes, such as from about 20 minutes to about 40 minutes. An isocyanide 73 and a suitable azide 74 are then added, and the reaction mixture is left for an amount of time effective to facilitate a reaction to form compound 75, such as from about 6 hours to greater than 48 hours, such as from about 12 hours to about 24 hours. One possible suitable azide is trimethylsilyl azide.

Without being bound to any particular theory, Scheme 13 provides one possible reaction mechanism for the reaction described in Scheme 12.

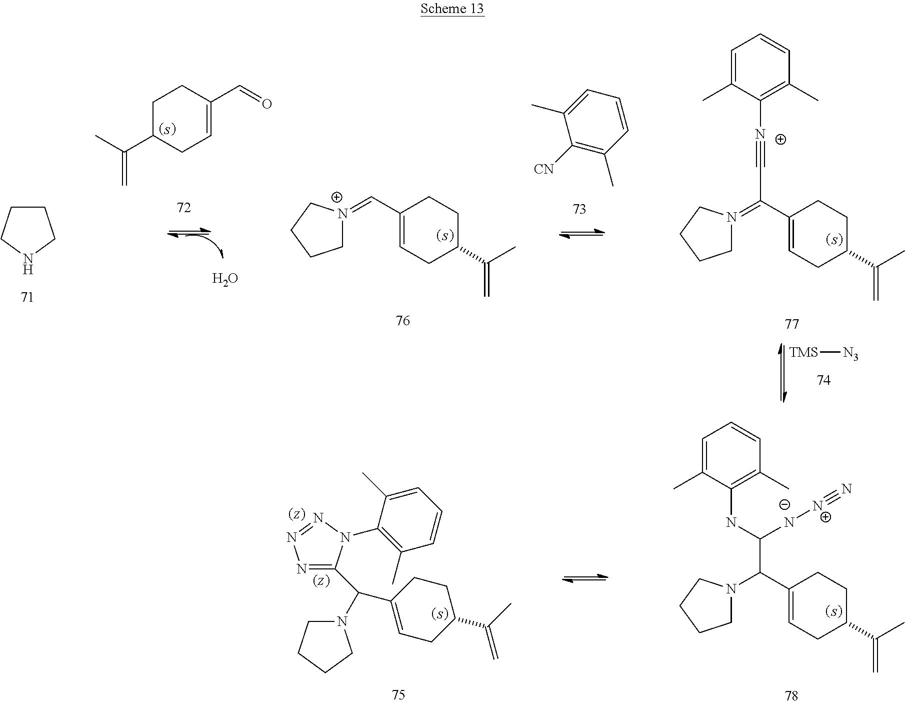

With reference to Scheme 13, the amine 71 reacts with the aldehyde 72 with the loss of water, to form an imine 76. The imine 76 then reacts with the isocyanide 73 to form an intermediate 77, which then reacts with the azide compound 74, to form an intermediate 78. The intermediate 78 then cyclizes to form the desired compound 75.

Another exemplary embodiment of a method of making a compound having the structure of formula 25 is shown in Scheme 14. The method is a modification of the method disclosed by Chen, et al. Synthesis, 2010, No. 9, 1505-1511, which is incorporated herein in its entirety.

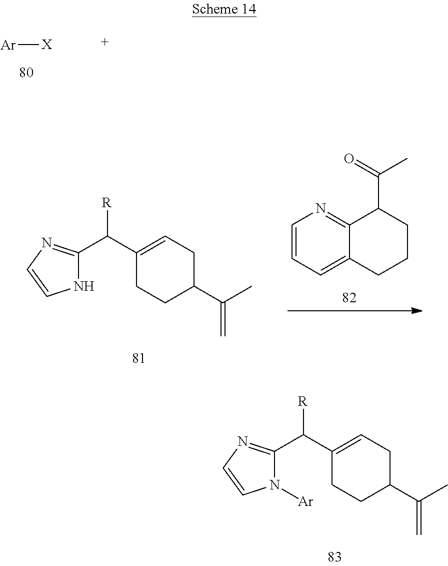

With reference to Scheme 14, an aromatic halide compound 80 is reacted with an imidazole compound 81 in the presence of a copper catalyst, such as copper (I) bromide and an additional compound 82 to form compound 83. The reaction is performed in a suitable solvent and in the presence of a suitable base. Suitable solvents include aprotic solvents such as DMSO or DMF. Suitable bases include any base that will facilitate the reaction, such as sodium carbonate, potassium carbonate, lithium carbonate or cesium carbonate. The reaction is conducted at a temperature effective to facilitate a reaction. In some embodiments, the effective temperature is from greater than 20° C. to greater than 120° C., such as from about 50° C. to about 80° C.

### VI. Embodiments of a Method of Using Farnesoid X Receptor Agonists to Treat or Prevent LADA

In some embodiments, farnesoid X receptor agonists, such as those disclosed herein, as well as compositions including such compounds, are used to treat or prevent LADA. Orally delivered fexaramine (Fex) (Downes et al., Mol Cell 11:1079-1092, 2003) is poorly absorbed, resulting in intestinally-restricted FXR activation. It is shown herein that Fex restores pancreatic β cell functions with robustly enhanced glucose-stimulated insulin secretion (GSIS) in diabetic mice without body weight changes. It is shown that Fex potentiates bioenergetics to enhance GLP-1 secretion in enteroendocrine L cells. Concomitantly, Fex increases gene expression of glucagon-like peptide-1 receptor (GLP-1R) in pancreatic β cells, resulting in restoration of GSIS in β cells to ameliorate hyperglycemia in ob/ob mice. Furthermore, Fex analogs, including, but not limited to, Fex-D, are more effective at glucose lowering than Fex. The beneficial systemic efficacy achieved with Fex and Fex-D suggests intestinal FXR agonist therapy as an approach in the treatment of LADA.

A. Treatment or Prevention of LADA

In some embodiments, a therapeutically effective amount of one or more FXR agonists (such as two or more, three or more, four or more, or five or more of the disclosed FXR agonists, such as 2, 3, 4, or 5 of the disclosed FXR agonists) is used in the treatment or prevention of LADA in subjects (for a review see Pipi et al., World J. Diabetes, 5:505-10, 2014, herein incorporated by reference). In some embodiments, one or more of the FXR agonists disclosed herein is administered to a gastrointestinal (GI) tract of the subject to activate FXR receptors in the intestines, and thereby treat or prevent LADA in the subject. Thus, in some embodiments the FXR agonist(s) is administered to, without limitation, the mouth (such as by injection or by ingestion by the subject), the esophagus, the stomach or the intestines themselves.

In some embodiments, orally delivered FXR agonists are ineffectively absorbed, resulting in intestinally-restricted FXR activation. In some embodiments, FXR activation is completely limited to the intestine. In some embodiments, administration of one or more FXR agonists does not result in significant activation in the liver or kidney. In other embodiments, some measurable extra-intestinal FXR activation occurs, however the FXR activation is considerably greater in the intestines than in other locations in the body, such as in the liver or kidney. In some embodiments, the FXR agonist is minimally absorbed. In some embodiments, the FXR agonist is directly administered to the intestines (such as to the distal ileum) of an individual in need thereof. In some embodiments, the FXR agonist is directly administered to the colon or the rectum (e.g., using a suppository) of an individual in need thereof. In some embodiments, the FXR agonist is administered orally, and less than 50%, less than 40%, less than 30%, less than 20%, less than 10%, less than 9%, less than 8%, less than 7%, less than 6%, less than 5%, less than 4%, less than 3%, less than 2%, or less than 1% of the FXR agonist is systemically absorbed. For example, systemic absorbion can be measured by determining serum levels of the FXR agonist following its administration, such as at least 30 minutes, at least 1 hour, at least 1.5 hours, at least 2 hours, at least 4 hours, or at least 8 hours following its administration. In some embodiments, the serum concentration of the FXR agonist in the subject remains below the compound's EC₅₀ following administration (such as a level at least 20%, at least 30%, at least 40%, or even at least 50% below the compound's EC₅₀).

In some examples, the method is a method of treating a subject having LADA. Patients with LADA can have hyperglycemia in the context of insulin resistance, but also have some of the immunological and clinical features of insulin-dependent diabetes mellitus, such as no insulin due to breakdown of pancreatic islet cells. Thus, changes in lifestyle (e.g., maintaining a healthy weight, exercising, eating sensibly) are no longer effective for such patients. The disclosed methods provide a treatment option for such patients, as the methods can restore pancreatic β-cell functions with enhanced glucose-stimulated insulin secretion (GSIS).

In some examples, the subject to be treated has a body mass index (BMI) of 25 of higher (or may have a normal BMI), a fasting blood glucose of 126 mg/dl or greater (e.g., is hyperglycemic), has a decreased number of functioning pancreatic beta cells, produces no insulin, have persistent islet cell antibodies, have high frequency of thyroid and gastric autoimmunity, have DR3 and DR4 human leukocyte antigen haplotypes, show progressive loss of beta cells, adult disease onset, defective glycaemic control, without tendency to ketoacidosis, have low levels of C-peptide, or combinations thereof.

In some examples, the subject to be treated has a BMI of 30 of higher, 35 or higher, or 40 or higher, such as 25-29, 30-34, 35-39, or 40 or more. However, in other examples, the subject to be treated has a normal BMI, such as 16.5-18.5 or 18.5 to 25. In some examples, the subject to be treated has a fasting blood glucose of at least 150 mg/dl, at least 300 mg/dl, or even at least 500 mg/dl. In some examples, the subject to be treated has a decreased number of functioning pancreatic beta cells, such as a decrease of at least 20%, at least 25%, at least 40%, at least 50%, at least 80%, at least 90%, or at least 95% relative to a non-diabetic (e.g., healthy) patient (for example as determined by measuring C-peptide or insulin levels). In some examples, the subject to be treated produces no detectable insulin (e.g., serum insulin). In some examples, the subject to be treated has persistent islet cell antibodies, such as islet cell antibodies (ICA), glutamic acid decarboxylase autoantibodies (GADA), insulinoma-associated (IA-2) autoantibodies (such as those against the IA-2 (256-760) fragment), anti-insulin antibodies (IAA), and/or zinc transporter autoantibodies (ZnT8). Glutamic acid decarboxylase (GAD) is a GABA-synthesizing enzyme that has two forms in humans, GAD65 and GAD67. Anti-GAD65 autoantibodies are the most typical connected with ICA reactivity. In some examples, a patient with LADA has GAD antibody positivity (>5 RU). In some examples, the subject to be treated has a high frequency of thyroid and gastric autoimmunity. In some examples, the subject to be treated has DR3 and DR4 human leukocyte antigen haplotypes. In some examples, the subject to be treated shows progressive loss of beta cells. In some examples, the subject to be treated has adult-onset diabetes (e.g., >35 years). In some examples, the subject to be treated defective glycaemic control. In some examples, the subject to be treated does not have a tendency to develop ketoacidosis. In some examples, the subject to be treated has low levels of C-peptide. In some examples, the subject to be treated displays, at least 2, at least 3, at least 4, at least 5, such as 1, 2, 3, 4, 5, 6, 7, 8, 9, 10 or all of such clinical parameters.

In some examples, the method is a method of preventing a subject having type II diabetes from developing LADA, such as a subject having type II diabetes but at risk to develop LADA. In some examples, a patient is determined to be at risk of developing LADA by measuring or detecting antibodies, such as anti-GAD, anti-IA2, and/or anti-insulin. The disclosed methods provide a preventative/protective option for such patients, as the methods can protect pancreatic p-cells from destruction. In some examples, the subject having type II diabetes and at risk to develop LADA has a BMI of 30 of higher, 35 or higher, or 40 or higher, such as 25-29, 30-34, 35-39, or 40 or more. However, in other examples, the subject having type II diabetes and at risk to develop LADA has a normal BMI, such as 16.5-18.5 or 18.5 to 25. In some examples, the subject to be treated has a fasting blood glucose of at least 150 mg/dl, at least 300 mg/dl, or even at least 500 mg/dl. In some examples, the subject having type II diabetes and at risk to develop LADA has a decreased number of functioning pancreatic beta cells, such as a decrease of at least 20%, at least 25%, at least 40%, at least 50%, at least 80%, at least 90%, or at least 95% relative to a non-diabetic (e.g., healthy) patient (for example a determined by measuring C-peptide and/or insulin levels). In some examples, the subject having type II diabetes and at risk to develop LADA produces reduced levels of detectable insulin (e.g., serum insulin), such as a decrease of at least 20%, at least 25%, at least 40%, at least 50%, at least 80%, at least 90%, or at least 95% relative to a non-diabetic (e.g., healthy) patient. In some examples, the subject having type II diabetes and at risk to develop LADA is insulin resistant. In some examples, the subject having type II diabetes and at risk to develop LADA has persistent islet cell antibodies, such as islet cell antibodies (ICA), glutamic acid decarboxylase autoantibodies (GADA), insulinoma-associated (IA-2) autoantibodies (such as those against the IA-2 (256-760) fragment), anti-insulin antibodies (IAA), and/or zinc transporter autoantibodies (ZnT8). Glutamic acid decarboxylase (GAD) is a GABA-synthesizing enzyme that has two forms in humans, GAD65 and GAD67. Anti-GAD65 autoantibodies are the most typical connected with ICA reactivity. In some examples, a patient with LADA has GAD antibody positivity (>5 RU). In some examples, the subject having type II diabetes and at risk to develop LADA has a high frequency of thyroid and gastric autoimmunity. In some examples, the subject having type II diabetes and at risk to develop LADA has DR3 and DR4 human leukocyte antigen haplotypes. In some examples, the subject having type II diabetes and at risk to develop LADA shows progressive loss of beta cell. In some examples, the subject having type II diabetes and at risk to develop LADA has adult-onset diabetes (e.g., >35 years). In some examples, the subject having type II diabetes and at risk to develop LADA has defective glycaemic control. In some examples, the subject having type II diabetes and at risk to develop LADA does not have a tendency to develop ketoacidosis. In some examples, the subject having type II diabetes and at risk to develop LADA has low levels of C-peptide. In some examples, the subject to be treated displays, at least 2, at least 3, at least 4, at least 5, such as 1, 2, 3, 4, 5, 6, 7, 8, 9, 10 or all of such clinical parameters.

In some examples, administration of one or more FXR agonists restores pancreatic β cell functions with enhanced glucose-stimulated insulin secretion (GSIS) in a subject, for example, without significant body weight changes (e.g., no more than a 10% increase or no more than a 5% increase in body weight). In some examples, such methods increase GSIS in the subject by at least 5%, at least 10%, at least 15%, at least 20%, at least 30% or even at least 50% (such as 5% to 50%, 5% to 25%, 10% to 20%, or 10% to 30%), for example relative to a subject not treated with the disclosed therapies. Methods of measuring GSIS are known, and exemplary methods are provided herein.

The disclosed methods in some examples enhance GLP-1 secretion in enteroendocrine L cells in a subject (such as a human). GLP-1 is an incretin derived from the transcription product of the proglucagon gene. The major source of GLP-1 in the body is the intestinal L cell that secretes GLP-1 as a gut hormone. The biologically active forms of GLP-1 include GLP-1-(7-37) and GLP-1-(7-36)NH₂ (HAEGTFTSDVSSYLEGQAAKEFIAWLVKGR; SEQ ID NO: 1) in humans, which result from selective cleavage of the proglucagon molecule. GLP-2 is a 33 amino acid peptide (HADGSFSDEMNTILDNLAARDFINWLIQTKITD; SEQ ID NO: 2) in humans. GLP-2 is created by specific post-translational proteolytic cleavage of proglucagon in a process that also liberates GLP-1. GLP agonists are “incretin mimetics” that can be used to treat type 2 diabetes. Examples include, but are not limited to: exenatide (Byetta/Bydureon), liraglutide (Victoza), lixisenatide (Lyxumia), and albiglutide (Tanzeum). In certain embodiments, the FXR agonist enhances the secretion of glucagon-like peptide-1 (GLP-1) and/or glucagon-like peptide-2 (GLP-2). In some examples, such methods increase GLP-1 secretion in enteroendocrine L cells in the subject by at least 5%, at least 10%, at least 15%, at least 20%, at least 30% or even at least 50% (such as 5% to 50%, 5% to 25%, 10% to 20%, or 10% to 30%), for example relative to a subject not treated with the disclosed therapies. Methods of measuring GLP-1 secretion are known, and exemplary methods are provided herein.

In some examples, administration of one or more FXR agonists increases the respiratory capacity (e.g., mitochondrial bioenergetics) in an intestinal L cell. In some examples, such methods increase respiratory capacity in an intestinal L cell by at least 5%, at least 10%, at least 15%, at least 20%, at least 30% or even at least 50% (such as 5% to 50%, 5% to 25%, 10% to 20%, or 10% to 30%), for example relative to a cell not treated with the disclosed therapies.

The disclosed methods in some examples increase gene expression of glucagon-like peptide-1 receptor (GLP-1R) in pancreatic β cells, resulting in restoration of GSIS in β cells to ameliorate hyperglycemia. In some examples, such methods increase GLP-1R in pancreatic β cells in the subject by at least 5%, at least 10%, at least 15%, at least 20%, at least 30% or even at least 50% (such as 5% to 50%, 5% to 25%, 10% to 20%, or 10% to 30%), for example relative to a subject not treated with the disclosed therapies. Methods of measuring gene expression (such as expression of GLP-1R) are known (e.g., detection of GLP-1R proteins and/or nucleic acids), and exemplary methods are provided herein.

The disclosed methods in some examples decrease the amount of serum insulin in the subject. Serum levels of insulin are decreased when blood glucose levels are lowered and insulin sensitivity is also increased. Thus, in some examples, the disclosed methods decrease the amount of serum insulin in a subject (such as a human). In some examples, such methods decrease serum insulin in the subject by at least 5%, at least 10%, at least 15%, at least 20%, at least 30%, at least 50% or even at least 75% (such as 5% to 50%, 5% to 25%, 10% to 20%, 10% to 70%, or 10% to 30%), for example relative to levels observed in a subject not treated with the disclosed therapies. Methods of measuring serum insulin are known, and exemplary methods are provided herein.

The disclosed methods in some examples decrease the amount of serum glucose in the subject. Thus, in some examples, the disclosed methods decrease the amount of serum glucose in a subject (such as a human). In some examples, such methods decrease serum glucose in the subject by at least 5%, at least 10%, at least 15%, at least 20%, at least 30%, at least 50% or even at least 75% (such as 5% to 50%, 5% to 25%, 10% to 20%, 10% to 70%, or 10% to 30%), for example relative to levels observed in a subject not treated with the disclosed therapies. Methods of measuring serum glucose are known, and exemplary methods are provided herein.

The disclosed methods in some examples decrease the amount of one or more markers of pancreatic beta cell damage, such as thioredoxin-interacting protein (Txnip) and inflammatory markers such as interleukin 1, in the subject. Txnip is a marker of pancreatic beta cell damage (OMIM 606599), and sequences are publicly available (e.g., GenBank Accession Nos. NP_006463.3, AAH93704.1, NP_001009935.1, NM_006472.4 and NM_001009935.2). Thus, in some examples, the disclosed methods decrease the amount of one or more markers of pancreatic beta cell damage (e.g., Txnip) in a subject (such as a human). In some examples, such methods decrease one or more markers of pancreatic beta cell damage, such as Txnip, in the subject by at least 5%, at least 10%, at least 15%, at least 20%, at least 30%, at least 50% or even at least 75% (such as 5% to 50%, 5% to 25%, 10% to 20%, 10% to 70%, or 10% to 30%), for example relative to levels observed in a subject not treated with the disclosed therapies. Methods of measuring such proteins are known (e.g., at the protein or nucleic acid level), and exemplary methods are provided herein.

In some embodiments, one or more FXR agonists are co-administered with one or more additional compounds or therapies, for treatment or prevention of a metabolic disorder. For example, one or more FXR agonists can be administered with an insulin sensitizing drug, an insulin secretagogue, an alpha-glucosidase inhibitor, a GLP agonist, a DPP-4 inhibitor (such as sitagliptin, vildagliptin, saxagliptin, linagliptin, anaglptin, teneligliptin, alogliptin, gemiglptin, or dutoglpitin), a catecholamine (such as epinephrine, norepinephrine, or dopamine), peroxisome proliferator-activated receptor (PPAR)-gamma agonist (e.g., a thiazolidinedione (TZD) [such as ioglitazone, rosiglitazone, rivoglitazone, or troglitazone], aleglitazar, farglitazar, muraglitazar, or tesaglitazar) or a combination thereof. Likewise, one or more FXR agonists can be administered with a statin, HMG-CoA reductase inhibitor, fish oil, fibrate, niacin or other treatment for dyslipidemia. In one example, the one or more FXR agonists can be administered with nicotinamide ribonucleoside and analogs thereof that promote NAD+ production of which is a substrate for many enzymatic reactions such as p450s which are a target of FXR (for examples see Yang et al., J. Med. Chem., 50:6458-61, 2007, herein incorporated by reference).

In some examples, the method also includes performing clinical assays on the subject to be administered the one or more FXR agonists. For example, the method can also include determining if the subject produces insulin (for example by measuring serum insulin); measuring the subject's BMI; measuring/determining the subject's blood glucose levels (such as while fasting); measuring/determining a level of C-peptide in the subject; measuring/determining a level of islet cell antibodies (ICA), glutamic acid decarboxylase autoantibodies (GADA), insulinoma-associated (IA-2) autoantibodies, anti-insulin antibodies (IAA), and/or zinc transporter autoantibodies (ZnT8) in the subject; or combinations thereof; measuring/detecting functional pancreatic beta cells (e.g., measured by detecting C-peptide); measuring/detecting frequency of thyroid and gastric autoimmunity; determining if the subject has DR3 and DR4 human leukocyte antigen haplotypes; and/or determining if the subject has a tendency to ketoacidosis. Such methods are routine, and exemplary methods are provided herein. For example, methods of detecting antibodies and proteins are routine, such as immunoassay, such as ELISA or RIA, microscopy, spectroscopy, and the like. In some examples, at least 2, at least 3, at least 4, at least 5, such as 1, 2, 3, 4, 5, 6, 7, 8, 9, 10 or all of such clinical parameters are measured.

In some examples, the method includes selecting a subject having or at risk for developing LADA for administration of one or more FXR agonists.

B. Administration

The particular mode of administration and the dosage regimen can be selected by the attending clinician, taking into account the particulars of the case (e.g. the subject, the disease, the disease state involved, the particular treatment, and whether the treatment is prophylactic). Treatment can involve daily or multi-daily or less than daily (such as weekly or monthly etc.) doses over a period of a few days to months, or even years. For example, a therapeutically effective amount of one or more FXR agonists can be administered in a single dose, twice daily, weekly, or in several doses, for example daily, or during a course of treatment. In a particular non-limiting example, treatment involves once daily dose or twice daily dose.

In some embodiments, one or more FXR agonists are administered orally. In some embodiments, one or more FXR agonists are administered as an ileal-pH sensitive release formulation that delivers the compound to the intestines, such as to the ileum of an individual. In some embodiments, one or more FXR agonists are administered as an enterically coated formulation. In some embodiments, oral delivery of one or more FXR agonists provided herein can include formulations, as are well known in the art, to provide prolonged or sustained delivery of the drug to the gastrointestinal tract by any number of mechanisms. These include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. The intended effect is to extend the time period over which the active drug molecule is delivered to the site of action (e.g., the intestines) by manipulation of the dosage form. Thus, enteric-coated and enteric-coated controlled release formulations are within the scope of the present disclosure. Suitable enteric coatings include cellulose acetate phthalate, polyvinylacetate phthalate, hydroxypropylmethylcellulose phthalate and anionic polymers of methacrylic acid and methacrylic acid methyl ester.

In some embodiments, one or more FXR agonists are administered before ingestion of food, such as at least 10 minutes, at least 15 minutes, at least 20 minutes, or at least 30 minutes before ingestion of food (such as 10-60 minutes or 10-30 minutes before ingesting food). In some embodiments of the methods described herein, one or more FXR agonists are administered less than about 60 minutes before ingestion of food. In some embodiments of the methods described above, one or more FXR agonists are administered less than about 30 minutes before ingestion of food. In some embodiments of the methods described herein, one or more FXR agonists are administered after ingestion of food. In some embodiments, the methods further include administration of an insulin sensitizing drug, an insulin secretagogue, an alpha-glucosidase inhibitor, a GLP agonist, a DPP-4 inhibitor (such as sitagliptin, vildagliptin, saxagliptin, linagliptin, anaglptin, teneligliptin, alogliptin, gemiglptin, or dutoglpitin), a catecholamine (such as epinephrine, norepinephrine, or dopamine), peroxisome proliferator-activated receptor (PPAR)-gamma agonist (e.g., a thiazolidinedione (TZD) [such as ioglitazone, rosiglitazone, rivoglitazone, or troglitazone], aleglitazar, farglitazar, muraglitazar, or tesaglitazar), a TGR5 agonist, a biguanide, or a combination thereof.

A FXR agonist containing composition administered can include at least one of a spreading agent or a wetting agent. In some embodiments, the absorption inhibitor is a mucoadhesive agent (e.g., a mucoadhesive polymer). In some embodiments, the mucoadhesive agent is selected from methyl cellulose, polycarbophil, polyvinylpyrrolidone, sodium carboxymethyl cellulose, and a combination thereof. In some embodiments, a pharmaceutical composition administered further includes an enteroendocrine peptide and/or an agent that enhances secretion or activity of an enteroendocrine peptide.

The pharmaceutical compositions that comprise one or more compounds disclosed herein can be formulated in unit dosage form, suitable for individual administration of precise dosages. In one non-limiting example, a unit dosage contains from about 1 mg to about 50 g of one or more compounds disclosed herein, such as about 10 mg to about 10 g, about 100 mg to about 10 g, about 100 mg to about 1 g, about 500 mg to about 5 g, or about 500 mg to about 1 g. In other examples, a therapeutically effective amount of one or more compounds disclosed herein is from about 0.01 mg/kg to about 500 mg/kg, for example, about 0.5 mg/kg to about 500 mg/kg, about 5 mg/kg to about 250 mg/kg, or about 50 mg/kg to about 100 mg/kg, such as at least 1 mg/kg, at least 10 mg/kg, at least 25 mg/kg, at least 50 mg/kg, or at least 100 mg/kg. In other examples, a therapeutically effective amount of one or more compounds disclosed herein is from about 50 mg/kg to about 250 mg/kg, for example about 100 mg/kg.

In some examples, the FXR agonist is effective faster and/or at a lower dose than Fex. For example, it is shown herein that Fex-D is effective at lowering blood glucose with half the dose in half the time (50 mg/kg for 2 weeks), as compared to Fex (100 mg/kg for 4 weeks). Thus, in some examples, the FXR agonist used is effective at a dose at least 20% less, at least 30%, less, at least 40% less, or at least 50% less than Fex. In some examples, the FXR agonist used is effective at a dose at least 20% faster, at least 30%, faster, at least 40% faster, or at least 50% faster than Fex. In some examples, the FXR agonist used is effective at a dose at least 20% faster, at least 30%, faster, at least 40% faster, or at least 50% faster than Fex and is effective at a dose at least 20% less, at least 30%, less, at least 40% less, or at least 50% less than Fex.

### Example 1

### Materials & Methods

A. Animals and Animal Care

Leptin-deficient ob/ob mice were purchased from Jackson laboratory (stock no. 000632). Each mouse consumed 100 mg per kg of body weight Fex (in corn oil) per day by oral gavage for 5 weeks. For in vivo analog study, each mouse consumed 50 mg per kg of body weight Fex-D (in corn oil) per day by oral gavage for 14 days. For GLP-1 receptor antagonist study, each mouse was daily treated with 100 μg per kg of body weight Exendin 9-39 amide (Abcam) via intraperitoneal injection for 2 weeks. For INT-777 study, each mouse was daily gavaged with 60 mg per kg of body weight INT-777 (in corn oil) for 5 weeks. For FGF15 study, each mouse was daily treated with 0.25 mg per kg of body weight FGF15 (in PBS with 0.1% BSA) via intravenous injection for 2 weeks. The sample sizes for all animal studies are indicated in each figure legend. All mice were housed in a specific pathogen-free facility with a 12-h light, 12-h dark cycle and given free access to food and water. All in vivo data were retrieved from 3 independent experimental animal cohorts. Core body temperature was measured with a clinical rectal thermometer (Thermalert model TH-5; Physitemp). For GTTs, 6-h fasted mice received 2 g of glucose per kg of body weight by oral gavage. Human insulin (Humilin, Eli Lilly) was used for insulin sensitivity test. Tail blood was drawn at indicated time intervals, and blood glucose level was measured with a One Touch Ultra glucometer (LifeScan). Body composition was measured with Echo MRI-100 body composition analyzer (Echo Medical Systems). All mice were randomly assigned to experimental groups for further analysis. No samples were excluded from the analysis.

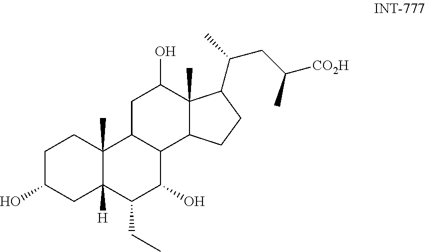 [SMILES: CC[C@H]1[C@@H](O)C2C3CCC([C@H](C)C[C@H](C)C(=O)O)[C@@]3(C)C(O)CC2[C@@]2(C)CC[C@@H](O)C[C@@H]12]

B. In Vivo Metabolic Phenotype Analysis

Real-time metabolic analyses were conducted in a Comprehensive Lab Animal Monitoring System (Columbus Instruments). CO₂ production, O₂ consumption, RQ (relative rates of carbohydrate versus fat oxidation) and ambulatory counts were determined for 4 consecutive days and nights, with at least 24-h for adaptation before data recording.

C. Isolated Pancreatic Islet Studies

Mouse pancreatic islets were isolated from ob/ob mice according to a method used for rats traditionally. Briefly, 0.5 mg/ml collagenase P (Roche) diluted in HBSS buffer was injected through the common bile duct and the perfused pancreas was dissected and incubated in water bath with 37° C. for 21 minutes. Digested exocrine cells and intact islets were separated using Histopaque-1077 (SIGMA) with centrifugation by 900 g for 15 minutes and intact islets were picked up manually.

D. Insulin Secretion Assay (Primary Mouse Pancreatic Islet and Human Islets)

Insulin release from intact islets was monitored using batch incubation methods.

Isolated pancreatic islets were cultured over-night with RPMI1940 supplemented with 10% (v/v) fetal bovine serum and 1% (v/v) Antibiotics-antimycotic solution, were pre-cultured at 37° C. for 30 minutes in Krebs Ringer bicarbonate buffer (KRBH) containing 129.4 mM NaCl, 3.7 mM KCl, 2.7 mM CaCl₂, 1.3 mM KH₂PO₄, 1.3 mM MgSO₄, 24.8 mM NaHCO₃ (equilibrated with 5% CO₂, 95% O₂, pH7.4), 10 mM HEPES and 0.2% (v/v) BSA (fraction V) with 3 mM glucose. N ext, the pancreatic islets were incubated for 30 minutes KRBH buffer (500 μl/10 islets) with 3 mM or 20 mM glucose with or without 100 nM Exendin-4 to determine insulin secretion levels. At the end of the incubation period, islets were pelleted by centrifugation, and aliquots of the buffer were sampled. The amounts of insulin were determined by Rat/mouse Insulin ELISA KIT (Millipore) for mice islets.

E. GLP-1 Secretion Assay

In vitro GLP-1 secretion assay was performed in human enteroendocrinal L cells (NCI-H716). For each secretion assay, cells were seeded at 1×10⁵ cells/ml (1 ml/well) in a matrigel-coated 24-well plate. Prior to GLP-1 secretion assay, cells were pre-treated with DMSO, 1 μM Fex, or 1 μM Fex-D overnight. Cells were deprived in Krebs buffer (with 0.1% BSA) with dipeptidyl peptidase (DPP)-4 inhibitor (100 M sitagliptin). GLP-1 was measured with a commercial kit (EZGLP1T-36K, Millipore). For in vivo GLP-1 secretion, each animal was gavaged with 25 mg per kg of body weight sitagliptin 60-minutes before glucose load. Blood was collected from the tail vein at the indicated intervals, and transferred to serum collection tubes containing sitagliptin.

F. Extracellular Oxygen Consumption Rate Assay

Briefly, NCI-H716 cells were seeded in XF 96-well cell culture microplates (Seahorse Bioscience) at 6.0×10⁴ cells per well in 150 μl of growth medium and then incubated at 37° C./5% CO₂ for 24 hr. A Seahorse Bioscience instrument model XF96 was used to measure the rate of change of dissolved oxygen in the media surrounding the cells. All procedures followed manufacturer's instructions.

G. RNA-Seq Library Generation

Total RNA was isolated from cell pellets treated with RNAlater using the RNA mini kit (Qiagen) and treated with DNaseI (Qiagen) for 30 minutes at room temperature. Sequencing libraries were prepared from 100-500 ng total RNA using the TruSeq RNA Sample Preparation Kit v2 (Illumina) according to the manufacturer's protocol. Briefly, mRNA was purified, fragmented, and used for first-, then second-strand cDNA synthesis followed by adenylation of 3′ ends. Samples were ligated to unique adapters and subjected to PCR amplification. Mulitplexed libraries were then validated using the 2100 BioAnalyzer (Agilent), normalized, and pooled for sequencing. RNA-Seq libraries prepared from two biological replicates for each experimental condition were sequenced on the Illumina HiSeq 2500 using bar-coded multiplexing and a 100 bp read length.

H. High-Throughput Sequencing and Analysis

Image analysis and base calling were performed with Illumina CASAVA-1.8.2. This yielded a median of 29.9M usable reads per sample. Short read sequences were mapped to a UCSC mm9 reference sequence using the RNA-seq aligner STAR. Known splice junctions from mm9 were supplied to the aligner and de novo junction discovery was also permitted. Differential gene expression analysis, statistical testing and annotation were performed using Cuffdiff 2. Transcript expression was calculated as gene-level relative abundance in fragments per kilobase of exon model per million mapped fragments and employed correction for transcript abundance bias. RNA-Seq results for genes of interest were also explored visually using the UCSC Genome Browser.

### Example 2

### Fex Improves Glucose Homeostasis in Ob/Ob Mice

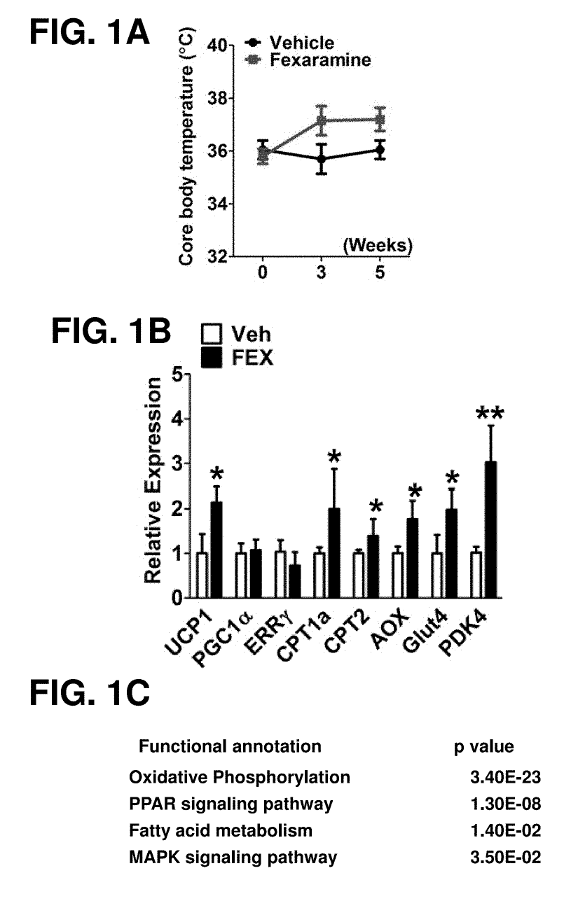

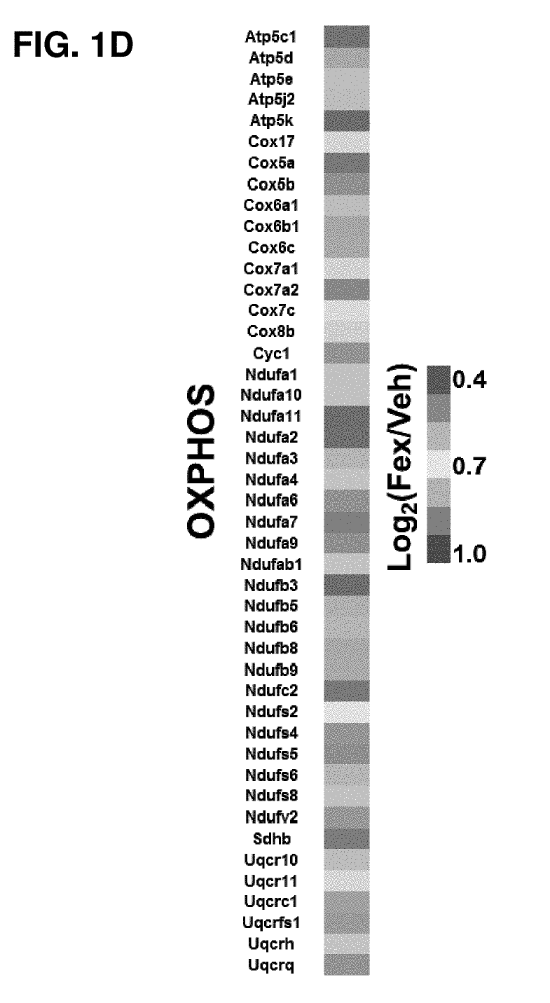

To investigate the effect of Fex treatment in a diabetic mouse model, leptin-deficient obese mice (ob/ob) were subjected to daily treatment by oral gavage (100 mg/kg) for 5 weeks. Fex increases energy expenditure to exhibit a reduction in weight gain in diet-induce obese (DIO) mice. Consistently, Fex-treated ob/ob mice exhibited an increase in core temperature (FIG. 1A). Gene expression analysis confirmed the induction of UCP1 as well as a number of genes involved in β-oxidation, thermogenesis and oxidative phosphorylation (FIG. 1B-1D).

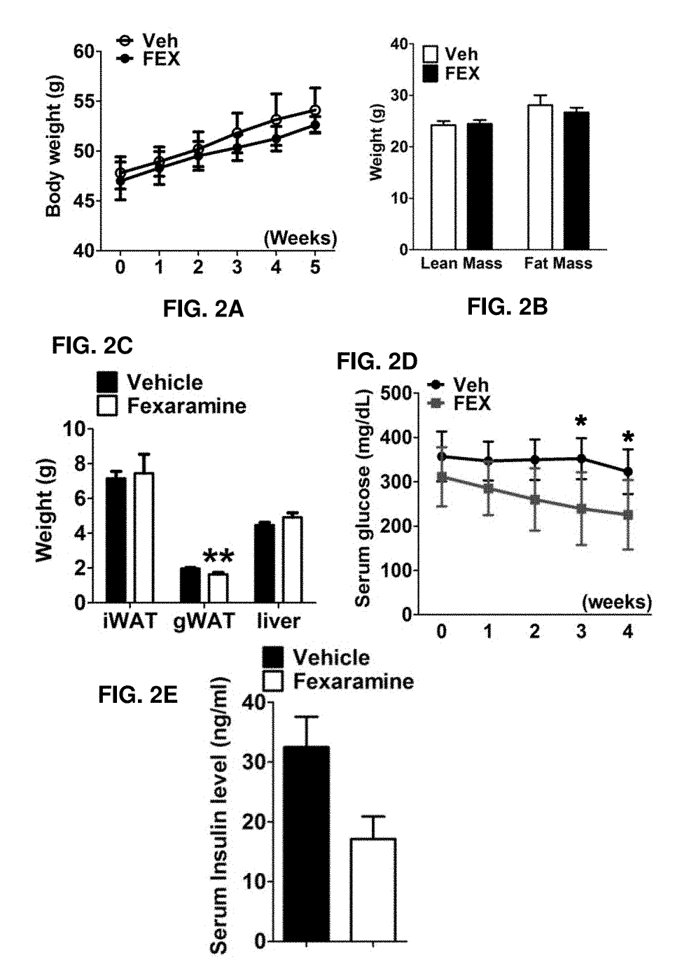

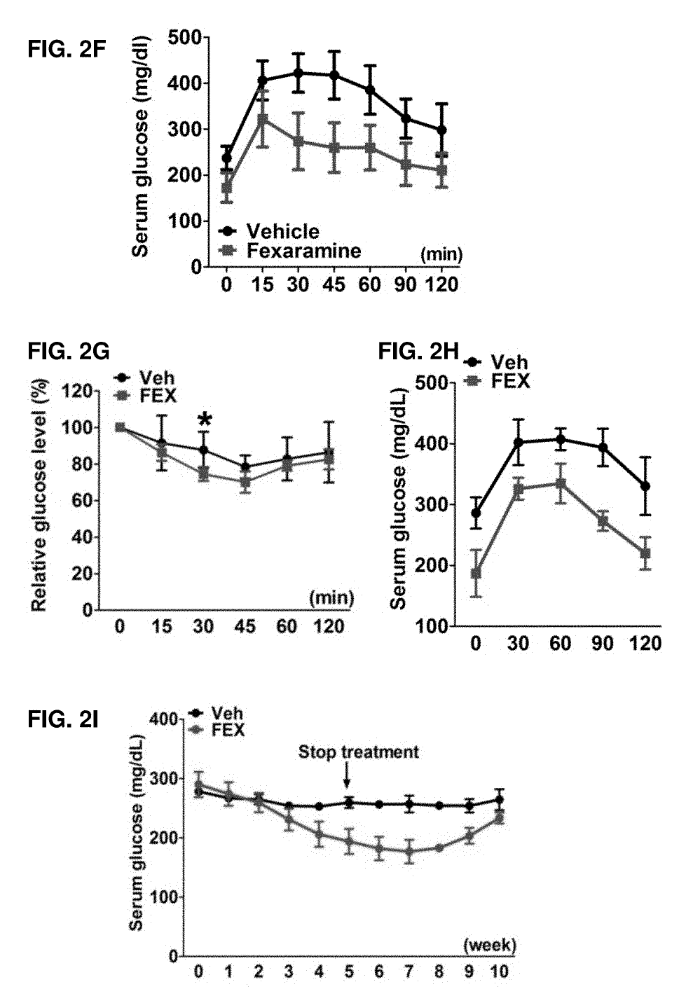

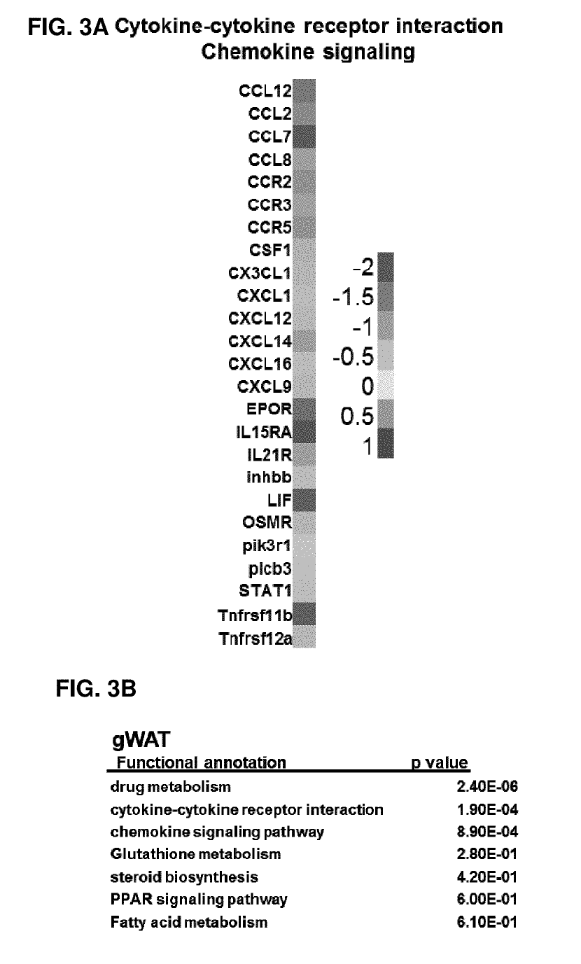

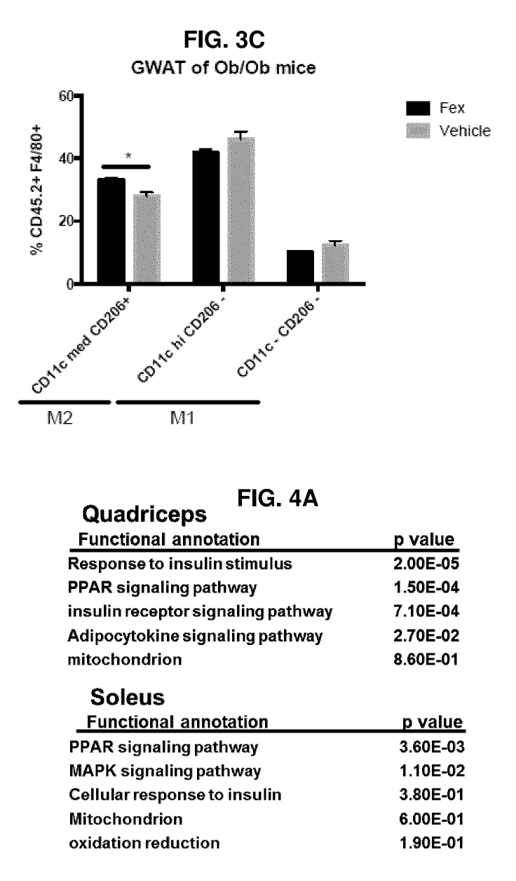

In marked contrast, Fex-treated ob/ob mice did not exhibit any changes of body weight gain (FIG. 2A) or lean/fat mass (FIG. 2B). While no difference was observed in the tissue weights of liver and inguinal fat (iWAT), gonadal fat (gWAT) was modestly but significantly reduced by Fex treatment (FIG. 2C). Consistent with reduced adiposity, the gene expression of inflammatory cytokines and chemokines were decreased in gWAT (FIGS. 3A and 3B). Furthermore, Fex treatment changed the relative proportions of immune cells in adipose tissues, most notably increasing the proportion of M2 macrophages in gWAT, indicating that Fex treatment suppressed inflammation in visceral adipose tissues (FIGS. 3A-3C).

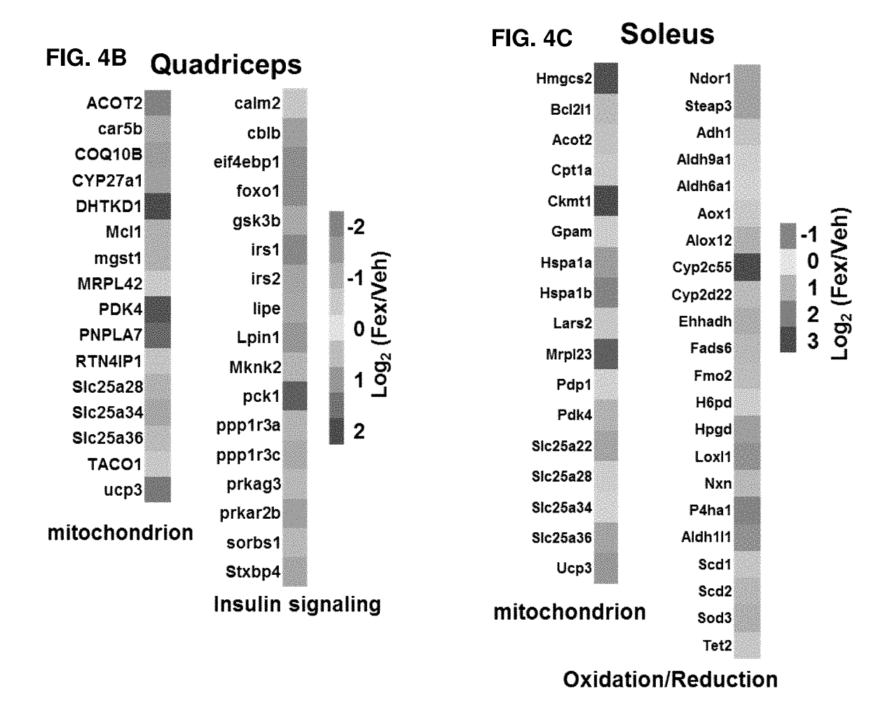

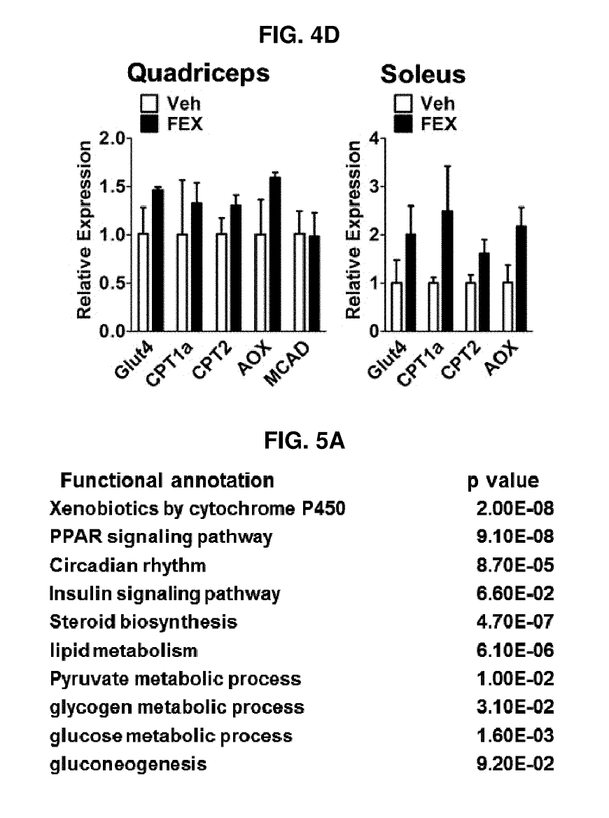

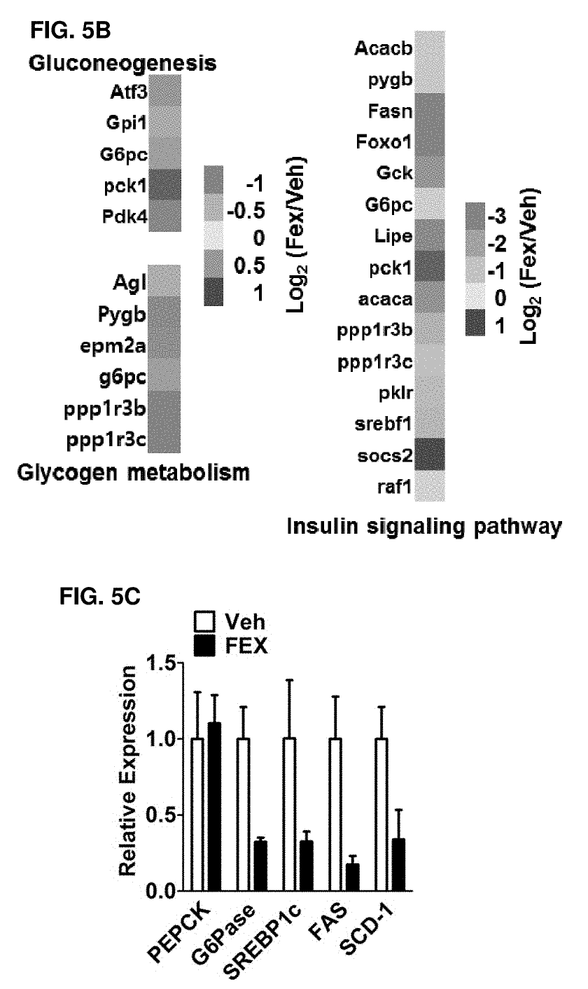

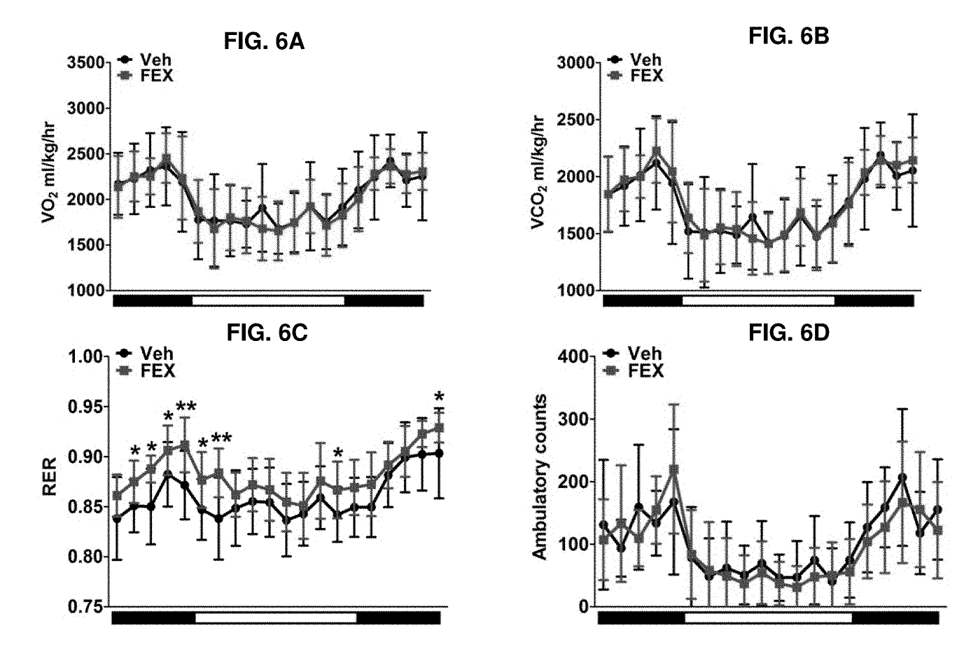

Reduction of inflammation in visceral fat improves glucose homeostasis, and thus the physiological effects of Fex on glucose homeostasis were examined in ob/ob mice. Fex-treated mice exhibited significant improvements in their endocrine and metabolic profiles including reduced glucose and insulin levels (FIGS. 2D and 2E). Furthermore, Fex treatment improved glucose tolerance and insulin sensitivity in ob/ob mice (FIGS. 2F and 2G). Transcriptomic analysis from skeletal muscle showed that gene involved in insulin signaling and mitochondrion were largely increased (FIGS. 4A-4D) whereas hepatic gluconeogenesis/lipogenesis were remarkably suppressed by Fex treatment (FIG. 2H and FIGS. 5A-5C). Notably, Fex-treated mice exhibited increased respiratory exchange ratio in the dark period (FIGS. 6A-6D).

Given that mice have nocturnal feeding behavior, these data indicate that Fex-treated ob/ob mice increased glucose uptake to utilize more glucose as an energy source in response to feeding. To confirm that Fex treatment improved glucose homeostasis, blood glucose levels were monitored for 4 weeks after Fex treatment was stopped. Serum glucose levels remained low for 3 weeks after cessation of treatment, and then gradually increased (FIG. 2I). These data demonstrate a marked improvement in glucose homeostasis upon Fex treatment in ob/ob mice.

### Example 3

### Fex Potentiates Bioenergetics in Enteroendocrinal L Cells to Enhance GLP-1 Secretion

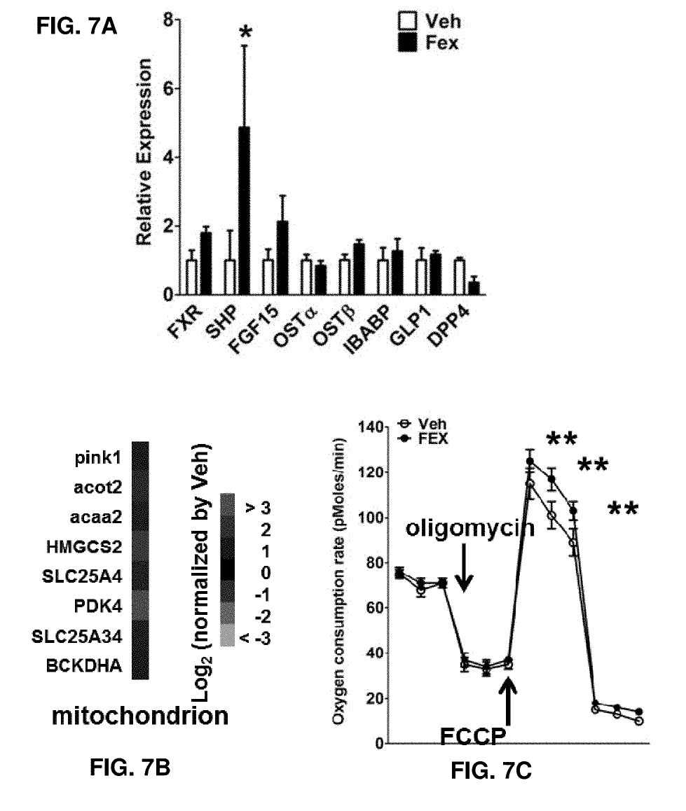

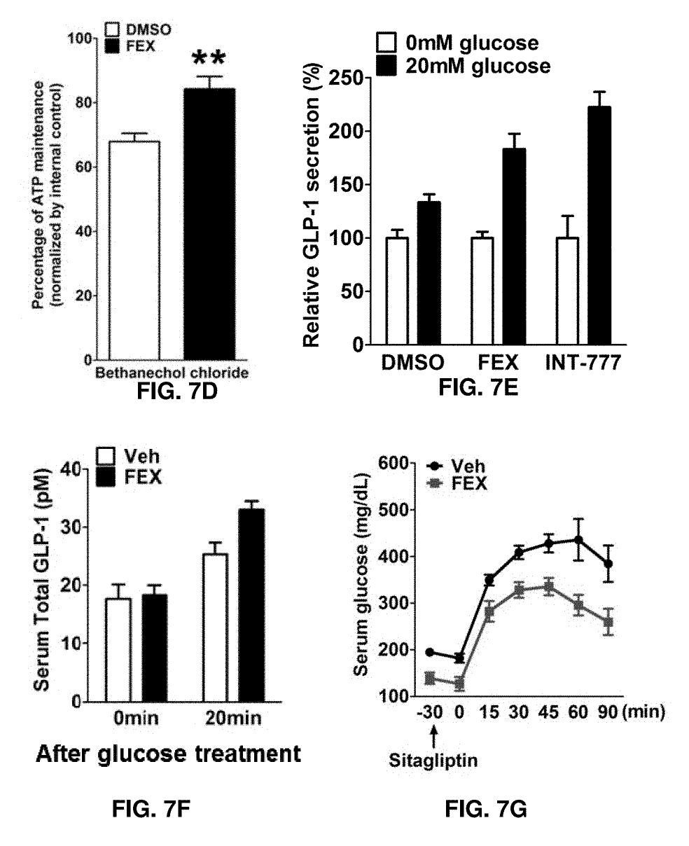

Classical FXR target genes SHP and FGF15 were markedly upregulated in the intestine upon Fex treatment (FIG. 7A). RNA-Seq of intestinal tissues was used to determine the mechanisms through which Fex contributes to systemic changes leading to improvements of glucose homeostasis. In addition to established FXR target genes, the induction of genes involved in mitochondrion was noted, potentially identifying additional FXR regulated genes (FIG. 7B). The induction of genes involved in mitochondrion is of particular interest because bioenergetics are important for the secretion of incretins such as glucagon-like peptide 1 (GLP-1) in enteroendocrinal L cells.

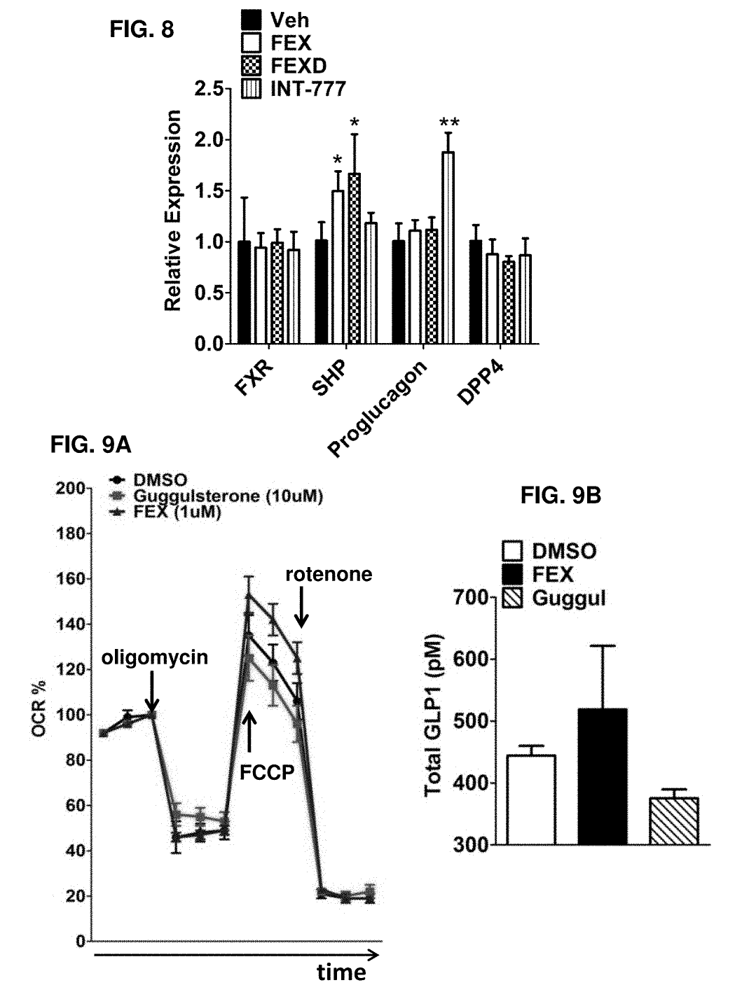

To demonstrate that Fex treatment has direct and positive effects on overall mitochondrial bioenergetics, the respiration of enteroendocrinal L cells treated with Fex or DMSO was determined on Seahorse XF96 analyzer (Seahorse Bioscience, North Billerica, Mass.). Gene expression analysis showed that functional FXR is robustly expressed in enteroendocrinal L cells, as indicated by the induction of SHP expression with Fex treatment (FIG. 8). While the basal respiratory rates were similar between treated cells, the maximum respiration rate induced by FCCP was 155% in the Fex-treated L cells (compared to 130% in the vehicle-treated L cells), indicating that transient Fex treatment led to increased respiratory capacity in intestinal L cells (FIG. 7C). Conversely, treatment with the FXR antagonist guggulsterone reduced basal respiratory rates and the FCCP-induced maximum respiration rate (FIGS. 9A and 9B).

To further examine how this increase in respiratory capacity affects L cell bioenergetics, the maintenance of ATP levels under energetic demanding conditions was evaluated. Human intestinal L cells treated with DMSO or Fex were incubated with the muscarinic receptor agonist bethanechol chloride and residual ATP levels determined. Fex treatment increased the residual ATP levels, consistent with the observed increase in respiratory capacity. Fex-treated L cells were able to maintain 83% of the initial ATP abundance whereas DMSO-treated L cells were able to maintain 67% (FIG. 7D). Thus, Fex enhances overall mitochondrial bioenergetics in enteroendocrinal L cells.

As glucose-stimulated GLP-1 secretion is an energy intensive process, and Fex enhances the energetic capacity of L cells, it was investigated whether Fex treatment could enhances GLP-1 secretion. While Fex treatment had no effect on proglucagon or DPP4 expression, the transcripts encoding the GLP-1 percursor and the peptidase degrading GLP-1, respectively (FIG. 8), L cells pretreated with Fex showed a significant increase in GLP-1 secretion in response to a glucose challenge (FIG. 7E). Similarly, treatment with INT-777, a synthetic TGR5 agonist, increased GLP-1 secretion (FIG. 7E).

To investigate whether Fex enhances GLP-1 secretion in vivo, mice were challenged with a DPP4 inhibitor, sitagliptin (25 mg/kg) prior to a glucose challenge. Fex-treated ob/ob mice displayed an increase of plasma GLP-1 level 20 minutes after the glucose challenge, and improved glucose tolerance (FIGS. 7F and 7G). These results indicate that Fex potentiates the bioenergetics in enteroendocrinal L cells to enhance GLP-1 secretion in response to a glucose challenge.

### Example 4

### Fex Restores Pancreatic β Cell Functions to Secrete Insulin in Response to Glucose in Ob/Ob Mice

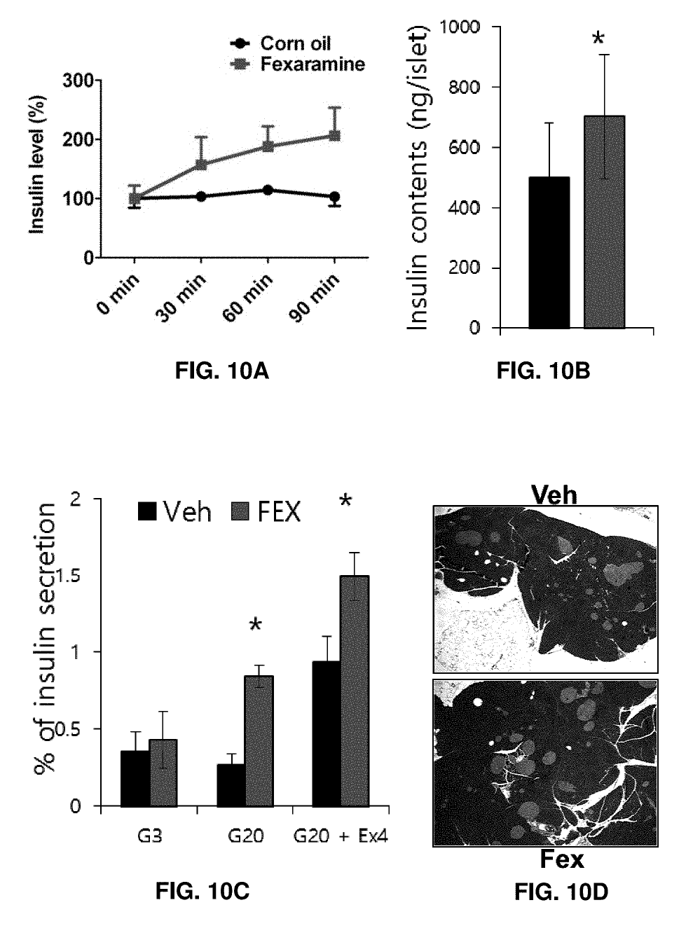

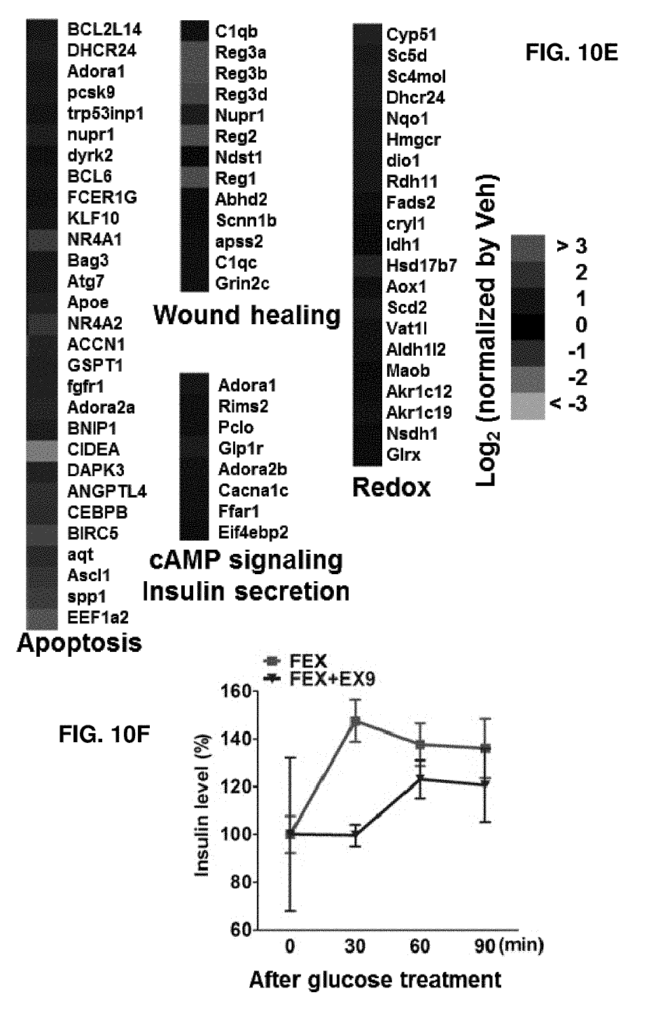

Insulin resistance can be associated with reduced 3 cell insulin secretion in response to glucose, while GLP-1 augments glucose-stimulated insulin secretion (GSIS). To examine whether Fex treatment improves GSIS, serum insulin levels were determined after a glucose challenge in ob/ob mice. A significant increase in serum insulin was observed in Fex-treated ob/ob mice (FIG. 10A). In addition, the insulin content was significantly higher in ex vivo islets from Fex-treated mice (FIG. 10B). Furthermore, ex vivo insulin secretion was dramatically increased in response to 20 mM glucose (FIG. 10D). The presence of a GLP-1 agonist, Exendin-4 (Ex-4) further potentiated insulin secretion (FIG. 10D), indicating that Fex is sufficient to enhance GSIS in pancreatic β cells from diabetic mice. Consistent with the higher insulin content, histological analysis showed that islet are larger in Fex-treated ob/ob mice (FIG. 10D). A comparison of the transcriptional changes clearly showed that Fex increased gene expressions involved in cAMP signaling/insulin secretion and wound healing pathway whereas it suppressed expression of genes involved in the apoptotic pathway, indicating that Fex improves pancreatic β cell physiology in ob/ob mice (FIG. 10E). Notably, the expression of the GLP-1 receptor was increased in Fex treated islets, potentially contributing to their increased glucose sensitivity (FIG. 10E).

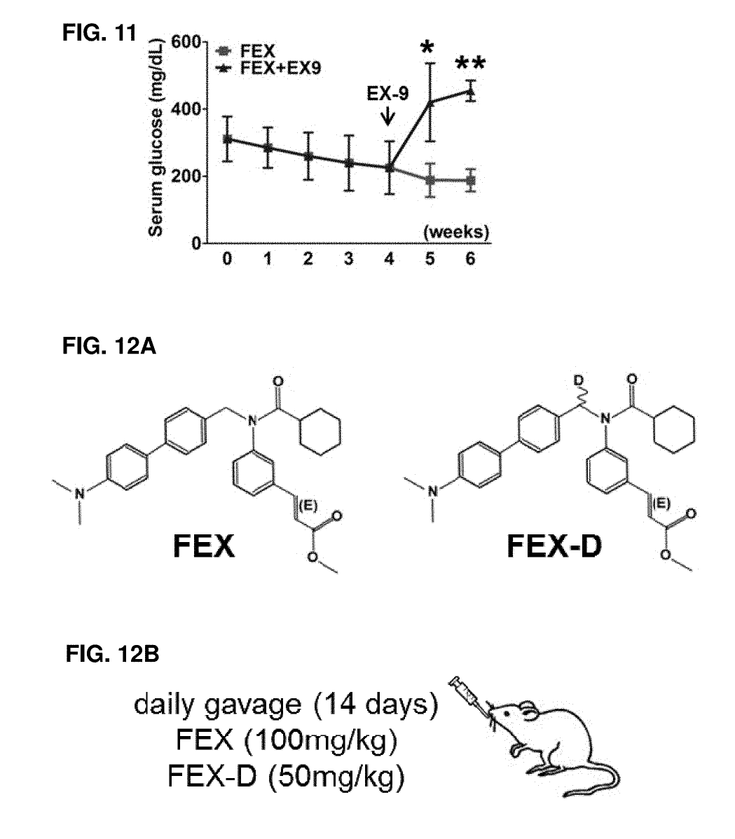

Previously, it has been reported that increased gene expression of GLP-1 receptor has been shown to enhance GLP-1 signaling in pancreatic β cells and improve insulin secretion in response to glucose. Thus, it was determined whether Fex potentiates GLP-1 signaling in islets to improve pancreatic β cell physiology in ob/ob mice. Fex-treated ob/ob mice were subjected to co-treatment with Exendin-9 (Ex-9), a GLP-1 receptor antagonist, for 2 weeks. Notably, the Fex-induced reduction in serum glucose was largely attenuated by Ex-9 co-treatment (FIG. 11). Furthermore, glucose stimulated insulin secretion was significantly impaired by Ex-9 treatment (FIG. 10F).

These results indicate that Fex treatment improves glucose homeostasis, at least in part, through enhancing the GLP-1 pathway.

### Example 5

### New Fex Analog is More Potent in Improving Pancreatic β Cell Functions in Ob/Ob Mice

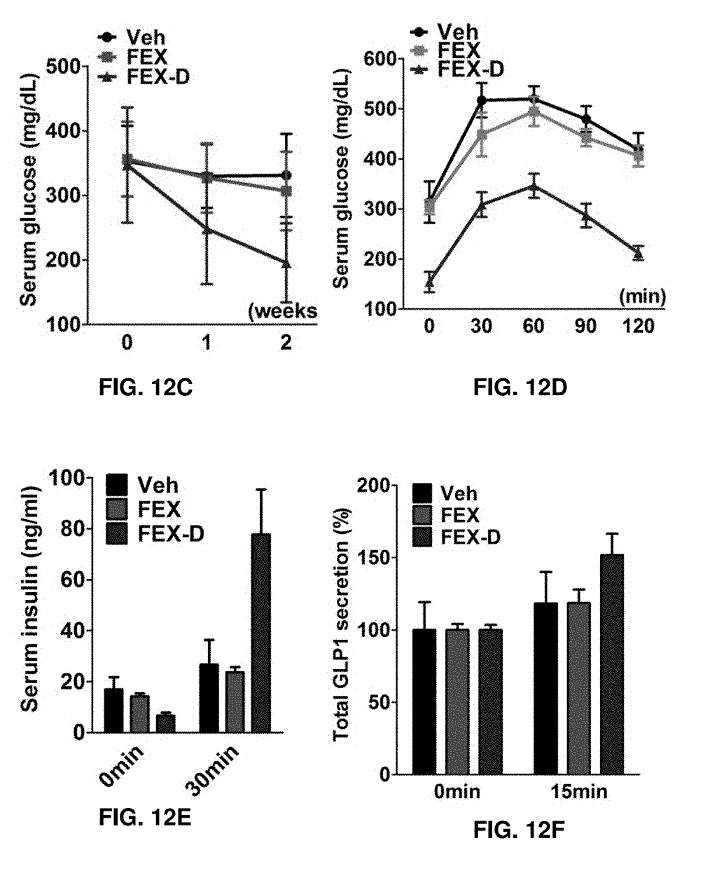

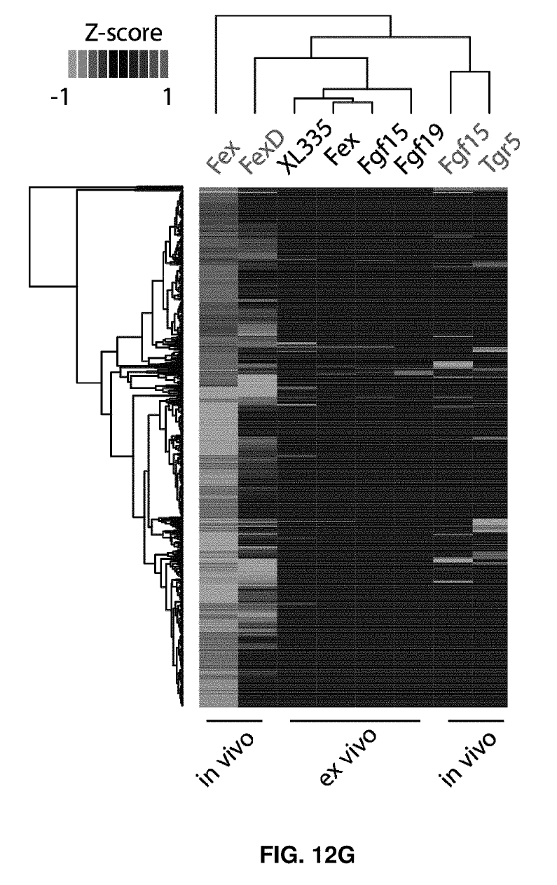

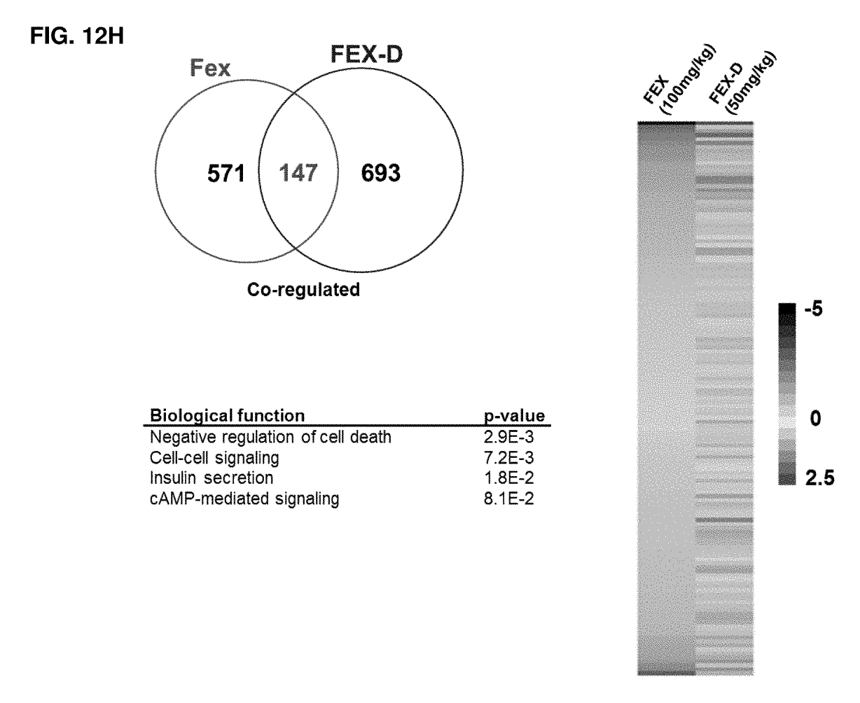

To find more efficacious FXR agonists, Fex was synthesized with a selective deuteration (Fex-D) proposed to reduce degradation by endogenous cytochrome P450 enzymes (FIG. 12A). Fex and Fex-D exhibited similar low in vitro permeability in human intestinal epithelial Caco2 cells during a 2 hour incubation. (Table 1).

TABLE 1
FEX analogues show low permeability in intestinal epithelial cells

| | Assay | Mean Pₐₚₚ, | Mean Pₐₚₚ, | Mean | | A-B |
|---|---|---|---|---|---|---|
| | Conc. | A-B | B-A | (B-A/A-B) | Mean % | Permeability |
| Compound | (μM) | (10⁻⁶ cm/s) | (10⁻⁶ cm/s) | Efflux Ratio | Recovery | Ranking |
| Fexaramine | 10 | 0.597 | 0.770 | NA | 149.7% | Lower |
| FEX-D | 10 | 0.110 | 0.274 | NA | 96.2% | Lower |
| NSSK 00024 | 10 | 0.0294 | 0.230 | NA | 192.0% | Lower |
| NSSK 00027 | 10 | 0.0295 | 0.0280 | NA | 138.2% | Lower |
| NSSK 00089 | 10 | 0.0962 | 0.0640 | NA | 67.1% | Lower |
| NSSK 00096 | 10 | 0.0104 | 0.0399 | NA | 69.9% | Lower |
| NSSK 00110 | 10 | 0.115 | 0.351 | NA | 118.3% | Lower |
| Controls: | | | | | | |
| Warfarin | 10 | 42.8 | 29.8 | 0.695 | 101.0% | Higher as |
| | | | | | | expected |
| Ranitidine | 10 | 0.274 | 0.922 | 3.36 | 92.7% | Lower as |
| | | | | | | expected |
| Talinolol | 10 | 0.422 | 4.41 | 10.5 | 92.4% | Effluxd as |
| | | | | | | expected |

Permeability Ranking: lower is <1 × 10⁻⁶ cm/s; higher is >1 × 10⁻⁶ cm/s.
An efflux ratio >2 indicates potential for the compound to be a substrate for Pgp or other active transporter.

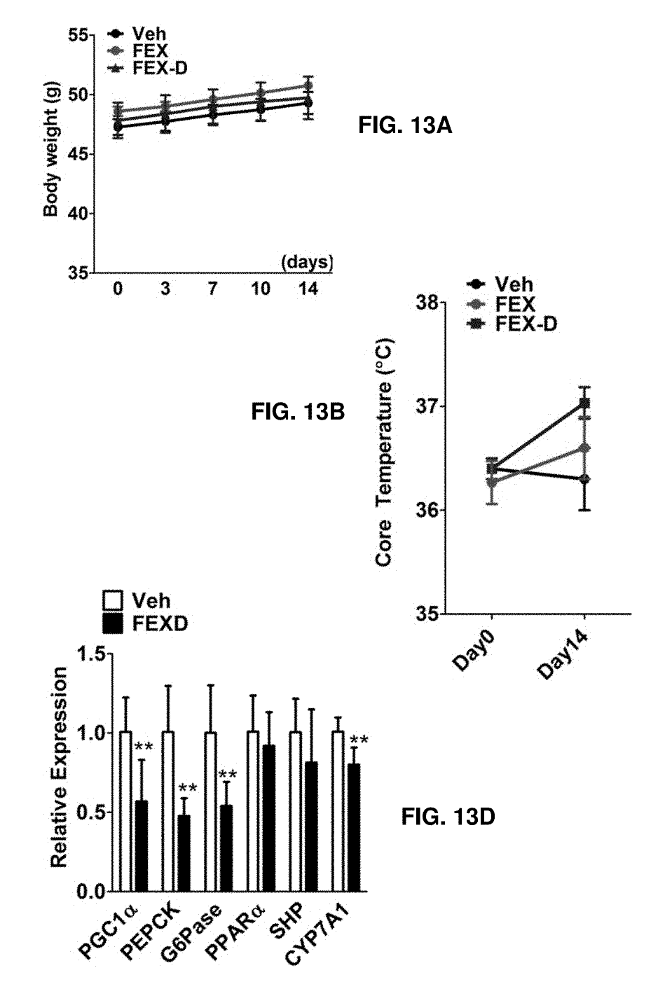

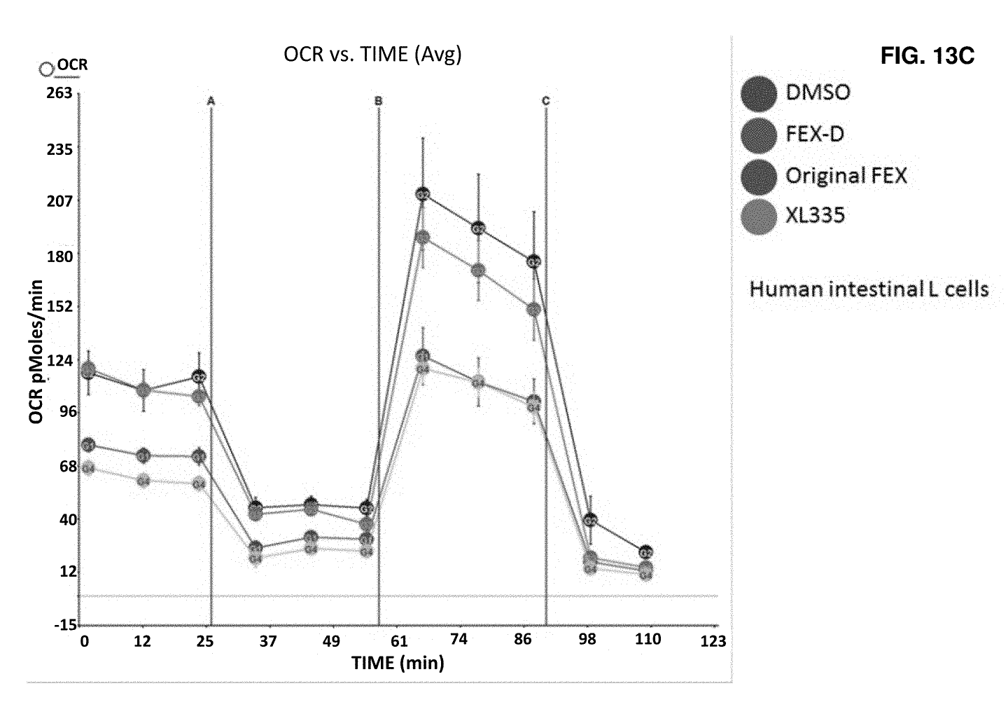

Orally-administered Fex-D significantly induced FXR target genes in intestine but not in the liver, consistent with it retaining intestinally-restricted activity (FIG. 13D). To compare the efficacies of Fex and Fex-D, ob/ob mice were treated with Fex (100 mg/kg/day) or Fex-D (at a reduced 50 mg/kg/day dose) for 2 weeks (FIG. 12B). Neither Fex nor Fex-D treatment led to any changes in body weight compared to vehicle treatment (FIG. 13A). While the reduced Fex treatment duration (2 weeks) had little effect, Fex-D treatment significantly reduced fasting glucose levels (FIG. 12C), and was accompanied by increased thermogenesis (FIG. 13B). Fex-D treated mice displayed dramatically improved glucose homeostasis, as seen in their glucose tolerance tests and in their reduced fasting insulin levels (FIGS. 12D and 12E). Furthermore, insulin secretion in response to a glucose challenge was significantly improved over Fex treatment (FIG. 12E), indicating that Fex-D is efficient in potentiating beneficial effects at a low dosage. Consistent with Fex-D treatment potentiated the metabolic capacity of enteroendocrinal L cells compared to Fex, Fex-D treatment enhanced GLP-1 secretion in ob/ob mice in response to glucose (FIG. 12F and FIG. 13C).

Next, primary islets were isolated from mice treated by Fex-D to compare gene expression changes. Strikingly, the two treatment regimes; Fex (100 mg/kg for 5 weeks) and Fex-D (50 mg/kg for 2 weeks), induced similar transcriptional changes in islets from ob/ob mice, including increased expression of the GLP-1 receptor and downstream genes (FIGS. 12G and 12H, Table 2). Interestingly, FGF15 injection upregulated reg family gene expression, but not GLP-1 receptor signaling (FIGS. 12G and 12H, Table 2) indicating that Fex and Fex-D enhance GLP-1 signaling in pancreatic β cells to improve cellular function in response to glucose in diabetic mice, which is independent with FGF15 signaling. Interestingly, the transcriptional changes induced in islets in vivo by intestional specific FXR activation (Fex and Fex-D) were not observed ex vivo (direct treatment of isolated islets with Fex, XL335, mFgf15 or hFgf19, FIG. 12G, Table 2). Furthermore, the expression of Txnip, a damage marker for pancreatic β cells, was dramatically reduced by Fex-D treatment, indicating that Fex-D treatment is efficient in restoring pancreatic β cell physiology in ob/ob diabetic mice (FIG. 12G) and reg families which is involved in beta cell regeneration (Table 3).

### Discussion

The results above demonstrate that intestinal FXR agonism is sufficient to improve glucose homeostasis and insulin resistance without significantly affecting body weight in a diabetic mouse model. Consistent with previous reports, Fex also enhances thermogenesis in BAT and reduces visceral adipose tissue inflammation in ob/ob mice. The improvements in glucose homeostasis appear to be mediated by an increase of GLP-1 secretion from enteroendocrinal L cells in response to glucose, leading to physiological improvements in pancreatic β cells and enhanced glucose-stimulated insulin secretion. These observations indicate that Fex potentiates the bioenergetics of enteroendocrinal L cells to increase GLP-1 secretion. Furthermore, Fex-D also exhibits similar physiological benefits, including enhanced GLP-1 secretion, indicating that intestinal FXR agonism potentiates bioenergetics to stimulate incretin secretion in enteroendocrinal L cells to ameliorate systemic glucose homeostasis.

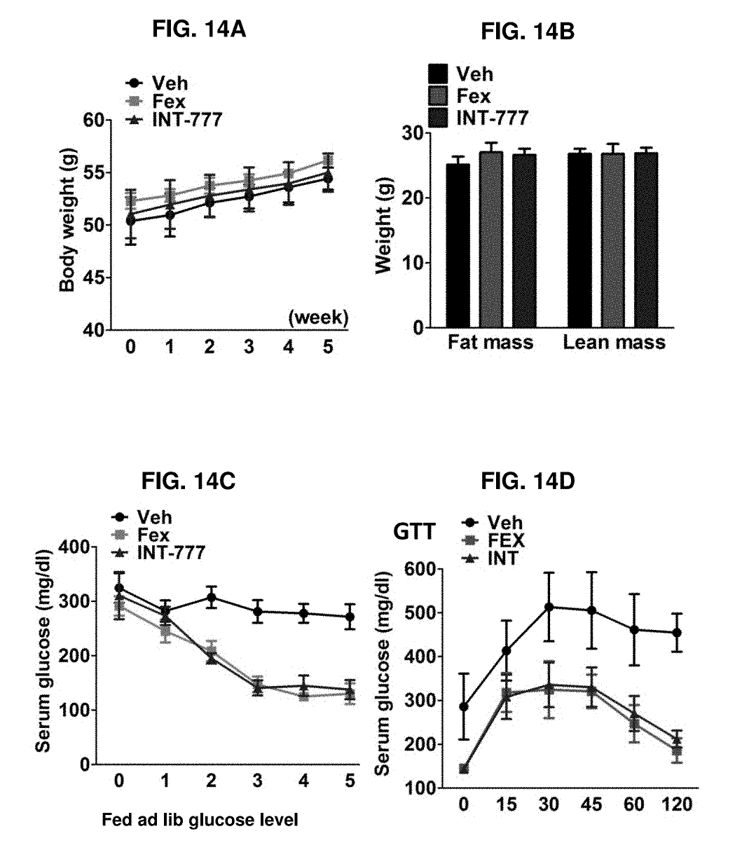

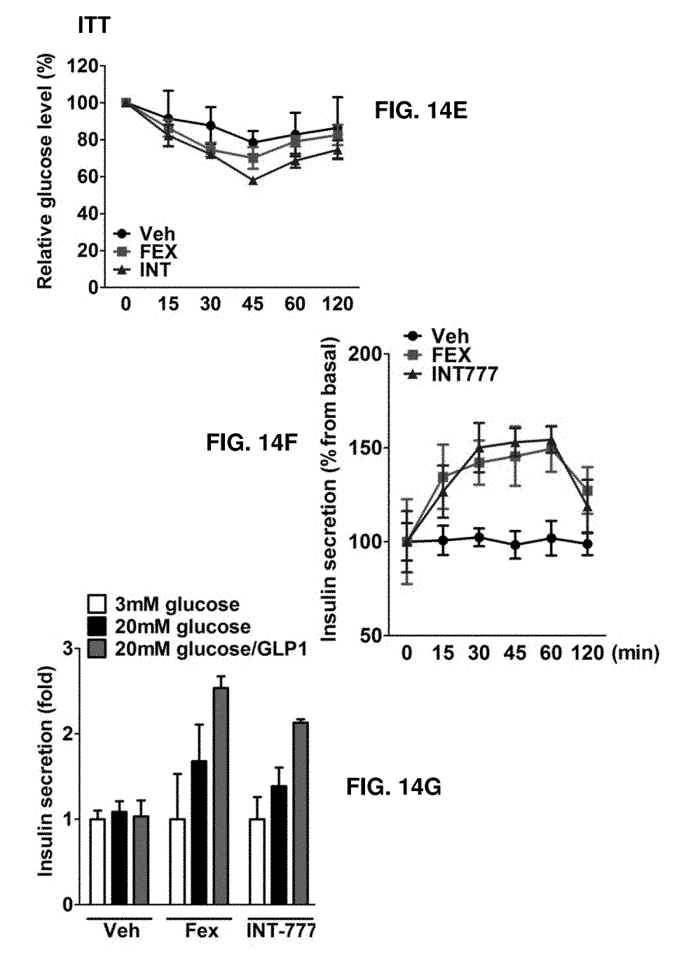

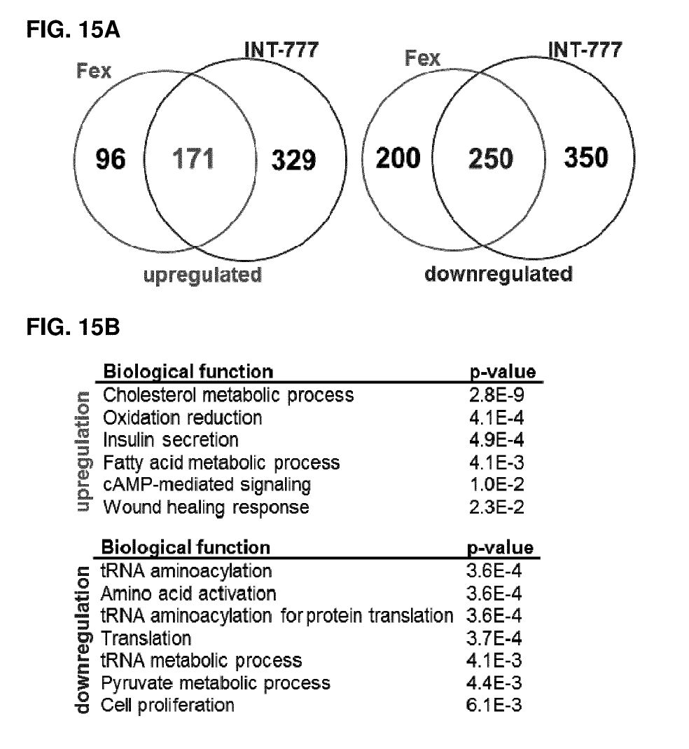

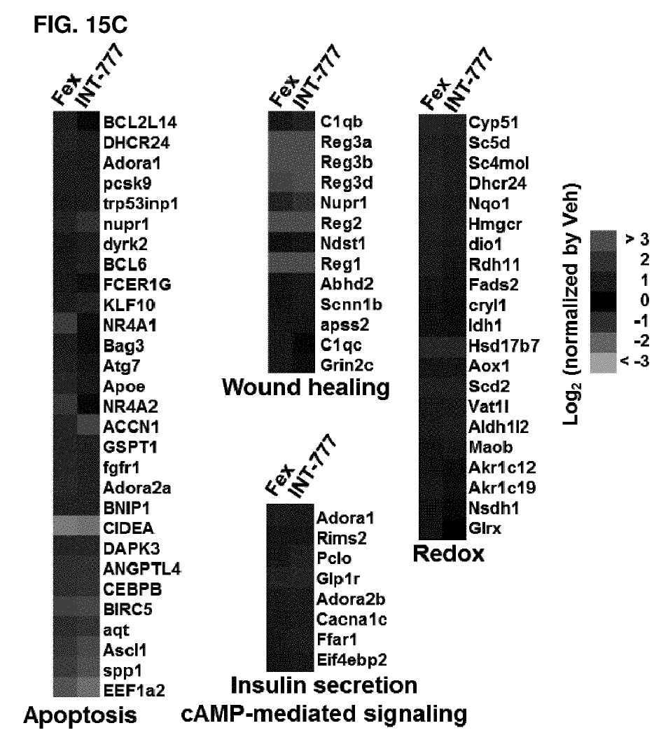

Fex treatment has been shown to change the relative composition of circulating bile acids. A reduction in hepatic CYP7A1 accompanied by an increase in CYP7B 1 expression shifts BA synthesis away from cholic acid towards chenodeoxyholic acid derivatives, most notably lithocholic acid, the most potent endogenous ligand for the G protein-coupled bile acid receptor TGR5. While it was previously reported that Fex does not activate TGR5, the pronounced changes in bile acid composition suggests that the TGR5 pathway may contribute to the observed physiological beneficial effects. The activation of TGR5 by a selective agonist INT-777 has been reported to stimulate the production and secretion of GLP-1 in enteroendocrinal L cells, leading to improvements in insulin secretion from pancreatic β cells. Notably, both treatments exhibited improvements in glucose-stimulated insulin secretion (FIG. 14F). Transcriptomic analysis of islets from Fex- or INT-777-treated ob/ob mice demonstrated a remarkably similar pattern of gene expression changes, including genes involved in cAMP-mediated signaling, oxidation/reduction and insulin secretion (FIGS. 15A-15C). Concomitantly, the expression of the GLP-1 receptor in islets was significantly increased by both Fex and INT-777 treatment (FIGS. 15A-15C), indicating that both Fex and INT-777 treatment enhance GLP-1 mediated signaling in pancreatic islets to stimulate insulin secretion in ob/ob mice.

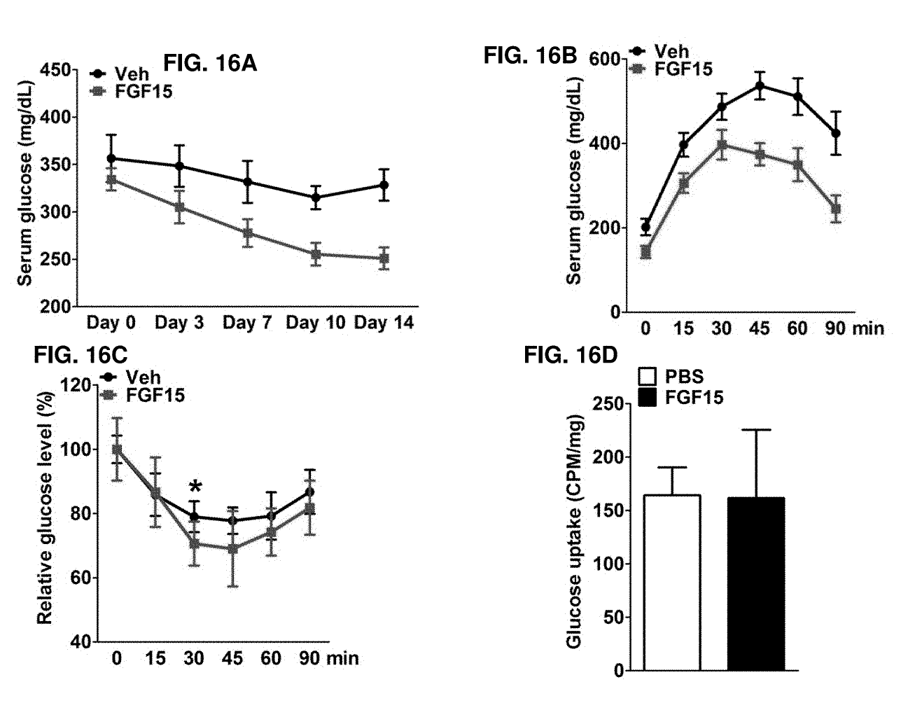

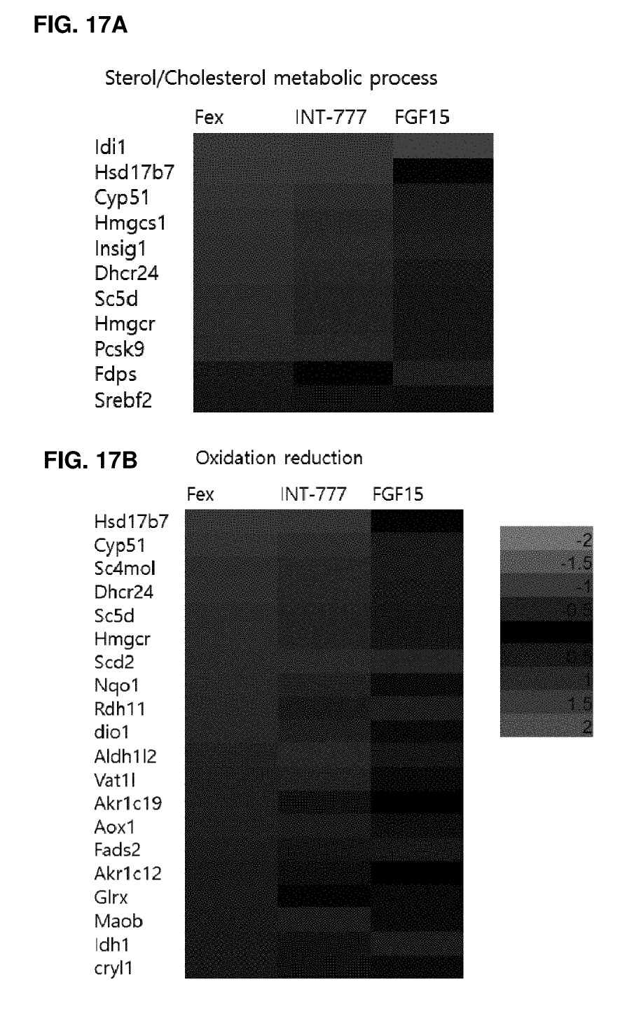

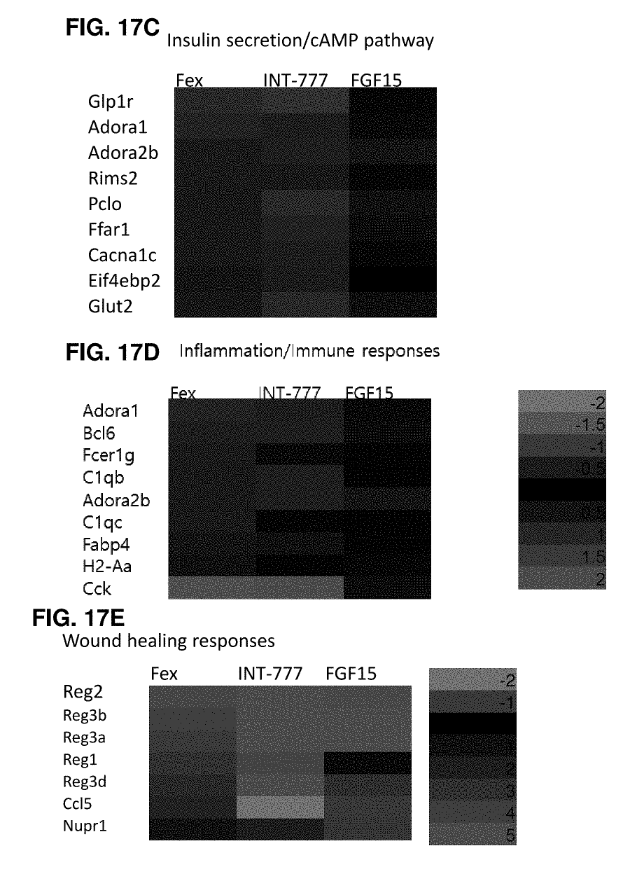

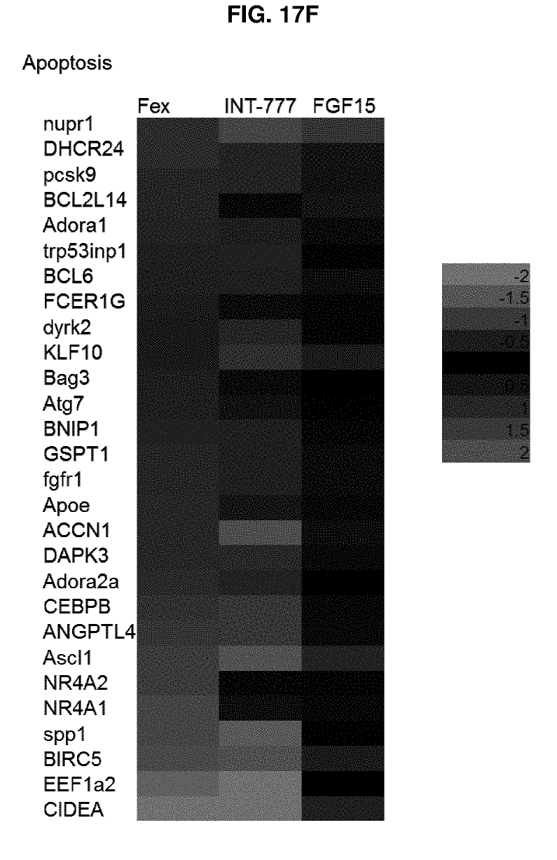

The systemic metabolic improvements induced in ob/ob mice by Fex treatment may be attributed in part, to the increase in FGF15 signaling. FGF15 has been shown to increase metabolic rate and improve glucose and lipid homeostasis without changes in food intake. However, the physiological impacts of FGF15 on pancreatic beta cells remain unclear. FGF15 treatment significantly reduced blood glucose levels as well as glucose sensitivity (as measured by glucose tolerance tests) without affecting glucose uptake in the skeletal muscle (FIGS. 16A-16D). Transcriptomic analyses of islets from vehicle- or FGF15-treated ob/ob mice establishes that FGF15 participates, at least partially, in the Fex-mediated improvements of pancreatic β cells in ob/ob mice (FIGS. 17A-17F).

These results uncover a new therapeutic avenue to manipulate GLP-1 signaling through intestinal activation of the nuclear receptor FXR. Though hypoglycemia has been rarely reported, an increased risk of pancreatitis or pancreatic cancer has been reported in patients treated with GLP-1 mimetic therapy. While GLP-1 mimetic therapy increases endogenous GLP-1 levels, Fex does not increase endogenous GLP-1 levels in a fasted-state. Instead, Fex potentiates GLP-1 secretion from enteroendocrinal L cells in response to glucose, and minimizes the chronic elevation of GLP-1 levels to reduce systemic activation of GLP-1 signaling. Taken together, these results indicate that intestinal FXR activation is a novel therapeutic strategy to treat LADA.

### Example 6

### Administration of Fexaramine and Guggulsterone

Glucagon-like peptide 1 (GLP-1) is a gut-derived peptide secreted by intestinal L cells after a meal, where it functions to potentiate glucose-stimulated insulin secretion, enhance β-cell growth and survival, and inhibit gastric emptying and food intake. The demonstrated glucose-lowering effects of GLP-1 have led to the approval of GLP-1 receptor agonists for the treatment of Type 2 diabetes. However, GLP-1 secretion is reduced in patients with type 2 diabetes, leading to interest in GLP-1 secretagogues as alternative therapies.

To examine the effects of FXR activity on the secretion of GLP-1, the metabolic changes induced in human L cells was measured after treatment with the FXR agonist, fexaramine and an FXR antagonist, guggulsterone. Treatment of L cells with fexaramine (1 μM for 24 hours) lead to an increase in the oxygen consumption rate (OCR), consistent with increased mitochondrial activity and consequently, an increased energetic state (FIG. 9A). The reverse effect was seen after treatment with the FXR antagonist, guggulsterone, with lower OCR after drug treatment. The ability of fexaramine to increase the energetic state of the L cells indicates that it can function as a GLP-1 secretagogue.

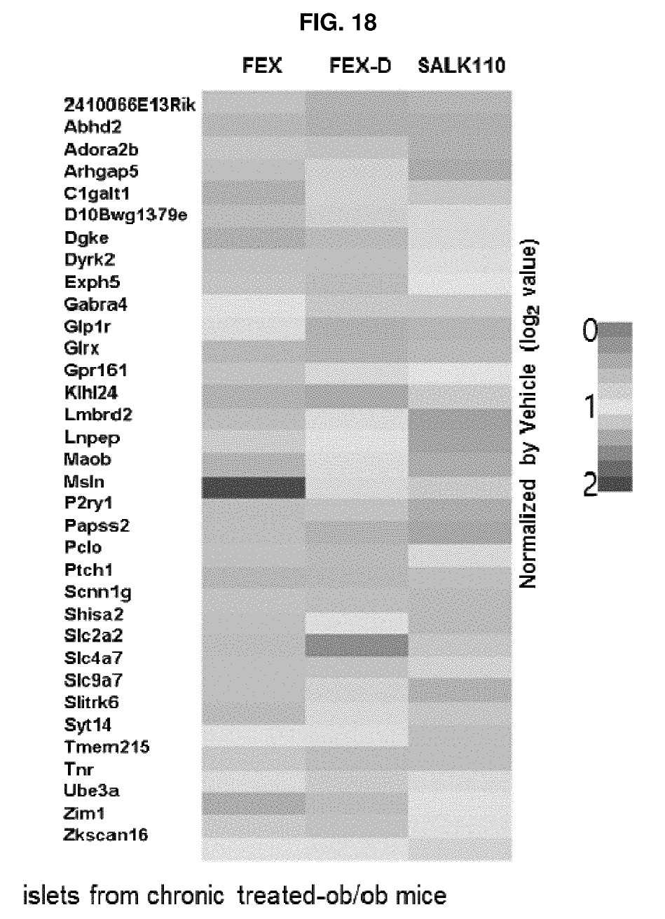

As shown in FIG. 18, fexaramine and the deuterated analogs induce a common set of genes in islets that are involved in intracellular signaling, insulin secretion, and regulation of exocytosis.

In view of the many possible embodiments to which the principles of the disclosure may be applied, it should be recognized that the illustrated embodiments are only examples of the disclosure and should not be taken as limiting the scope of the invention. Rather, the scope of the invention is defined by the following claims. We therefore claim as our invention all that comes within the scope and spirit of these claims.

We claim:

1. A method of treating or preventing latent autoimmune diabetes of adults (LADA) in a subject, comprising administering to a gastrointestinal tract of the subject a therapeutically effective amount of one or more farnesoid X receptor (FXR) agonists, thereby activating FXR receptors in the intestine of the subject and treating or preventing LADA in the subject.

2. The method of claim 1, wherein the one or more FXR agonists is minimally absorbed systemically.

3. The method of claim 1, wherein the method substantially restores pancreatic beta cell function in the subject, increases glucose-stimulated insulin secretion (GSIS), increases glucagon-like peptide 1 (GLP1) secretion in enteroendrocrine L cells of the subject, increases expression of glucagon-like peptide-1 receptor (GLP-1R) in pancreatic beta cells of the subject, or combinations thereof, relative to no administration of the one or more FXR agonists.

4. The method of claim 1, wherein the method improves glucose homeostasis in the subject.

5. The method of claim 1, further comprising detecting one or more markers of pancreatic beta cell damage in the subject.

6. The method of claim 5, wherein the one or more markers of pancreatic beta cell damage comprises thioredoxin-interacting protein (Txnip).

7. The method of claim 1, wherein the one or more FXR agonists has the following structure: 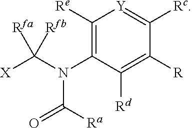
wherein,
R is selected from 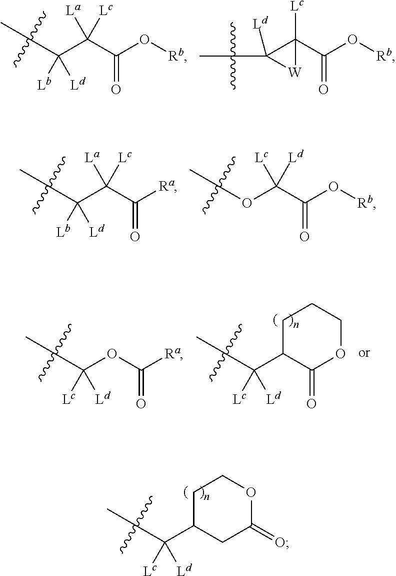
R^{a} is selected from aryl, heteroaryl, alkyl, alkenyl, cycloalkyl, heterocyclic, or polycyclic;
R^{b} is selected from hydrogen, alkyl, alkenyl, or cycloalkyl; Y is CR^{g}, N or N—O (N-oxide);
R^{c}, R^{d}, R^{e} and R^{g} are each independently selected from hydrogen, deuterium, halide, alkyl, alkenyl, alkoxy, alkylthio, amino, sulfonyl, aminosulfonyl, aminocarbonyl, acyl, hydroxyl or nitro;
R^{fa} and R^{fb} are each independently selected from hydrogen, deuterium, halide or alkyl;
L^{a} and L^{b} are each independently selected from hydrogen, deuterium, alkyl or cycloalkyl, or together form a pi-bond;
L^{c} and L^{d} are each independently selected from hydrogen, deuterium, alkyl or cycloalkyl;
W is selected from O or —(C(L^{c})(L^{d}))ₛ-;
s is 1, 2, 3, 4, 5 or 6;
n is 0 or 1; and
X is aryl, heterocyclic or heteroaryl.

8. The method of claim 1, wherein the one or more FXR agonists is deuterated.

9. The method of claim 8, wherein the one or more FXR agonists is

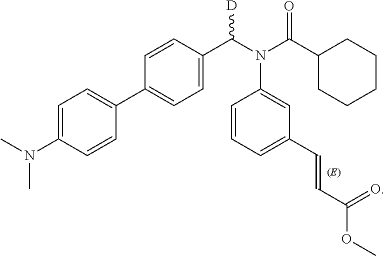

10. The method of claim 1, wherein the subject has a body mass index (BMI) of 25 of higher, is hyperglycemic, produces no insulin or is insulin resistant, has a decreased number of pancreatic beta cells, has persistent islet cell antibodies, has high frequency of thyroid and gastric autoimmunity, has DR3 and DR4 human leukocyte antigen haplotypes, shows progressive loss of beta cells, has adult disease onset, has low levels of C-peptide, or combinations thereof.

11. The method of claim 1, wherein the subject is a mammal.

12. The method of claim 11, wherein the mammal is a human.

13. The method claim 1, wherein the one or more FXR agonists is administered in combination with a therapeutically effective amount of one or more additional therapeutic compounds.

14. The method of claim 13, wherein the one or more additional therapeutic compounds is an insulin-sensitizing drug, an insulin secretagogue, an alpha-glucosidase inhibitor, an amylin agonist, a dipeptidyl-peptidase 4 (DPP-4) inhibitor, a glucagon-like peptide (GLP) agonist, meglitinide, sulfonylurea, a peroxisome proliferator-activated receptor (PPAR)-gamma agonist, nicotinamide ribonucleoside, analogs of nicotinamide ribonucleoside, or combinations thereof.

15. The method of claim 14, wherein the PPAR-gamma agonist is a thiazolidinedione (TZD), aleglitazar, farglitazar, muraglitazar, or tesaglitazar.

16. The method of claim 15, wherein the TZD is pioglitazone, rosiglitazone, rivoglitazone, or troglitazone.

17. The method of claim 1, wherein the one or more FXR agonists is administered daily, twice daily, every other day, bi-weekly, weekly, or monthly.

18. The method of claim 1, wherein the one or more FXR agonists is administered at a dose of at least 1 mg/kg.

19. The method of claim 1, wherein the method further comprises:
determining if the subject produces insulin;
determining a level of C-peptide in the subject;
determining a level of islet cell antibodies (ICA), glutamic acid decarboxylase autoantibodies (GADA), insulinoma-associated (IA-2) autoantibodies, and/or zinc transporter autoantibodies (ZnT8) in the subject; or
combinations thereof.
